(19) United States
(12) Patent Application Publication
(10) Pub. No.: US 2023/0183191 A1
(43) Pub. Date: Jun. 15, 2023

(54) NITROGEN-CONTAINING COMPOUND, ELECTRONIC ELEMENT, AND ELECTRONIC DEVICE

(71) Applicant: Shaanxi Lighte Optoelectronics Material Co., Ltd., Xi'an (CN)

(72) Inventors: Lingang LI, Xi'an (CN); Tiantian MA, Xi'an (CN); Lei YANG, Xi'an (CN)

(21) Appl. No.: 18/011,696

(22) PCT Filed: Aug. 6, 2021
(86) PCT No.: PCT/CN2021/111340

§ 371 Date: Dec. 20, 2022

(30) Foreign Application Priority Data
Aug. 28, 2020 (CN) 202010888603.5

(51) Int. Cl. C07D 251/24; H10K 85/60; C07D 409/04; C07D 403/04; C07D 213/57; C07D 401/04; C07D 401/10; C07F 7/08; H10K 85/40; C07D 409/10; C07D 405/10; C07D 239/26; C07D 405/04; H10K 50/16

(52) U.S. Cl. CPC C07D 251/24; H10K 85/654; H10K 85/652; C07D 409/04; H10K 85/6576; C07D 403/04; H10K 85/6572; C07D 213/57; C07D 401/04; C07D 401/10; C07F 7/0812; H10K 85/40; C07D 409/10; H10K 85/6574; C07D 405/10; C07D 239/26; C07D 405/04; H10K 50/16; C07B 2200/05

(57) ABSTRACT

The present disclosure belongs to the field of organic light-emitting materials, and provides a nitrogen-containing compound, an electronic element and an electronic device. The structure of the nitrogen-containing compound is shown as Formula 1, wherein X₁, X₂ and X₃ are each independently selected from C(H) or N, and at least one of the three is selected from N; and Ar₁ and Ar₂ are each independently selected from a substituted or unsubstituted aryl with 6 to 40 carbon atoms. The nitrogen-containing compound can improve performance of the electronic element.

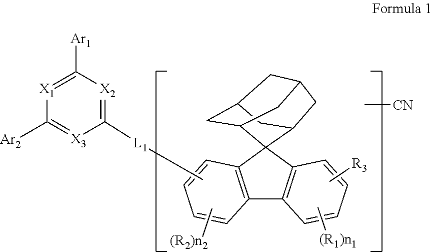

### CROSS REFERENCE TO RELATED APPLICATIONS

The present disclosure claims the priority of Chinese patent application with the application No. 202010888603.5 filed on Aug. 28, 2020, and the full content of the above Chinese patent application is incorporated herein as a part of this application.

### TECHNICAL FIELD

The present disclosure relates to the technical field of organic materials, in particular to a nitrogen-containing compound, an electronic element containing the nitrogen-containing compound, and an electronic device containing the electronic element.

### BACKGROUND

With development of the electronic technology and progress of material science, the application range of electronic elements for realizing electroluminescence or photoelectric conversion becomes more and more extensive. Such electronic elements usually include a cathode and an anode which are disposed oppositely, and a functional layer disposed between the cathode and the anode. The functional layer is composed of multiple organic or inorganic film layers, and generally includes an energy conversion layer, a hole transporting layer disposed between the energy conversion layer and the anode, and an electron transporting layer disposed between the energy conversion layer and the cathode.

For example, when the electronic element is an organic electroluminescent device, it generally includes an anode, a hole transporting layer, an organic light-emitting layer serving as an energy conversion layer, an electron transporting layer and a cathode which are stacked sequentially. When the voltage is applied to the cathode and the anode, an electric field is generated between the cathode and the anode. Under the action of the electric field, electrons at the cathode side move towards the organic light-emitting layer and holes at the anode side move towards the organic light-emitting layer, the electrons and the holes combine at the organic light-emitting layer to form excitons. The excitons are in an excited state to release energy outwards, so that the organic light-emitting layer emits light outwards.

Generally, electron transporting materials have poor stability and low transporting efficiency when used in organic electroluminescent devices, for the hole-electron transmission cannot truly get an equilibrium, resulting in reduced light-emitting efficiency and shortened service life of the device.

At present, although a large number of organic electroluminescent materials with excellent performance have been developed, such as the compound used in the organic electroluminescent device which disclosed in the patent document CN107431141A, it is still necessary to continue developing novel materials so that the performance of the electronic element would be further improved.

### SUMMARY

The objective of the present disclosure is to provide a nitrogen-containing compound, an electronic element and an electronic device, so as to improve performance of the electronic element and the electronic device.

To achieve above objective of the present disclosure, the present disclosure adopts the following technical solutions:

According to a first aspect of the present disclosure, a nitrogen-containing compound is provided, and the structure of the nitrogen-containing compound is shown as Formula 1:

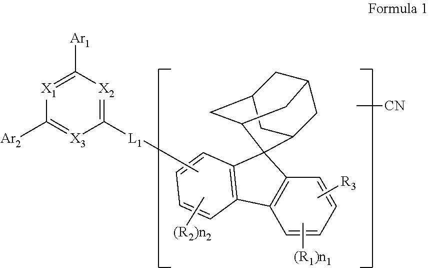

wherein X₁, X₂ and X₃ are the same or different, and each independently represent C(H) or N, and at least one of the three is N;

R₁ and R₂ are the same or different, and are each independently selected from deuterium, halogen group, a trialkylsilyl with 3 to 12 carbon atoms, an aryl with 6 to 20 carbon atoms, a heteroaryl with 3 to 18 carbon atoms, an alkyl with 1 to 10 carbon atoms, a cycloalkyl with 3 to 10 carbon atoms, a haloalkyl with 1 to 10 carbon atoms, an alkoxy with 1 to 10 carbon atoms, or an alkylthio with 1 to 10 carbon atoms; n₁ represents the number of R₁, n₁ is selected from 0, 1, 2 or 3, and when n₁ is greater than 1, any two R₁ are the same or different; and n₂ represents the number of R₂, n₂ is selected from 0, 1, 2 or 3, and when n₂ is greater than 1, any two R₂ are the same or different;

R₃ is selected from hydrogen, an aryl with 6 to 25 carbon atoms, or a heteroaryl with 5 to 20 carbon atoms;

L₁ is selected from single bond, a substituted or unsubstituted arylene with 6 to 30 carbon atoms, or a substituted or unsubstituted heteroarylene with 3 to 30 carbon atoms;

Ar₁ and Ar₂ are the same or different, and are each independently selected from a substituted or unsubstituted aryl with 6 to 40 carbon atoms, or a substituted or unsubstituted heteroaryl with 3 to 30 carbon atoms;

the substituents in L₁, Ar₁ and Ar₂ are the same or different, and are each independently selected from deuterium, halogen group, a trialkylsilyl with 3 to 12 carbon atoms, an aryl with 6 to 20 carbon atoms, a heteroaryl with 3 to 18 carbon atoms, an alkyl with 1 to 10 carbon atoms, a cycloalkyl with 3 to 10 carbon atoms, a haloalkyl with 1 to 10 carbon atoms, an alkoxy with 1 to 10 carbon atoms, or an alkylthio with 1 to 10 carbon atoms; and in L₁, Ar₁ and Ar₂, when there are two substituents on a same atom, optionally, the two substituents are connected to each other to form a 5- to 15-membered saturated or unsaturated ring together with the atom to which they are commonly connected.

According to a second aspect of the present disclosure, an electronic element is provided, including an anode and a cathode which are disposed oppositely, and a functional layer disposed between the anode and the cathode. The functional layer contains the above nitrogen-containing compound.

According to a third aspect of the present disclosure, an electronic device is provided, including the above electronic element.

The compound of the present disclosure takes an adamantane spirofluorene group as a parent nucleus which is connected with a nitrogen-containing heteroaromatic ring meanwhile. The parent nucleus increases an electron density of the nitrogen-containing heteroaromatic ring, so that the whole molecule has a strong polarity. Further, inventors found in the study that after introduction of cyano on the parent nucleus, the nitrogen-containing compound can further improve the performance of OLED devices. The reason may be that a strong electronegativity and a sigma-donor effect of the cyano increase bond strength of C—C on the parent nucleus, so that the whole molecule gains a more appropriate HOMO/LUMO energy level and molecular dipole moment. The compound of the present disclosure can further improve service life of the device under a condition of ensuring that the OLED device has a lower driving voltage and maintaining efficiency.

### BRIEF DESCRIPTION OF THE DRAWINGS

The above and other features and advantages of the present disclosure will become more apparent by describing examples in detail with reference to accompanying drawings.

FIG. 1 is a schematic structural diagram of an organic electroluminescent device according to one embodiment of the present disclosure.

FIG. 2 is a schematic structural diagram of a photoelectric conversion device according to one embodiment of the present disclosure.

FIG. 3 is a schematic structural diagram of an electronic device according to one embodiment of the present disclosure.

FIG. 4 is a schematic structural diagram of an electronic device according to another embodiment of the present disclosure.

### REFERENCE NUMERALS

100, anode; 200, cathode; 300, functional layer; 310, hole injection layer; 321, hole transporting layer; 322, electron blocking layer; 330, organic electroluminescent layer; 340, hole blocking layer; 350, electron transporting layer; 360, electron injection layer; 370, photoelectric conversion layer; 400, first electronic device; and 500, second electronic device.

### DETAILED DESCRIPTION

Examples will now be described more fully with reference to the accompanying drawings. However, examples may be implemented in various forms and should not be construed as being limited to the examples set forth herein; rather, these examples are provided, so that the present disclosure will be more thorough and complete, and the concept of the example is fully conveyed to the skilled in the art. The described features, structures, or characteristics may be combined in one or more examples in any suitable manner. In the following description, numerous specific details are provided so as to give a thorough understanding of the embodiments of the present disclosure.

In the drawings, thicknesses of regions and layers may be exaggerated for clarity. The same reference numerals in the accompanying drawings denote the same or similar structures, and thus their detailed descriptions will be omitted.

The described features, structures, or characteristics may be combined in one or more embodiments in any suitable manner. In the following description, numerous specific details are provided so as to give a thorough understanding of the embodiment of the present disclosure. However, the skilled in the art will appreciate that the technical solutions of the present disclosure may be practiced without one or more of the specific details, or other methods, components, materials and the like may be employed. In other cases, well-known structures, materials or operations are not shown or described in detail to avoid obscuring the main technical idea of the present disclosure.

In a first aspect, the present disclosure provides a nitrogen-containing compound with a structure being shown in a Formula 1:

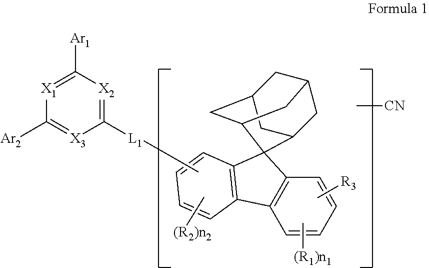

wherein X₁, X₂ and X₃ are the same or different, and each independently represent C(H) or N, and at least one of the three is N;

R₁ and R₂ are the same or different, and are each independently selected from deuterium, halogen group, a trialkylsilyl with 3 to 12 carbon atoms, an aryl with 6 to 20 carbon atoms, a heteroaryl with 3 to 18 carbon atoms, an alkyl with 1 to 10 carbon atoms, a cycloalkyl with 3 to 10 carbon atoms, a haloalkyl with 1 to 10 carbon atoms, an alkoxy with 1 to 10 carbon atoms, or an alkylthio with 1 to 10 carbon atoms; n₁ represents the number of R₁, n₁ is selected from 0, 1, 2 or 3, and when n₁ is greater than 1, any two R₁ are the same or different; and n₂ represents the number of R₂, n₂ is selected from 0, 1, 2 or 3, and when n₂ is greater than 1, any two R₂ are the same or different;

R₃ is selected from hydrogen, an aryl with 6 to 25 carbon atoms, or a heteroaryl with 5 to 20 carbon atoms;

L₁ is selected from single bond, a substituted or unsubstituted arylene with 6 to 30 carbon atoms, or a substituted or unsubstituted heteroarylene with 3 to 30 carbon atoms;

Ar₁ and Ar₂ are the same or different, and are each independently selected from a substituted or unsubstituted aryl with 6 to 40 carbon atoms, or a substituted or unsubstituted heteroaryl with 3 to 30 carbon atoms;

substituents in L₁, Ar₁ and Ar₂ are the same or different, and are each independently selected from deuterium, halogen group, a trialkylsilyl with 3 to 12 carbon atoms, an aryl with 6 to 20 carbon atoms, a heteroaryl with 3 to 18 carbon atoms, an alkyl with 1 to 10 carbon atoms, a cycloalkyl with 3 to 10 carbon atoms, a haloalkyl with 1 to 10 carbon atoms, an alkoxy with 1 to 10 carbon atoms, or an alkylthio with 1 to 10 carbon atoms; and in L₁, Ar₁ and Ara, when there are two substituents on a same atom, optionally, the two substituents are connected to each other to form a 5- to 15-membered saturated or unsaturated ring together with the atom to which they are commonly connected.

In the present disclosure, in the Formula 1,

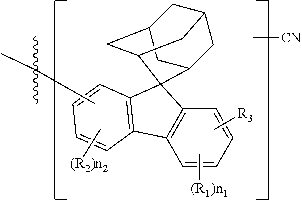

represents that cyano (—CN) may be connected to a structure

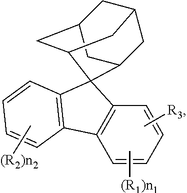

for example, it may be connected to a fused benzene ring, and may also be connected to R₃ and R₁, R₂ (if any). It should be understood that when the cyano is connected to a benzene ring corresponding to R₁, n₁ is selected from 0, 1 or 2, and when the cyano is connected to a benzene ring corresponding to R₂, n₂ is selected from 0, 1 or 2. In addition, when the cyano is connected to R₃ and R₃ is H, it should be understood that the cyano is directly connected to a benzene ring corresponding to R₃.

Optionally, the structure of the nitrogen-containing compound is selected from at least one of Formulae 1-1 to 1-3:

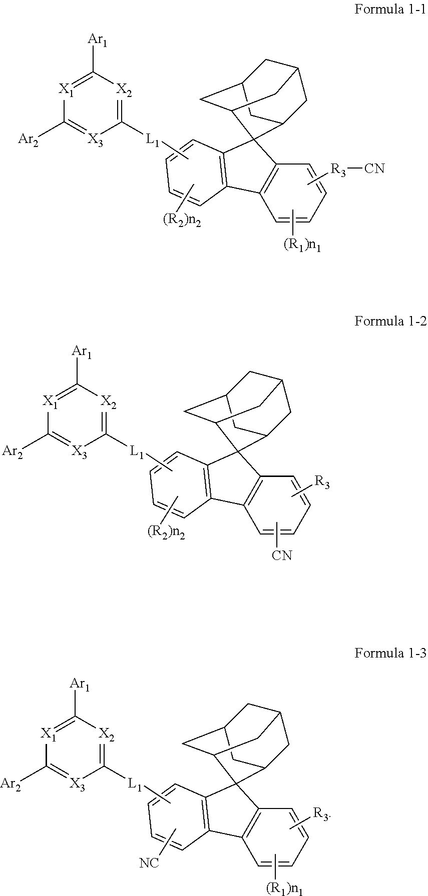

In the present disclosure, one of X₁, X₂ and X₃ is an N atom, or two of X₁, X₂ and X₃ are the N atoms, or all of X₁, X₂ and X₃ are the N atoms.

The employed description manners “each . . . is independently”, “ . . . is each independently” and “ . . . is independently selected from” employed in the present disclosure can be interchanged, and should be understood in a broad sense, which means that in different groups, specific options expressed between the same symbols do not influence each other, or means that in the same group, specific options expressed between the same symbols do not influence each other. For example, the meaning of

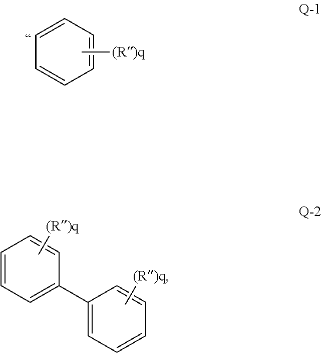

wherein each q is independently 0, 1, 2 or 3, and each R″ is independently selected from hydrogen, deuterium, fluorine and chlorine” is as follows: a formula Q-1 represents that q substituents R″ exist on a benzene ring, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the present disclosure, the terms “optional” and “optionally” mean that the subsequently described event or environment may, but does not have to occur, and the description includes an occasion where the event or environment occurs or does not occur. For example, “optionally, two adjacent substituents XX form a ring” means that the two substituents may form a ring but does not have to form a ring, including: a scenario where the two adjacent substituents form the ring and a scenario where the two adjacent substituents do not form the ring.

In the present disclosure, the number of carbon atoms of L₁, Ar₁ and Ar₂ refers to the number of all the carbon atoms. For example, if L₁ is selected from a substituted arylene with 7 carbon atoms, then the total number of all the carbon atoms in the arylene and the substituents is 7.

In the present disclosure, the aryl refers to an optional functional group or substituent derived from an aromatic carbocyclic ring. The aryl may be a monocyclic aryl (e.g., phenyl) or a polycyclic aryl. In other words, aryl can be a monocyclic aryl, a fused-ring aryl, two or more monocyclic aryls conjugatedly connected through carbon-carbon bonds, a monocyclic aryl and fused-ring aryl conjugatedly connected through carbon-carbon bonds, or two or more fused-ring aryls conjugatedly connected through carbon-carbon bonds. That is, unless otherwise noted, two or more aromatic groups conjugatedly connected through carbon-carbon bonds may also be regarded as aryl of the present disclosure. The fused-ring aryl may include, for example, a bicyclic fused aryl (e.g., naphthyl), a tricyclic fused aryl (e.g., phenanthryl, fluorenyl, anthryl), and the like. The aryl does not contain heteroatoms such as B, N, O, S, P, Se, Si and the like. It should be noted that biphenyl, terphenyl, 9,9-dimethylfluorenyl, 9,9-diphenylfluorenyl, spirobifluorenyl, and the like are all regarded as aryl in the present disclosure. Examples of aryl further include, but are not limited to, phenyl, naphthyl, fluorenyl, anthryl, phenanthryl, biphenyl, terphenyl, benzo[9,10]phenanthryl, pyrenyl, benzofluoranthenyl, chrysenyl, and the like. In the present disclosure, the involved arylene refers to a divalent group formed by further loss of one hydrogen atom from the aryl.

In the present disclosure, the substituted aryl may be one or two or more hydrogen atoms in the aryl replaced by groups such as deuterium atom, halogen group, aryl, heteroaryl, trialkylsilyl, alkyl, cycloalkyl, alkoxy, alkylthio and the like. Specific examples of aryl substituted by heteroaryl include, but are not limited to, phenyl substituted by dibenzofuranyl, phenyl substituted by dibenzothienyl, phenyl substituted by pyridyl, phenyl substituted by carbazolyl, phenyl substituted by N-phenylcarbazolyl, etc. It should be understood that the number of carbon atoms in the substituted aryl refers to the total number of carbon atoms in the aryl and the substituent on the aryl. For example, the substituted aryl with 18 carbon atoms refers to the total number of carbon atoms in the aryl and the substituted being 18.

In the present disclosure, the heteroaryl refers to a monovalent aromatic ring containing at least one (e.g., 1, 2, 3, 4, 5 or 6) heteroatom in a ring or its derivative, and the heteroatom may be at least one of B, O, N, P, Si, Se and S. The heteroaryl may be a monocyclic heteroaryl or a polycyclic heteroaryl. In other words, the heteroaryl may be a single aromatic ring system or a plurality of aromatic ring systems conjugatedly connected through carbon-carbon bonds, and any aromatic ring system is an aromatic monocyclic ring or an aromatic fused ring. Illustratively, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridinopyrimidyl, pyridinopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuryl, phenanthrolinyl, isoxazolyl, thiadiazolyl, phenothiazinyl, silafluorenyl, dibenzofuryl, N-phenylcarbazolyl, N-pyridylcarbazolyl, N-methylcarbazolyl, and the like, but is not limited to this. Where thienyl, furyl, phenanthrolinyl and the like are heteroaryl of the single aromatic ring system, and N-phenylcarbazolyl and N-pyridylcarbazolyl are heteroaryl of a plurality of ring systems conjugatedly connected through carbon-carbon bonds. In the present disclosure, the involved heteroarylene refers to a divalent group formed by further loss of one hydrogen atom from the heteroaryl.

In the present disclosure, the substituted heteroaryl may be one or two or more hydrogen atoms in the heteroaryl are replaced by groups such as deuterium atom, halogen group, aryl, heteroaryl, trialkylsilyl, alkyl, cycloalkyl, alkoxy, and alkylthio and the like. Specific examples of heteroaryl substituted by aryl include, but are not limited to, dibenzofuranyl substituted by phenyl, dibenzothienyl substituted by phenyl, pyridyl substituted by phenyl, etc. It should be understood that the number of carbon atoms in the substituted heteroaryl refers to the total number of carbon atoms in the heteroaryl and a substituent on the heteroaryl.

In the present disclosure, an involved nonlocalized connecting bond refers to a single bond “ [symbol] ” extending from a ring system, which means that one end of the connecting bond may be connected with any position in the ring system through which the bond penetrates, and the other end of the connecting bond is connected with the remaining part of a compound molecular structure.

For example, as shown in the following formula (f), naphthyl represented by the formula (f) is connected with other positions of a molecule through two nonlocalized connecting bonds penetrating through a dicyclic ring, and its represented meaning includes any one possible connecting mode as shown in formulae (f-1) to (f-10).

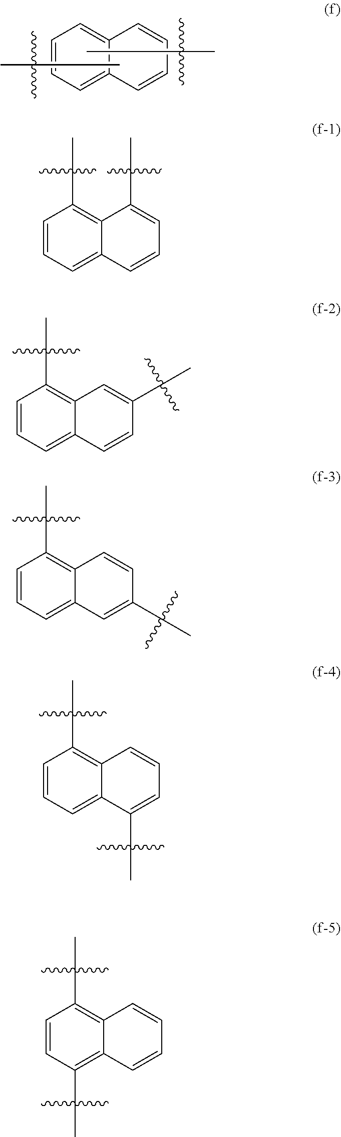

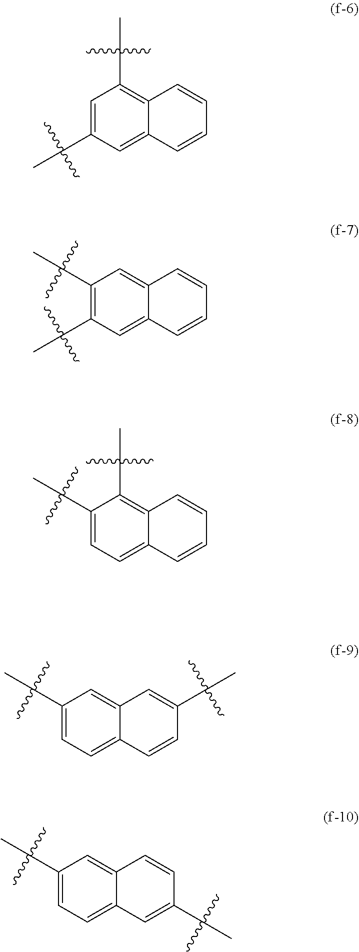 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

For another embodiment, as shown in the following formula (X′), phenanthryl represented by the formula (X′) is connected with other positions of the molecule through one nonlocalized connecting bond extending from the middle of a benzene ring on one side, and its represented meaning includes any one possible connecting mode as shown in formulae (X′-1) to (X′-4).

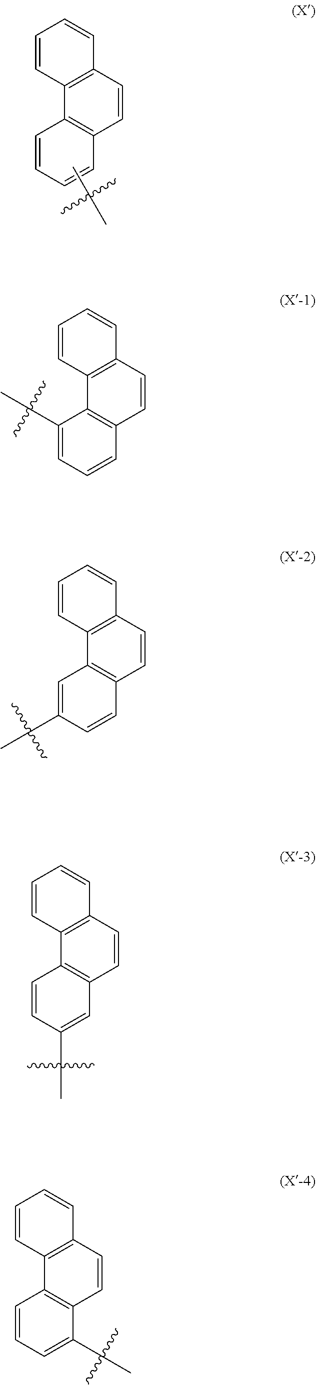

An nonlocalized substituent in the present disclosure, refers to a substituent connected through a single bond extending from the center of the ring system, which means that the substituent may be connected at any possible position in the ring system. For example, as shown in the following formula (Y), a substituent R′ represented by the formula (Y) is connected with a quinoline ring through one nonlocalized connecting bond, and its represented meaning includes any one possible connecting mode as shown in formulae (Y-1) to (Y-7).

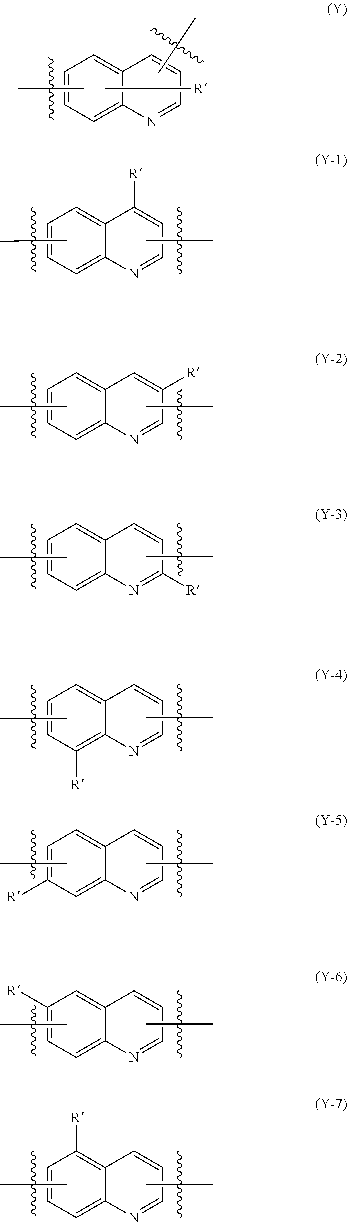

In the present disclosure, the alkyl with 1 to 10 carbon atoms may include straight-chain alkyl with 1 to 10 carbon atoms and branched-chain alkyl with 3 to 10 carbon atoms, and the number of carbon atoms may be, for example, 1, 2, 3, 4, 5, 6, 7, 8, 9 or 10. Specific examples of the alkyl include, but are not limited to, methyl, ethyl, n-propyl, isopropyl, n-butyl, isobutyl, tert-butyl, n-pentyl, iso-pentyl, neopentyl, cyclopentyl, n-hexyl, heptyl, n-octyl, 2-ethylhexyl, nonyl, decyl, 3,7-dimethyloctyl and the like.

In the present disclosure, the halogen group may include fluorine, iodine, bromine, chlorine and the like.

In the present disclosure, the number of carbon atoms of the aryl serving as a substituent may be 6 to 20. The number of carbon atoms of the aryl with 6 to 20 carbon atoms is, for example, 6, 10 (naphthyl), 12, 14, 15, 16, 18, or 20 and the like. Specific examples of the aryl with 6 to 20 carbon atoms include, but are not limited to, phenyl, naphthyl, biphenyl, 9,9-dimethylfluorenyl and the like.

In the present disclosure, the number of carbon atoms of the heteroaryl with 3 to 20 carbon atoms is, for example, 5, 8, 12, 15, 18, or 20 and the like. The number of carbon atoms of the heteroaryl serving as a substituent may be 3 to 18, and the number of carbon atoms is, for example, 5, 8, 12, 15, 18 and the like. Specific examples of the heteroaryl serving as the substituent include, but are not limited to, pyridyl, quinolyl, dibenzofuranyl, dibenzothienyl, carbazolyl and the like.

In the present disclosure, specific examples of the trialkylsilyl with 3 to 12 carbon atoms include, but are not limited to, trimethylsilyl, triethylsilyl and the like.

In the present disclosure, specific examples of the cycloalkyl with 3 to 10 carbon atoms include, but are not limited to, cyclopentyl, cyclohexyl, adamantyl and the like.

Optionally, L₁ is selected from single bond, a substituted or unsubstituted arylene with 6 to 25 carbon atoms, or a substituted or unsubstituted heteroarylene with 5 to 20 carbon atoms. For example, L₁ is selected from single bond, a substituted or unsubstituted arylene with 6, 7, 8, 9, 10, 11, 12, 13, 14, 15, 16, 17, 18, 19, 20, 21, 22, 23, 24 or 25 carbon atoms, or a substituted or unsubstituted heteroarylene with 5, 7, 8, 9, 10, 11, 12, 13, 14, 15, 16, 17, 18, 19 or 20 carbon atoms.

Further optionally, L₁ is selected from single bond, a substituted or unsubstituted arylene with 6 to 15 carbon atoms, or a substituted or unsubstituted heteroarylene with 5 to 18 carbon atoms.

Optionally, a substituent of L₁ is selected from: deuterium, fluorine, a trialkylsilyl with 3 to 7 carbon atoms, an alkyl with 1 to 4 carbon atoms, a haloalkyl with 1 to 4 carbon atoms, an alkoxy with 1 to 4 carbon atoms, an alkylthio with 1 to 4 carbon atoms, phenyl, or a cycloalkyl with 5 to 10 carbon atoms. Specific examples of the substituent of L₁ include, but are not limited to, deuterium, fluorine, methyl, ethyl, n-propyl, isopropyl, tert-butyl, phenyl, naphthyl, pyridyl, trifluoromethyl, methoxyl, trimethylsilyl, cyclohexyl, cyclopentyl and the like.

In some embodiments, L₁ is selected from single bond, and the group consisting of groups shown in formulae j-1 to j-12:

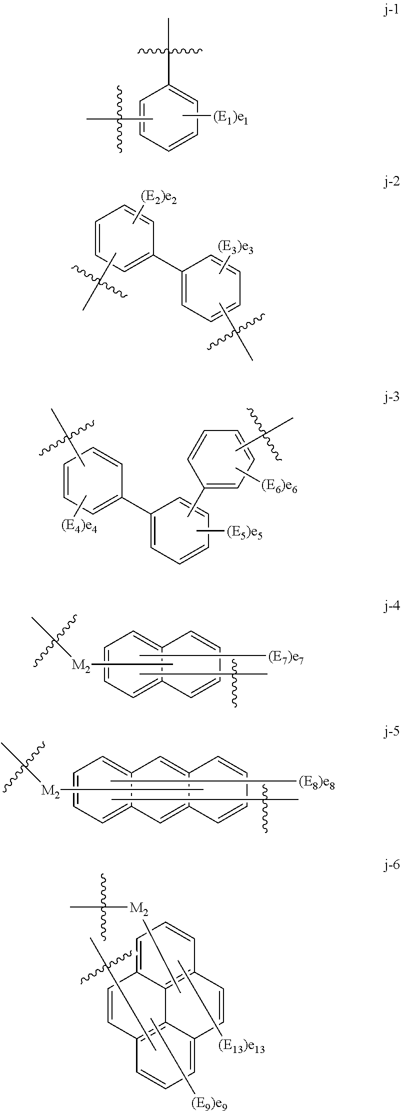

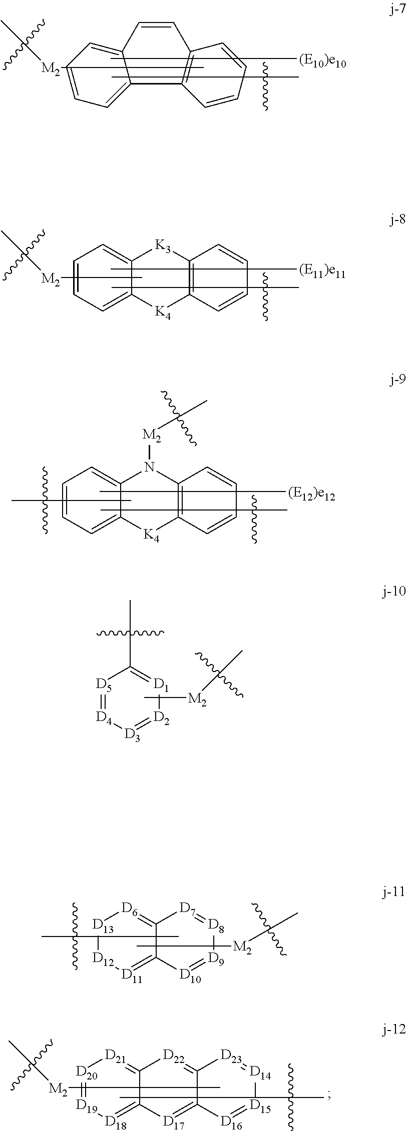

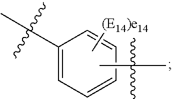

wherein M₂ is selected from single bond or

D₁ to D₅ are each independently selected from N or C(F₅), and at least one of D₁ to D₅ is selected from N; and when two or more of D₁ to D₅ are selected from C(F₅), any two F₅ are the same or different;

D₆ to D₁₃ are each independently selected from N or C(F₆), and at least one of D₆ to D₁₃ is selected from N; and when two or more of D₆ to D₁₃ are selected from C(F₆), any two F₆ are the same or different;

D₁₄ to D₂₃ are each independently selected from N or C(F₇), and at least one of D₁₄ to D₂₃ is selected from N; and when two or more of D₁₄ to D₂₃ are selected from C(F₇), any two F₇ are the same or different;

E₁ to E₁₄ and F₅ to F₇ are each independently selected from: hydrogen, deuterium, fluorine, chlorine, bromine, a heteroaryl with 3 to 18 carbon atoms, an aryl with 6 to 18 carbon atoms, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, or an alkylthio with 1 to 10 carbon atoms;

e₁ to e₁₄ are represented by eᵣ, E₁ to E₁₄ are represented by Eᵣ, r is a variable and represents any integer from 1 to 14, and eᵣ represents the number of a substituent Eᵣ; when r is selected from 1, 2, 3, 4, 5, 6, 9, 13 or 14, eᵣ is selected from 1, 2, 3 or 4; when r is selected from 7 or 11, eᵣ is selected from 1, 2, 3, 4, 5 or 6; when r is 12, eᵣ is selected from 1, 2, 3, 4, 5, 6 or 7; when r is selected from 8 or 10, eᵣ is selected from 1, 2, 3, 4, 5, 6, 7 or 8; and when eᵣ is greater than 1, any two Eᵣ are the same or different;

K₃ is selected from O, S, Se, N(E₂₀), C(E₂₁E₂₂), or Si(E₂₁E₂₂), wherein E₂₀, E₂₁, and E₂₂ are each independently selected from: an aryl with 6 to 18 carbon atoms, a heteroaryl with 3 to 18 carbon atoms, an alkyl with 1 to 10 carbon atoms, or a cycloalkyl with 3 to 10 carbon atoms, or the E₂₁ and the E₂₂ are connected to each other to form a 5- to 15-membered saturated or unsaturated ring together with the atom to which they are commonly connected; and

K₄ is selected from single bond, O, S, Se, N(E₂₃), C(E₂₄E₂₅), or Si(E₂₄E₂₅), wherein E₂₃, E₂₄, and E₂₅ are each independently selected from: an aryl with 6 to 18 carbon atoms, a heteroaryl with 3 to 18 carbon atoms, an alkyl with 1 to 10 carbon atoms, or a cycloalkyl with 3 to 10 carbon atoms, or the E₂₄ and the E₂₅ are connected to each other to form a 5- to 15-membered saturated or unsaturated ring together with the atom to which they are commonly connected.

In the present disclosure, in the above two groups of E₂₁ and E₂₂ and the above E₂₄ and E₂₅, the ring formed by the interconnection of the two groups in each group is the 5- to 15-membered saturated or unsaturated ring. For example, in the formula j-8, when K₄ and M₂ are both single bonds, E₁₁ is hydrogen, e₁₁=6, K₃ is C(E₂₁E₂₂), and E₂₄ and E₂₅ are connected to each other to form a 5-membered saturated ring together with the atoms to which they are commonly connected, the formula j-8 may be

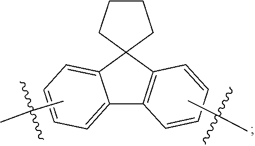

similarly, the formula j-8 may also be

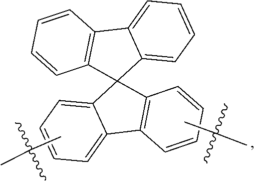

that is, E₂₁ and E₂₂ are connected to each other to form a 13-membered unsaturated ring together with the atom to which they are commonly connected. Ring formation definition of H₂₃, H₂₄, H₂₆ and H₂₇ is similar to ring formation definition of E₂₁, E₂₂, E₂₄ and E₂₅in the following description, which is not repeated here.

In the present disclosure, D₁ to D₂₃ may be represented by Dₓ₁, where x₁ represents a variable and is an integer from 1 to 23. For example, when x₁ is 5, Dₓ₁ is D₅. F₅ to F₇ may be represented by F_{y1}, wherein y₁ represents a variable and is an integer from 5 to 7. For example, when y₁ is 7, F_{y1} is above F₇. It should be understood that when Dₓ₁ is C(F_{y1}) and F_{y1} is hydrogen, Dₓ₁ in the corresponding formula is presented in a form of a C atom. Taking the formula j-10 as an example, when D₁ is N and D₂ to D₅ are all CH (F₅ is H), the formula j-10 is represented as

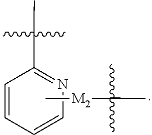

Further, when M₂ is single bond, the formula j-10 is represented as

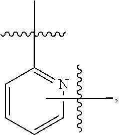

and a more specific structure may be, for example:

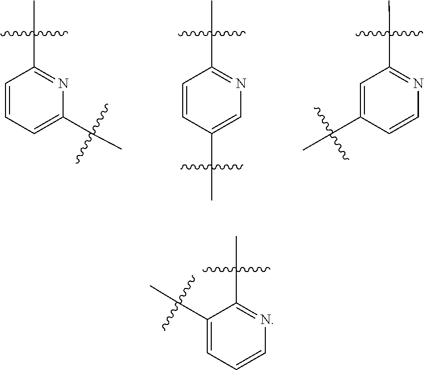 [SMILES: *c1cccc(*)n1 ; *c1ccc(*)nc1 ; *c1ccnc(*)c1 ; *c1cccnc1*]

Explanations of G₁ to G₂₃ below are similar to those of D₁ to D₂₃, which are not repeated here.

Optionally, L₁ is selected from single bond, a substituted or unsubstituted phenylene, a substituted or unsubstituted naphthylene, a substituted or unsubstituted biphenylene, a substituted or unsubstituted terphenylene, a substituted or unsubstituted 9,9-dimethylfluorenylene, a substituted or unsubstituted anthrylene, a substituted or unsubstituted phenanthrylene, a substituted or unsubstituted carbazolylene, a substituted or unsubstituted dibenzofuranylene, a substituted or unsubstituted dibenzothienylene, a substituted or unsubstituted pyridylene, or a ylene/ylidene group formed by two or three of these groups are connecting through single bond. Further optionally, the substituents in L₁ are each independently selected from: deuterium, fluorine, an alkyl with 1 to 4 carbon atoms, a trialkylsilyl with 3 to 7 carbon atoms, a cycloalkyl with 5 to 10 carbon atoms, phenyl, naphthyl, pyridyl and the like.

Optionally, L₁ is selected from single bond, or a substituted or unsubstituted group T₁, wherein the unsubstituted group T₁ is selected from the group consisting of the following groups:

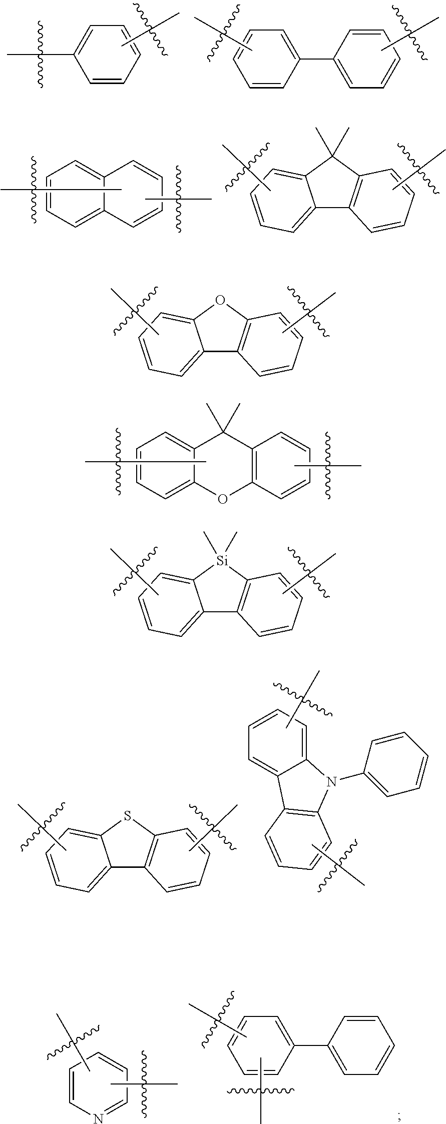

and the substituted group T₁ has one or two or more substituents, and the substituents are each independently selected from deuterium, fluorine, a trialkylsilyl with 3 to 7 carbon atoms, an alkyl with 1 to 4 carbon atoms, a fluoroalkyl with 1 to 4 carbon atoms, a cycloalkyl with 5 to 10 carbon atoms, an alkoxy with 1 to 4 carbon atoms, or an alkylthio with 1 to 4 carbon atoms; and when the number of the substituents is greater than 1, the substituents are the same or different.

According to one more specific embodiment, L₁ is selected from single bond or the group consisting of the following groups:

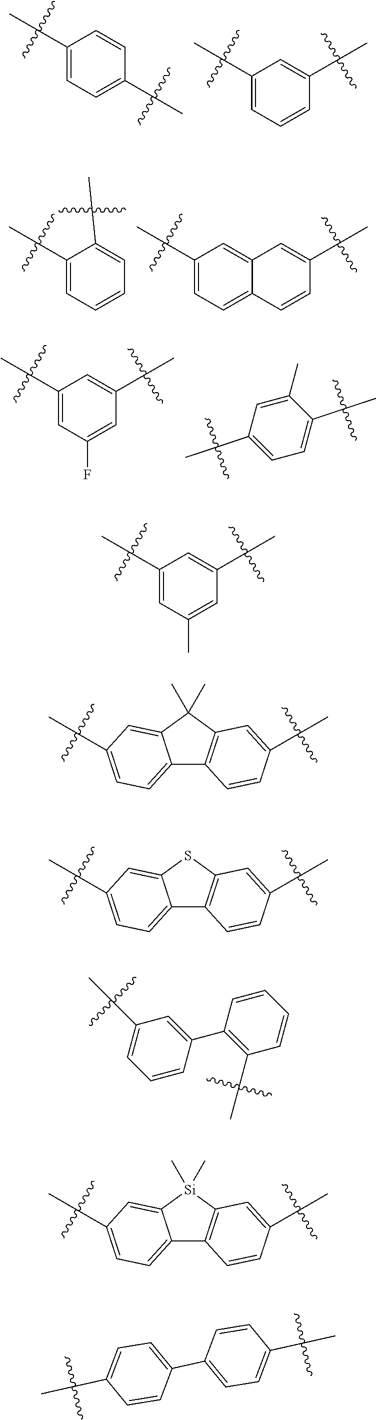

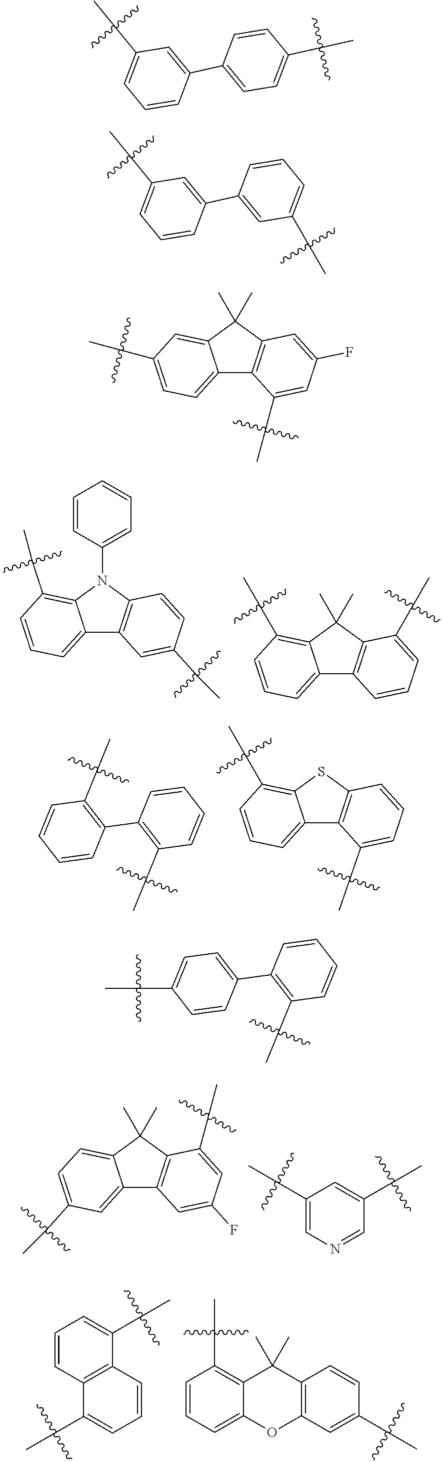

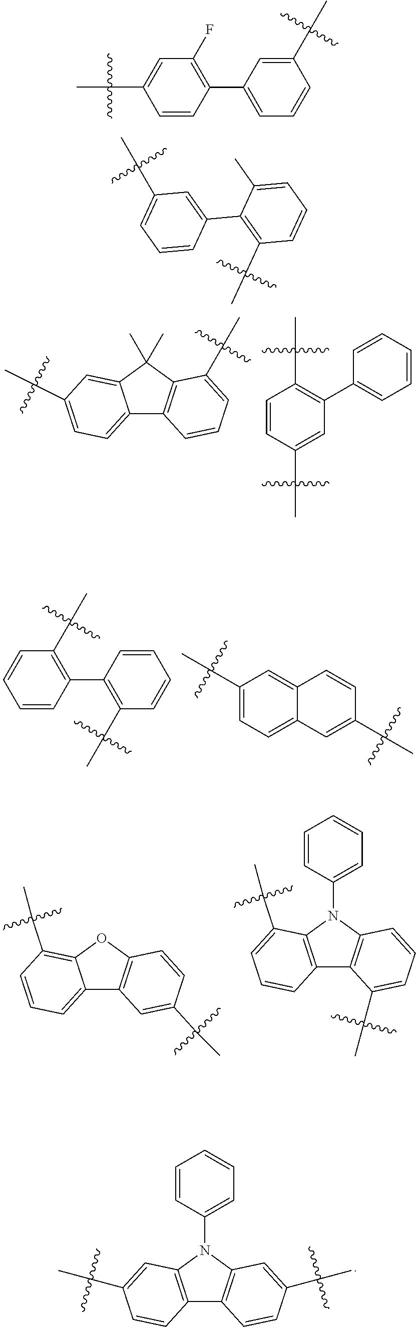

Optionally, Ar₁ and Ar₂ are the same or different, and are each independently selected from a substituted or unsubstituted aryl with 6 to 25 carbon atoms, or a substituted or unsubstituted heteroaryl with 5 to 25 carbon atoms. For example, Ar₁ and Ar₂ are each independently selected from a substituted or unsubstituted aryl with 6, 7, 8, 9, 10, 11, 12, 13, 14, 15, 16, 17, 18, 19, 20, 21, 22, 23, 24 or 25 carbon atoms, or a substituted or unsubstituted heteroaryl with 5, 7, 8, 9, 10, 11, 12, 13, 14, 15, 16, 17, 18, 19, 20, 21, 22, 23, 24 or 25 carbon atoms.

Further optionally, Ar₁ and Ar₂ are each independently selected from a substituted or unsubstituted aryl with 6 to 15 carbon atoms, or a substituted or unsubstituted heteroaryl with 5 to 18 carbon atoms.

Optionally, substituents in Ar₁ and Ar₂ are each independently selected from: deuterium, tritium, fluorine, a trialkylsilyl with 3 to 7 carbon atoms, an alkyl with 1 to 4 carbon atoms, a cycloalkyl with 5 to 10 carbon atoms, a haloalkyl with 1 to 4 carbon atoms, an alkoxy with 1 to 4 carbon atoms, an alkylthio with 1 to 4 carbon atoms, an aryl with 6 to 12 carbon atoms, or pyridyl. Specific examples of each substituent in Ar₁ and Ar₂ include, but are not limited to, deuterium, fluorine, trimethylsilyl, methyl, ethyl, n-propyl, isopropyl, tert-butyl, trifluoromethyl, methoxyl, methylthio, phenyl, naphthyl, pyridyl, cyclopentyl, cyclohexyl and the like.

According to one embodiment, Ar₁ and Ar₂ are the same or different, and are each independently selected from groups as shown in the following formulae i-1 to i-14:

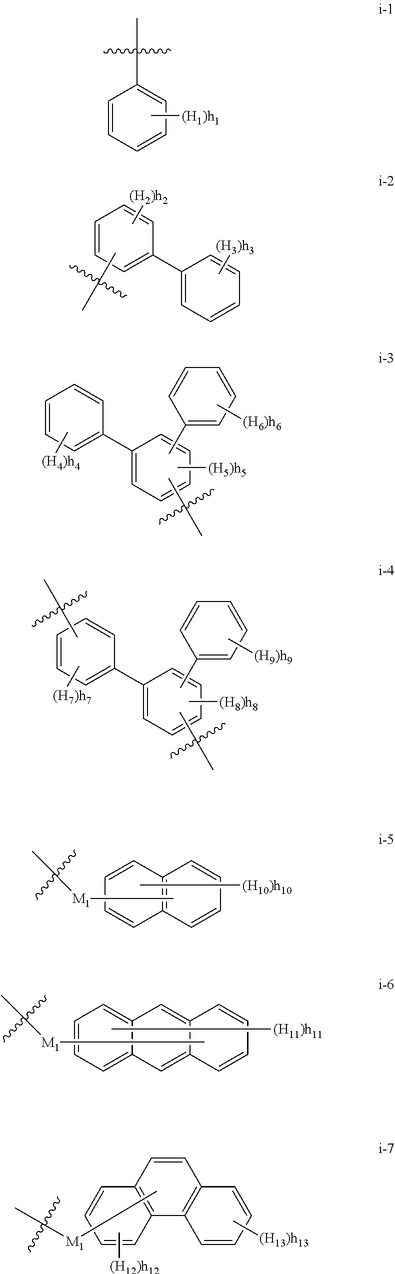

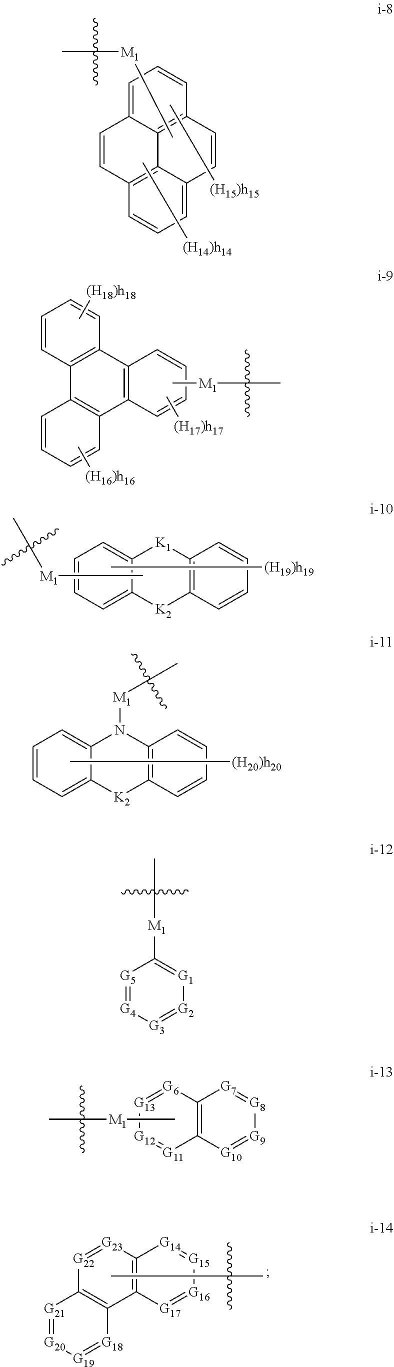

wherein M₁ is selected from single bond or

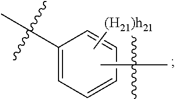

G₁ to G₅ are each independently selected from N or C(F₁), and at least one of G₁ to G₅ is selected from N; and when two or more of G₁ to G₅ are selected from C(F₁), any two F₁ are the same or different;

G₆ to G₁₃ are each independently selected from N or C(F₂), and at least one of G₆ to G₁₃ is selected from N; and when two or more of G₆ to G₁₃ are selected from C(F₂), any two F₂ are the same or different;

G₁₄ to G₂₃ are each independently selected from N or C(F₃), and at least one of G₁₄ to G₂₃ is selected from N; and when two or more of G₁₄ to G₂₃ are selected from C(F₃), any two F₃ are the same or different;

H₁ is selected from hydrogen, deuterium, fluorine, chlorine, bromine, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, or an alkylthio with 1 to 10 carbon atoms;

H₂ to H₉, and H₂₁ are each independently selected from: hydrogen, deuterium, fluorine, chlorine, bromine, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, or a heteroaryl with 3 to 18 carbon atoms;

H₁₀ to H₂₀ and F₁ to F₄ are each independently selected from: hydrogen, deuterium, fluorine, chlorine, bromine, a trialkylsilyl with 3 to 12 carbon atoms, an alkyl with 1 to 10 carbon atoms, a haloalkyl with 1 to 10 carbon atoms, a cycloalkyl with 3 to 10 carbon atoms, an alkoxy with 1 to 10 carbon atoms, an alkylthio with 1 to 10 carbon atoms, an aryl with 6 to 18 carbon atoms, or a heteroaryl with 3 to 18 carbon atoms;

h₁ to h₂₁ are represented by hₖ, H₁ to H₂₁ are represented by Hₖ, k is a variable and represents any integer from 1 to 21, and hₖ represents the number of a substituent Hₖ; wherein when k is selected from 5 or 17, hₖ is selected from 1, 2 or 3; when k is selected from 2, 7, 8, 12, 15, 16, 18 or 21, hₖ is selected from 1, 2, 3 or 4; when k is selected from 1, 3, 4, 6, 9 or 14, hₖ is selected from 1, 2, 3, 4 or 5; when k is 13, hₖ is selected from 1, 2, 3, 4, 5 or 6; when k is selected from 10 or 19, hₖ is selected from 1, 2, 3, 4, 5, 6 or 7; when k is 20, hₖ is selected from 1, 2, 3, 4, 5, 6, 7 or 8; when k is 11, hₖ is selected from 1, 2, 3, 4, 5, 6, 7, 8 or 9; and when hₖ is greater than 1, any two Hₖ are the same or different;

K₁ is selected from O, S, Se, N(H₂₂), C(H₂₃H₂₄), or Si(H₂₃H₂₄), wherein H₂₂, H₂₃, and H₂₄ are each independently selected from: an aryl with 6 to 18 carbon atoms, a heteroaryl with 3 to 18 carbon atoms, an alkyl with 1 to 10 carbon atoms, or a cycloalkyl with 3 to 10 carbon atoms, or the H₂₃ and the H₂₄ are connected to each other to form a 5- to 15-membered saturated or unsaturated ring together with the atom to which they are commonly connected; and

K₂ is selected from single bond, O, S, Se, N(H₂₅), C(H₂₆H₂₇), or Si(H₂₆H₂₇), wherein H₂₅, H₂₆, and H₂₇ are each independently selected from: an aryl with 6 to 18 carbon atoms, a heteroaryl with 3 to 18 carbon atoms, an alkyl with 1 to 10 carbon atoms, or a cycloalkyl with 3 to 10 carbon atoms, or the H₂₆ and the H₂₇ are connected to each other to form a 5- to 15-membered saturated or unsaturated ring together with the atom to which they are commonly connected.

Optionally, Ar₁ and Ar₂ are the same or different, and are each independently selected from a substituted or unsubstituted group V, wherein the unsubstituted group V is selected from the group consisting of the following groups:

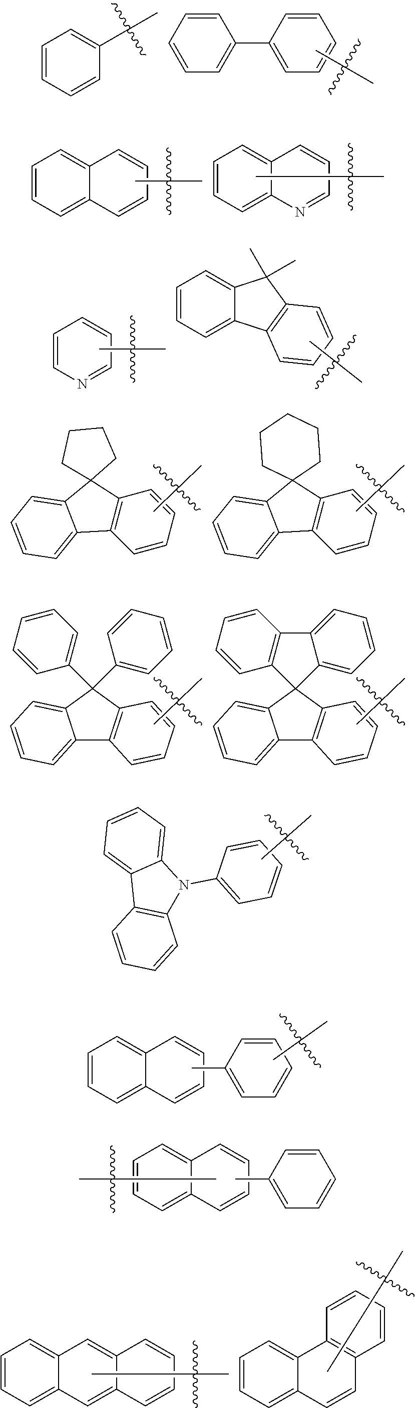

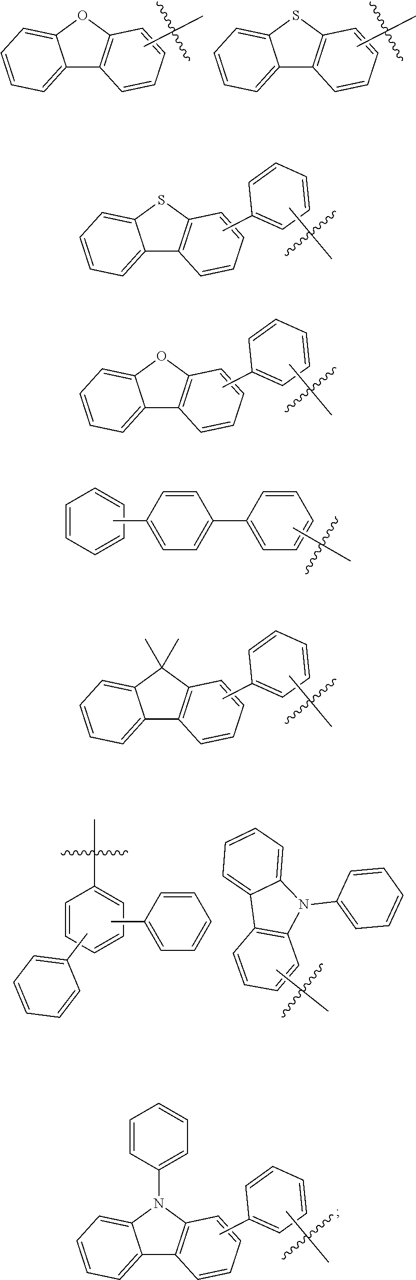

and the substituted group V has one or two or more substituents, and the substituents are each independently selected from deuterium, fluorine, a trialkylsilyl with 3 to 7 carbon atoms, an alkyl with 1 to 4 carbon atoms, a haloalkyl with 1 to 4 carbon atoms, a cycloalkyl with 5 to 10 carbon atoms, an alkoxy with 1 to 4 carbon atoms, or an alkylthio with 1 to 4 carbon atoms; and when the number of the substituents is greater than 1, the substituents are the same or different.

According to one more specific embodiment, Ar₁ and Ar₂ are the same or different, and are each independently selected from the group consisting of the following groups:

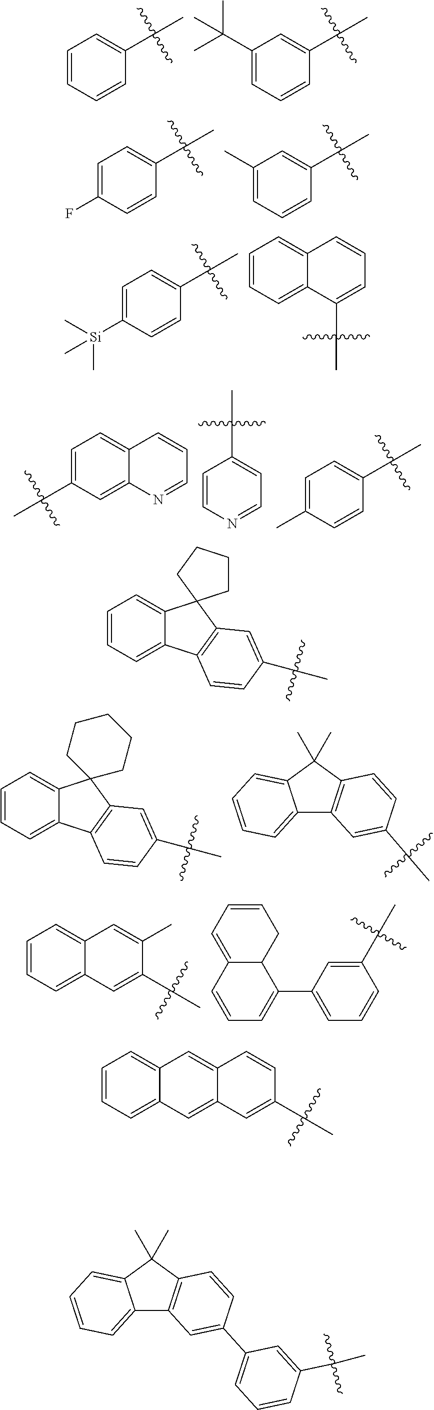

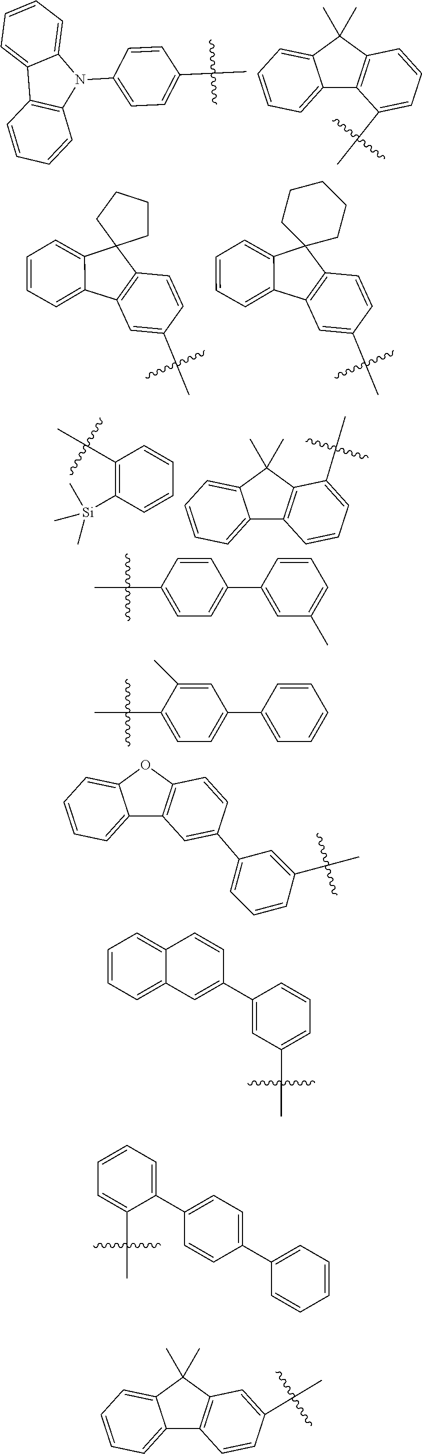

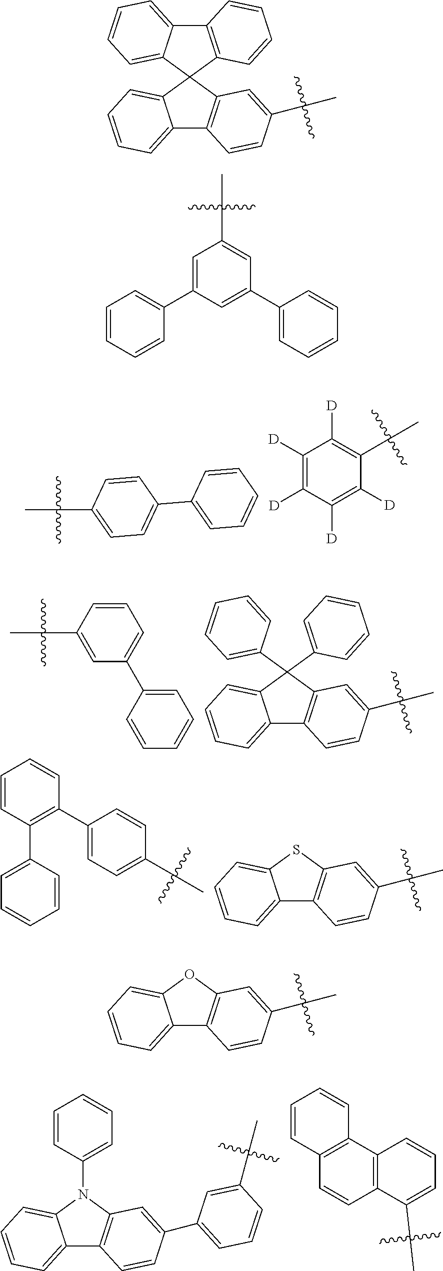

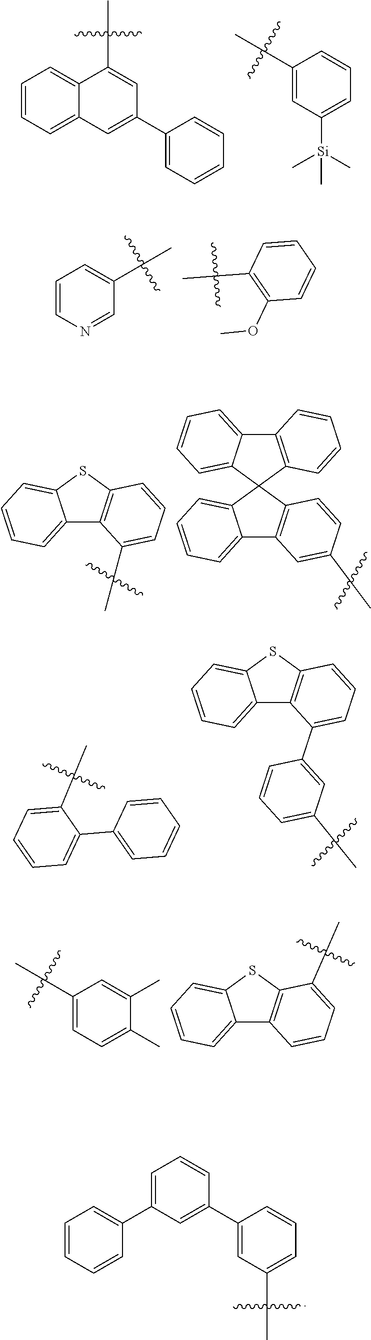

Optionally, R₁ and R₂ are the same or different, and are each independently selected from deuterium, fluorine, a trialkylsilyl with 3 to 7 carbon atoms, an aryl with 6 to 15 carbon atoms, a heteroaryl with 5 to 12 carbon atoms, an alkyl with 1 to 4 carbon atoms, a cycloalkyl with 5 to 10 carbon atoms, a fluoroalkyl with 1 to 4 carbon atoms, an alkoxy with 1 to 4 carbon atoms, or an alkylthio with 1 to 4 carbon atoms. Specific examples of each of R₁ and R₂ include, but are not limited to, deuterium, fluorine, trimethylsilyl, phenyl, naphthyl, biphenyl, 9,9-dimethylfluorenyl, pyridyl, dibenzofuranyl, dibenzothienyl, methyl, ethyl, n-propyl, isopropyl, tert-butyl, cyclopentyl, cyclohexyl, trifluoromethyl, methoxyl, or methylthio.

Optionally, R₃ is selected from hydrogen, or selected from an aryl with 6, 10, 12, 14, 15, 18 or 25 carbon atoms, and selected from a heteroaryl with 5, 9, 12, 14 or 18 carbon atoms.

According to one embodiment, R₃ may be selected from hydrogen, phenyl, naphthyl, biphenyl, carbazolyl, N-phenylcarbazolyl, dibenzothienyl, dibenzofuranyl, pyridyl, quinolyl, isoquinolyl, phenanthryl, anthryl, 9,9-dimethylfluorenyl, 9,9-diphenylfluorenyl, spirobifluorenyl or 9,9-dimethyl-9H-9-silafluorenyl.

Optionally, R₃ is selected from hydrogen, or the group consisting of the following groups:

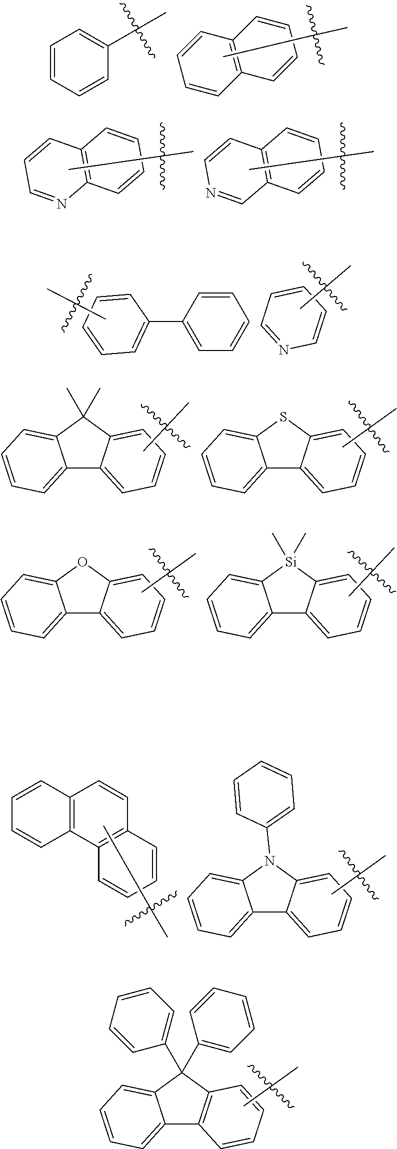

According to one preferred embodiment, the structure of the nitrogen-containing compound is shown in the formula 1-1, and R₃ is selected from the above aryl or heteroaryl. The inventor found in study that, in this case, the nitrogen-containing compound is applied to the OLED device as an electron transporting layer material, which can further improve the service life of the device.

Optionally, in the formula 1-1,

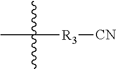

is selected from the group consisting of the following groups:

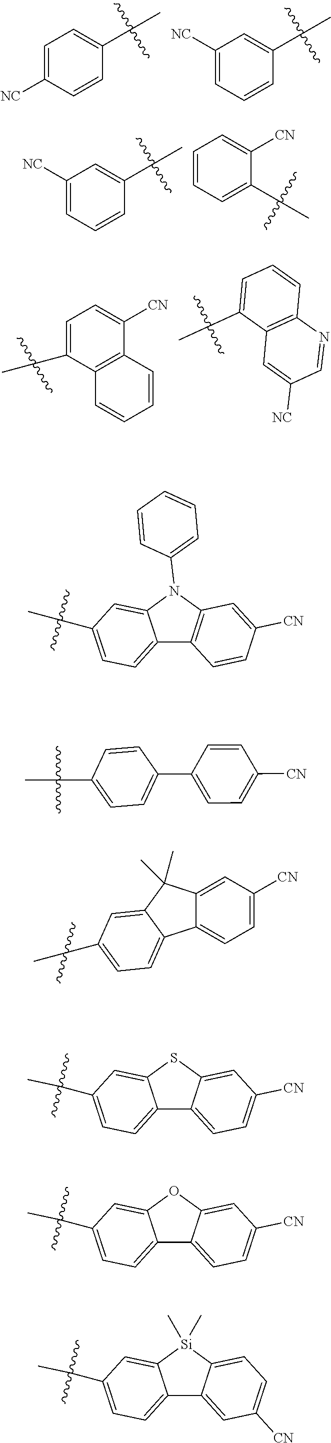

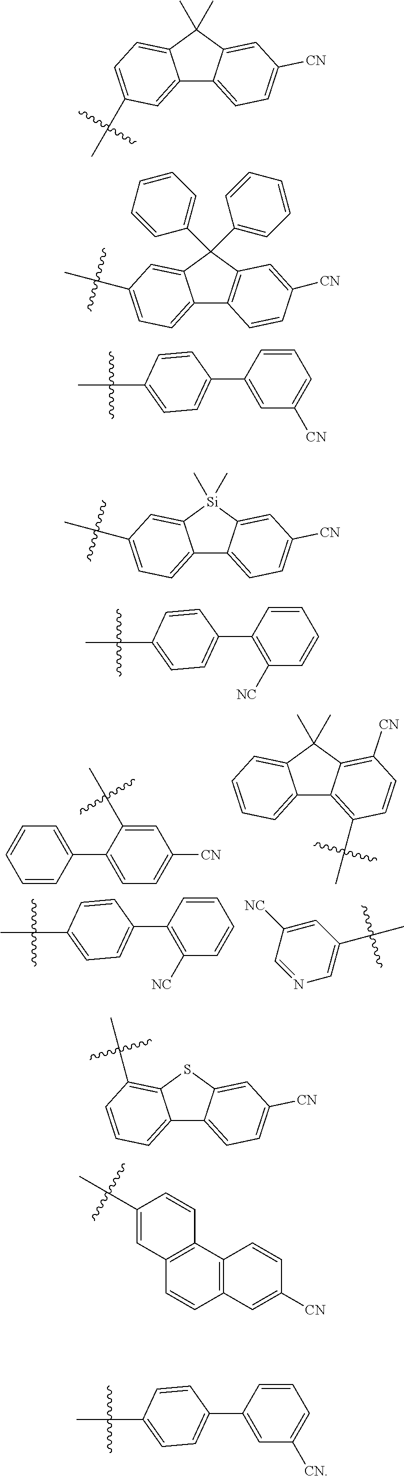

Optionally, the nitrogen-containing compound is selected from the group consisting of the following compounds:

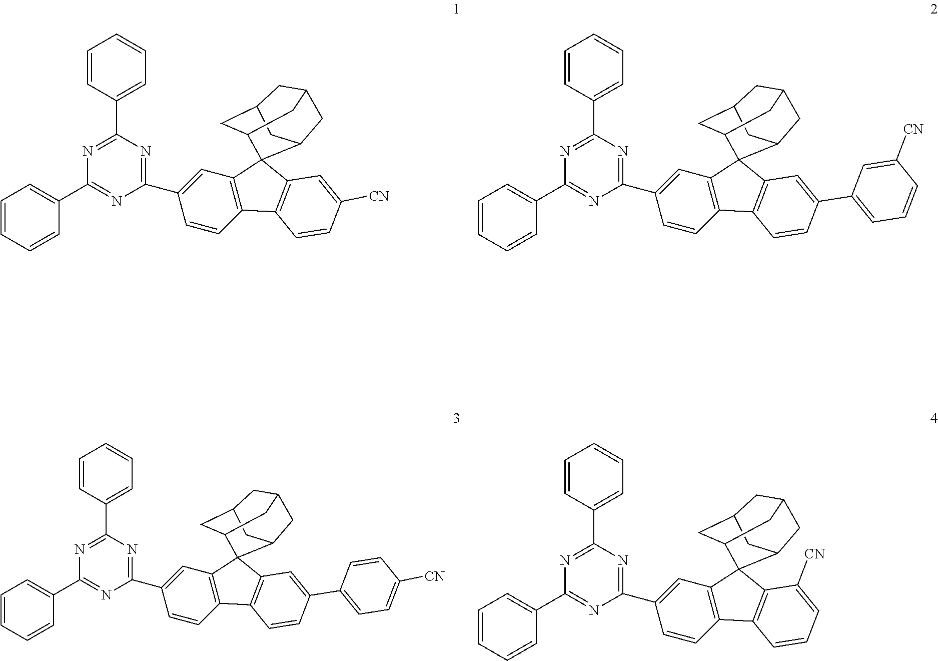 [SMILES: N#Cc1ccc2c(c1)C1(c3cc(-c4nc(-c5ccccc5)nc(-c5ccccc5)n4)ccc3-2)C2CC3CC(C2)CC1C3 ; N#Cc1cccc(-c2ccc3c(c2)C2(c4cc(-c5nc(-c6ccccc6)nc(-c6ccccc6)n5)ccc4-3)C3CC4CC(C3)CC2C4)c1 ; N#Cc1ccc(-c2ccc3c(c2)C2(c4cc(-c5nc(-c6ccccc6)nc(-c6ccccc6)n5)ccc4-3)C3CC4CC(C3)CC2C4)cc1 ; N#Cc1cccc2c1C1(c3cc(-c4nc(-c5ccccc5)nc(-c5ccccc5)n4)ccc3-2)C2CC3CC(C2)CC1C3]

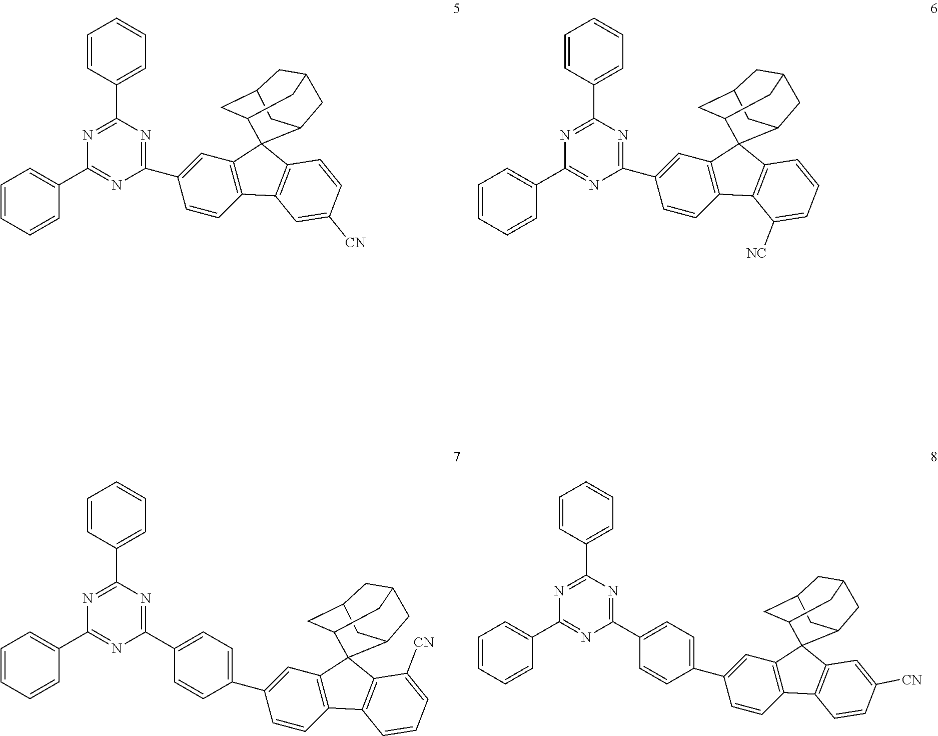 [SMILES: N#Cc1ccc2c(c1)-c1ccc(-c3nc(-c4ccccc4)nc(-c4ccccc4)n3)cc1C21C2CC3CC(C2)CC1C3 ; N#Cc1cccc2c1-c1ccc(-c3nc(-c4ccccc4)nc(-c4ccccc4)n3)cc1C21C2CC3CC(C2)CC1C3 ; N#Cc1cccc2c1C1(c3cc(-c4ccc(-c5nc(-c6ccccc6)nc(-c6ccccc6)n5)cc4)ccc3-2)C2CC3CC(C2)CC1C3 ; N#Cc1ccc2c(c1)C1(c3cc(-c4ccc(-c5nc(-c6ccccc6)nc(-c6ccccc6)n5)cc4)ccc3-2)C2CC3CC(C2)CC1C3]

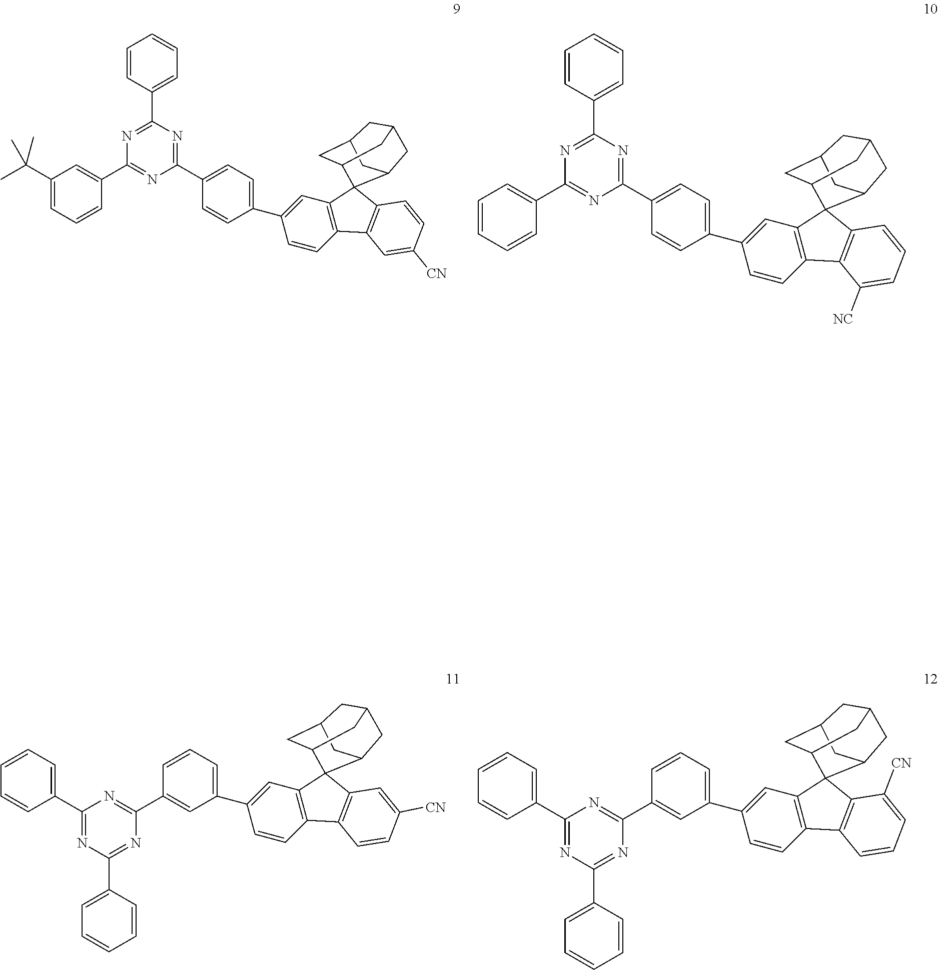 [SMILES: CC(C)(C)c1cccc(-c2nc(-c3ccccc3)nc(-c3ccc(-c4ccc5c(c4)C4(c6ccc(C#N)cc6-5)C5CC6CC(C5)CC4C6)cc3)n2)c1 ; N#Cc1cccc2c1-c1ccc(-c3ccc(-c4nc(-c5ccccc5)nc(-c5ccccc5)n4)cc3)cc1C21C2CC3CC(C2)CC1C3 ; N#Cc1ccc2c(c1)C1(c3cc(-c4cccc(-c5nc(-c6ccccc6)nc(-c6ccccc6)n5)c4)ccc3-2)C2CC3CC(C2)CC1C3 ; N#Cc1cccc2c1C1(c3cc(-c4cccc(-c5nc(-c6ccccc6)nc(-c6ccccc6)n5)c4)ccc3-2)C2CC3CC(C2)CC1C3]

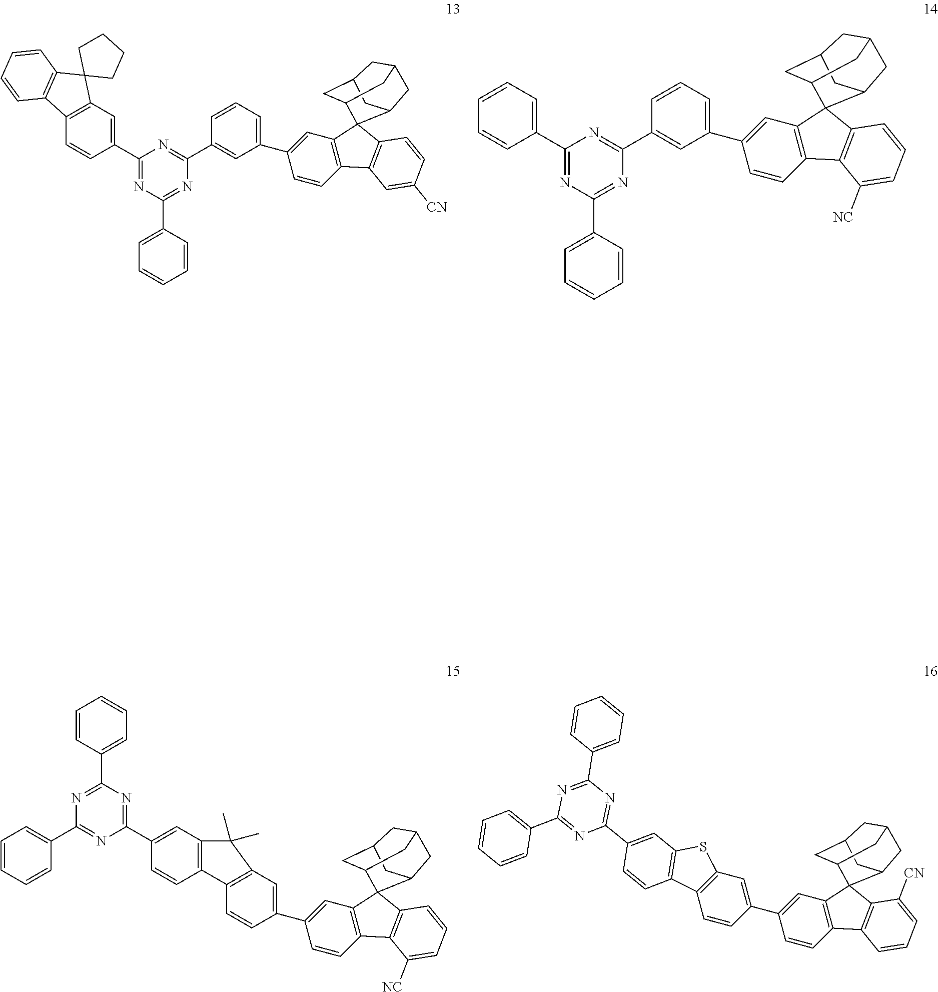 [SMILES: N#Cc1ccc2c(c1)-c1ccc(-c3cccc(-c4nc(-c5ccccc5)nc(-c5ccc6c(c5)C5(CCCC5)c5ccccc5-6)n4)c3)cc1C21C2CC3CC(C2)CC1C3 ; N#Cc1cccc2c1-c1ccc(-c3cccc(-c4nc(-c5ccccc5)nc(-c5ccccc5)n4)c3)cc1C21C2CC3CC(C2)CC1C3 ; CC1(C)c2cc(-c3ccc4c(c3)C3(c5cccc(C#N)c5-4)C4CC5CC(C4)CC3C5)ccc2-c2ccc(-c3nc(-c4ccccc4)nc(-c4ccccc4)n3)cc21 ; N#Cc1cccc2c1C1(c3cc(-c4ccc5c(c4)sc4cc(-c6nc(-c7ccccc7)nc(-c7ccccc7)n6)ccc45)ccc3-2)C2CC3CC(C2)CC1C3]

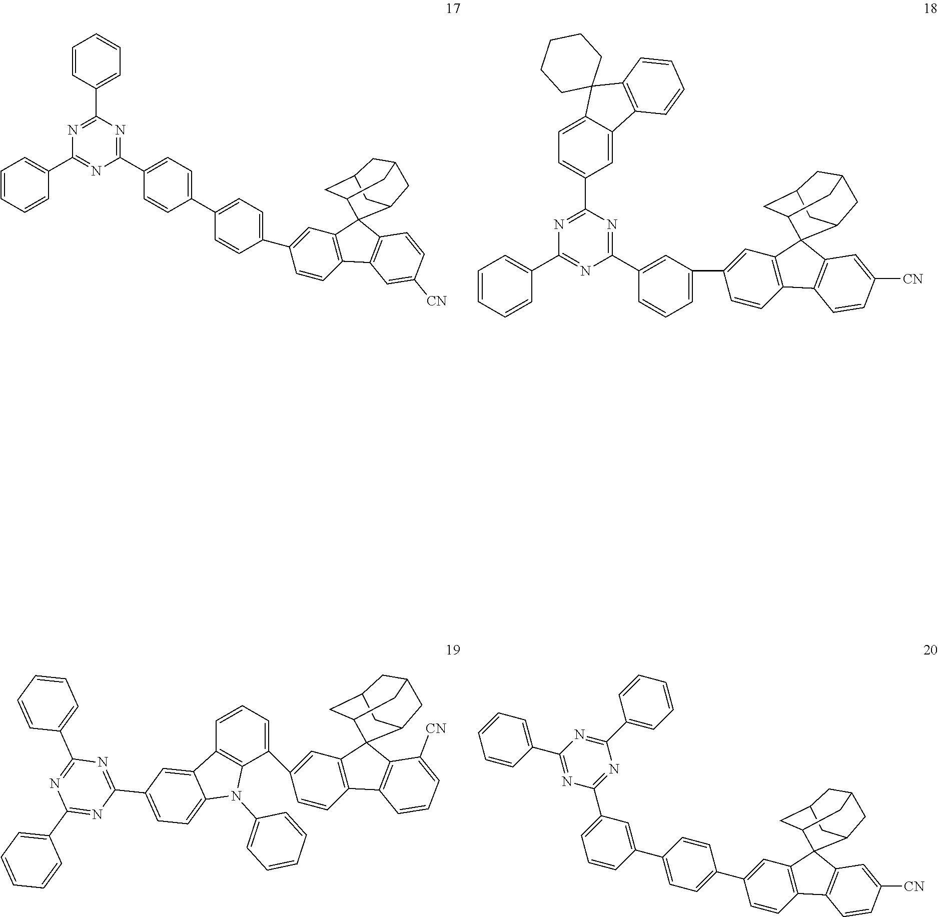 [SMILES: N#Cc1ccc2c(c1)-c1ccc(-c3ccc(-c4ccc(-c5nc(-c6ccccc6)nc(-c6ccccc6)n5)cc4)cc3)cc1C21C2CC3CC(C2)CC1C3 ; N#Cc1ccc2c(c1)C1(c3cc(-c4cccc(-c5nc(-c6ccccc6)nc(-c6ccc7c(c6)-c6ccccc6C76CCCCC6)n5)c4)ccc3-2)C2CC3CC(C2)CC1C3 ; N#Cc1cccc2c1C1(c3cc(-c4cccc5c6cc(-c7nc(-c8ccccc8)nc(-c8ccccc8)n7)ccc6n(-c6ccccc6)c45)ccc3-2)C2CC3CC(C2)CC1C3 ; N#Cc1ccc2c(c1)C1(c3cc(-c4ccc(-c5cccc(-c6nc(-c7ccccc7)nc(-c7ccccc7)n6)c5)cc4)ccc3-2)C2CC3CC(C2)CC1C3]

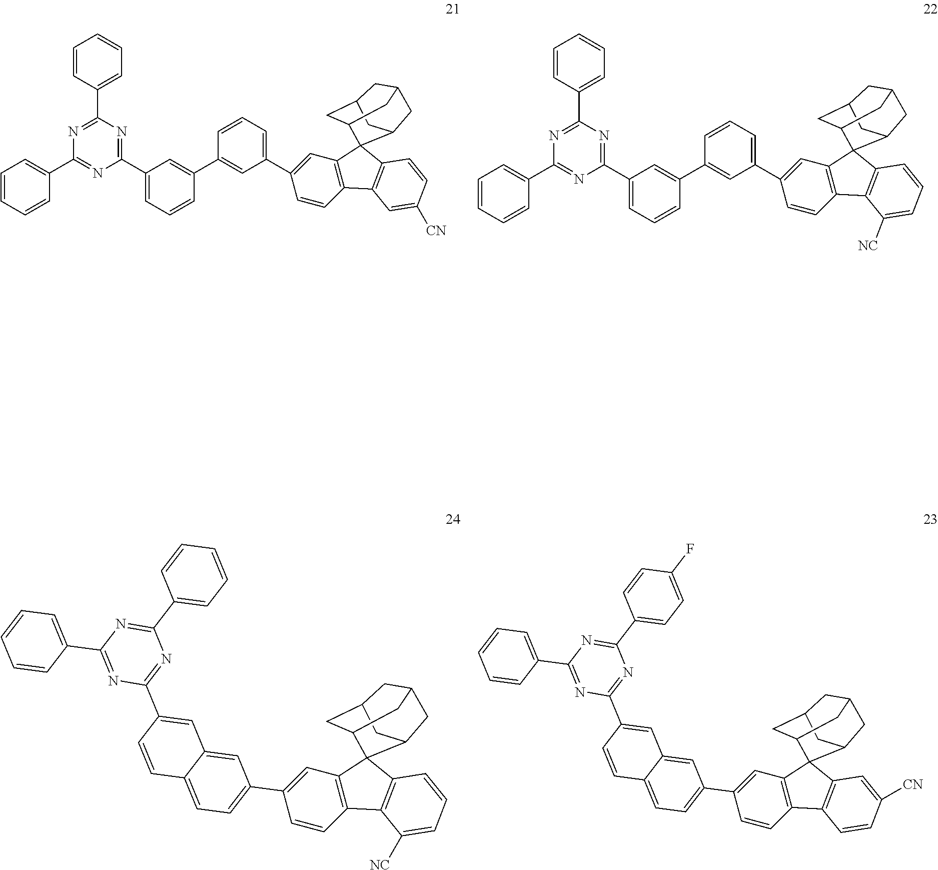 [SMILES: N#Cc1ccc2c(c1)-c1ccc(-c3cccc(-c4cccc(-c5nc(-c6ccccc6)nc(-c6ccccc6)n5)c4)c3)cc1C21C2CC3CC(C2)CC1C3 ; N#Cc1cccc2c1-c1ccc(-c3cccc(-c4cccc(-c5nc(-c6ccccc6)nc(-c6ccccc6)n5)c4)c3)cc1C21C2CC3CC(C2)CC1C3 ; N#Cc1cccc2c1-c1ccc(-c3ccc4ccc(-c5nc(-c6ccccc6)nc(-c6ccccc6)n5)cc4c3)cc1C21C2CC3CC(C2)CC1C3 ; N#Cc1ccc2c(c1)C1(c3cc(-c4ccc5ccc(-c6nc(-c7ccccc7)nc(-c7ccc(F)cc7)n6)cc5c4)ccc3-2)C2CC3CC(C2)CC1C3]

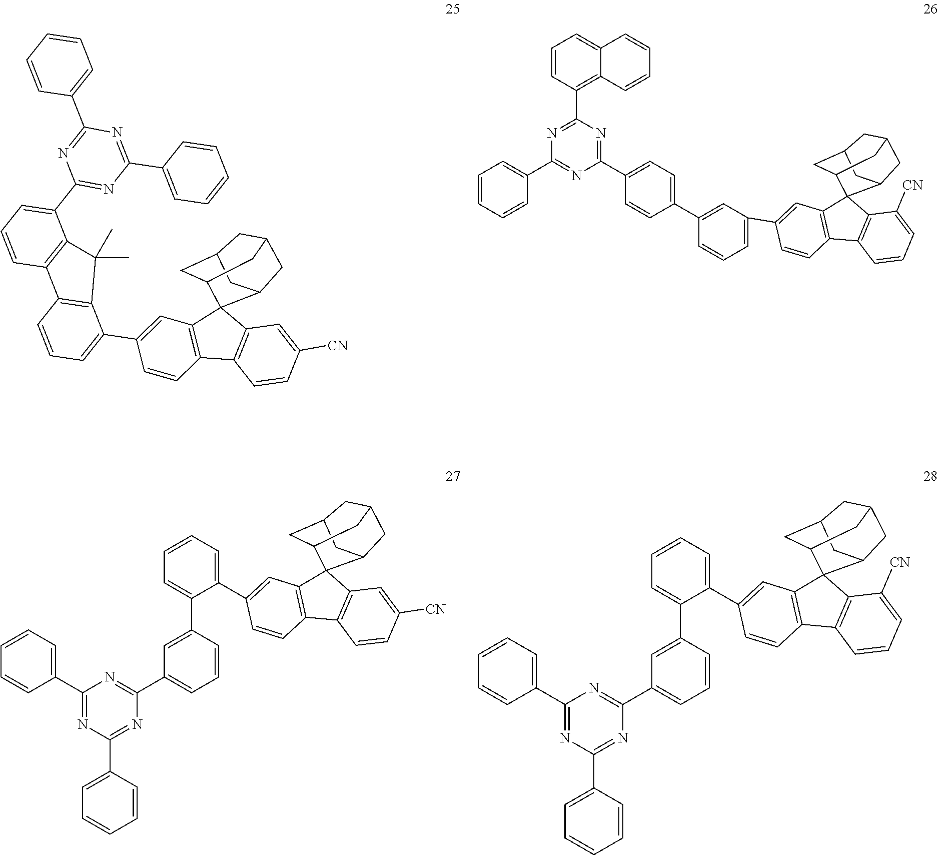 [SMILES: CC1(C)c2c(-c3ccc4c(c3)C3(c5cc(C#N)ccc5-4)C4CC5CC(C4)CC3C5)cccc2-c2cccc(-c3nc(-c4ccccc4)nc(-c4ccccc4)n3)c21 ; N#Cc1cccc2c1C1(c3cc(-c4cccc(-c5ccc(-c6nc(-c7ccccc7)nc(-c7cccc8ccccc78)n6)cc5)c4)ccc3-2)C2CC3CC(C2)CC1C3 ; N#Cc1ccc2c(c1)C1(c3cc(-c4ccccc4-c4cccc(-c5nc(-c6ccccc6)nc(-c6ccccc6)n5)c4)ccc3-2)C2CC3CC(C2)CC1C3 ; N#Cc1cccc2c1C1(c3cc(-c4ccccc4-c4cccc(-c5nc(-c6ccccc6)nc(-c6ccccc6)n5)c4)ccc3-2)C2CC3CC(C2)CC1C3]

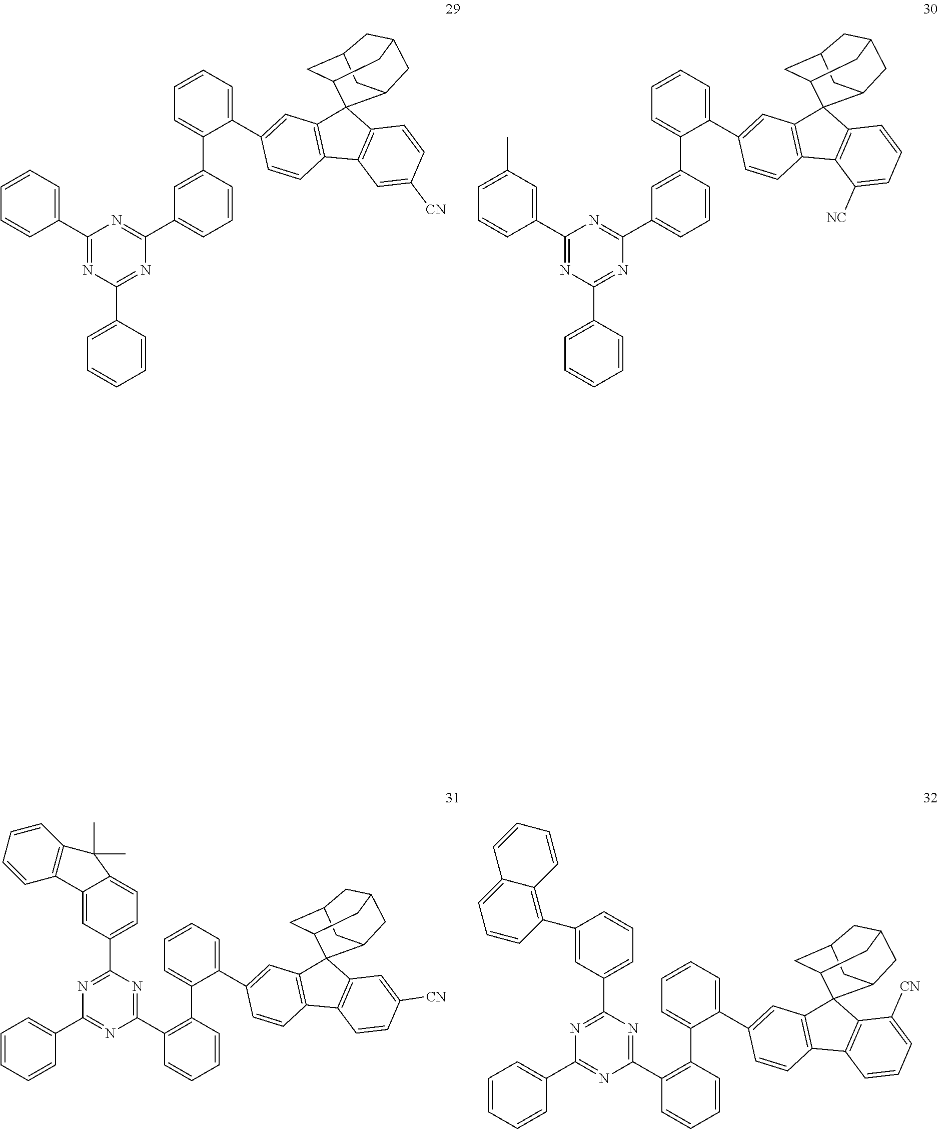 [SMILES: N#Cc1ccc2c(c1)-c1ccc(-c3ccccc3-c3cccc(-c4nc(-c5ccccc5)nc(-c5ccccc5)n4)c3)cc1C21C2CC3CC(C2)CC1C3 ; Cc1cccc(-c2nc(-c3ccccc3)nc(-c3cccc(-c4ccccc4-c4ccc5c(c4)C4(c6cccc(C#N)c6-5)C5CC6CC(C5)CC4C6)c3)n2)c1 ; CC1(C)c2ccccc2-c2cc(-c3nc(-c4ccccc4)nc(-c4ccccc4-c4ccccc4-c4ccc5c(c4)C4(c6cc(C#N)ccc6-5)C5CC6CC(C5)CC4C6)n3)ccc21 ; N#Cc1cccc2c1C1(c3cc(-c4ccccc4-c4ccccc4-c4nc(-c5ccccc5)nc(-c5cccc(-c6cccc7ccccc67)c5)n4)ccc3-2)C2CC3CC(C2)CC1C3]

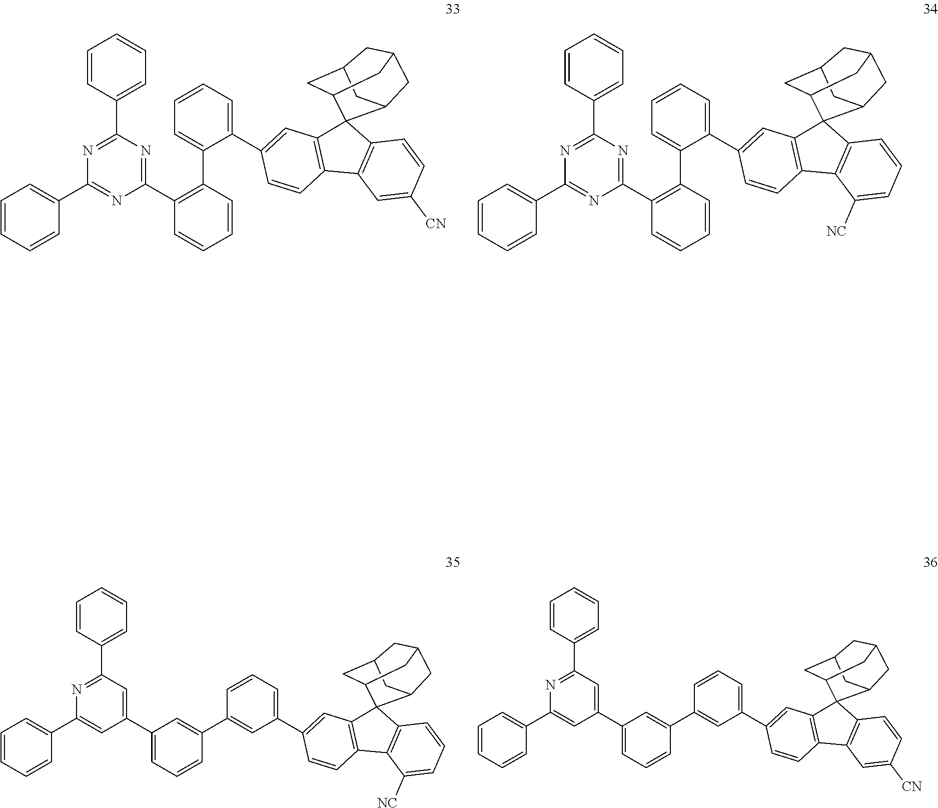

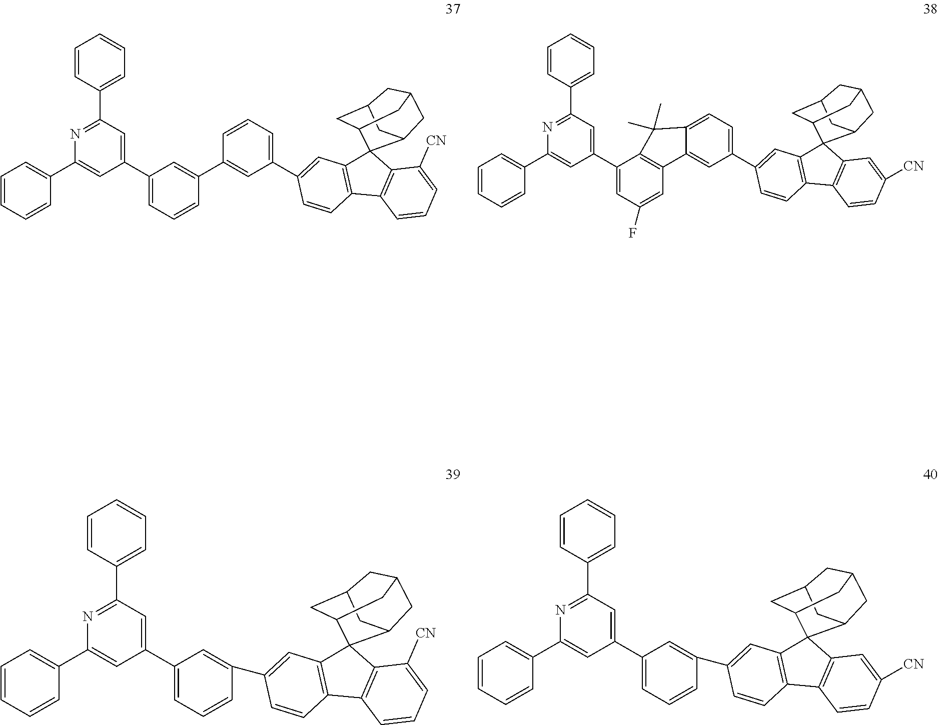 [SMILES: N#Cc1cccc2c1C1(c3cc(-c4cccc(-c5cccc(-c6cc(-c7ccccc7)nc(-c7ccccc7)c6)c5)c4)ccc3-2)C2CC3CC(C2)CC1C3 ; CC1(C)c2ccc(-c3ccc4c(c3)C3(c5cc(C#N)ccc5-4)C4CC5CC(C4)CC3C5)cc2-c2cc(F)cc(-c3cc(-c4ccccc4)nc(-c4ccccc4)c3)c21 ; N#Cc1cccc2c1C1(c3cc(-c4cccc(-c5cc(-c6ccccc6)nc(-c6ccccc6)c5)c4)ccc3-2)C2CC3CC(C2)CC1C3 ; N#Cc1ccc2c(c1)C1(c3cc(-c4cccc(-c5cc(-c6ccccc6)nc(-c6ccccc6)c5)c4)ccc3-2)C2CC3CC(C2)CC1C3]

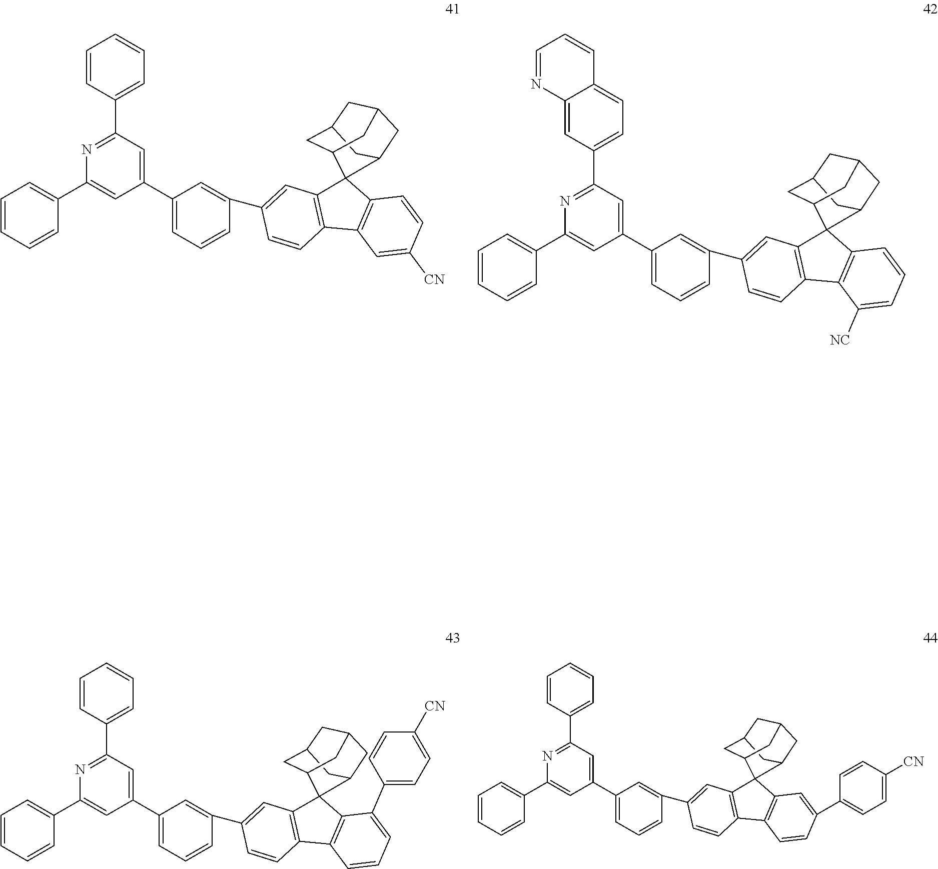 [SMILES: N#Cc1ccc2c(c1)-c1ccc(-c3cccc(-c4cc(-c5ccccc5)nc(-c5ccccc5)c4)c3)cc1C21C2CC3CC(C2)CC1C3 ; N#Cc1cccc2c1-c1ccc(-c3cccc(-c4cc(-c5ccccc5)nc(-c5ccc6cccnc6c5)c4)c3)cc1C21C2CC3CC(C2)CC1C3 ; N#Cc1ccc(-c2cccc3c2C2(c4cc(-c5cccc(-c6cc(-c7ccccc7)nc(-c7ccccc7)c6)c5)ccc4-3)C3CC4CC(C3)CC2C4)cc1 ; N#Cc1ccc(-c2ccc3c(c2)C2(c4cc(-c5cccc(-c6cc(-c7ccccc7)nc(-c7ccccc7)c6)c5)ccc4-3)C3CC4CC(C3)CC2C4)cc1]

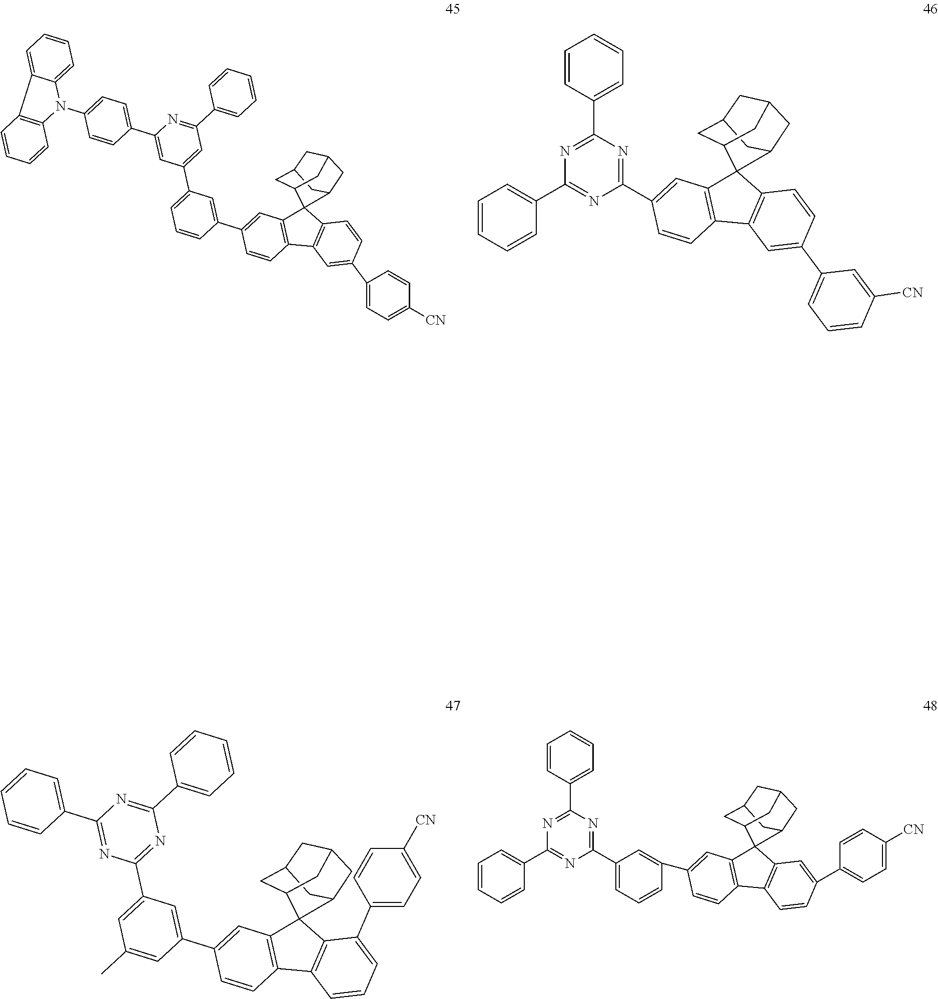 [SMILES: N#Cc1ccc(-c2ccc3c(c2)-c2ccc(-c4cccc(-c5cc(-c6ccccc6)nc(-c6ccc(-n7c8ccccc8c8ccccc87)cc6)c5)c4)cc2C32C3CC4CC(C3)CC2C4)cc1 ; N#Cc1cccc(-c2ccc3c(c2)-c2ccc(-c4nc(-c5ccccc5)nc(-c5ccccc5)n4)cc2C32C3CC4CC(C3)CC2C4)c1 ; Cc1cc(-c2ccc3c(c2)C2(c4c(-c5ccc(C#N)cc5)cccc4-3)C3CC4CC(C3)CC2C4)cc(-c2nc(-c3ccccc3)nc(-c3ccccc3)n2)c1 ; N#Cc1ccc(-c2ccc3c(c2)C2(c4cc(-c5cccc(-c6nc(-c7ccccc7)nc(-c7ccccc7)n6)c5)ccc4-3)C3CC4CC(C3)CC2C4)cc1]

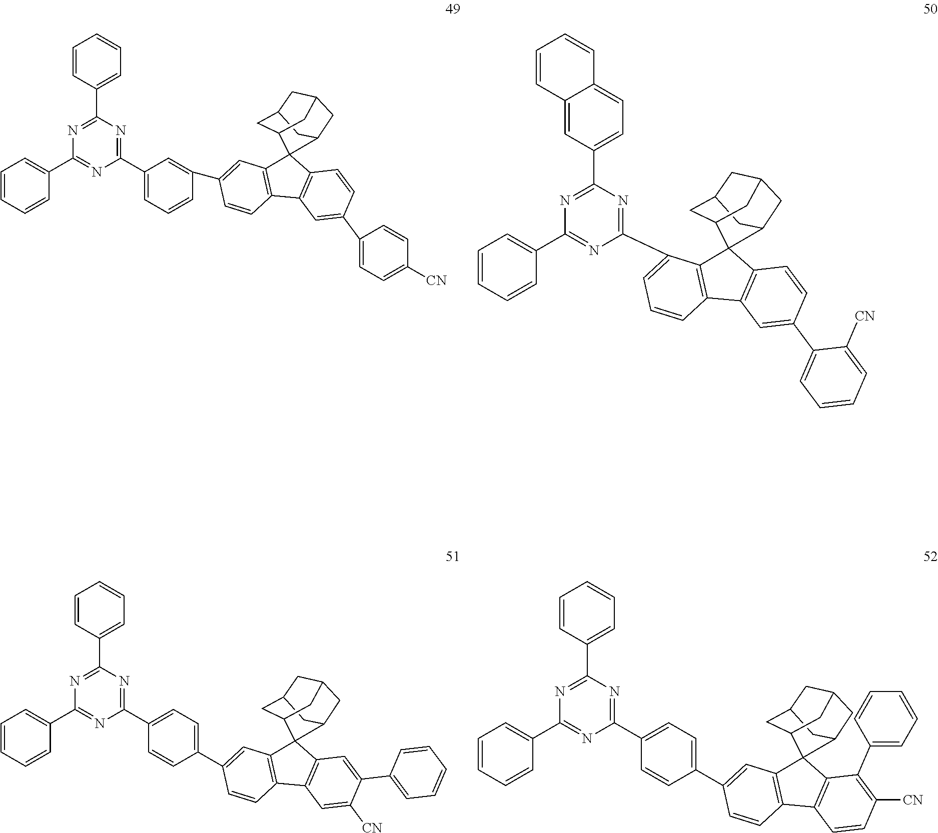

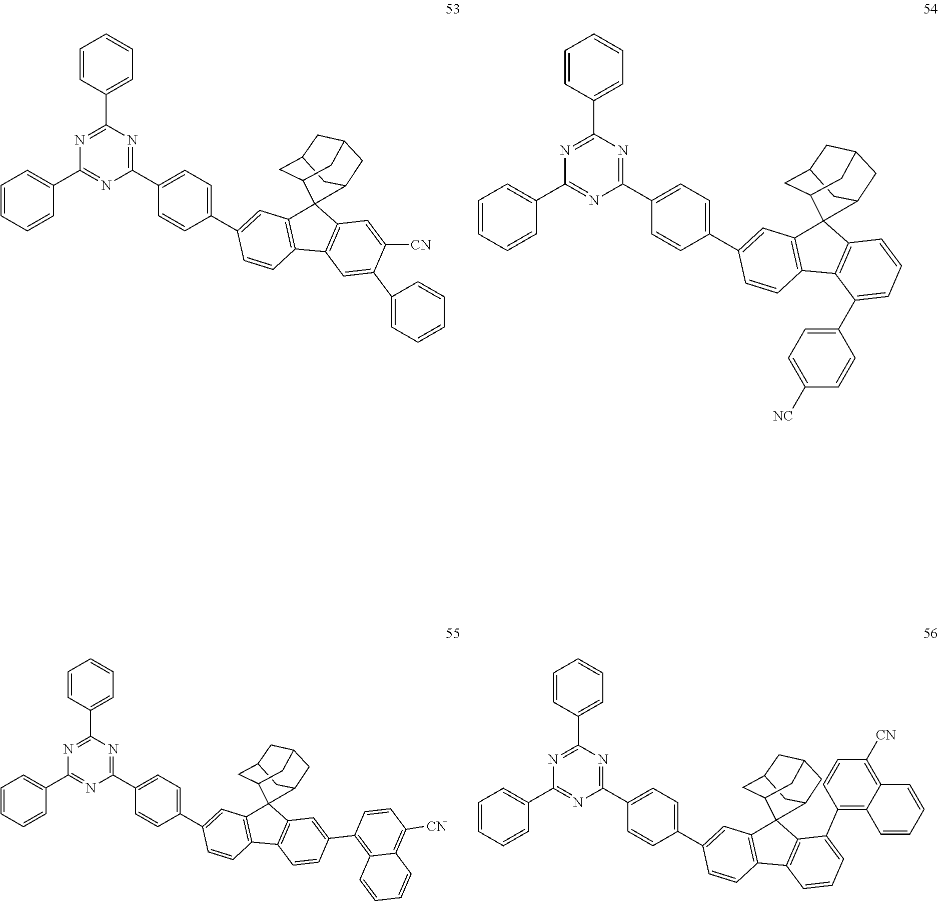 [SMILES: N#Cc1cc2c(cc1-c1ccccc1)-c1ccc(-c3ccc(-c4nc(-c5ccccc5)nc(-c5ccccc5)n4)cc3)cc1C21C2CC3CC(C2)CC1C3 ; N#Cc1ccc(-c2cccc3c2-c2ccc(-c4ccc(-c5nc(-c6ccccc6)nc(-c6ccccc6)n5)cc4)cc2C32C3CC4CC(C3)CC2C4)cc1 ; N#Cc1ccc(-c2ccc3c(c2)C2(c4cc(-c5ccc(-c6nc(-c7ccccc7)nc(-c7ccccc7)n6)cc5)ccc4-3)C3CC4CC(C3)CC2C4)c2ccccc12 ; N#Cc1ccc(-c2cccc3c2C2(c4cc(-c5ccc(-c6nc(-c7ccccc7)nc(-c7ccccc7)n6)cc5)ccc4-3)C3CC4CC(C3)CC2C4)c2ccccc12]

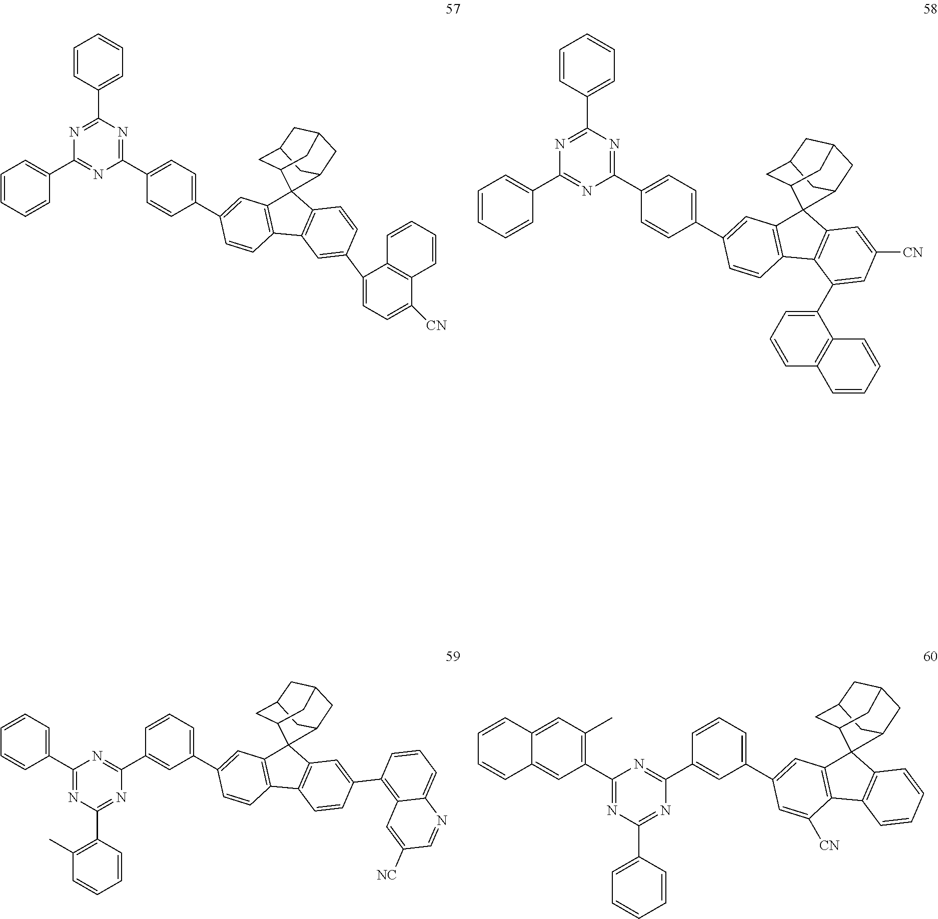 [SMILES: N#Cc1ccc(-c2ccc3c(c2)-c2ccc(-c4ccc(-c5nc(-c6ccccc6)nc(-c6ccccc6)n5)cc4)cc2C32C3CC4CC(C3)CC2C4)c2ccccc12 ; N#Cc1cc(-c2cccc3ccccc23)c2c(c1)C1(c3cc(-c4ccc(-c5nc(-c6ccccc6)nc(-c6ccccc6)n5)cc4)ccc3-2)C2CC3CC(C2)CC1C3 ; Cc1ccccc1-c1nc(-c2ccccc2)nc(-c2cccc(-c3ccc4c(c3)C3(c5cc(-c6cccc7ncc(C#N)cc67)ccc5-4)C4CC5CC(C4)CC3C5)c2)n1 ; Cc1cc2ccccc2cc1-c1nc(-c2ccccc2)nc(-c2cccc(-c3cc(C#N)c4c(c3)C3(c5ccccc5-4)C4CC5CC(C4)CC3C5)c2)n1]

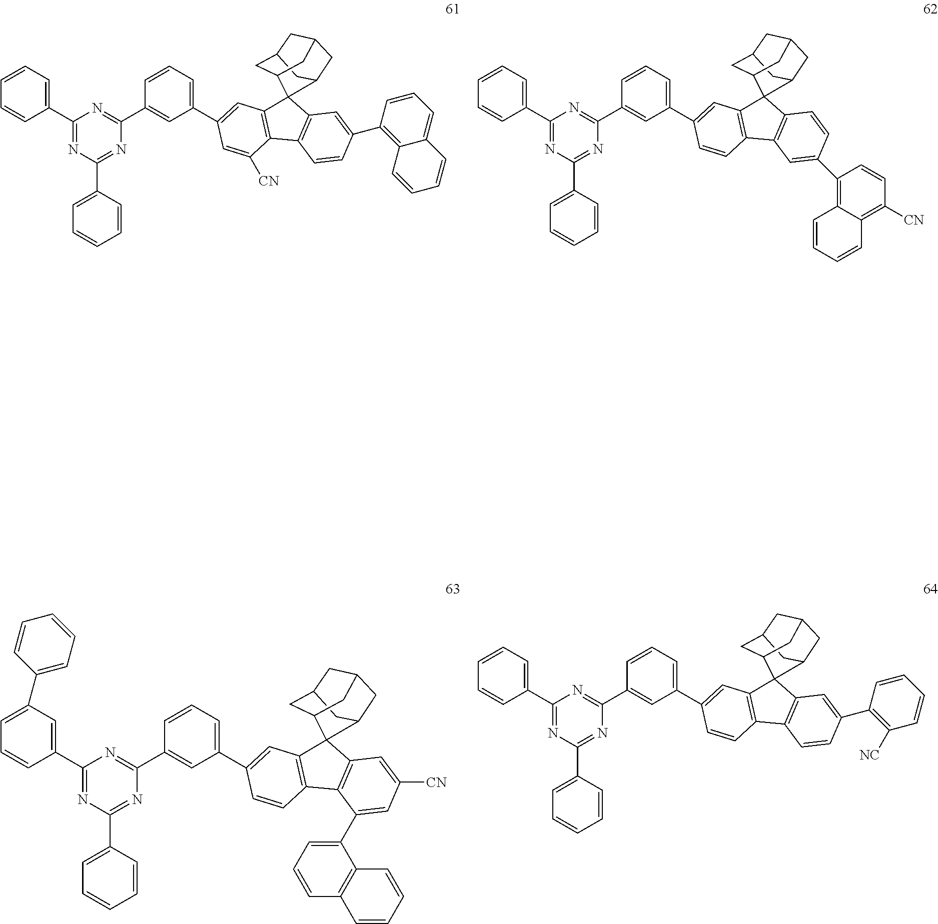 [SMILES: N#Cc1cc(-c2cccc(-c3nc(-c4ccccc4)nc(-c4ccccc4)n3)c2)cc2c1-c1ccc(-c3cccc4ccccc34)cc1C21C2CC3CC(C2)CC1C3 ; N#Cc1ccc(-c2ccc3c(c2)-c2ccc(-c4cccc(-c5nc(-c6ccccc6)nc(-c6ccccc6)n5)c4)cc2C32C3CC4CC(C3)CC2C4)c2ccccc12 ; N#Cc1cc(-c2cccc3ccccc23)c2c(c1)C1(c3cc(-c4cccc(-c5nc(-c6ccccc6)nc(-c6cccc(-c7ccccc7)c6)n5)c4)ccc3-2)C2CC3CC(C2)CC1C3 ; N#Cc1ccccc1-c1ccc2c(c1)C1(c3cc(-c4cccc(-c5nc(-c6ccccc6)nc(-c6ccccc6)n5)c4)ccc3-2)C2CC3CC(C2)CC1C3]

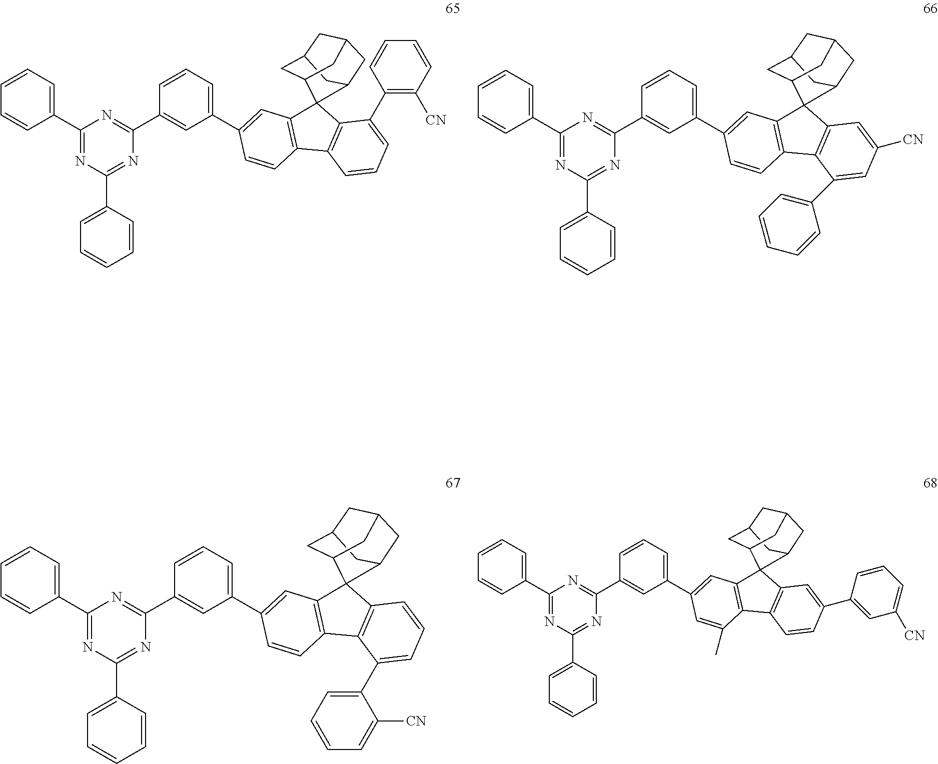 [SMILES: N#Cc1ccccc1-c1cccc2c1C1(c3cc(-c4cccc(-c5nc(-c6ccccc6)nc(-c6ccccc6)n5)c4)ccc3-2)C2CC3CC(C2)CC1C3 ; N#Cc1cc(-c2ccccc2)c2c(c1)C1(c3cc(-c4cccc(-c5nc(-c6ccccc6)nc(-c6ccccc6)n5)c4)ccc3-2)C2CC3CC(C2)CC1C3 ; N#Cc1ccccc1-c1cccc2c1-c1ccc(-c3cccc(-c4nc(-c5ccccc5)nc(-c5ccccc5)n4)c3)cc1C21C2CC3CC(C2)CC1C3 ; Cc1cc(-c2cccc(-c3nc(-c4ccccc4)nc(-c4ccccc4)n3)c2)cc2c1-c1ccc(-c3cccc(C#N)c3)cc1C21C2CC3CC(C2)CC1C3]

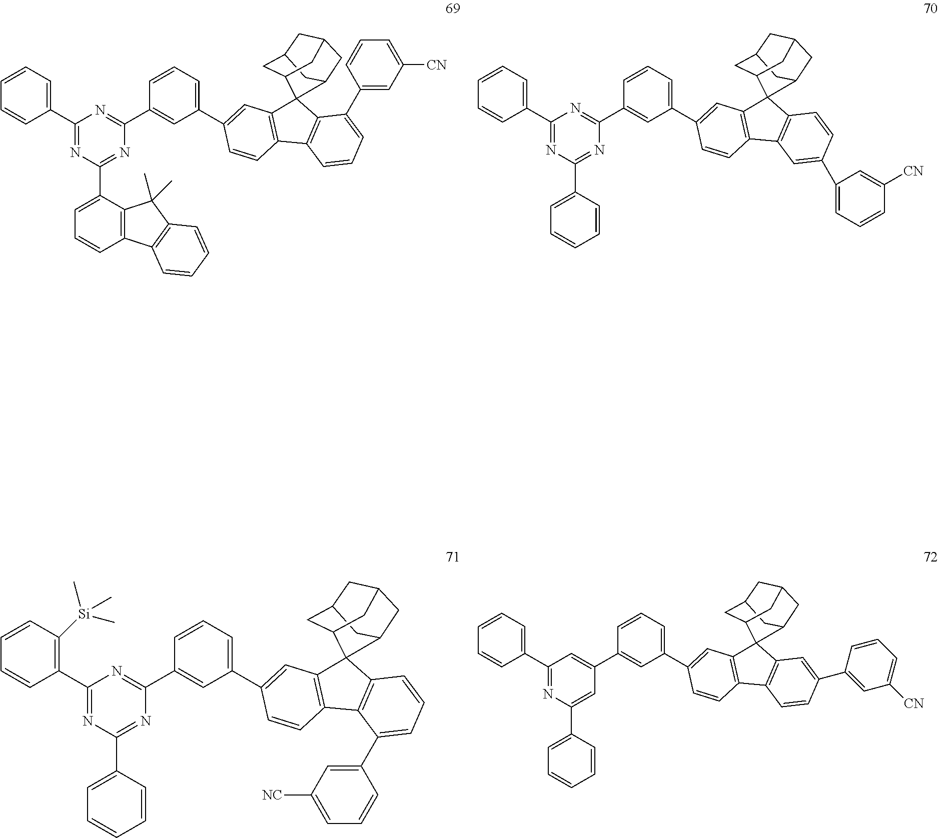 [SMILES: CC1(C)c2ccccc2-c2cccc(-c3nc(-c4ccccc4)nc(-c4cccc(-c5ccc6c(c5)C5(c7c(-c8cccc(C#N)c8)cccc7-6)C6CC7CC(C6)CC5C7)c4)n3)c21 ; N#Cc1cccc(-c2ccc3c(c2)-c2ccc(-c4cccc(-c5nc(-c6ccccc6)nc(-c6ccccc6)n5)c4)cc2C32C3CC4CC(C3)CC2C4)c1 ; C[Si](C)(C)c1ccccc1-c1nc(-c2ccccc2)nc(-c2cccc(-c3ccc4c(c3)C3(c5cccc(-c6cccc(C#N)c6)c5-4)C4CC5CC(C4)CC3C5)c2)n1 ; N#Cc1cccc(-c2ccc3c(c2)C2(c4cc(-c5cccc(-c6cc(-c7ccccc7)nc(-c7ccccc7)c6)c5)ccc4-3)C3CC4CC(C3)CC2C4)c1]

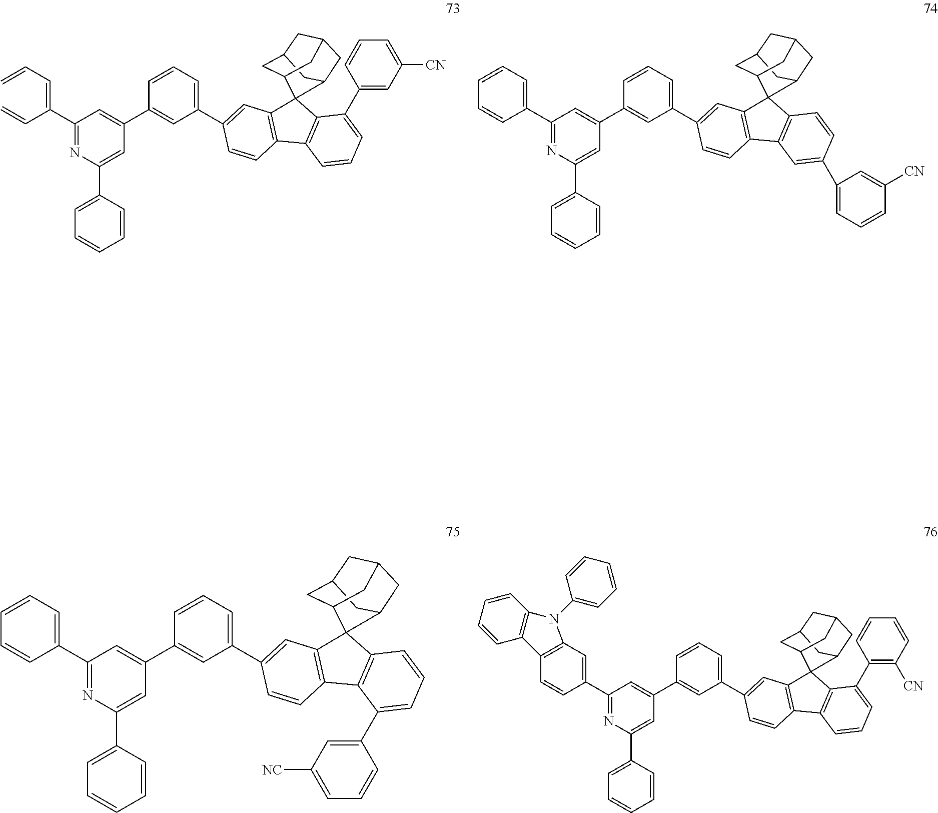 [SMILES: C=C/C=C(\C=C)c1cc(-c2cccc(-c3ccc4c(c3)C3(c5c(-c6cccc(C#N)c6)cccc5-4)C4CC5CC(C4)CC3C5)c2)cc(-c2ccccc2)n1 ; N#Cc1cccc(-c2ccc3c(c2)-c2ccc(-c4cccc(-c5cc(-c6ccccc6)nc(-c6ccccc6)c5)c4)cc2C32C3CC4CC(C3)CC2C4)c1 ; N#Cc1cccc(-c2cccc3c2-c2ccc(-c4cccc(-c5cc(-c6ccccc6)nc(-c6ccccc6)c5)c4)cc2C32C3CC4CC(C3)CC2C4)c1 ; N#Cc1ccccc1-c1cccc2c1C1(c3cc(-c4cccc(-c5cc(-c6ccccc6)nc(-c6ccc7c8ccccc8n(-c8ccccc8)c7c6)c5)c4)ccc3-2)C2CC3CC(C2)CC1C3]

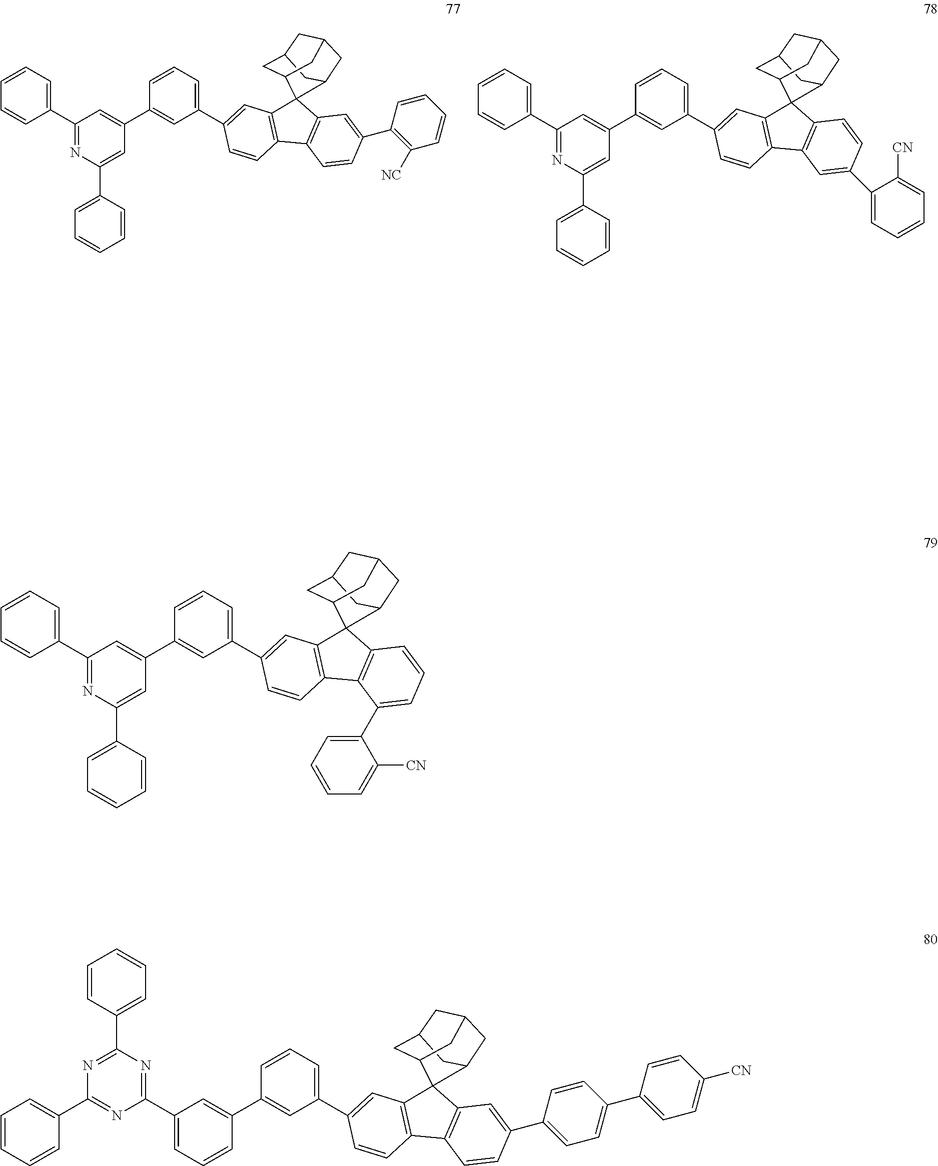

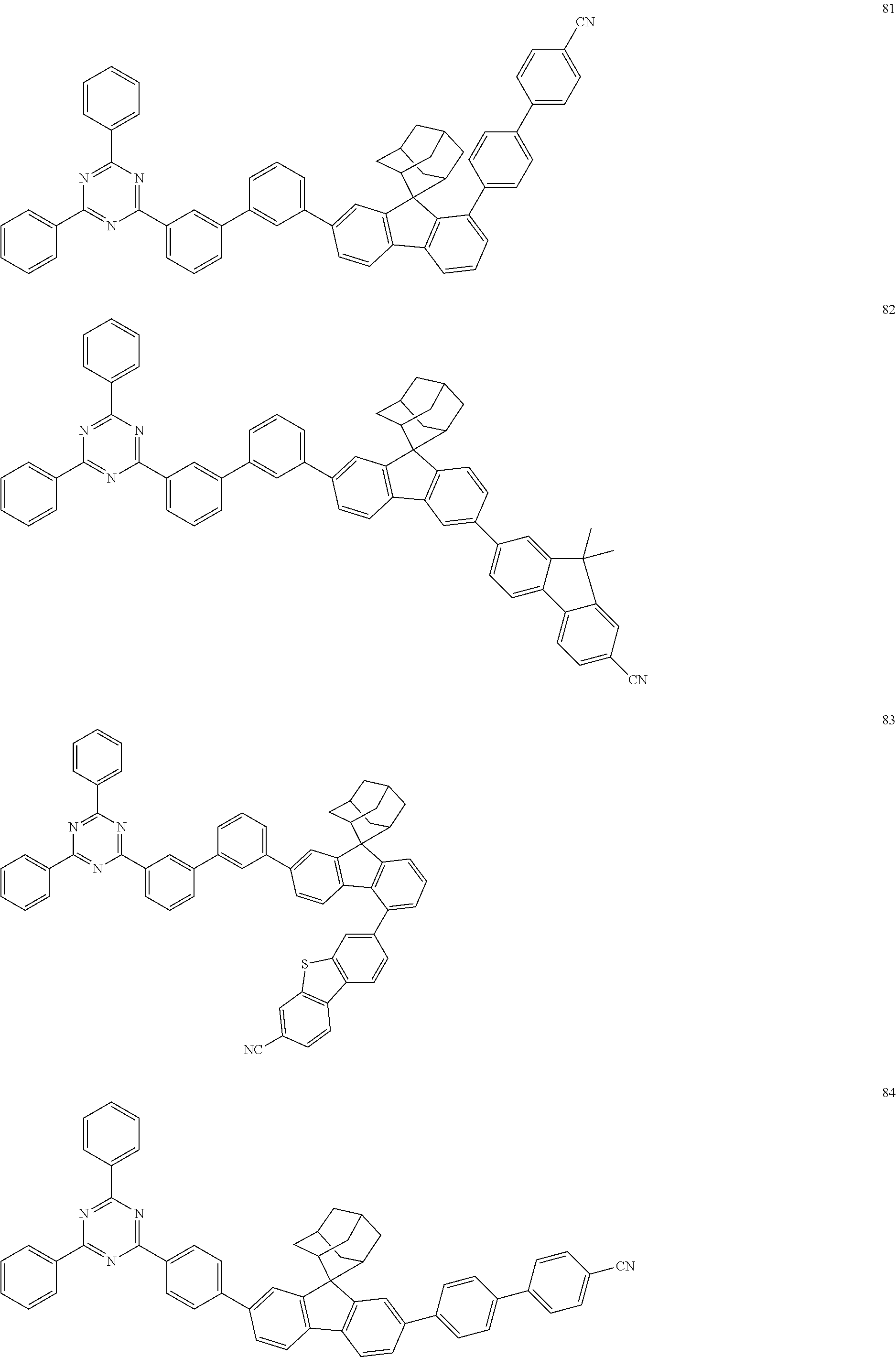 [SMILES: N#Cc1ccc(-c2ccc(-c3cccc4c3C3(c5cc(-c6cccc(-c7cccc(-c8nc(-c9ccccc9)nc(-c9ccccc9)n8)c7)c6)ccc5-4)C4CC5CC(C4)CC3C5)cc2)cc1 ; CC1(C)c2cc(C#N)ccc2-c2ccc(-c3ccc4c(c3)-c3ccc(-c5cccc(-c6cccc(-c7nc(-c8ccccc8)nc(-c8ccccc8)n7)c6)c5)cc3C43C4CC5CC(C4)CC3C5)cc21 ; N#Cc1ccc2c(c1)sc1cc(-c3cccc4c3-c3ccc(-c5cccc(-c6cccc(-c7nc(-c8ccccc8)nc(-c8ccccc8)n7)c6)c5)cc3C43C4CC5CC(C4)CC3C5)ccc12 ; N#Cc1ccc(-c2ccc(-c3ccc4c(c3)C3(c5cc(-c6ccc(-c7nc(-c8ccccc8)nc(-c8ccccc8)n7)cc6)ccc5-4)C4CC5CC(C4)CC3C5)cc2)cc1]

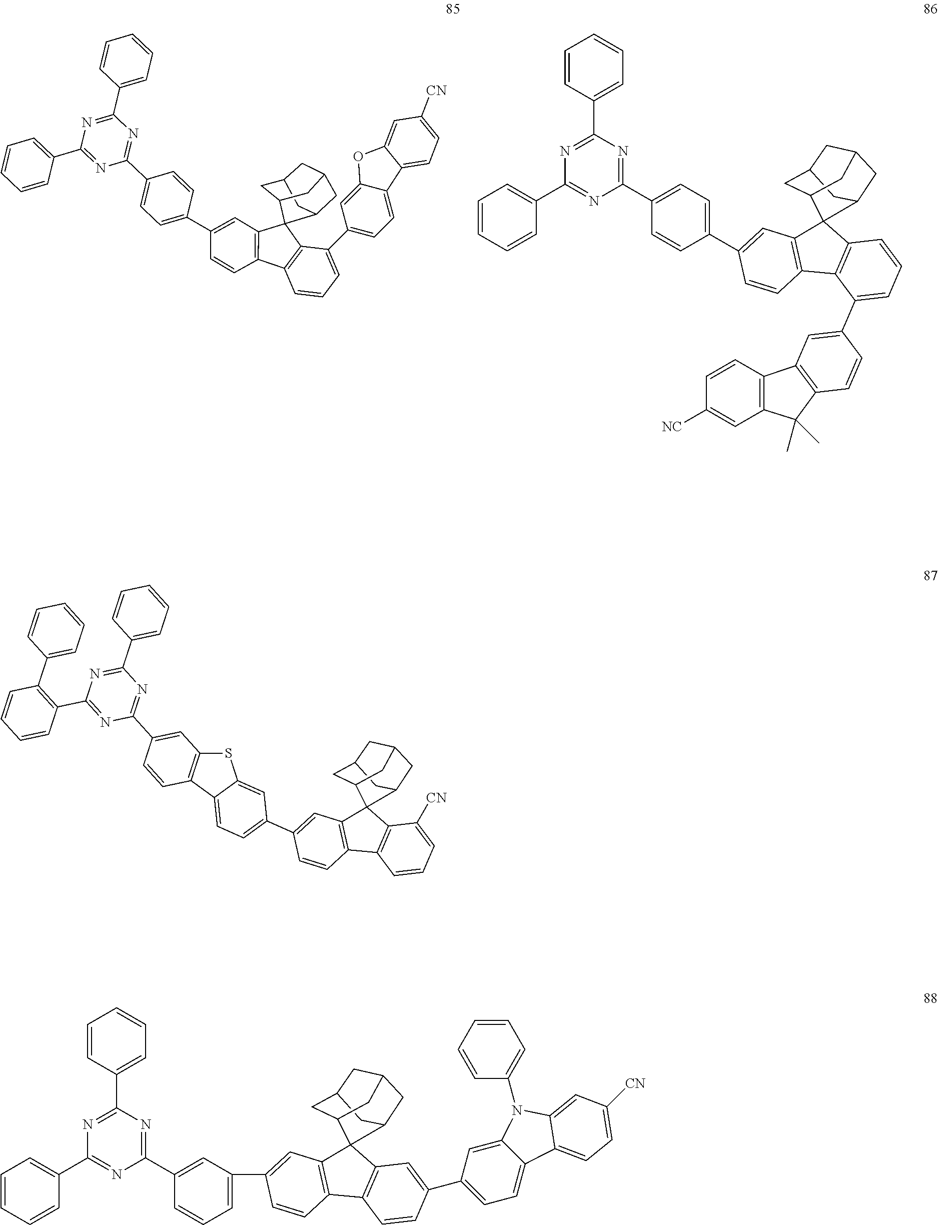 [SMILES: N#Cc1ccc2c(c1)oc1cc(-c3cccc4c3C3(c5cc(-c6ccc(-c7nc(-c8ccccc8)nc(-c8ccccc8)n7)cc6)ccc5-4)C4CC5CC(C4)CC3C5)ccc12 ; CC1(C)c2ccc(-c3cccc4c3-c3ccc(-c5ccc(-c6nc(-c7ccccc7)nc(-c7ccccc7)n6)cc5)cc3C43C4CC5CC(C4)CC3C5)cc2-c2ccc(C#N)cc21 ; N#Cc1cccc2c1C1(c3cc(-c4ccc5c(c4)sc4cc(-c6nc(-c7ccccc7)nc(-c7ccccc7-c7ccccc7)n6)ccc45)ccc3-2)C2CC3CC(C2)CC1C3 ; N#Cc1ccc2c3ccc(-c4ccc5c(c4)C4(c6cc(-c7cccc(-c8nc(-c9ccccc9)nc(-c9ccccc9)n8)c7)ccc6-5)C5CC6CC(C5)CC4C6)cc3n(-c3ccccc3)c2c1]

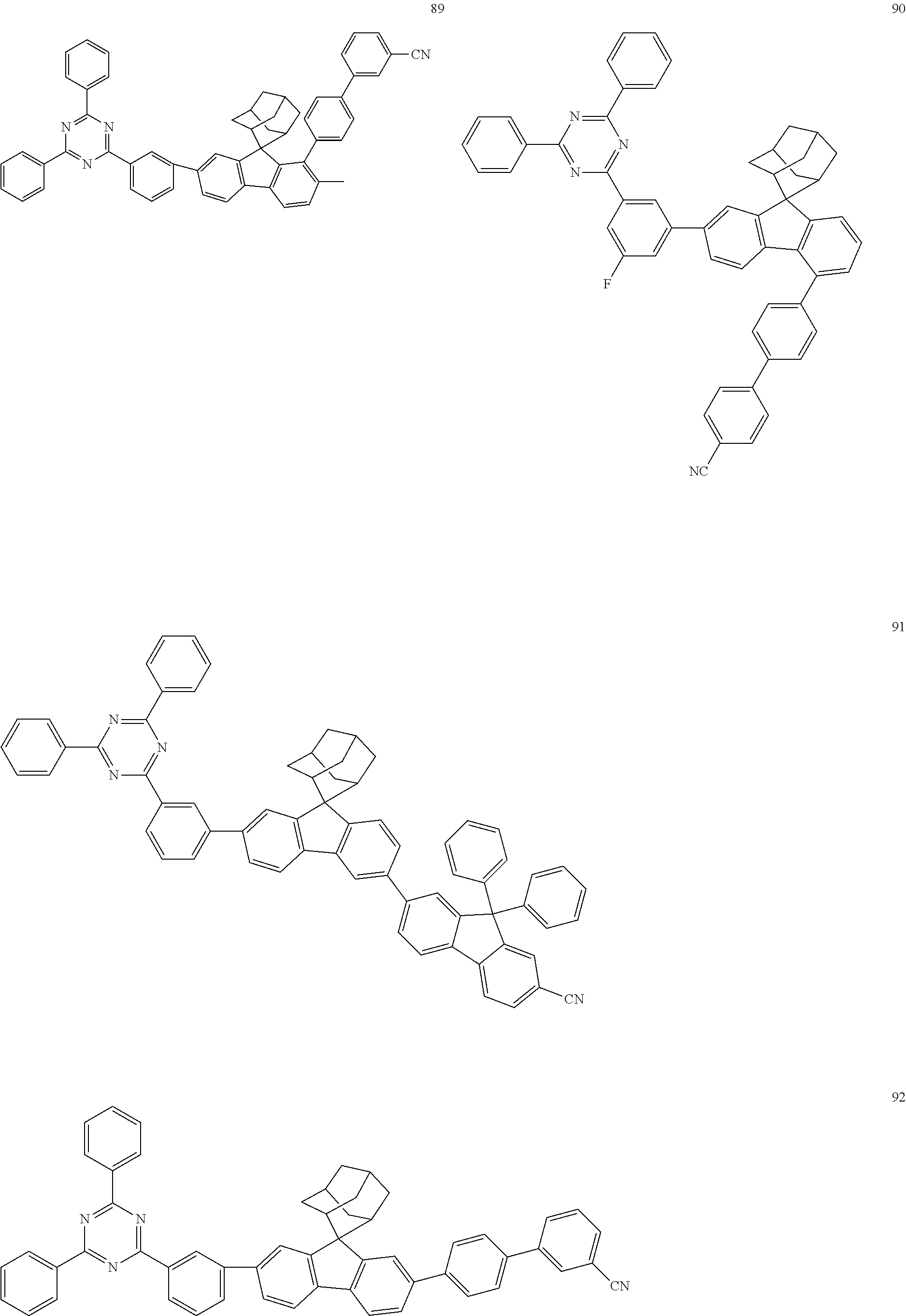 [SMILES: Cc1ccc2c(c1-c1ccc(-c3cccc(C#N)c3)cc1)C1(c3cc(-c4cccc(-c5nc(-c6ccccc6)nc(-c6ccccc6)n5)c4)ccc3-2)C2CC3CC(C2)CC1C3 ; N#Cc1ccc(-c2ccc(-c3cccc4c3-c3ccc(-c5cc(F)cc(-c6nc(-c7ccccc7)nc(-c7ccccc7)n6)c5)cc3C43C4CC5CC(C4)CC3C5)cc2)cc1 ; N#Cc1ccc2c(c1)C(c1ccccc1)(c1ccccc1)c1cc(-c3ccc4c(c3)-c3ccc(-c5cccc(-c6nc(-c7ccccc7)nc(-c7ccccc7)n6)c5)cc3C43C4CC5CC(C4)CC3C5)ccc1-2 ; N#Cc1cccc(-c2ccc(-c3ccc4c(c3)C3(c5cc(-c6cccc(-c7nc(-c8ccccc8)nc(-c8ccccc8)n7)c6)ccc5-4)C4CC5CC(C4)CC3C5)cc2)c1]

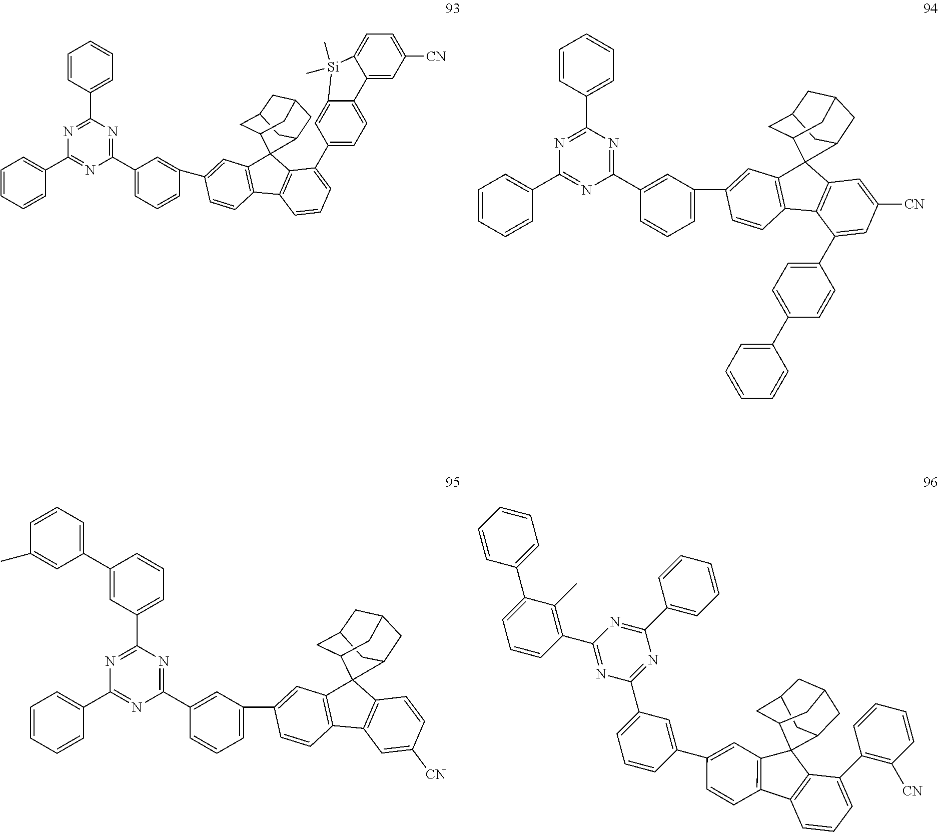 [SMILES: C[Si]1(C)c2ccc(C#N)cc2-c2ccc(-c3cccc4c3C3(c5cc(-c6cccc(-c7nc(-c8ccccc8)nc(-c8ccccc8)n7)c6)ccc5-4)C4CC5CC(C4)CC3C5)cc21 ; N#Cc1cc(-c2ccc(-c3ccccc3)cc2)c2c(c1)C1(c3cc(-c4cccc(-c5nc(-c6ccccc6)nc(-c6ccccc6)n5)c4)ccc3-2)C2CC3CC(C2)CC1C3 ; Cc1cccc(-c2cccc(-c3nc(-c4ccccc4)nc(-c4cccc(-c5ccc6c(c5)C5(c7ccc(C#N)cc7-6)C6CC7CC(C6)CC5C7)c4)n3)c2)c1 ; Cc1c(-c2ccccc2)cccc1-c1nc(-c2ccccc2)nc(-c2cccc(-c3ccc4c(c3)C3(c5c(-c6ccccc6C#N)cccc5-4)C4CC5CC(C4)CC3C5)c2)n1]

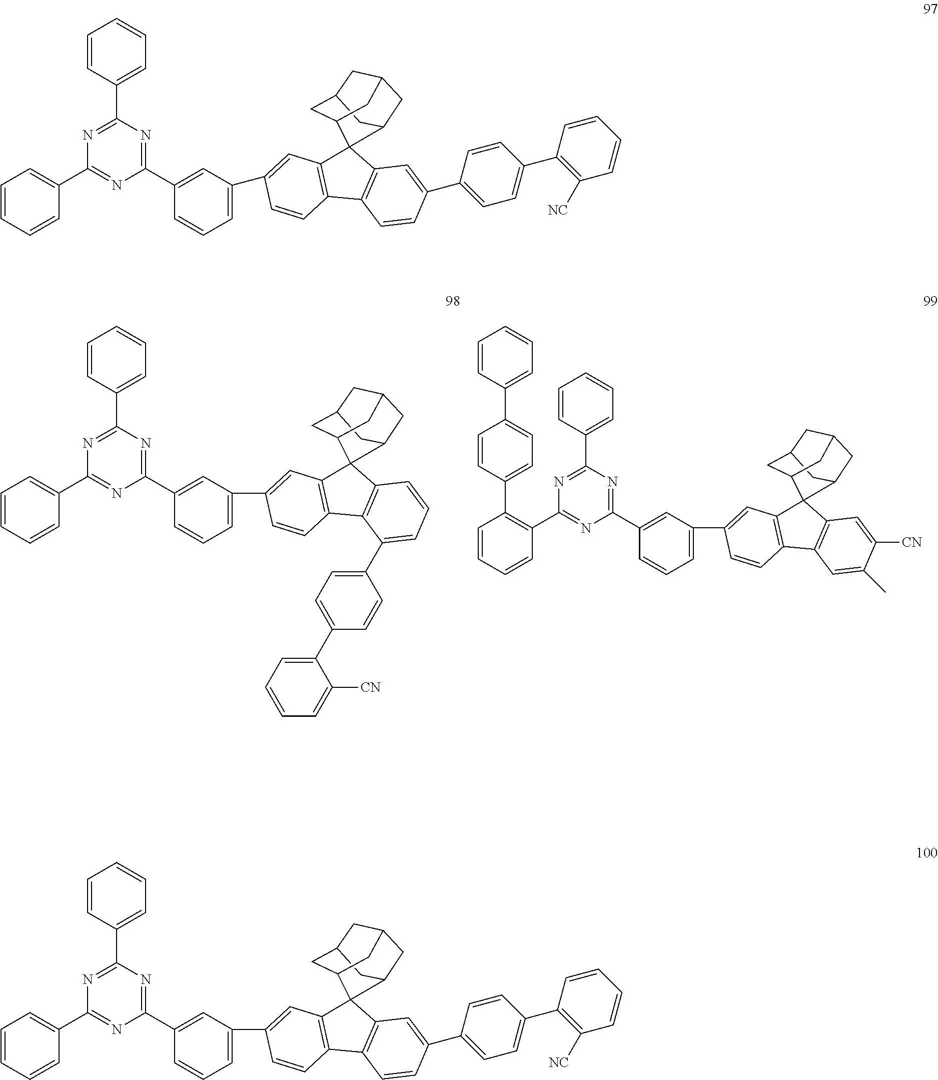 [SMILES: N#Cc1ccccc1-c1ccc(-c2ccc3c(c2)C2(c4cc(-c5cccc(-c6nc(-c7ccccc7)nc(-c7ccccc7)n6)c5)ccc4-3)C3CC4CC(C3)CC2C4)cc1 ; N#Cc1ccccc1-c1ccc(-c2cccc3c2-c2ccc(-c4cccc(-c5nc(-c6ccccc6)nc(-c6ccccc6)n5)c4)cc2C32C3CC4CC(C3)CC2C4)cc1 ; Cc1cc2c(cc1C#N)C1(c3cc(-c4cccc(-c5nc(-c6ccccc6)nc(-c6ccccc6-c6ccc(-c7ccccc7)cc6)n5)c4)ccc3-2)C2CC3CC(C2)CC1C3 ; N#Cc1ccccc1-c1ccc(-c2ccc3c(c2)C2(c4cc(-c5cccc(-c6nc(-c7ccccc7)nc(-c7ccccc7)n6)c5)ccc4-3)C3CC4CC(C3)CC2C4)cc1]

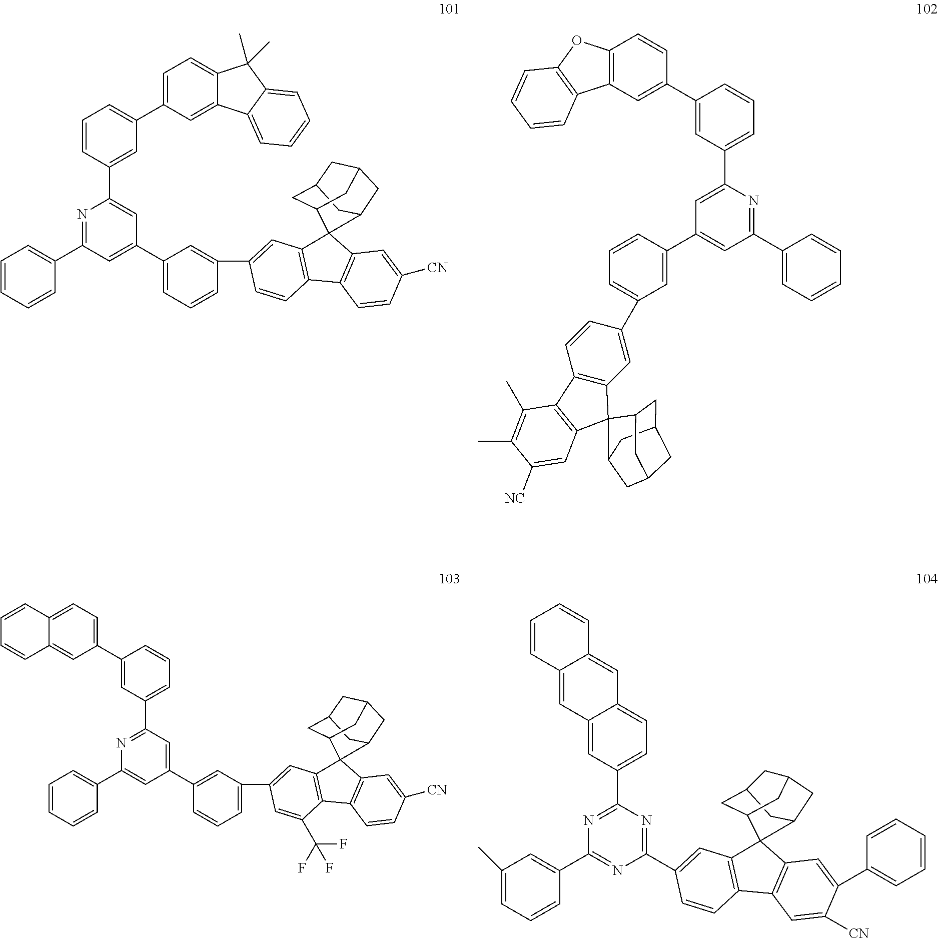 [SMILES: CC1(C)c2ccccc2-c2cc(-c3cccc(-c4cc(-c5cccc(-c6ccc7c(c6)C6(c8cc(C#N)ccc8-7)C7CC8CC(C7)CC6C8)c5)cc(-c5ccccc5)n4)c3)ccc21 ; Cc1c(C#N)cc2c(c1C)-c1ccc(-c3cccc(-c4cc(-c5ccccc5)nc(-c5cccc(-c6ccc7oc8ccccc8c7c6)c5)c4)c3)cc1C21C2CC3CC(C2)CC1C3 ; N#Cc1ccc2c(c1)C1(c3cc(-c4cccc(-c5cc(-c6ccccc6)nc(-c6cccc(-c7ccc8ccccc8c7)c6)c5)c4)cc(C(F)(F)F)c3-2)C2CC3CC(C2)CC1C3 ; Cc1cccc(-c2nc(-c3ccc4c(c3)C3(c5cc(-c6ccccc6)c(C#N)cc5-4)C4CC5CC(C4)CC3C5)nc(-c3ccc4cc5ccccc5cc4c3)n2)c1]

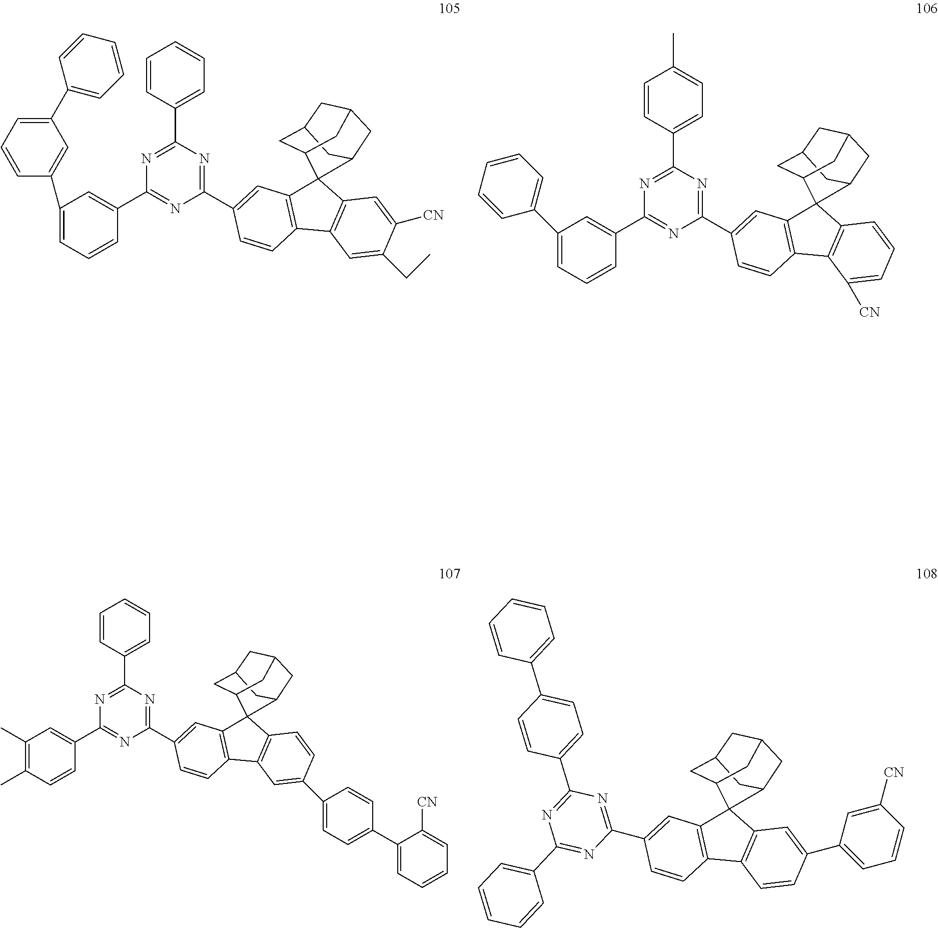

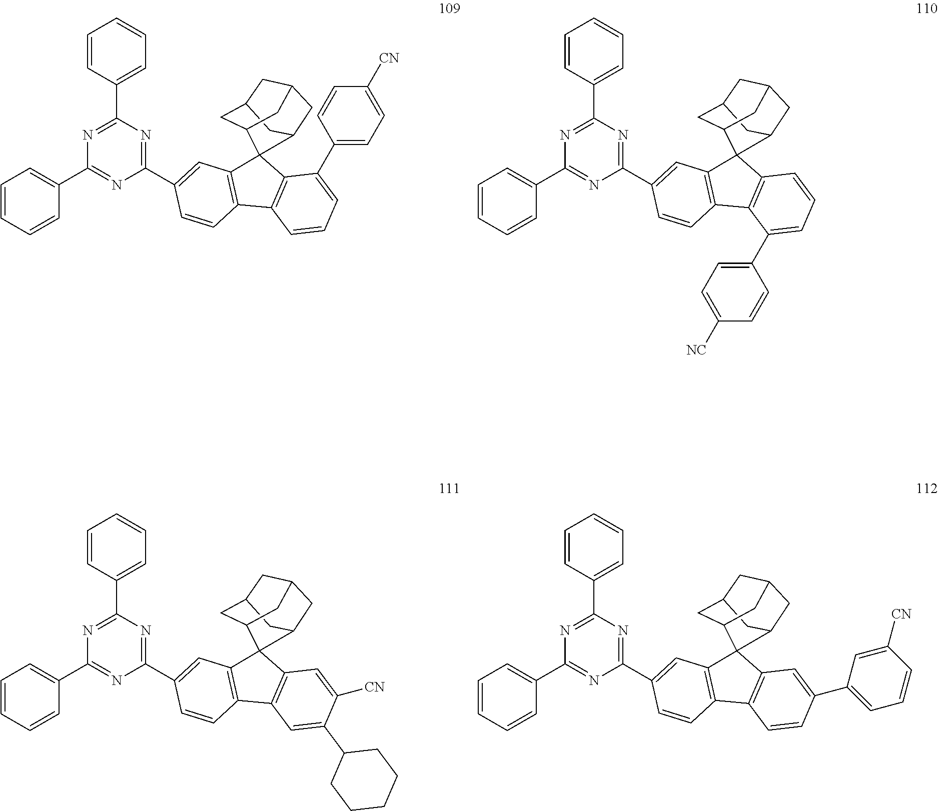 [SMILES: N#Cc1ccc(-c2cccc3c2C2(c4cc(-c5nc(-c6ccccc6)nc(-c6ccccc6)n5)ccc4-3)C3CC4CC(C3)CC2C4)cc1 ; N#Cc1ccc(-c2cccc3c2-c2ccc(-c4nc(-c5ccccc5)nc(-c5ccccc5)n4)cc2C32C3CC4CC(C3)CC2C4)cc1 ; N#Cc1cc2c(cc1C1CCCCC1)-c1ccc(-c3nc(-c4ccccc4)nc(-c4ccccc4)n3)cc1C21C2CC3CC(C2)CC1C3 ; N#Cc1cccc(-c2ccc3c(c2)C2(c4cc(-c5nc(-c6ccccc6)nc(-c6ccccc6)n5)ccc4-3)C3CC4CC(C3)CC2C4)c1]

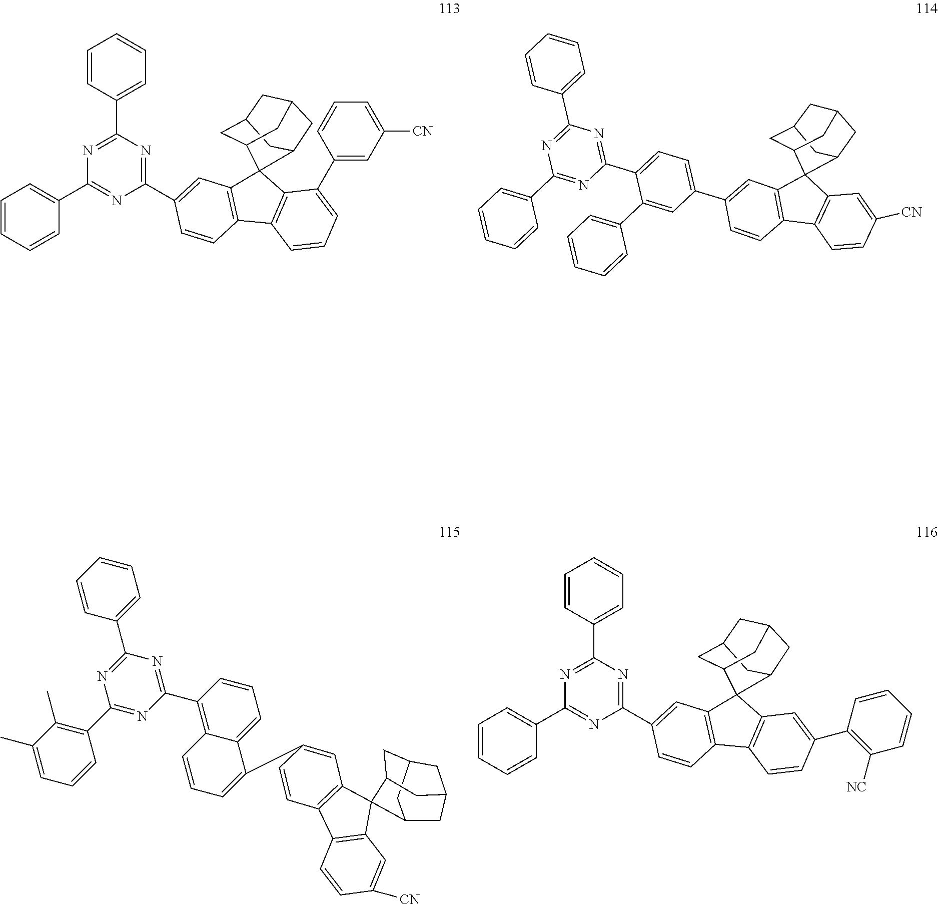

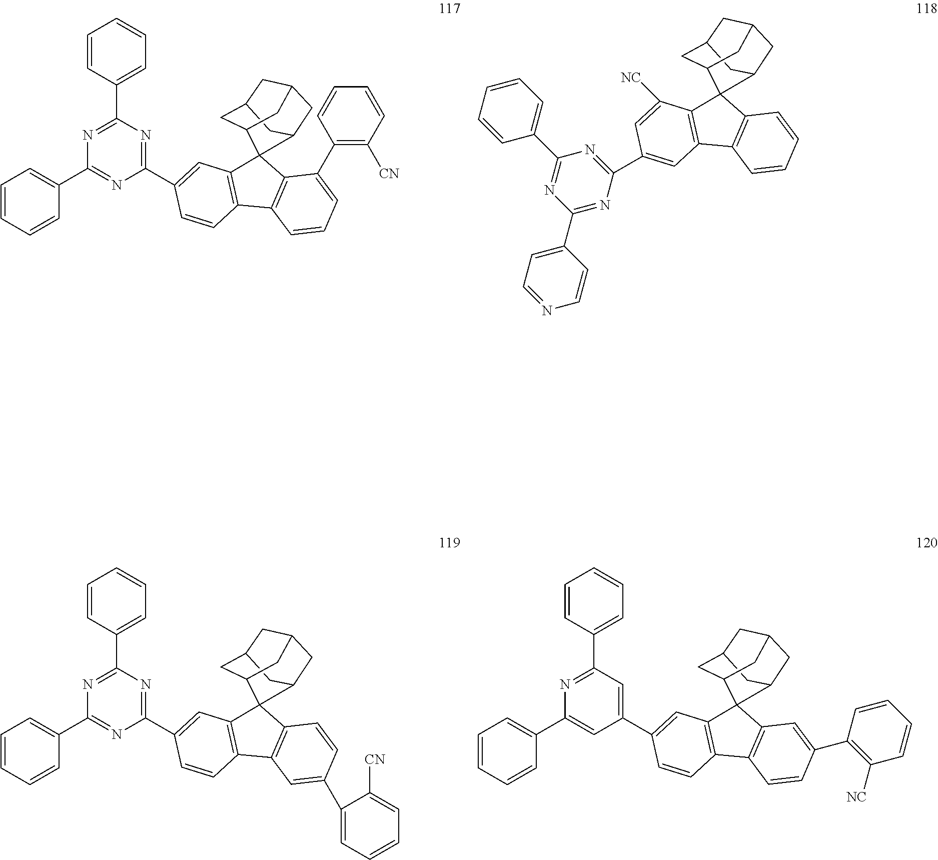 [SMILES: N#Cc1ccccc1-c1cccc2c1C1(c3cc(-c4nc(-c5ccccc5)nc(-c5ccccc5)n4)ccc3-2)C2CC3CC(C2)CC1C3 ; N#Cc1cc(-c2nc(-c3ccccc3)nc(-c3ccncc3)n2)cc2c1C1(c3ccccc3-2)C2CC3CC(C2)CC1C3 ; N#Cc1ccccc1-c1ccc2c(c1)-c1ccc(-c3nc(-c4ccccc4)nc(-c4ccccc4)n3)cc1C21C2CC3CC(C2)CC1C3 ; N#Cc1ccccc1-c1ccc2c(c1)C1(c3cc(-c4cc(-c5ccccc5)nc(-c5ccccc5)c4)ccc3-2)C2CC3CC(C2)CC1C3]

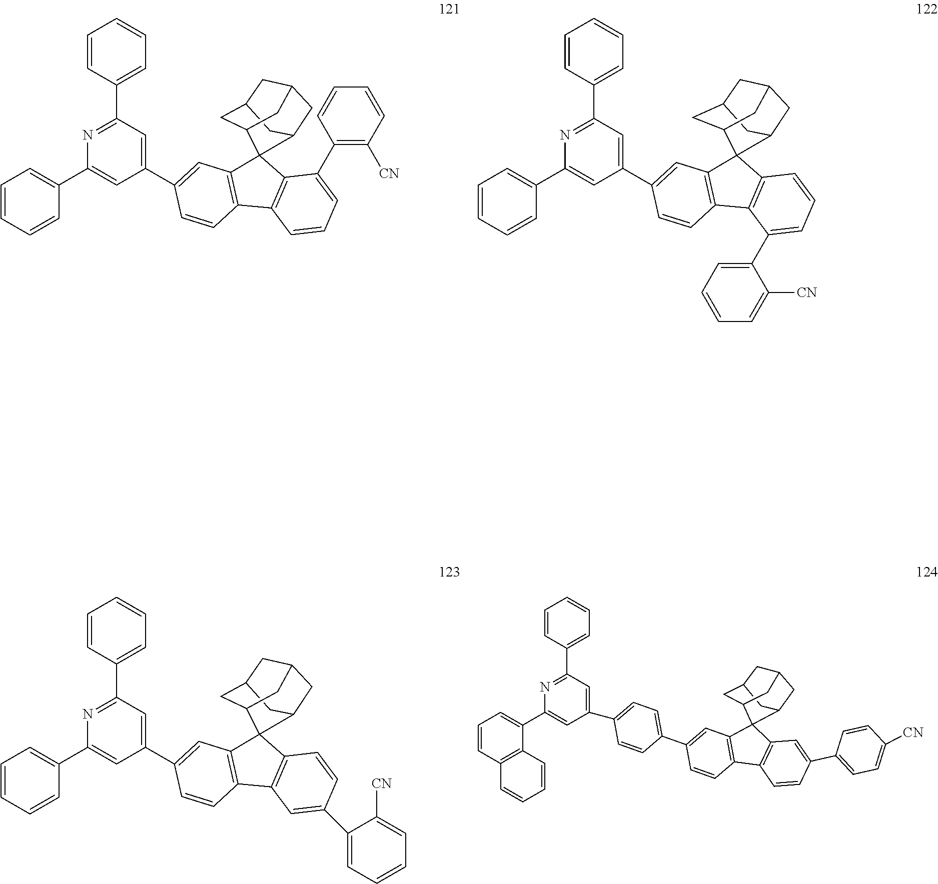 [SMILES: N#Cc1ccccc1-c1cccc2c1C1(c3cc(-c4cc(-c5ccccc5)nc(-c5ccccc5)c4)ccc3-2)C2CC3CC(C2)CC1C3 ; N#Cc1ccccc1-c1cccc2c1-c1ccc(-c3cc(-c4ccccc4)nc(-c4ccccc4)c3)cc1C21C2CC3CC(C2)CC1C3 ; N#Cc1ccccc1-c1ccc2c(c1)-c1ccc(-c3cc(-c4ccccc4)nc(-c4ccccc4)c3)cc1C21C2CC3CC(C2)CC1C3 ; N#Cc1ccc(-c2ccc3c(c2)C2(c4cc(-c5ccc(-c6cc(-c7ccccc7)nc(-c7cccc8ccccc78)c6)cc5)ccc4-3)C3CC4CC(C3)CC2C4)cc1]

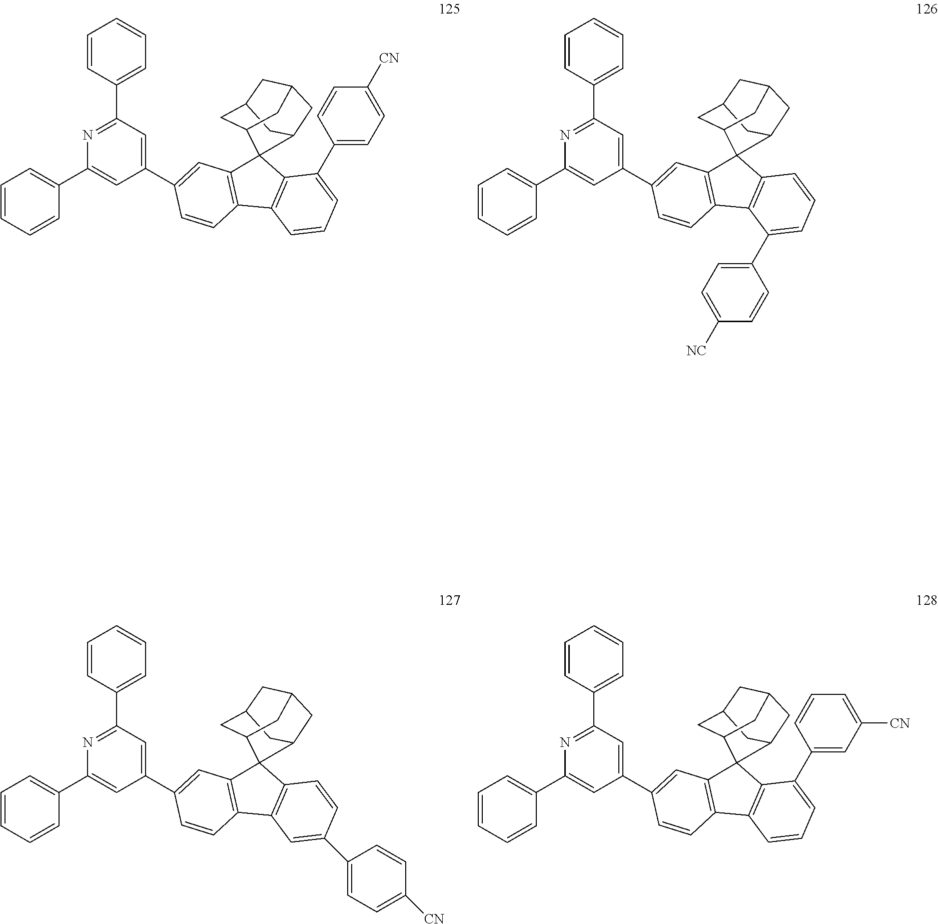 [SMILES: N#Cc1ccc(-c2cccc3c2C2(c4cc(-c5cc(-c6ccccc6)nc(-c6ccccc6)c5)ccc4-3)C3CC4CC(C3)CC2C4)cc1 ; N#Cc1ccc(-c2cccc3c2-c2ccc(-c4cc(-c5ccccc5)nc(-c5ccccc5)c4)cc2C32C3CC4CC(C3)CC2C4)cc1 ; N#Cc1ccc(-c2ccc3c(c2)-c2ccc(-c4cc(-c5ccccc5)nc(-c5ccccc5)c4)cc2C32C3CC4CC(C3)CC2C4)cc1 ; N#Cc1cccc(-c2cccc3c2C2(c4cc(-c5cc(-c6ccccc6)nc(-c6ccccc6)c5)ccc4-3)C3CC4CC(C3)CC2C4)c1]

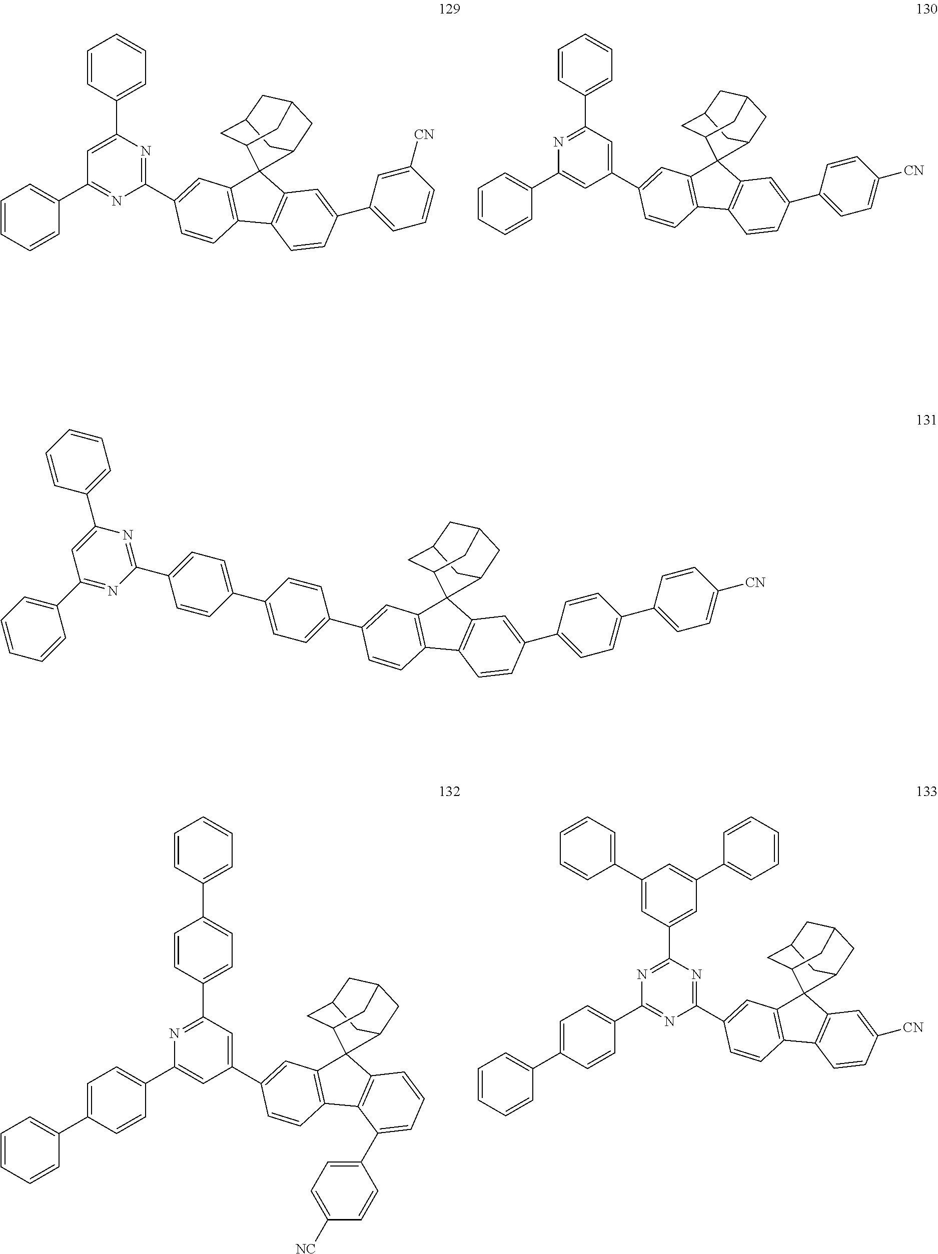

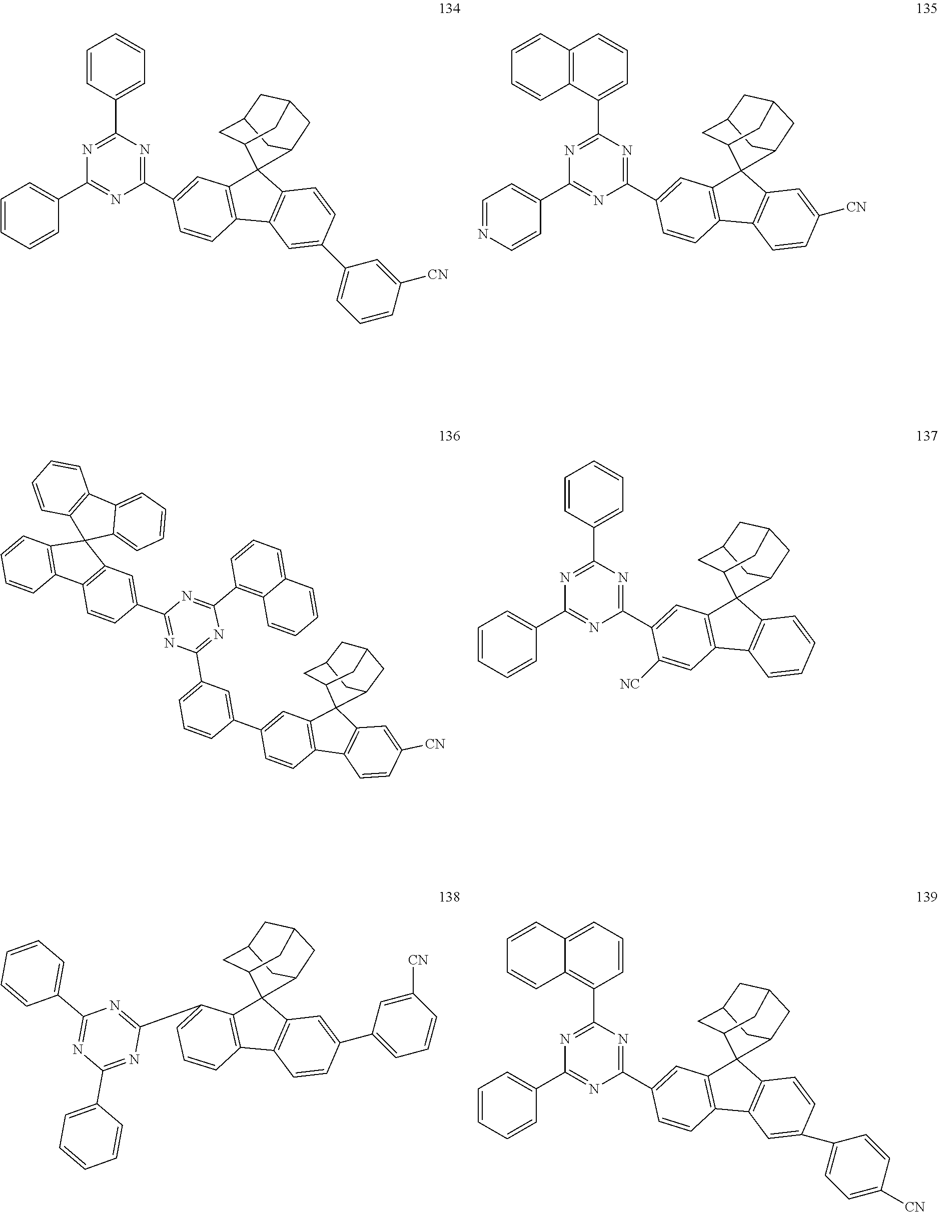

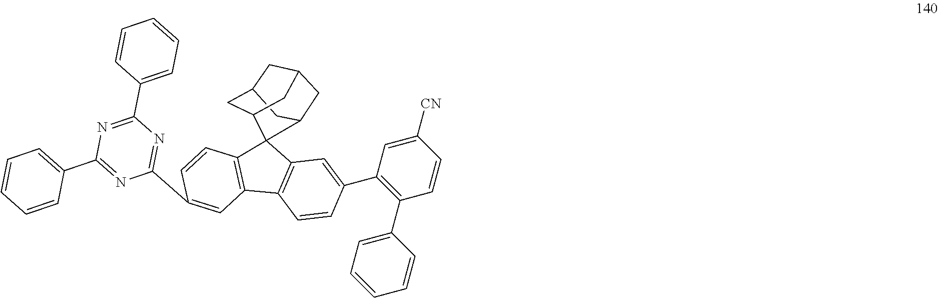 [SMILES: N#Cc1ccc(-c2ccccc2)c(-c2ccc3c(c2)C2(c4ccc(-c5nc(-c6ccccc6)nc(-c6ccccc6)n5)cc4-3)C3CC4CC(C3)CC2C4)c1]

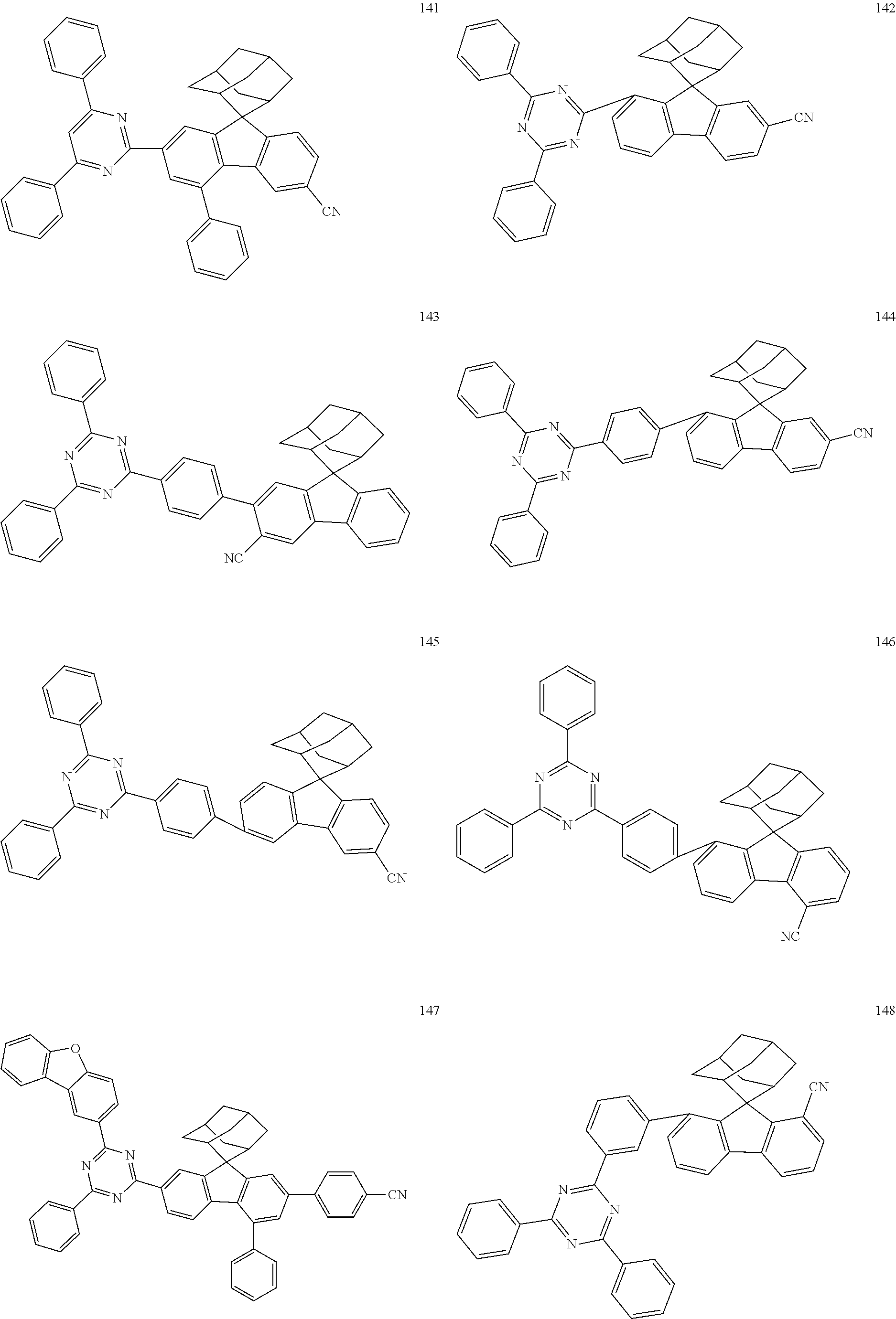

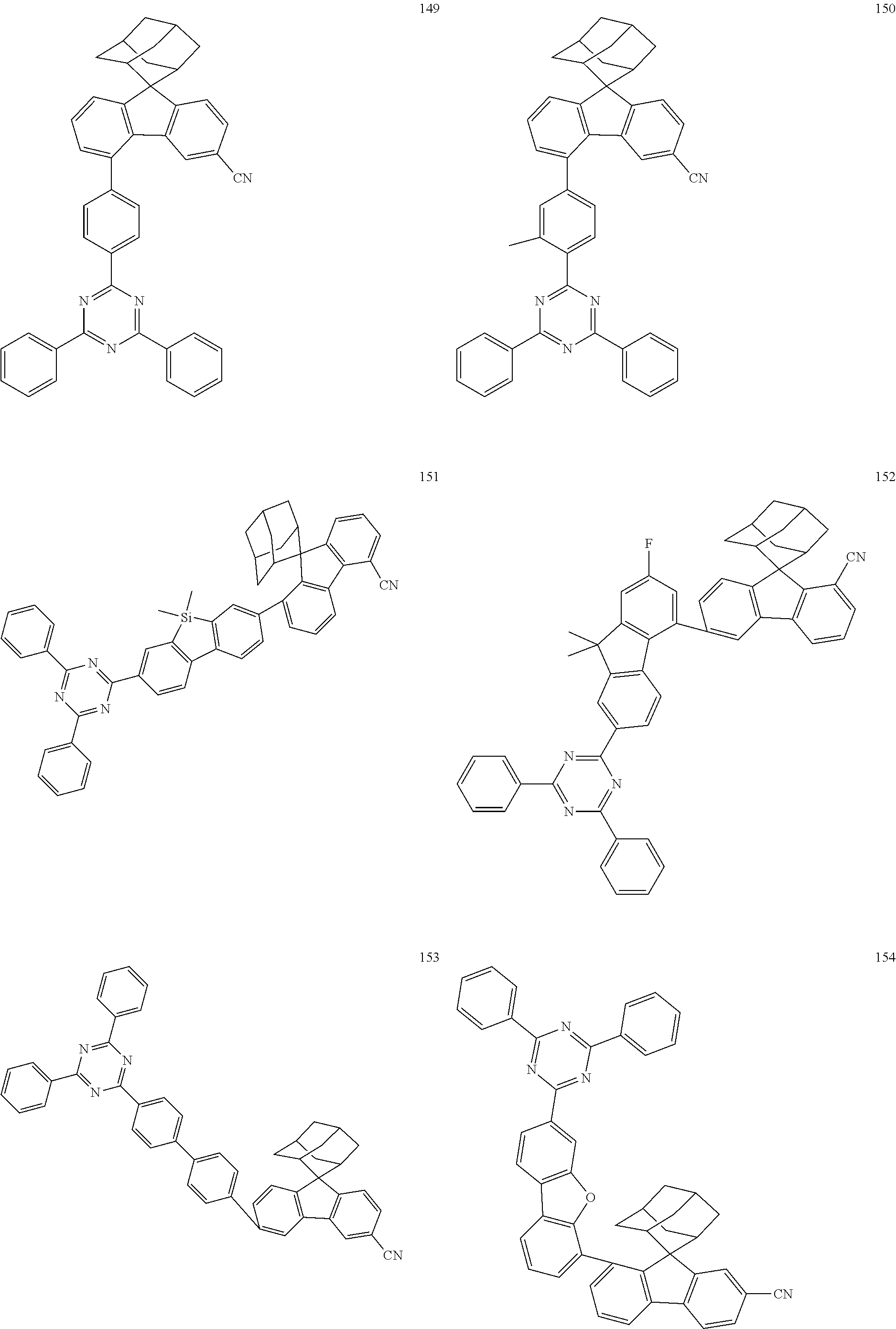

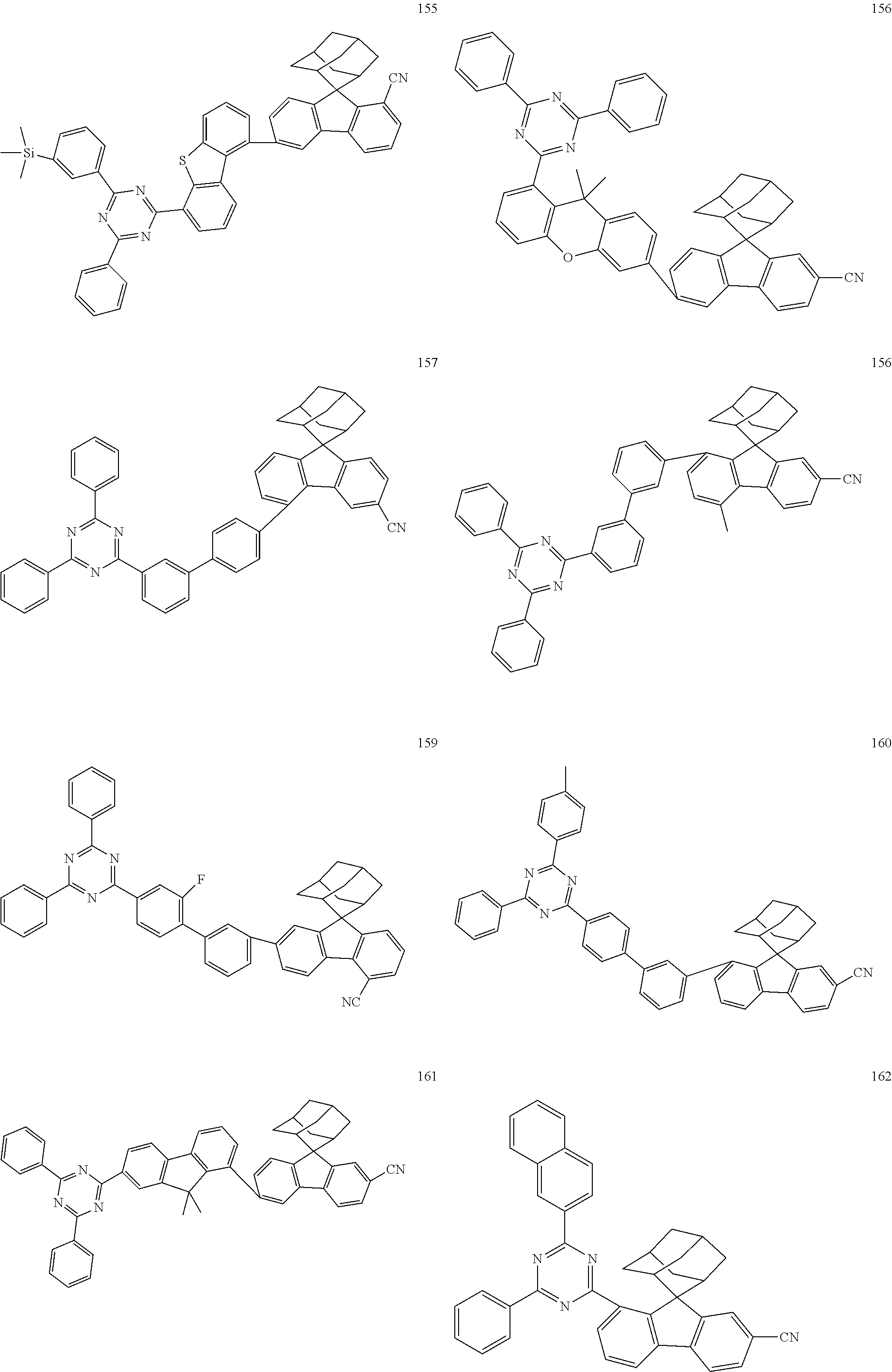

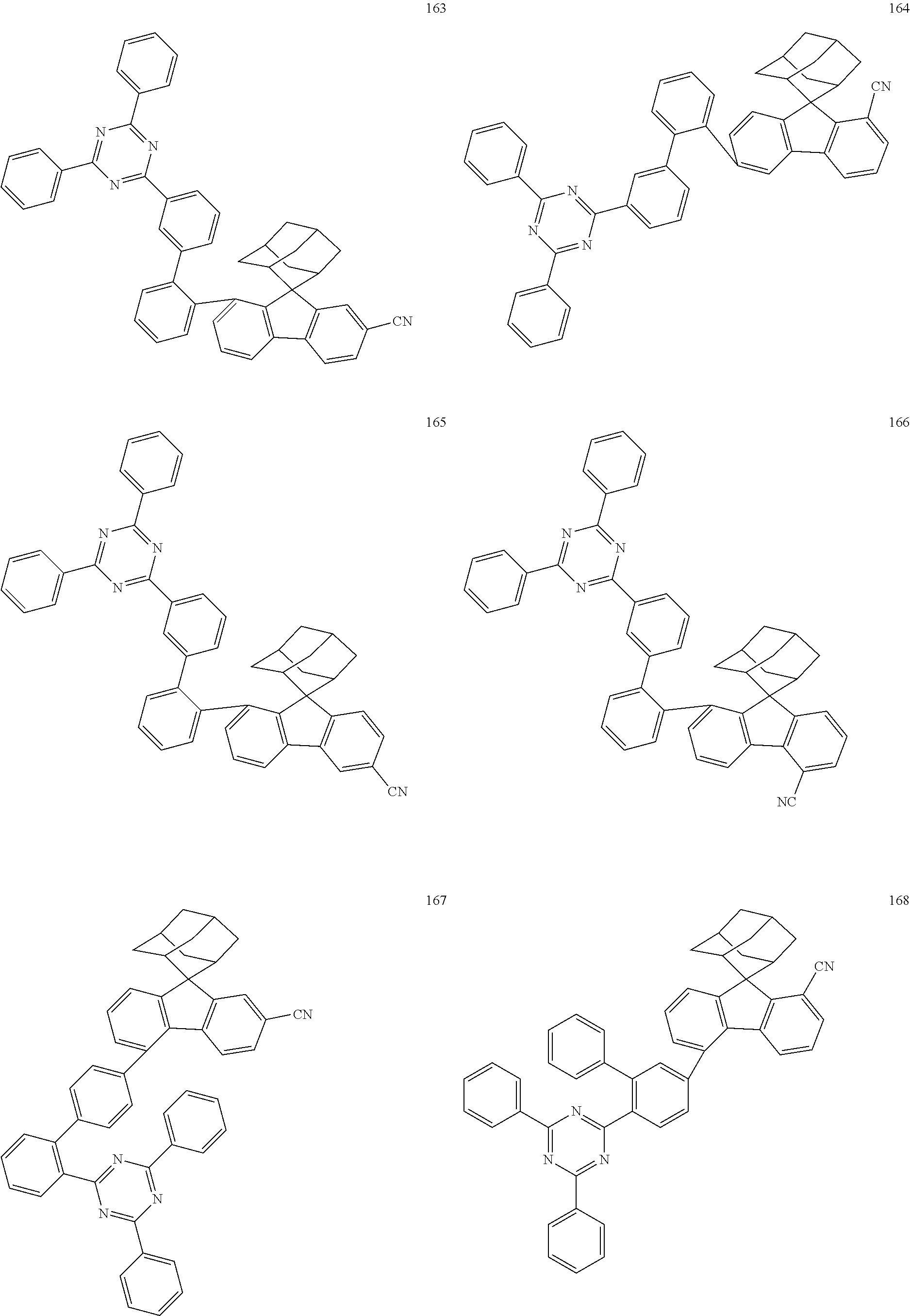

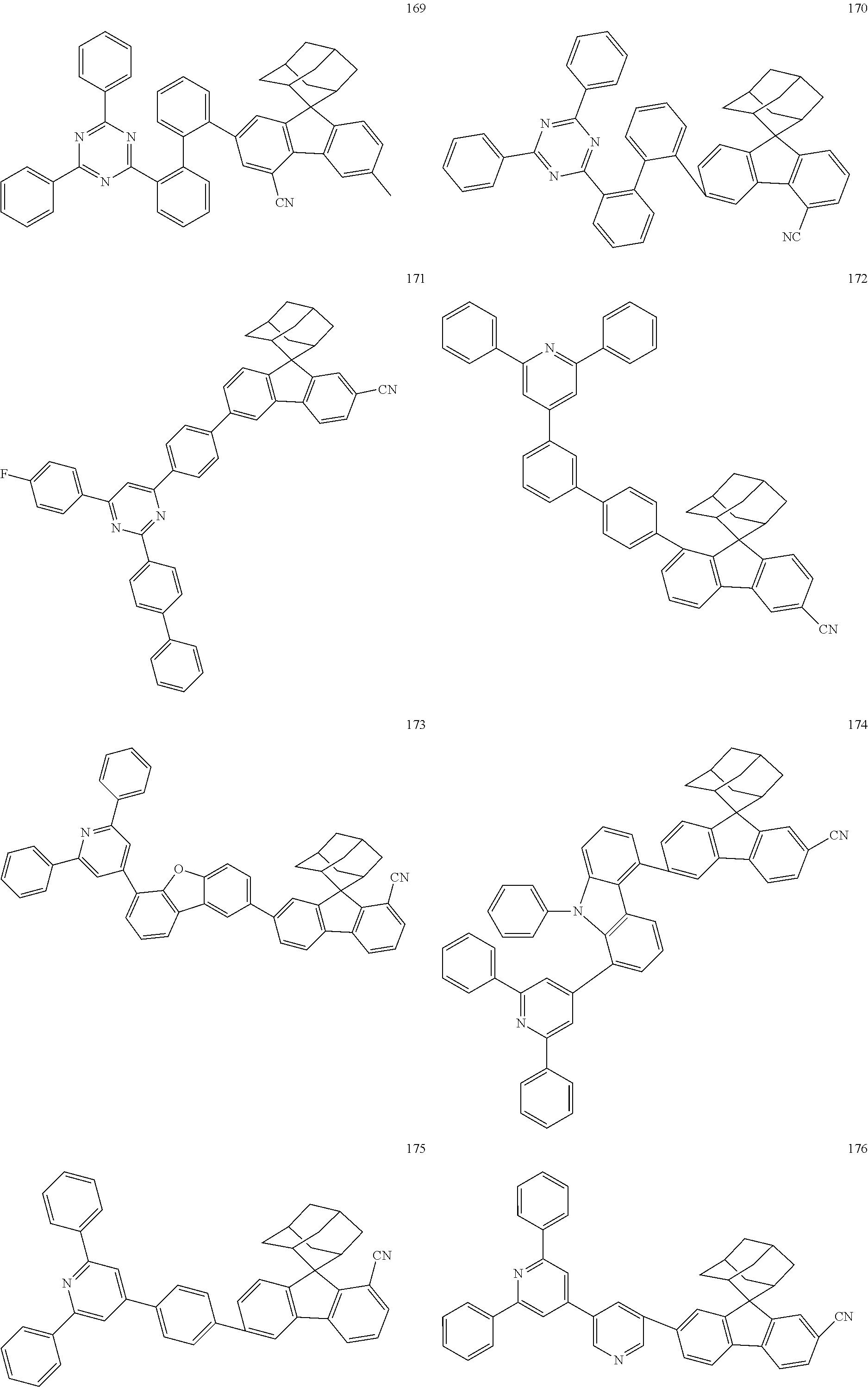

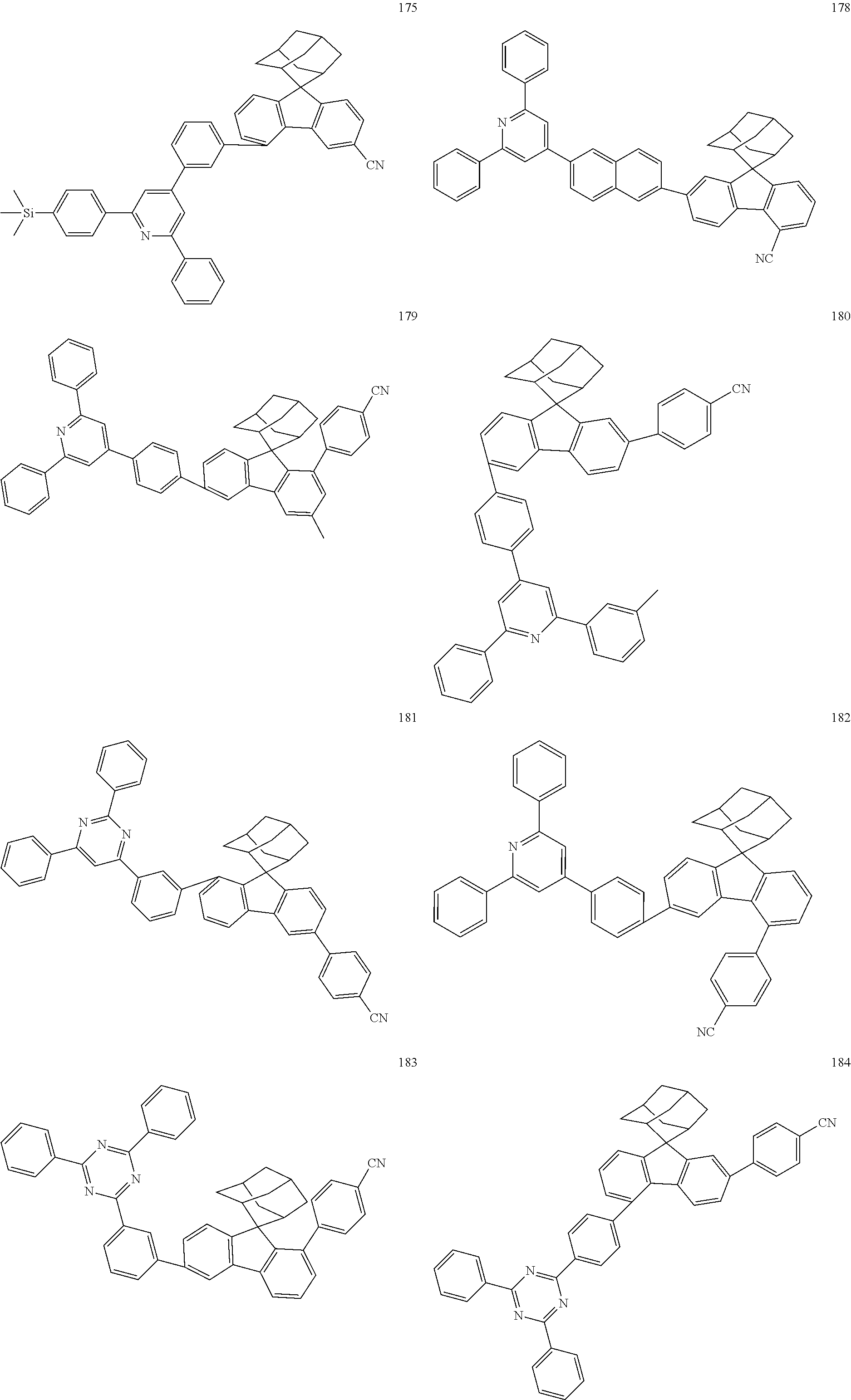

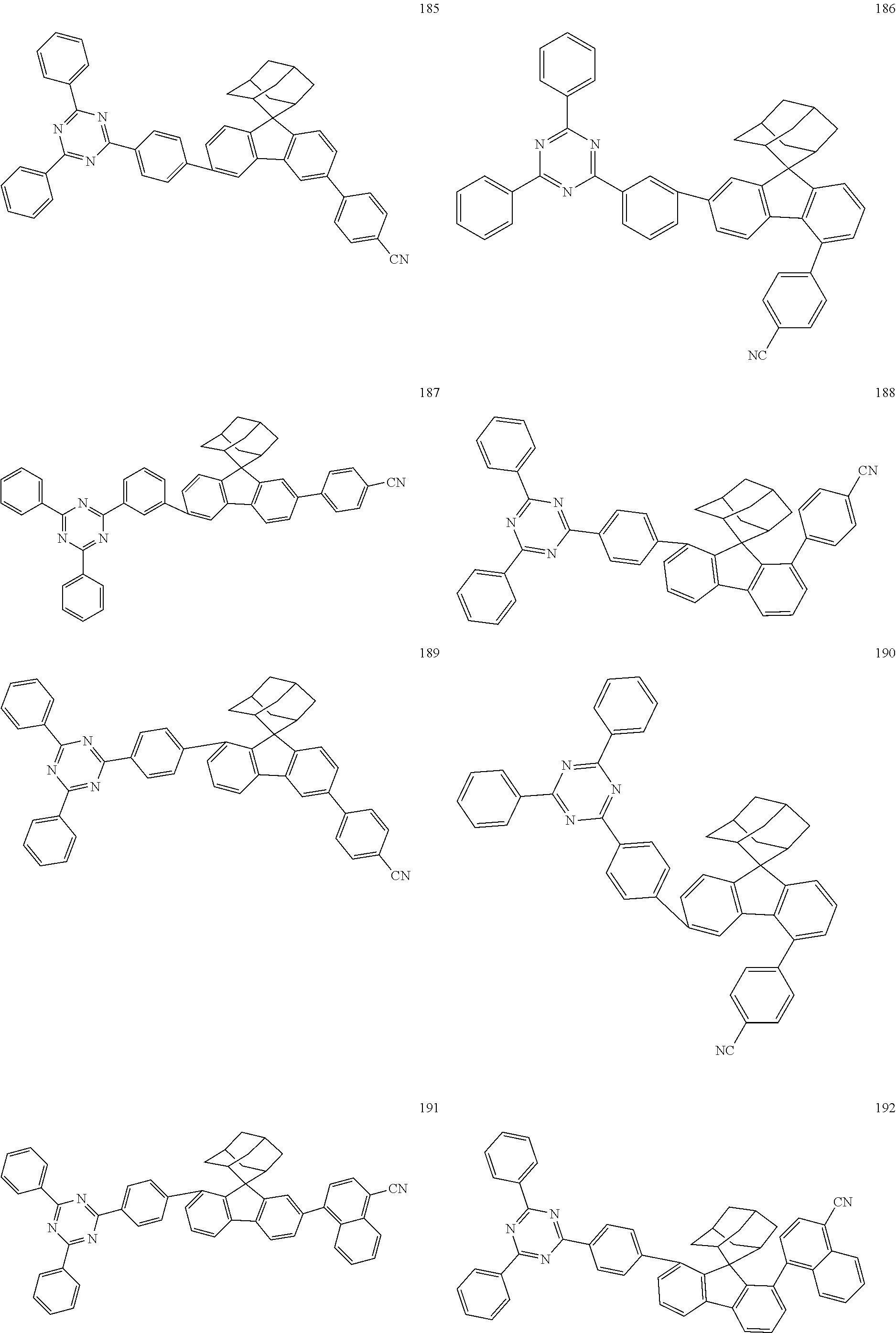

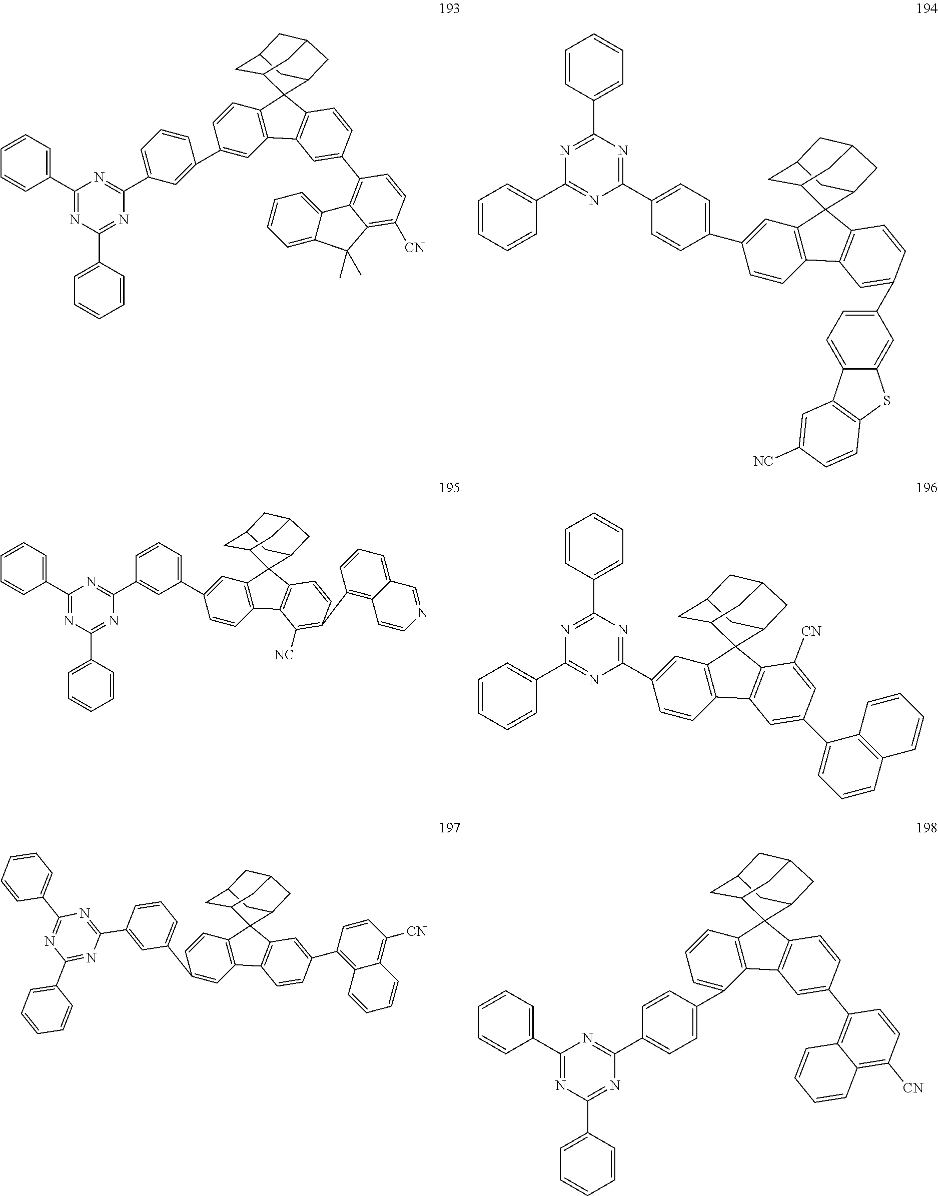

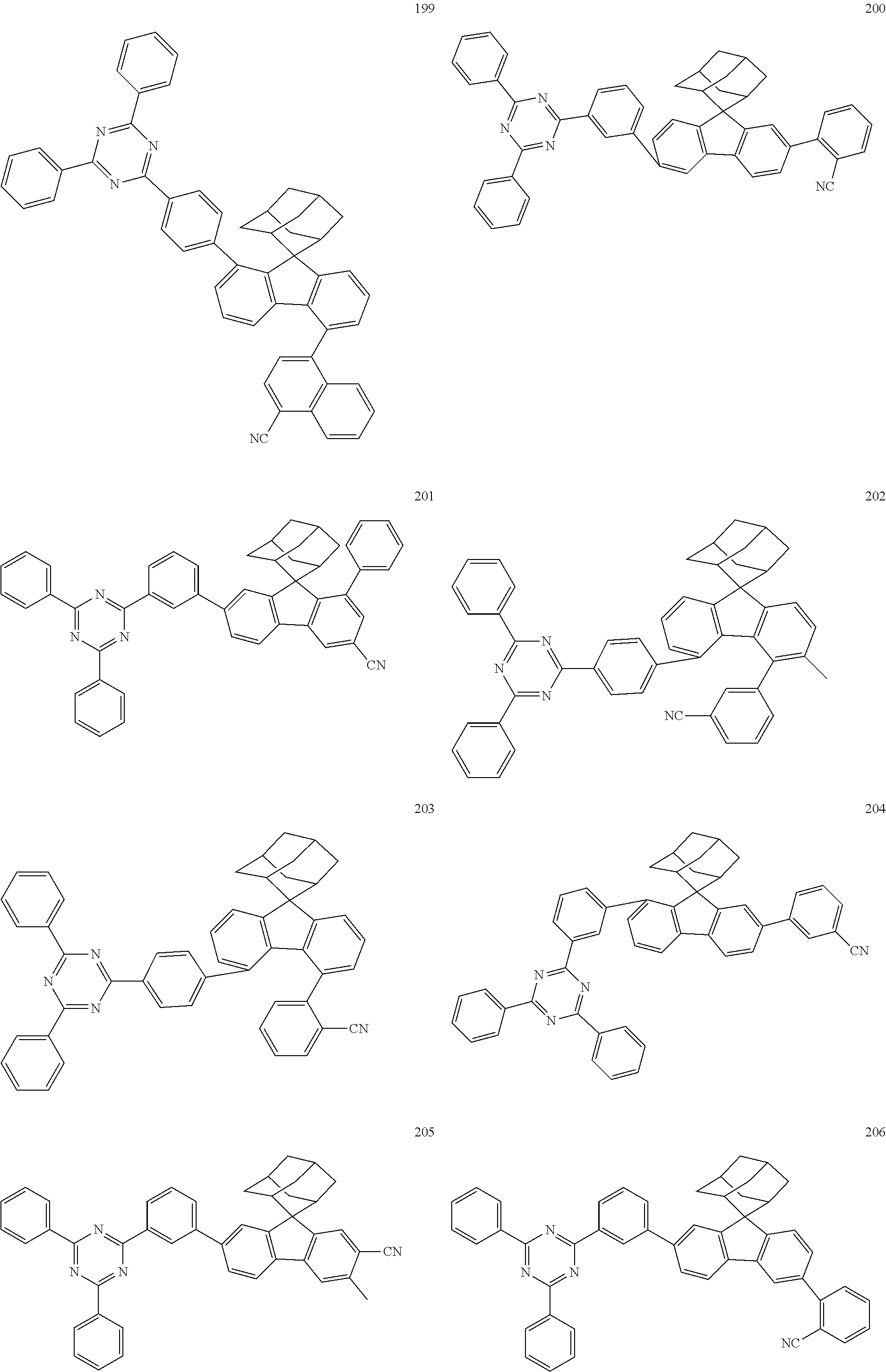

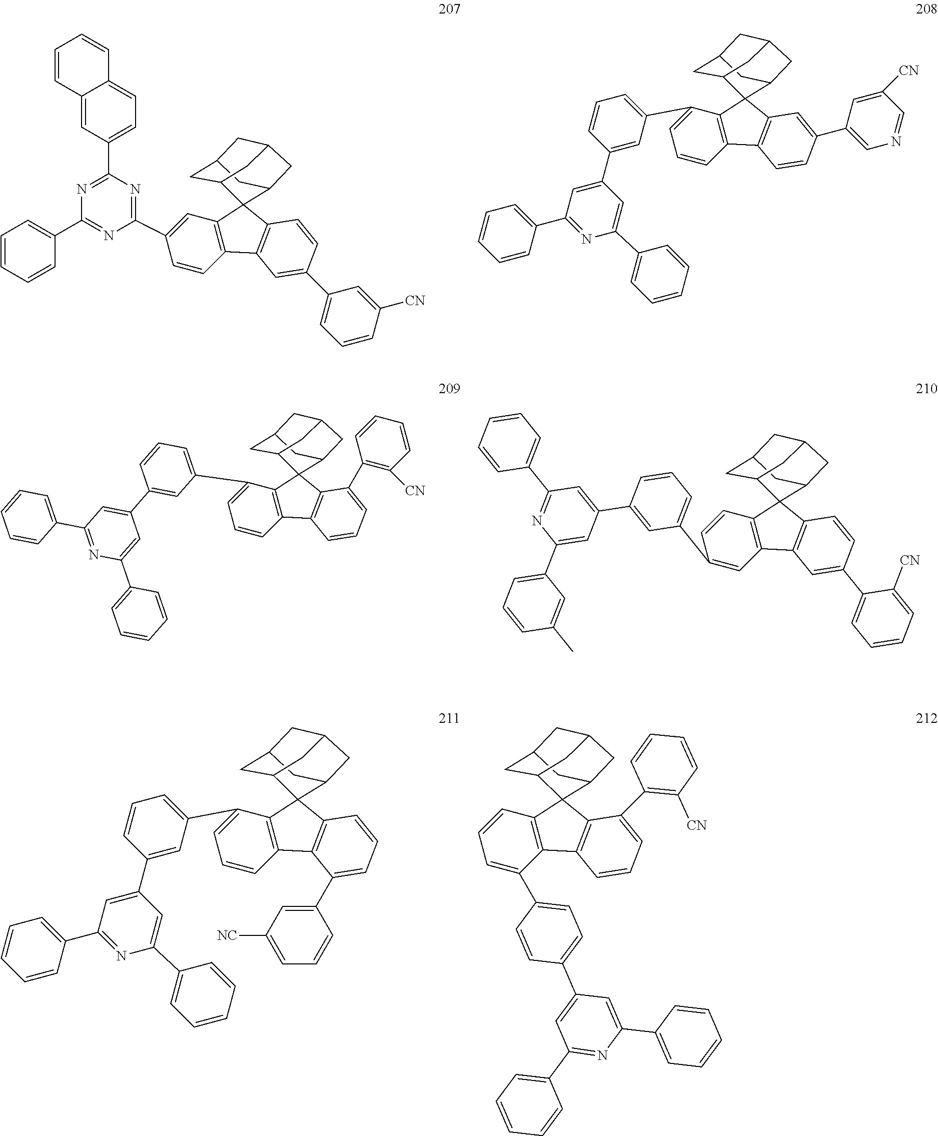

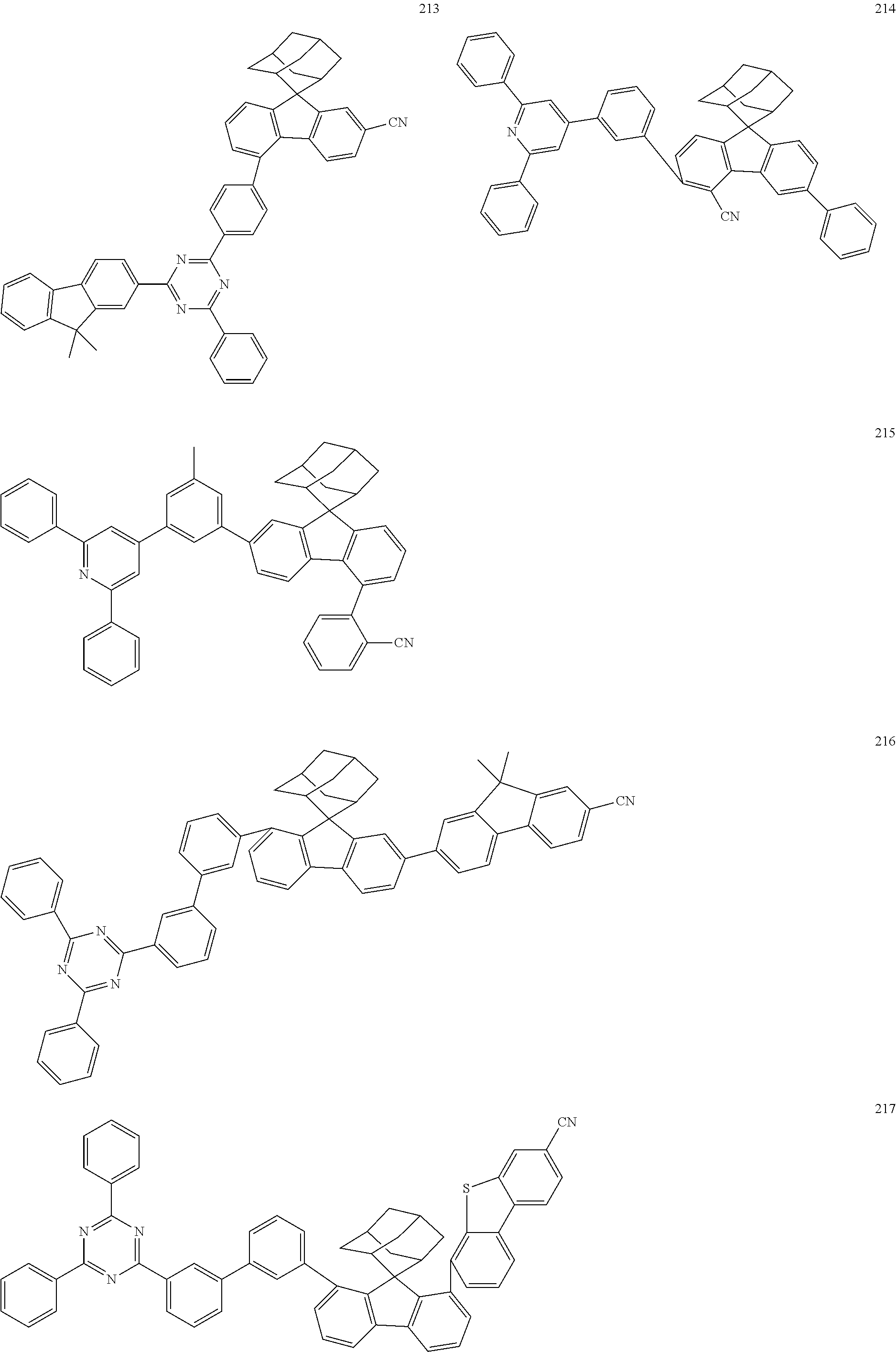

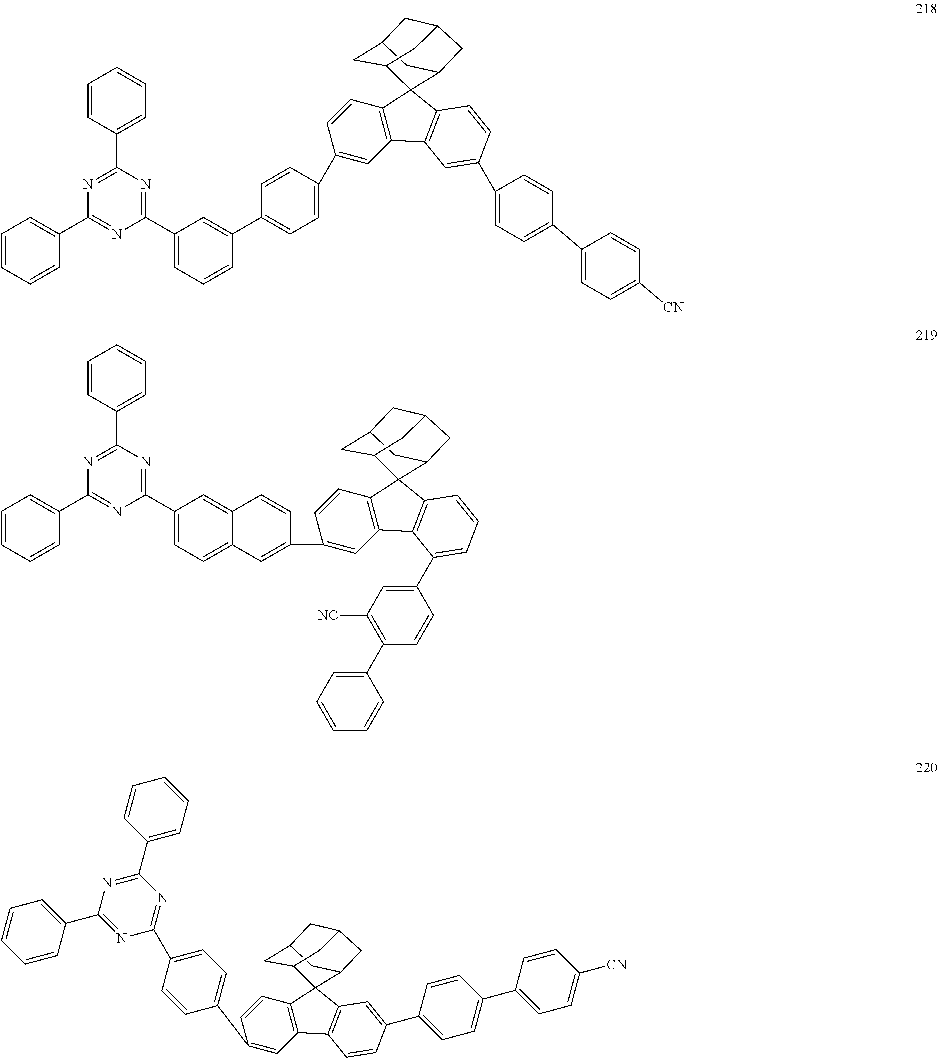 [SMILES: N#Cc1ccc(-c2ccc(-c3ccc4c(c3)-c3cc(-c5ccc(-c6cccc(-c7nc(-c8ccccc8)nc(-c8ccccc8)n7)c6)cc5)ccc3C43C4CC5CC(C4)CC3C5)cc2)cc1 ; N#Cc1cc(-c2cccc3c2-c2cc(-c4ccc5cc(-c6nc(-c7ccccc7)nc(-c7ccccc7)n6)ccc5c4)ccc2C32C3CC4CC(C3)CC2C4)ccc1-c1ccccc1 ; N#Cc1ccc(-c2ccc(-c3ccc4c(c3)C3(c5ccc(-c6ccc(-c7nc(-c8ccccc8)nc(-c8ccccc8)n7)cc6)cc5-4)C4CC5CC(C4)CC3C5)cc2)cc1]

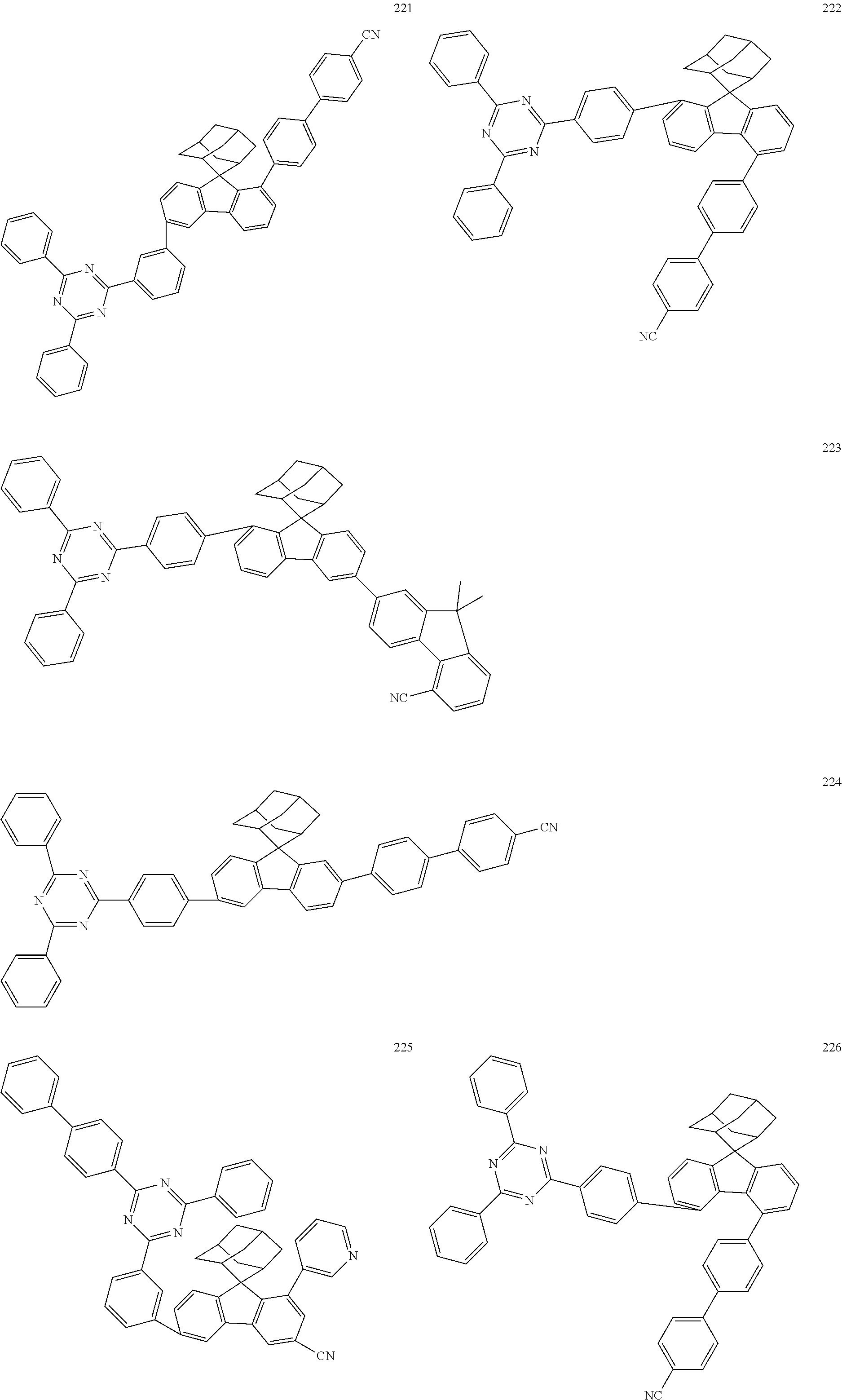

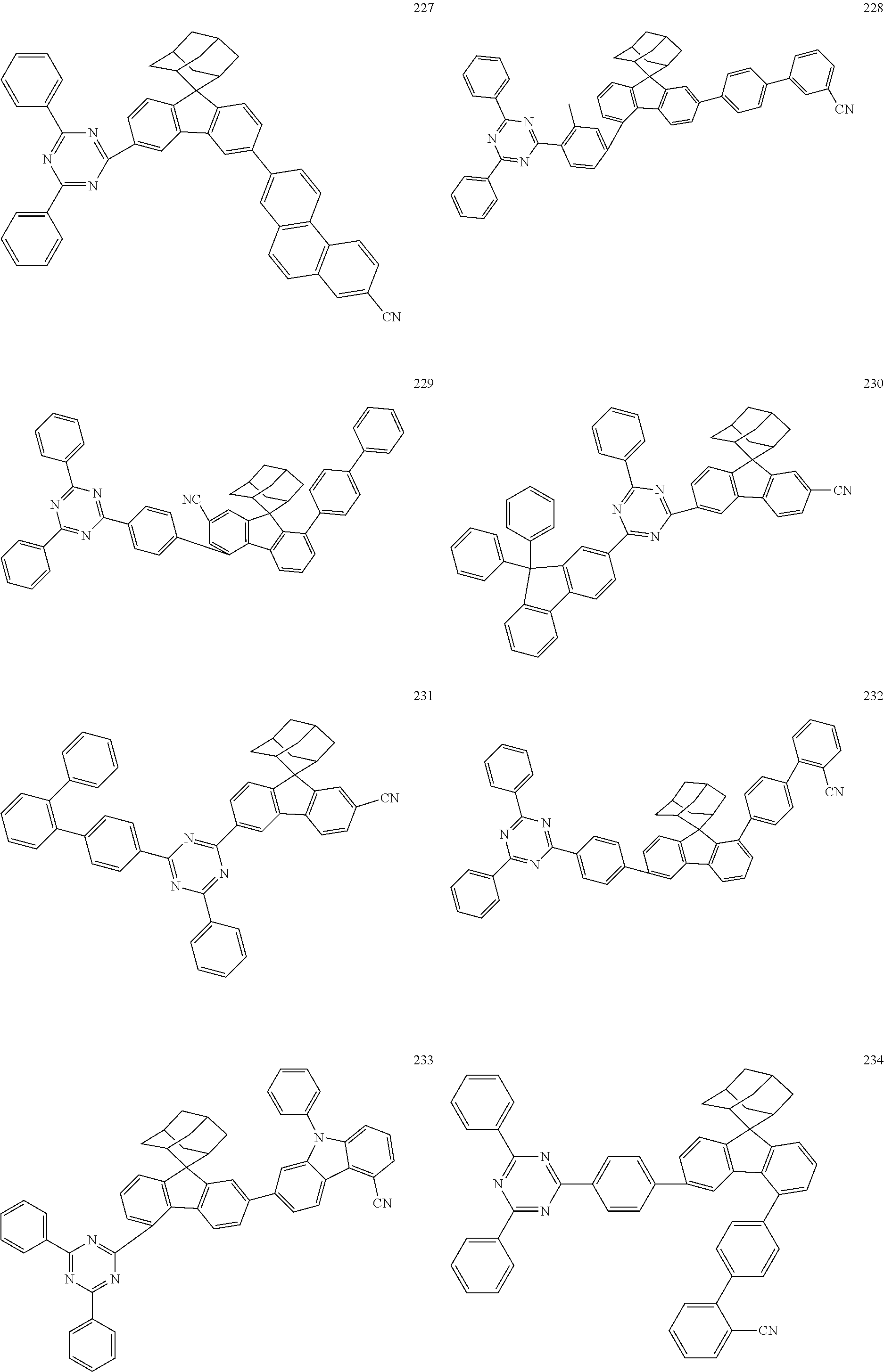

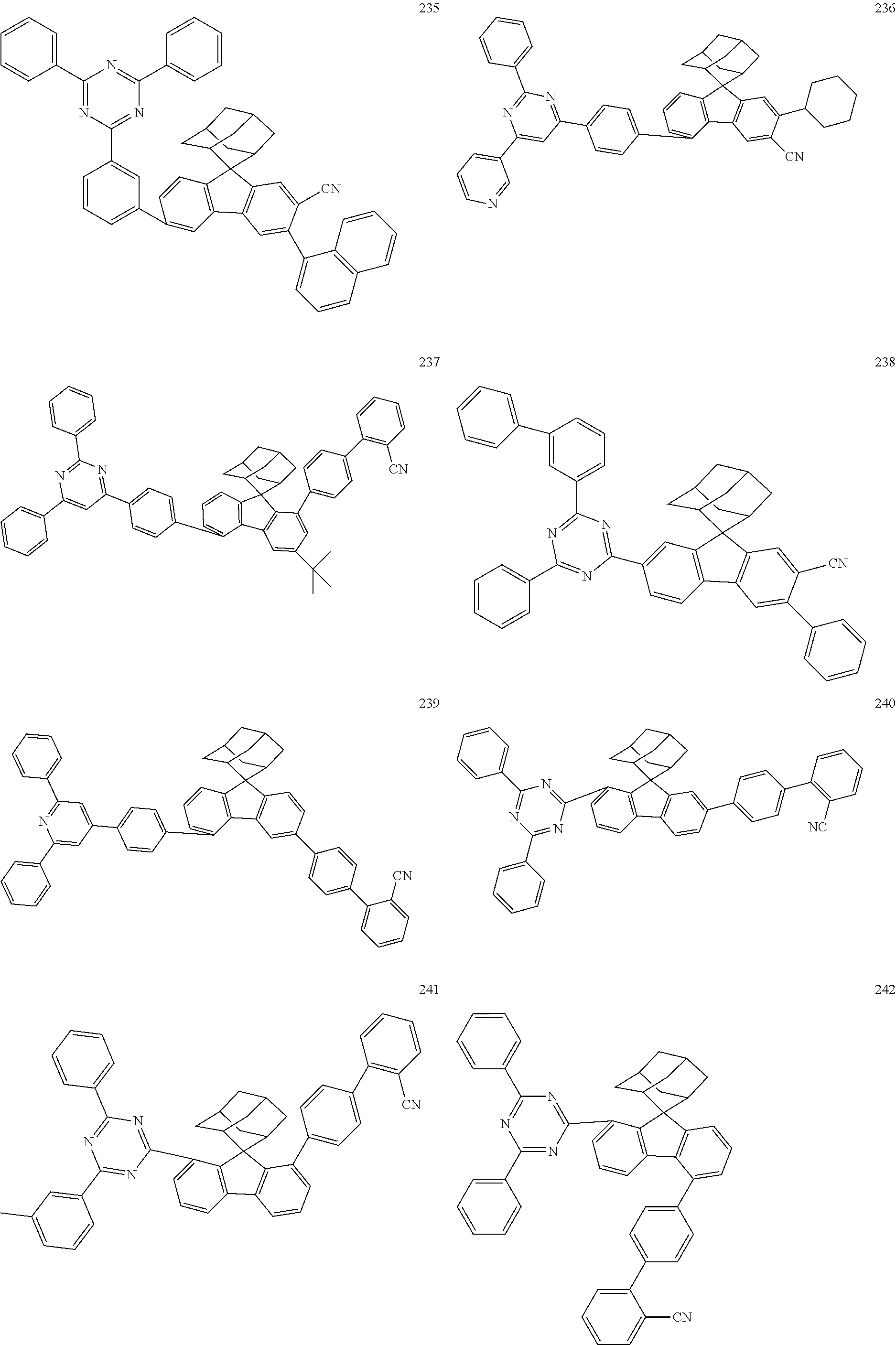

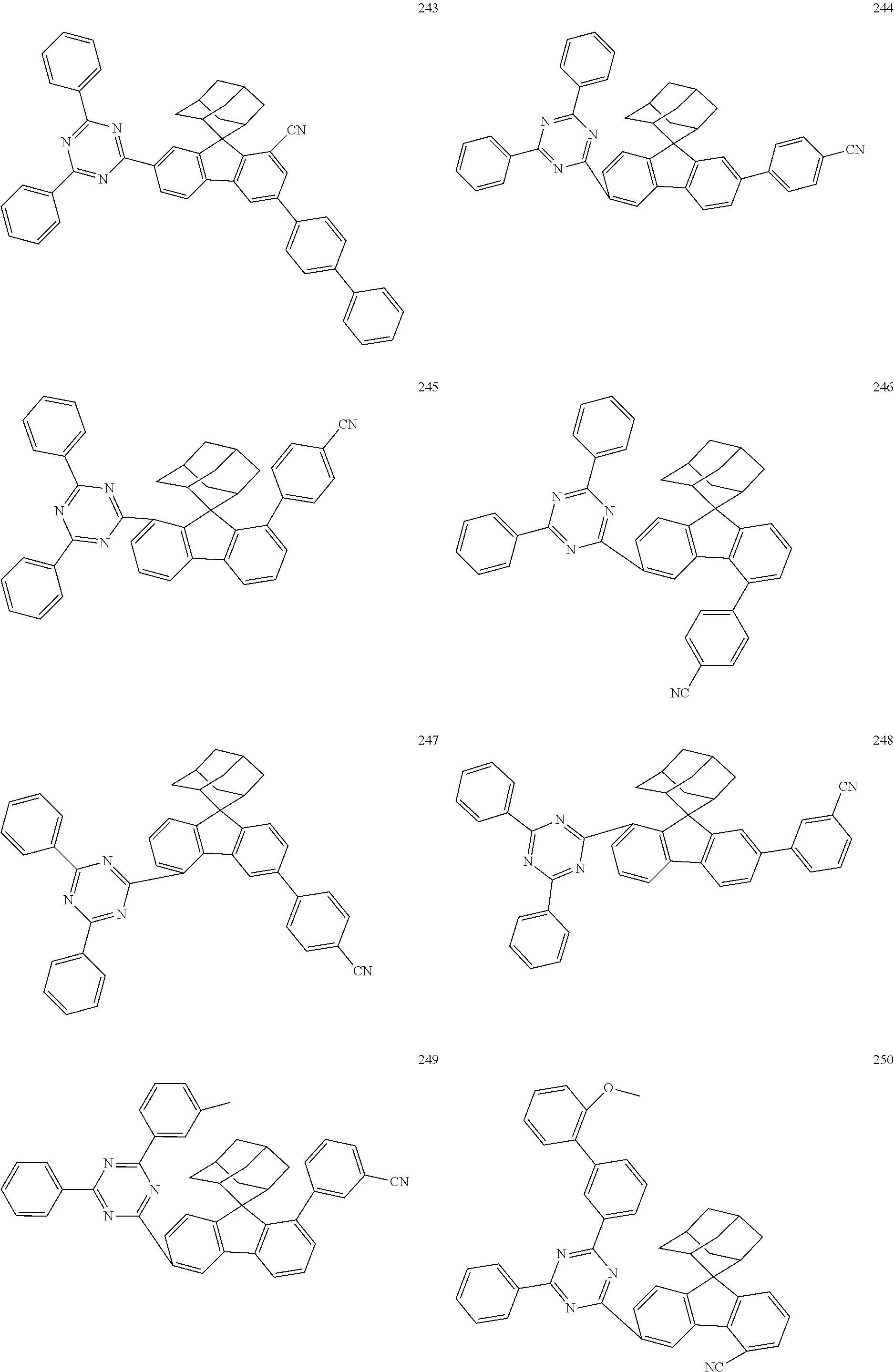

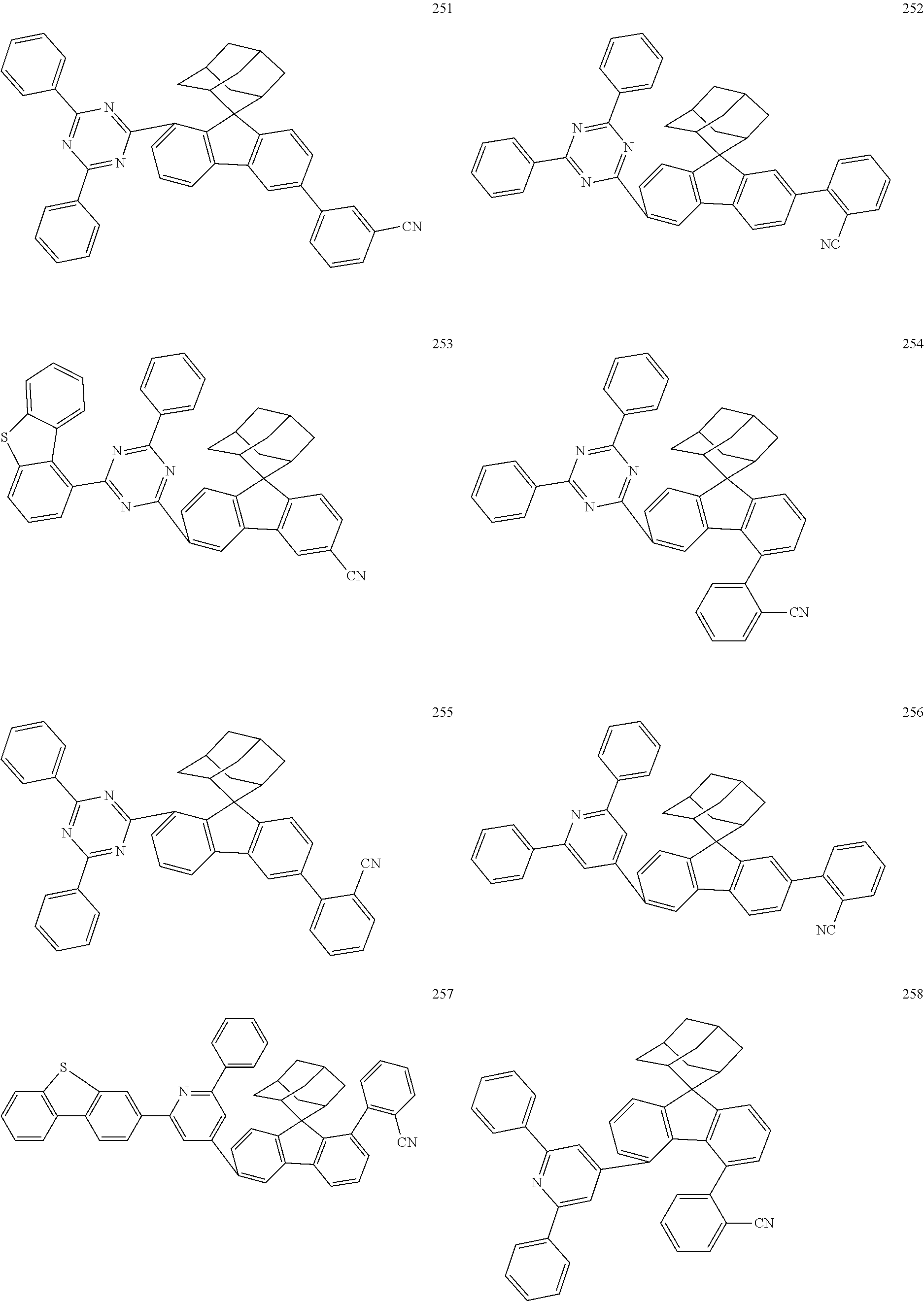

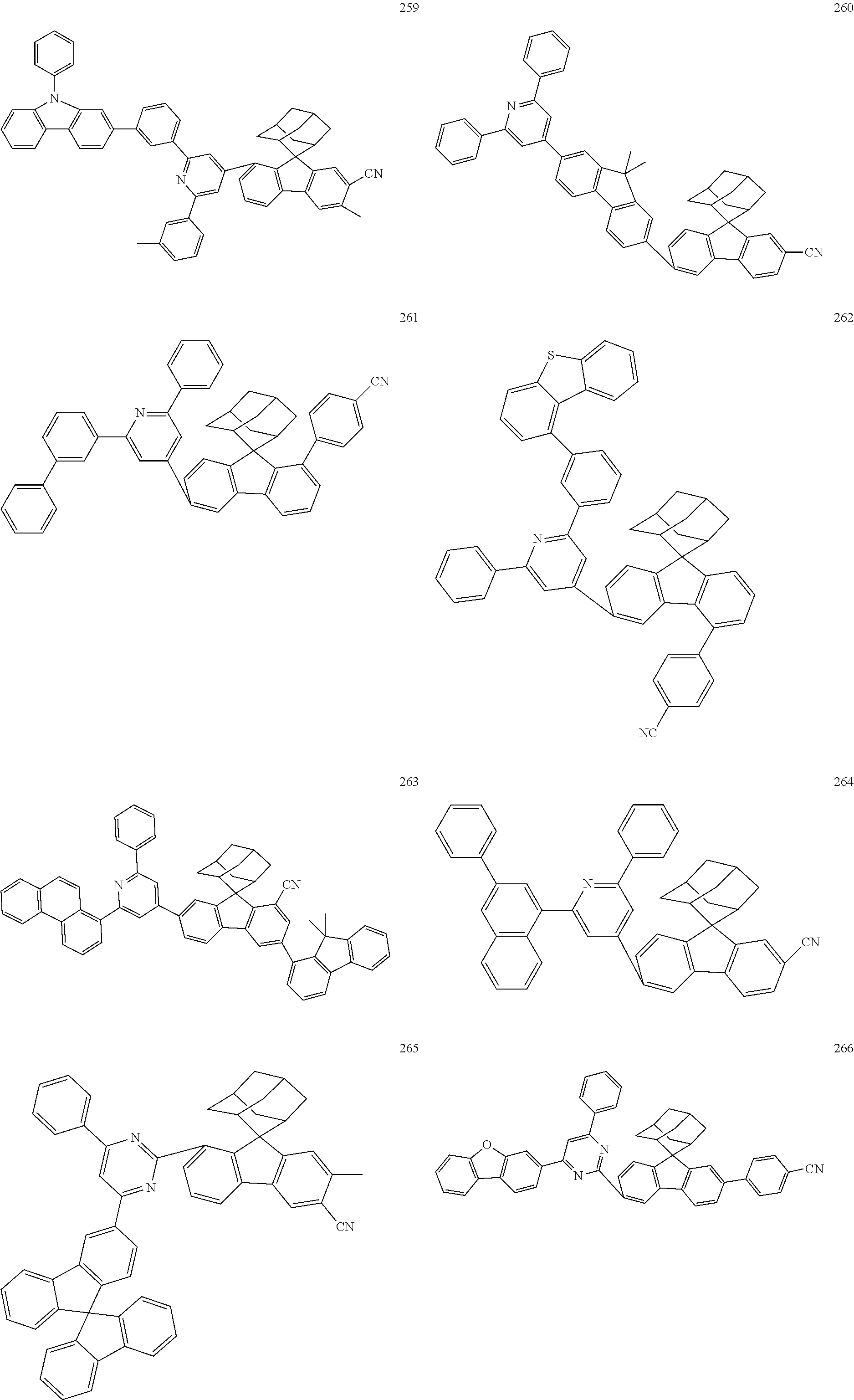

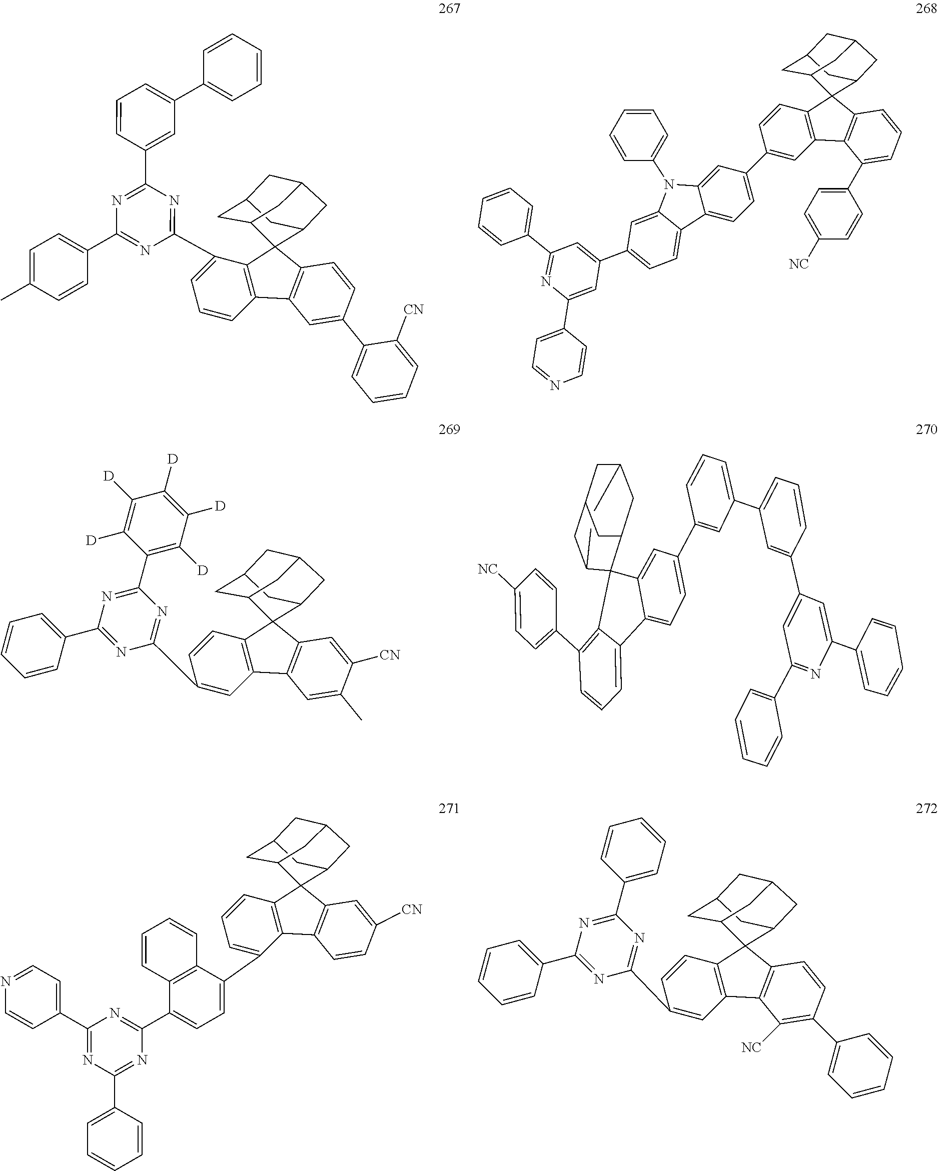

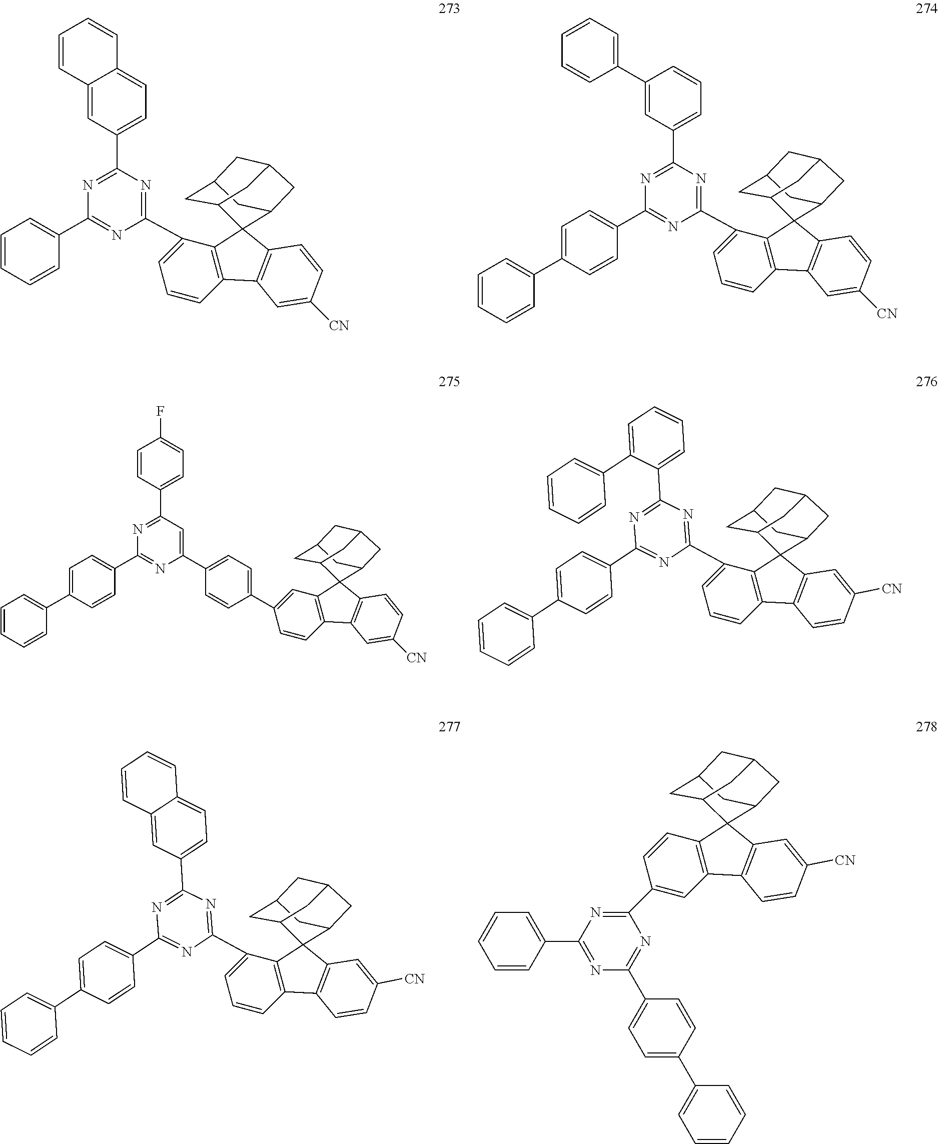

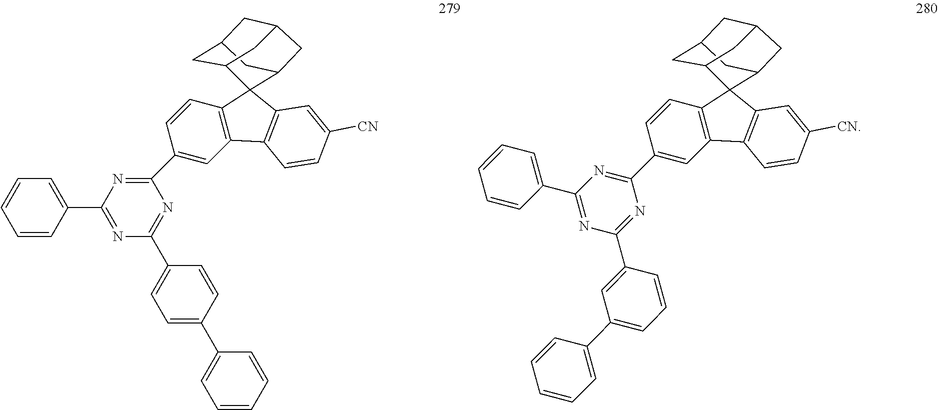 [SMILES: N#Cc1ccc2c(c1)C1(c3ccc(-c4nc(-c5ccccc5)nc(-c5ccc(-c6ccccc6)cc5)n4)cc3-2)C2CC3CC(C2)CC1C3 ; N#Cc1ccc2c(c1)C1(c3ccc(-c4nc(-c5ccccc5)nc(-c5cccc(-c6ccccc6)c5)n4)cc3-2)C2CC3CC(C2)CC1C3]

The present disclosure does not specifically limit a synthesis method of the nitrogen-containing compound provided, and the skilled in the art can determine a suitable synthesis method according to the compound structure of the present disclosure in combination with a preparation method provided in a synthesis example section. In other words, the synthesis example section of the present disclosure provides a method for preparing the nitrogen-containing compound in an example manner, and raw materials employed can be obtained commercially or by methods well known in the art. The skilled in the art can obtain all the nitrogen-containing compounds provided in the present disclosure according to these example preparation methods.

In a second aspect, the present disclosure provides an electronic element that can realize photoelectric conversion or electro-optical conversion. The electronic element includes an anode and a cathode which are disposed oppositely, and a functional layer disposed between the anode and the cathode. The functional layer contains the nitrogen-containing compound of the present disclosure. Optionally, the functional layer includes an electron transporting layer, and the electron transporting layer includes the nitrogen-containing compound of the present disclosure.

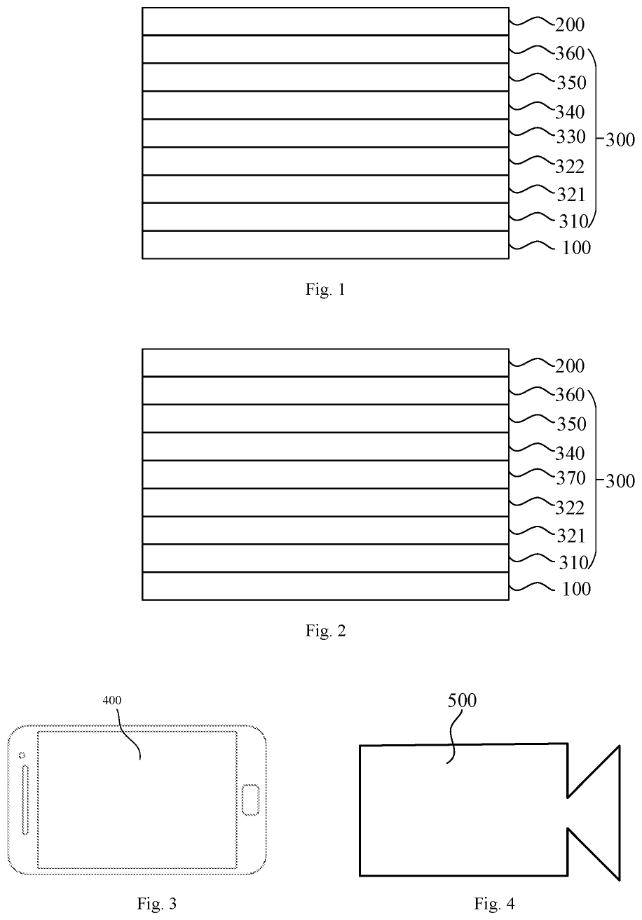

According to one embodiment, the electronic element is an organic electroluminescent device. As shown in FIG. 1, the organic electroluminescent device includes an anode 100 and a cathode 200 which are disposed oppositely, and a functional layer 300 disposed between the anode 100 and the cathode 200. The functional layer 300 contains the nitrogen-containing compound provided by the present disclosure.

Optionally, the functional layer 300 includes an electron transporting layer 350, and the electron transporting layer 350 contains the nitrogen-containing compound provided by the present disclosure. The electron transporting layer 350 may be composed of the nitrogen-containing compound provided by the present disclosure, or may be composed of the nitrogen-containing compound provided by the present disclosure and other materials together. According to one preferred embodiment, the electron transporting layer 350 contains the nitrogen-containing compound of the present disclosure and LiQ.

Optionally, the organic electroluminescent device may include an anode 100, a hole transporting layer 321, an electron blocking layer 322, an organic electroluminescent layer 330, an electron transporting layer 350 and a cathode 200 which are stacked sequentially.

Optionally, the anode 100 includes the following anode material, which is preferably a material with a large work function that facilitates hole injection into the functional layer. Specific examples of the anode material include: metals such as nickel, platinum, vanadium, chromium, copper, zinc and gold or their alloys; metal oxides such as zinc oxide, indium oxide, indium tin oxide (ITO) and indium zinc oxide (IZO); combined metal and oxides such as ZnO:Al or SnO₂:Sb; or conducting polymers such as poly(3-methylthiophene), poly[3,4-(ethylene-1,2-dioxy)thiophene] (PEDT), polypyrrole and polyaniline, but are not limited to these. Preferably, it includes a transparent electrode containing an indium tin oxide (ITO) as an anode.

Optionally, the hole transporting layer 321 may include one or more hole transporting materials, and the hole transporting materials may be selected from a carbazole polymer, a carbazole-linked triarylamine compound or other types of compounds, which is not particularly limited in the present disclosure. For example, the hole transporting layer 321 is composed of a compound HT-01.

Optionally, the electron blocking layer 322 includes one or more electron blocking materials, and the electron blocking materials may be selected from a carbazole polymer or other types of compounds, which is not particularly limited in the present disclosure. For example, in some embodiments of the present disclosure, the electron blocking layer 322 is composed of a compound HT-02.

Optionally, the organic electroluminescent layer 330 may be composed of a single light-emitting material, or may include a host material and a guest material. Optionally, the organic electroluminescent layer 330 is composed of the host material and the guest material. Holes injected into the light-emitting layer and electrons injected into the light-emitting layer may be recombined in the light-emitting layer to form excitons, the excitons transfer energy to the host material, and the host material transfers energy to the guest material, so that the guest material can emit light. The host material of the organic electroluminescent layer 330 may be a metal chelate compound, a bis-styryl derivative, an aromatic amine derivative, a dibenzofuran derivative or other types of materials, which is not particularly limited in the present disclosure. For example, the host material of the organic electroluminescent layer 330 may be BH-01, and the guest material may be a compound having a condensed aryl ring or its derivative, a compound having a heteroaryl ring or its derivative, an aromatic amine derivative or other materials, which is not particularly limited in the present disclosure. For example, the guest material may be BD-01.

Optionally, the cathode 200 includes the following cathode material, which is a material with a small work function that facilitates electron injection into the functional layer. Specific examples of the cathode material include: metals such as magnesium, calcium, sodium, potassium, titanium, indium, yttrium, lithium, gadolinium, aluminum, silver, tin and lead or their alloys; or a multilayer material such as LiF/Al, Liq/Al, LiO₂/Al, LiF/Ca, LiF/Al, and BaF₂/Ca, but are not limited to these. Preferably, it includes a metal electrode containing silver and magnesium as the cathode.

Optionally, as shown in FIG. 1, a hole injection layer 310 may further be disposed between the anode 100 and the hole transporting layer 321 to enhance a capability of injecting holes into the hole transporting layer. The hole injection layer 310 may be selected from a benzidine derivative, a starburst arylamine compound, a phthalocyanine derivative or other materials, which is not particularly limited in the present disclosure. For example, the hole injection layer 310 may be composed of F4-TCNQ.

Optionally, as shown in FIG. 1, an electron injection layer 360 may further be disposed between the cathode 200 and the electron transporting layer 350 to enhance a capability of injecting electrons into the electron transporting layer 350. The electron injection layer 360 may include inorganic materials such as alkali metal sulfide and alkali metal halide, or may include a complex of alkali metal and organic matter. For example, the electron injection layer 360 may include LiQ.

Optionally, a hole blocking layer 340 may further or may not be disposed between the organic electroluminescent layer 330 and the electron transporting layer 350.

Specific structures of the above HT-01, HT-02, BH-01, BD-01, F4-TCNQ and LiQ may refer to the specific examples below.

According to another embodiment, the electronic element may be a photoelectric conversion device. As shown in FIG. 2, the photoelectric conversion device may include an anode 100 and a cathode 200 which are disposed oppositely, and a functional layer 300 disposed between the anode 100 and the cathode 200. The functional layer 300 contains the nitrogen-containing compound provided by the present disclosure.

Optionally, the functional layer 300 includes an electron transporting layer 350, and the electron transporting layer 350 contains the nitrogen-containing compound provided by the present disclosure. The electron transporting layer 350 may be composed of the nitrogen-containing compound provided by the present disclosure, or may be composed of the nitrogen-containing compound provided by the present disclosure and other materials together.

Optionally, as shown in FIG. 2, the photoelectric conversion device may include an anode 100, a hole transporting layer 321, an electron blocking layer 322, a photoelectric conversion layer 370 serving as an energy conversion layer, an electron transporting layer 350 and a cathode 200 which are stacked sequentially. The nitrogen-containing compound provided by the present disclosure may be applied to the electron transporting layer 350 of the photoelectric conversion device, which can effectively improve a light-emitting efficiency and service life of the photoelectric conversion device, and increase an open circuit voltage of the photoelectric conversion device.

Optionally, a hole injection layer 310 may further be disposed between the anode 100 and the hole transporting layer 321. An electron injection layer 360 may further be disposed between the cathode 200 and the electron transporting layer 350. A hole blocking layer 340 may further be disposed between the photoelectric conversion layer 370 and the electron transporting layer 350.

In the present disclosure, the photoelectric conversion device may be, for example, a solar cell, especially an organic thin-film solar cell. For example, in one embodiment of the present disclosure, as shown in FIG. 2, the solar cell includes an anode 100, a hole transporting layer 321, an electron blocking layer 322, a photoelectric conversion layer 370, an electron transporting layer 350 and a cathode 200 which are stacked sequentially, wherein the electron transporting layer 350 contains the nitrogen-containing compound of the present disclosure.

A third aspect of the present disclosure provides an electronic device, including the electronic element according to the second aspect of the present disclosure.

According to one embodiment, as shown in FIG. 3, the electronic device is a first electronic device 400, and the first electronic device 400 includes the above organic electroluminescent device. The first electronic device 400 may be, for example, a display device, a lighting device, an optical communication device, or other types of electronic devices, which may include, for example, but not limited to a computer screen, a mobile phone screen, a television, electronic paper, an emergency lamp, an optical module and the like.

According to another embodiment, as shown in FIG. 4, the electronic device is a second electronic device 500, and the second electronic device 500 includes the above photoelectric conversion device. The second electronic device 500 may be, for example, a solar power generation device, a photodetector, a fingerprint identification device, an optical module, a CCD camera, or other types of electronic devices.

A synthesis method of an organic compound of the present disclosure is specifically described below with reference to a synthesis example. Unless otherwise specified, the employed raw materials can be obtained commercially or prepared by methods well known in the art.

### Synthesis of Intermediate

### 1. Synthesis of Intermediate A-I

All the intermediates IM A-I are synthesized by referring to the following synthetic general formula.

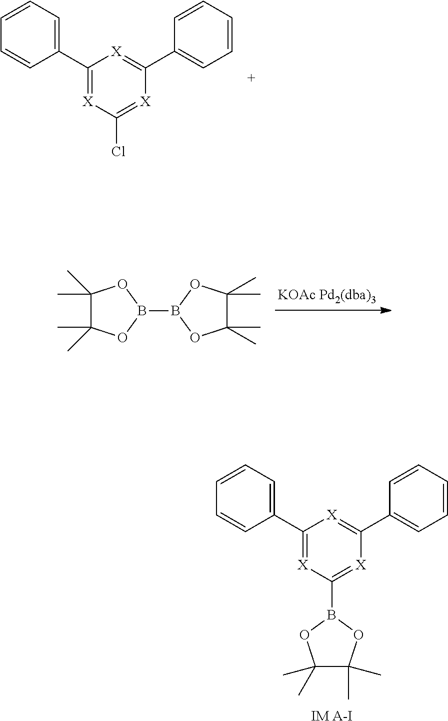

Wherein each one of the three X independently represents C(H) or N, and at least one of the three is N.

### 1) Synthesis of IM A-1

Taking IM A-1 as an example to illustrate the synthesis of IM A-I:

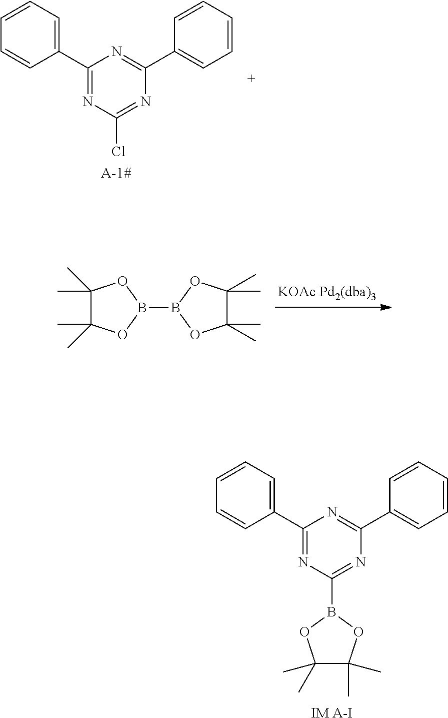

Raw materials A-1 # (10.0 g, 37.5 mmol), bis(pinacolato)diboron (11.4 g, 45 mmol), 7.34 g of potassium acetate, 0.34 g of tris(dibenzylideneacetone)dipalladium, and 0.24 g of 2-dicyclohexylphosphino-2′,4′,6′-triisopropylbiphenyl were added to 100 mL of toluene which were mixed as reactants, the reactants were heated to 108° C. and reacted for 2 hours and obtained reaction solution, the reaction solution was washed with water, passed through a toluene column, and recrystallized to obtain 7.3 g of the intermediate IM A-1 (28.2 mmol, yield 75%).

### 2) Synthesis of IM A-2 to IM A-4

Other IM A-I are synthesized with reference to the synthesis method of IM A-1. The difference is that A-1 # is replaced with a raw material 1. The employed raw material 1, the synthesized IM A-I and the yield of IM A-I are shown in Table 1.

TABLE 1

| Raw material 1 | IM A-I | Yield (%) |
|---|---|---|
| 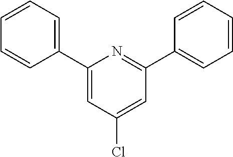 [SMILES: Clc1cc(-c2ccccc2)nc(-c2ccccc2)c1] | 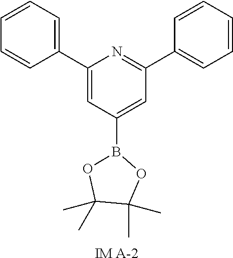 | 82 |
| 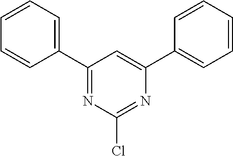 [SMILES: Clc1nc(-c2ccccc2)cc(-c2ccccc2)n1] | 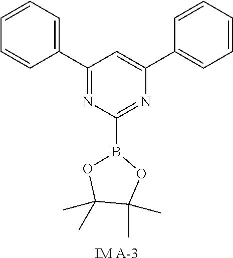 | 65 |
| 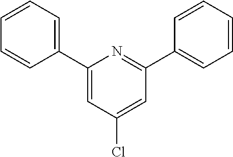 [SMILES: Clc1cc(-c2ccccc2)nc(-c2ccccc2)c1] | 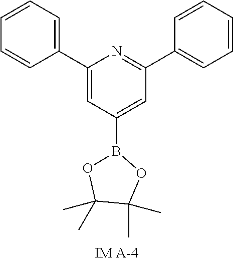 | 72 |

### 2.1 Synthesis of Intermediate IM B-I

All the intermediates IM B-I are synthesized by referring to the following synthetic general formula.

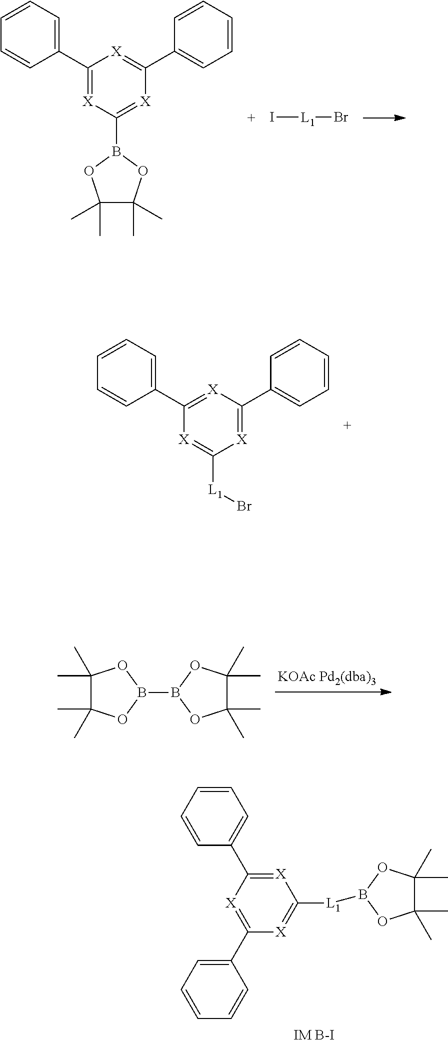

### 1) Synthesis of IM B-1

Taking IM B-1 as an example to illustrate the synthesis of IM B-I:

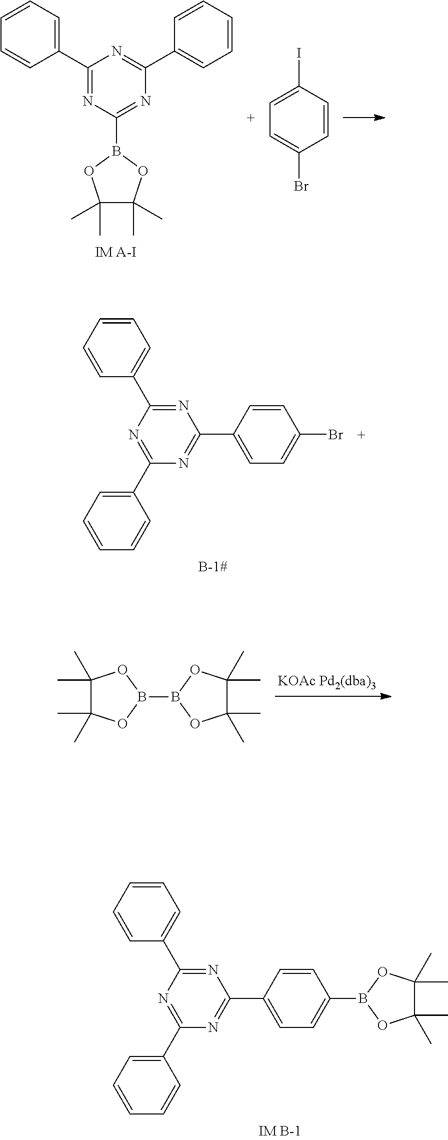

(1) IM A-1 (12 g, 33.4 mmol) was taken, and p-bromoiodobenzene (9.45 g, 33.4 mmol), 9.21 g of potassium carbonate, 1.5 g of tetrabutylammonium bromide, 0.38 g of tetrakis(triphenylphosphine)palladium, 80 mL of toluene, 40 mL of ethanol, and 40 mL of water were added and mixed as reactants. Under nitrogen protection, the reactants were heated to 72° C. and reacted for 10 h, the reaction solution was washed with water, passed through a toluene column, and recrystallized to obtain 10.36 g of a product B-1 # (26.7 mmol, yield 80%).

(2) B-1 # (10 g, 25.7 mmol) was taken, and bis(pinacolato)diboron (7.85 g, 30.8 mmol), 5.03 g of potassium acetate, 0.24 g of tris(dibenzylideneacetone)dipalladium, 0.15 g of 2-dicyclohexylphosphino-2′,4′,6′-triisopropylbiphenyl and 80 mL of toluene were added and mixed as reactants. The reactants were heated to 108° C. and reacted for 2 h and obtained a reaction solution, the reaction solution was washed with water, passed through a toluene column, and recrystallized to obtain 8.7 g of the intermediate IM B-1 (20.0 mmol, yield 78%).

### 2) Synthesis of IM B-2 to IM B-6

Other IM B-I are synthesized with reference to the method of IM B-1. The differences include replacing IM A-1 in the step (1) with a IM A-I and replacing p-bromoiodobenzene with a raw material 2. The employed raw materials and the correspondingly synthesized intermediates and their yields are shown in Table 2.

TABLE 2

| IM A-I | Raw material 2 | IM B-I | Yield (%) |
|---|---|---|---|
| IM A-3 | 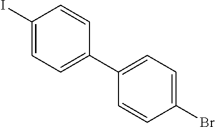 [SMILES: Brc1ccc(-c2ccc(I)cc2)cc1] | 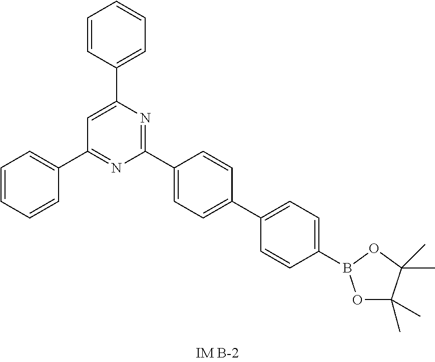 | 71 |
| IM A-1 | 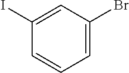 | 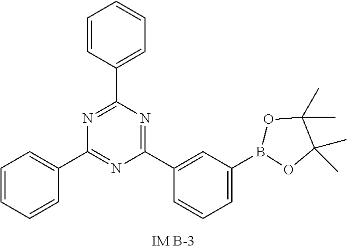 | 85 |
| IM A-1 | 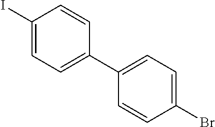 [SMILES: Brc1ccc(-c2ccc(I)cc2)cc1] | 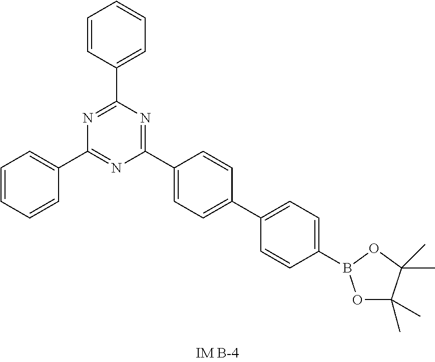 | 68 |
| IM A-1 | 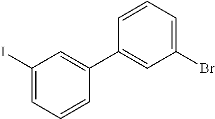 [SMILES: Brc1cccc(-c2cccc(I)c2)c1] | 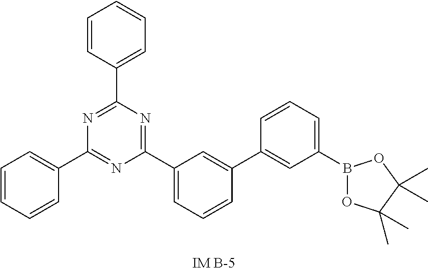 | 76 |
| IM A-4 | 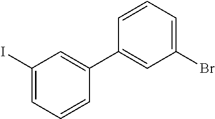 [SMILES: Brc1cccc(-c2cccc(I)c2)c1] | 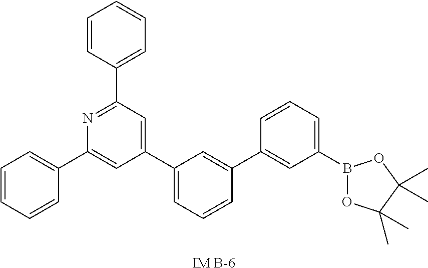 | 79 |

### 2.2 Preparation of Intermediate IM B1-1

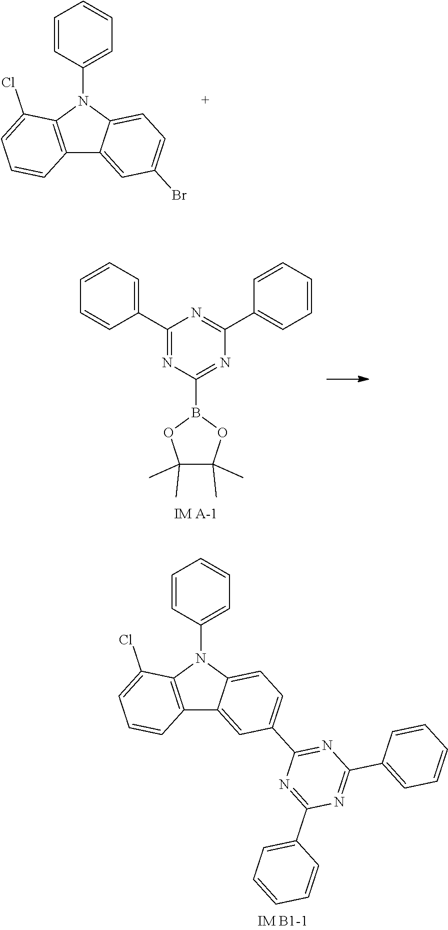

6-bromo-1-chloro-9-phenylcarbazole (7.12 g, 20 mmol), IM A-1 (7.18 g, 20 mmol), 60 mL of toluene, 30 mL of ethanol, 20 mL of water, 0.64 g of tetrabutylammonium bromide, and 5.52 g of potassium carbonate were weighed and then added 1.15 g of tetrakis(triphenylphosphine)palladium under nitrogen protection, which were mixed as reactants, the temperature of reactants was raised to reflux for 12 h, the reaction solution was poured into water, washed with water and extracted with toluene to obtain organic phase, and then all the extracted organic phase solution were gathered, dried, and concentrated to obtain a solid product IM B1-1 (7.0 g, yield 69.8%).

### 3. Synthesis of Intermediate IM C

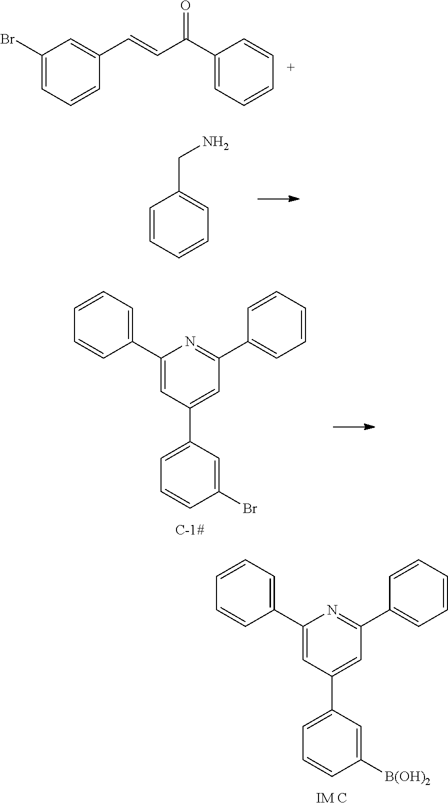 [SMILES: O=C(/C=C/c1cccc(Br)c1)c1ccccc1 ; NCc1ccccc1 ; Brc1cccc(-c2cc(-c3ccccc3)nc(-c3ccccc3)c2)c1 ; OB(O)c1cccc(-c2cc(-c3ccccc3)nc(-c3ccccc3)c2)c1]

Under nitrogen protection, 3-bromochalcone (28.7 g, 100 mmol), 200 mL of xylene, and benzylamine (13.9 g; 130 mmol) were added sequencely and stirred, and then 6.0 g of trifluoromethanesulfonic acid was added dropwise. The reactant mixture was heated to 115° C. to 120° C. and after reacted for 18 h with stirring, a detection of reaction was started, that is, a sample was taken per hour until the value of LC (Liquid Chromatography) content of C-1 # increases up to LC>45% and basically not changed, the reaction may be stopped. Then the reaction solution was poured into water, extracted with dichloroethane, dried and concentrated to obtain an oily substance, after that the oily substance was recrystallized with ethanol (1 g of oily substance: 4 mL of ethanol) to obtain an off-white solid C-1 # (12.35 g, yield 32%) with LC>95%.

Under nitrogen protection C-1 # (11.58 g, 30 mmol) and 100 mL of tetrahydrofuran were mixed in sequence as a reaction system, then the reaction system was cooled down to −90° C. to −80° C. with stirred. After stabilization in −90° C. to −80° C., n-butyllithium (36 mmol) was added dropwise into the reaction system, and then the temperature was kept for about 1.5 h until the value of LC of the raw material C-1 # was LC<1%, 10.35 g of tributyl borate was started to be added dropwise, after that the temperature was kept at −90° C. to −78° C. for 2 h and then raised naturally. After 2 h, the above mentioned detection was performed to monitor the reaction, that is, the reaction was stopped when the value of LC content of IM C increase up to LC>85%. The reaction solution was poured into water, stirring was performed for 15 minutes, and then was set for separation. An organic phase from the separation was washed with water for several times until a white solid was precipitated, and the white solid was filtered and then dried (40-45° C.; 4h) so as to obtain 9.79 g of the intermediate IM C (27.9 mmol, yield 93%).

### 4. Synthesis of Intermediate IM D

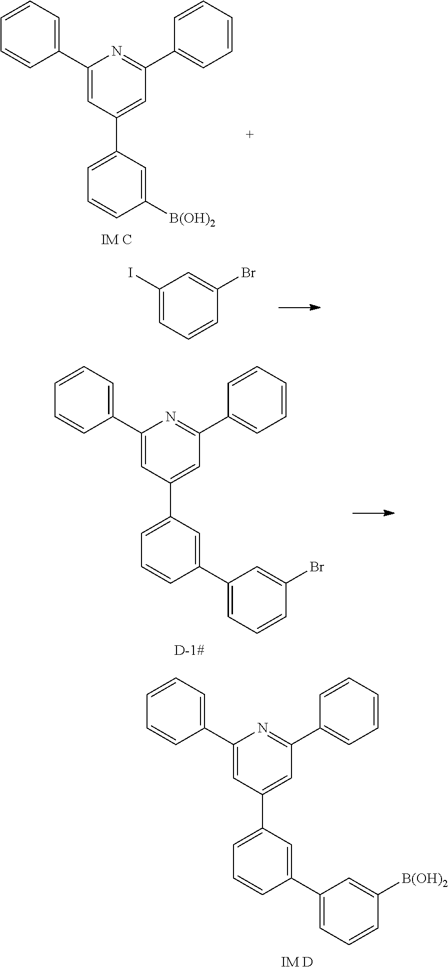 [SMILES: OB(O)c1cccc(-c2cc(-c3ccccc3)nc(-c3ccccc3)c2)c1 ; Brc1cccc(I)c1 ; Brc1cccc(-c2cccc(-c3cc(-c4ccccc4)nc(-c4ccccc4)c3)c2)c1 ; OB(O)c1cccc(-c2cccc(-c3cc(-c4ccccc4)nc(-c4ccccc4)c3)c2)c1]

Under nitrogen protection, IM C (14.04 g, 40 mmol), m-bromoiodobenzene (11.32 g, 40 mmol), 11.04 g of potassium carbonate, 1.28 g of tetrabutylammonium bromide, 80 mL of toluene, 40 mL of ethanol, and 40 mL of water were added in sequence as a mixture, after heating and stirring to 50° C., 0.46 g of tetrakis(triphenylphosphine)palladium was added into the mixture, and then heated to a reflux reaction. After 12 h, the reaction was monitored. When the IM C content reached to LC<1% and the D-1 # content reached to LC>90%, the reaction was stopped, and recrystallization was performed with cyclohexane to obtain a crude solid. (m crude solid: v cyclohexane=1 g: 20 mL, with heating reflux for 1 h so that the crude solid was dealt by solvent extraction). Then insolubles were filtered off and the filtrate was passed through an insulating column (75° C.), solution passing through the column was concentrated and cooled to 15° C. for crystallization. Then filtering was performed after the 2-hour crystallization to obtain 11.08 g of white solid powder D-1 #, LC>98% (24 mmol, yield 60%).

IM D is synthesized according to the steps of IM C. The difference is that C-1 # is replaced by D-1 # (30 mmol of feedstock) to obtain IM D (10.63 g, 24.9 mmol, yield 82%).

### 5.1 Synthesis of Intermediate IM E-I

All the intermediates IM E-I are synthesized by referring to the following synthetic general formula.

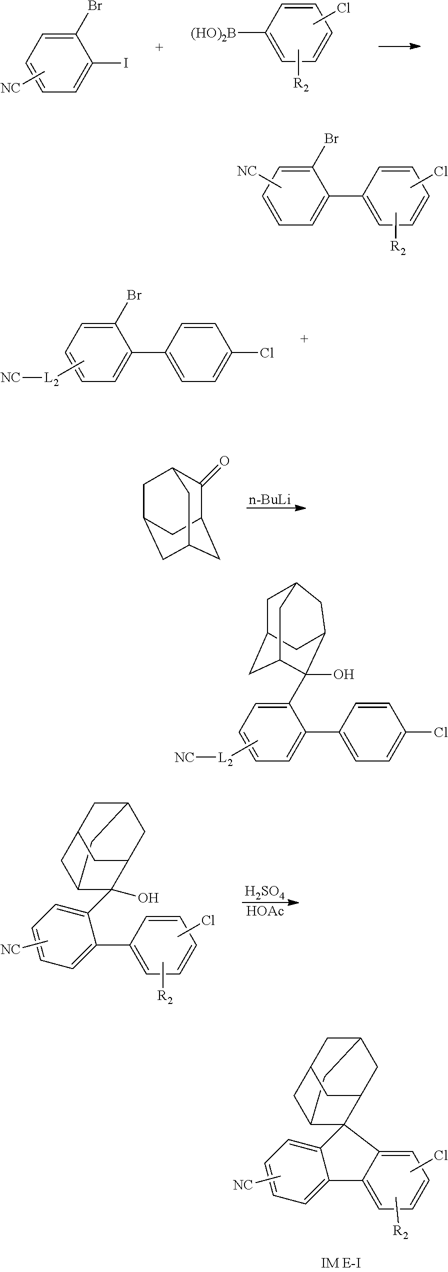

### 1) Synthesis of IM E-1

Taking IM E-1 as an example to illustrate the synthesis of IM E-I:

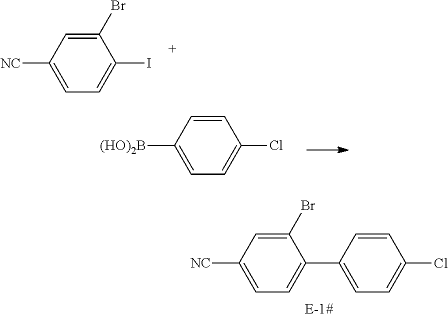 [SMILES: N#Cc1ccc(I)c(Br)c1 ; OB(O)c1ccc(Cl)cc1 ; N#Cc1ccc(-c2ccc(Cl)cc2)c(Br)c1]

(1) 2-bromo-4 cyanoiodobenzene (30.7 g, 100 mmol) and 4-chlorobenzeneboronic acid (15.6 g, 100 mmol) were added into 1.15 g of tetrakis(triphenylphosphine)palladium and 27.6 g of potassium carbonate under nitrogen protection, 200 mL of toluene, 150 mL of ethanol, and 100 mL of water were added so as to obtain a mixture, the mixture was heated to 72° C. and stirred for 3 h; then cooled down to the room temperature and obtained a reaction solution. The reaction solution was washed with water, then magnesium sulfate was added for drying, and the filtrate after filtered was further removed solvent under reduced pressure, and obtained a crude product. The crude product was purified by recrystallization using toluene (1 g of the crude product: 10 mL of toluene) to obtain 16.35 g of a white solid product E-1 # (56 mmol, yield 56%).

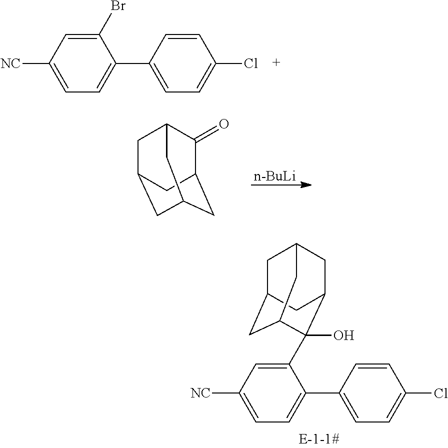

(2) E-1 # (14.62 g, 50 mmol) was taken to be dissolved in 150 mL of tetrahydrofuran as a mixture, then a liquid nitrogen ethanol bath was used to cool down the temperature of the mixture to −85° C., after that n-butyllithium (30 ml, 60 mmol) was added dropwise, and the temperature was kept for 2 hours after dropwise adding so as to obtain a reaction solution. Adamantanone (7.5 g, 50 mmol) was added dropwise to the reaction solution and the temperature was kept for 2 h, then the reaction solution was poured into water, and after-treatment was performed to obtain a crude solid, which is recrystallized with toluene to obtain 15.46 g of a product E-1-1 # (42.5 mmol, yield 85%).

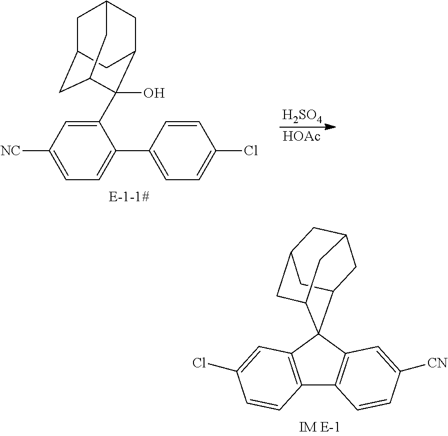 [SMILES: N#Cc1ccc(-c2ccc(Cl)cc2)c(C2(O)C3CC4CC(C3)CC2C4)c1 ; "H2SO4" ; N#Cc1ccc2c(c1)C1(c3cc(Cl)ccc3-2)C2CC3CC(C2)CC1C3 ; "HOAc"]

(3) E-1-1 # (14.5 g, 40 mmol) was dissolved in 100 mL of glacial acetic acid and 20 mL of concentrated sulfuric acid (98 wt %) was added, then heated to 65° C., and a reaction was completed in 5 hours so as to obtain a reaction solution. The reaction solution was poured into water, neutralized to neutrality with sodium hydroxide, and an organic phase which was extracted from the neutralized reaction solution with toluene was dried, concentrated, and recrystallized to obtain 9.95 g of an intermediate IM E-1 (28.8 mmol, yield 72%).

### 2) Synthesis of IM E-2 to IM E-8

Other IM E-I is synthesized with reference to the method for IM E-1. The differences include replacing 2-bromo-4 cyanoiodobenzene with a raw material 4, replacing 4-chlorobenzeneboronic acid with a raw material 5. The employed main raw materials and the correspondingly synthesized intermediate structures and final yield are shown in Table 3.

TABLE 3

| | | Structure | | |
|---|---|---|---|---|
| | | synthesized in step | | Yield |
| Raw material 4 | Raw material 5 | (1) | IM E-I | (%) |
| 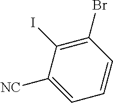 | 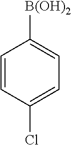 | 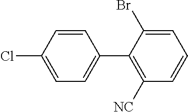 [SMILES: N#Cc1cccc(Br)c1-c1ccc(Cl)cc1] | 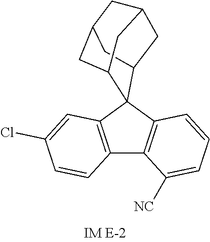 [SMILES: N#Cc1cccc2c1-c1ccc(Cl)cc1C21C2CC3CC(C2)CC1C3] | 64 |
| 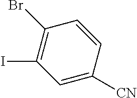 | | 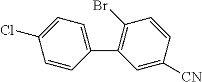 [SMILES: N#Cc1ccc(Br)c(-c2ccc(Cl)cc2)c1] | 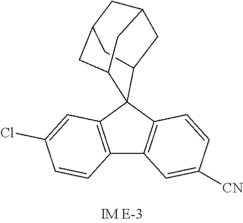 [SMILES: N#Cc1ccc2c(c1)-c1ccc(Cl)cc1C21C2CC3CC(C2)CC1C3] | 68 |
| 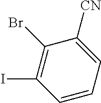 | | 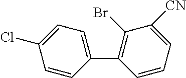 [SMILES: N#Cc1cccc(-c2ccc(Cl)cc2)c1Br] | 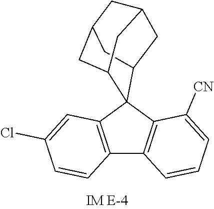 [SMILES: N#Cc1cccc2c1C1(c3cc(Cl)ccc3-2)C2CC3CC(C2)CC1C3] | 66 |
| 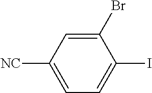 | 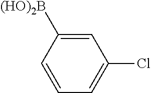 | 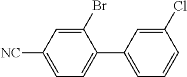 [SMILES: N#Cc1ccc(-c2cccc(Cl)c2)c(Br)c1] | 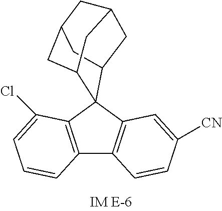 [SMILES: N#Cc1ccc2c(c1)C1(c3c(Cl)cccc3-2)C2CC3CC(C2)CC1C3] | 22 |
| 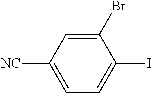 | 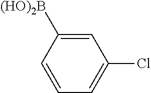 | 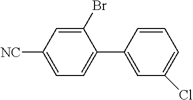 [SMILES: N#Cc1ccc(-c2cccc(Cl)c2)c(Br)c1] | 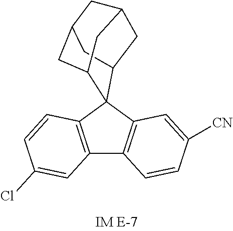 [SMILES: N#Cc1ccc2c(c1)C1(c3ccc(Cl)cc3-2)C2CC3CC(C2)CC1C3] | 41 |
| 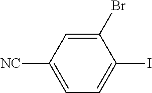 | 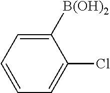 | 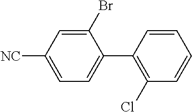 [SMILES: N#Cc1ccc(-c2ccccc2Cl)c(Br)c1] | 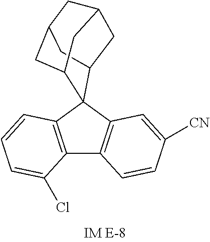 [SMILES: N#Cc1ccc2c(c1)C1(c3cccc(Cl)c3-2)C2CC3CC(C2)CC1C3] | 70 |

### 5.2 Preparation Method of Intermediate IM E1-4

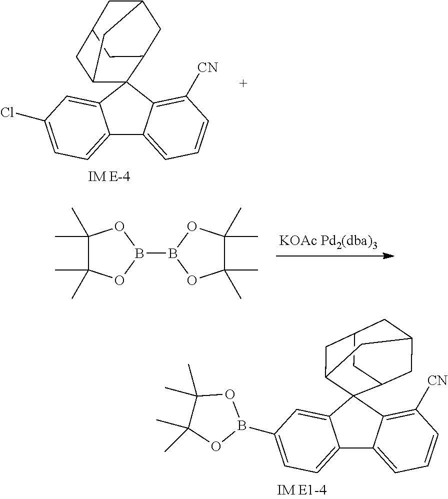

(2) IM E-4 (12.9 g, 37.5 mmol), bis(pinacolato)diboron (11.4 g, 45 mmol), 7.36 g of potassium acetate, 0.39 g of tris(dibenzylideneacetone)dipalladium, 0.24 g of 2-dicyclohexylphosphino-2′,4′,6′-triisopropylbiphenyl and 120 mL of toluene were mixed as reactants. The temperature was raised to 108° C. for reaction, the reaction was completed after 2 h and a reaction solution was obtained. The reaction solution was washed with water, passed through a toluene column, and recrystallized to obtain 11.6 g of the intermediate IM E₁-4 (26.5 mmol, yield 70%).

### 6. Synthesis of Intermediate IM F-I

All the intermediates IM F-I are synthesized by referring to the following synthetic general formula.

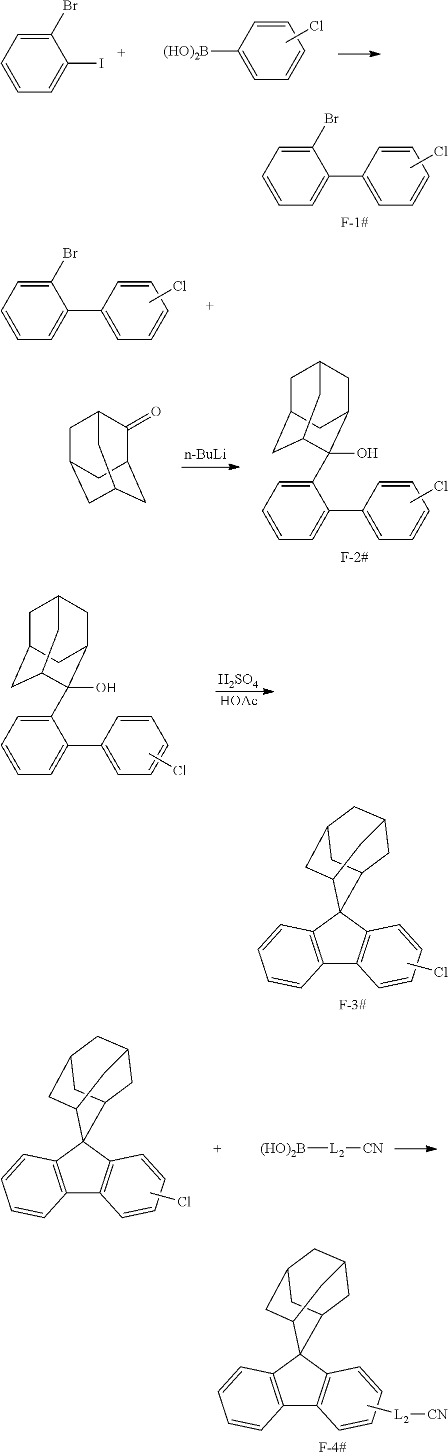

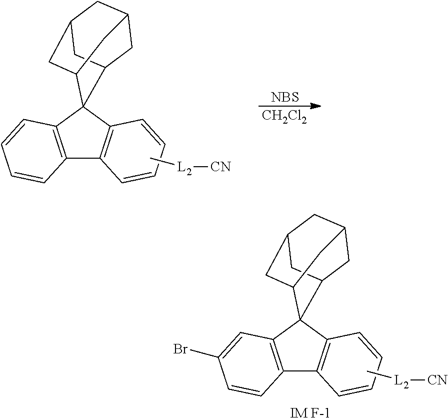

Wherein L² is selected from arylene with 6 to 12 carbon atoms.

### 1) Synthesis of IM F-1

Taking IM F-1 as an example to illustrate the synthesis of all IM F-I:

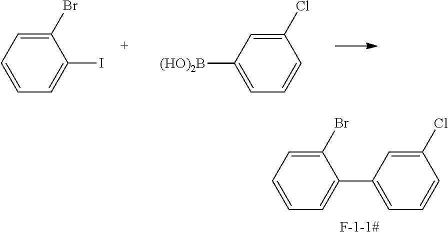 [SMILES: Brc1ccccc1I ; OB(O)c1cccc(Cl)c1 ; Clc1cccc(-c2ccccc2Br)c1]

(1) Under nitrogen protection, o-bromoiodobenzene (28.3 g, 100 mmol), 3-chlorobenzeneboronic acid (15.6 g, 100 mmol), 1.15 g of tetrakis(triphenylphosphine)palladium, 27.6 g of potassium carbonate, 200 mL of toluene, 150 mL of ethanol, and 100 mL of water were mixed and heated to 75° C. with stirring for 3 h, and then cooling down to the room temperature and a reaction solution was obtained, the reaction solution was washed with water and then separated to obtained an organic phase, the organic phase was dried by adding magnesium sulfate and filtered, and the filtrate was removed solvent under reduced pressure. A crude product was purified by recrystallization using toluene (1 g of the crude product: 10 mL of toluene) to obtain 15.48 g of a white solid product F-1-1 # (58 mmol, yield 58%).

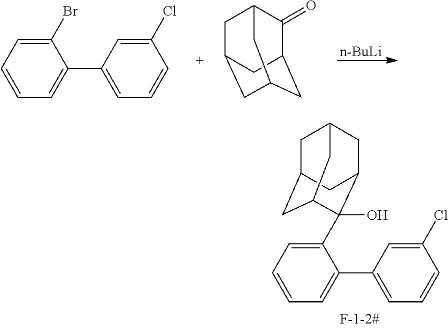

(2) F-1-1 # (14.62 g, 50 mmol) was dissolved in 150 mL of tetrahydrofuran before cooling down to −85° C. through a liquid nitrogen ethanol bath. When it has cooled down, n-butyllithium (30 ml, 60 mmol) was added dropwise, and the temperature was kept for 2 hours after dropwise adding, and a reaction solution was obtained. Adamantanone (7.5 g, 50 mmol) was added dropwise to the reaction solution, and the temperature was kept for 2.5 hours after dropwise adding, after that the reaction solution was poured into water for after-treatment, and solid was precipitated. Then a crude solid was obtained by filtration, and recrystallized with toluene to obtain 13.52 g of solid F-1-2 # (40 mmol, yield 80%).

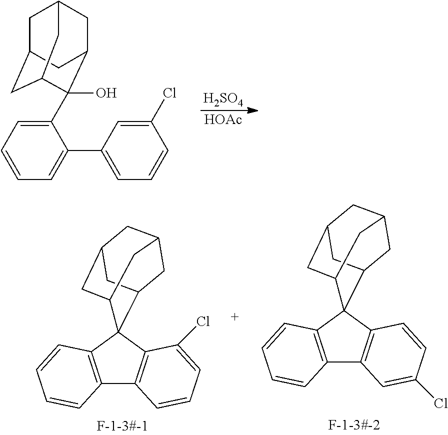

(3) After F-1-2 # (33.8 g, 100 mmol) was dissolved in 100 mL of glacial acetic acid, 20 mL of concentrated sulfuric acid was added. Then heated to 65° C., and a reaction was completed in 5 hours. The reaction solution was poured into water, neutralized to neutrality with sodium hydroxide, and an organic phase was extracted with toluene. The organic phase was dried, concentrated, and separated by column chromatography to obtain two products F-1-3 #-1 (9.6 g, yield 30%) and its isomer F-1-3 #-2 (16 g, yield 50%).

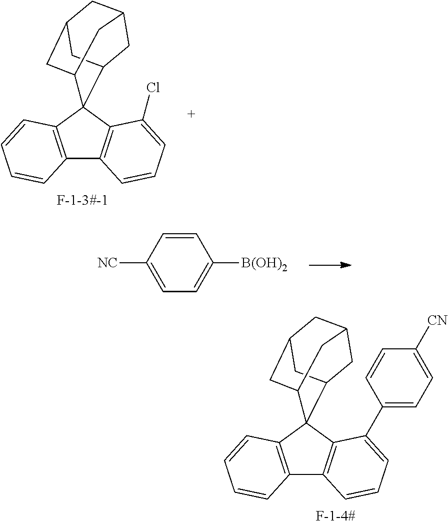 [SMILES: Clc1cccc2c1C1(c3ccccc3-2)C2CC3CC(C2)CC1C3 ; N#Cc1ccc(B(O)O)cc1 ; N#Cc1ccc(-c2cccc3c2C2(c4ccccc4-3)C3CC4CC(C3)CC2C4)cc1]

(4) F-1-3 #-1 (16 g, 50 mmol) and 4-cyanophenylboronic acid (7.3 g, 50 mmol) were mixed with 0.35 g of bis(di-tert-butyl(4-dimethylaminophenyl)phosphine)dichloropalladium (II), 13.8 g of potassium carbonate under nitrogen protection, 100 mL of toluene, 75 mL of ethanol, and 50 mL of water were added so as to obtain a mixture, after that heated to 75° C., and stirred for 3 h; then cooled down to the room temperature. The reaction solution was washed with water before separated to obtain an organic phase. The organic phase was dried by adding magnesium sulfate, and was filtered to obtain a filtrate, and then remove solvent remained in the filtrate under reduced pressure. The crude product was purified by recrystallization using toluene (1 g of the crude product: 6 mL of toluene) to obtain 11.6 g of a white solid product F-1-4 # (30 mmol, yield 60%).

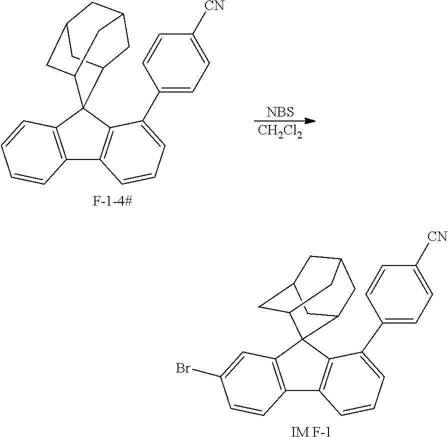

(5) F-1-4 # (9.67 g, 25 mmol), 80 mL of dichloromethane, NBS (4.45 g, 25 mmol) was mixed stirred at room temperature for 10 h, then reaction ends, and the reaction solution was poured into water, washed with water, and extracted with dichloromethane. After separation, drying and concentration a crude solid was obtained, which was further recrystallized with toluene to obtain 6.99 g of an intermediate IM F-1 (15 mmol, yield 60%).

### 2) Synthesis of IM F-2 to IMF-12

Other intermediates IM F-I were synthesized with reference to the method of IM F-1. The differences including replacing 3-chlorobenzeneboronic acid in step (1) with a raw material 6, and replacing the 4-cyanophenylboronic acid in step (4) with a raw material 7. And the employed raw materials and the intermediate synthesized in the main steps are shown in Table 4.

TABLE 4

| | | Main product* |
|---|---|---|
| Raw material 6 | Raw material 7 | synthesized in step (3) |
| 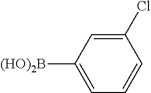 | 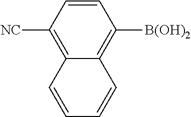 [SMILES: N#Cc1ccc(B(O)O)c2ccccc12] | 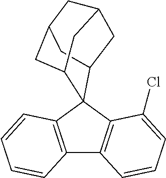 [SMILES: Clc1cccc2c1C1(c3ccccc3-2)C2CC3CC(C2)CC1C3] |
| 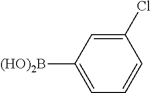 | 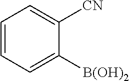 [SMILES: N#Cc1ccccc1B(O)O] | |
| 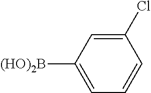 | 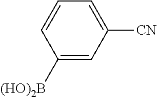 [SMILES: N#Cc1cccc(B(O)O)c1] | |
| 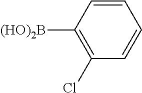 | 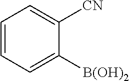 [SMILES: N#Cc1ccccc1B(O)O] | 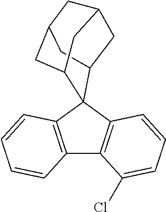 [SMILES: Clc1cccc2c1-c1ccccc1C21C2CC3CC(C2)CC1C3] |
| 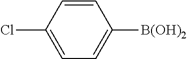 | 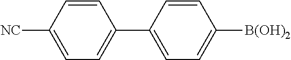 [SMILES: N#Cc1ccc(-c2ccc(B(O)O)cc2)cc1] | 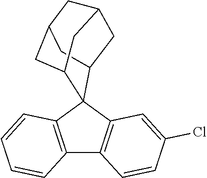 [SMILES: Clc1ccc2c(c1)C1(c3ccccc3-2)C2CC3CC(C2)CC1C3] |
| 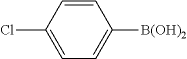 | 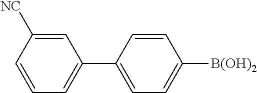 [SMILES: N#Cc1cccc(-c2ccc(B(O)O)cc2)c1] | |
| 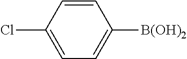 | 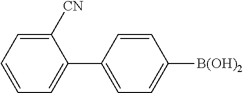 [SMILES: N#Cc1ccccc1-c1ccc(B(O)O)cc1] | |
| 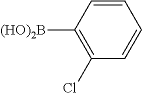 | 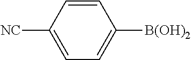 [SMILES: N#Cc1ccc(B(O)O)cc1] | 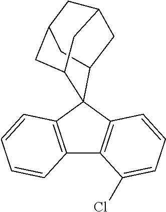 [SMILES: Clc1cccc2c1-c1ccccc1C21C2CC3CC(C2)CC1C3] |
| 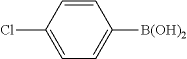 | 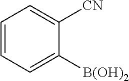 [SMILES: N#Cc1ccccc1B(O)O] | 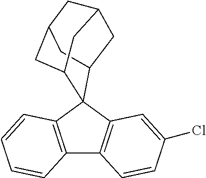 [SMILES: Clc1ccc2c(c1)C1(c3ccccc3-2)C2CC3CC(C2)CC1C3] |
| 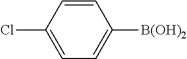 | 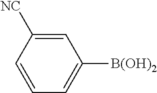 [SMILES: N#Cc1cccc(B(O)O)c1] | |
| 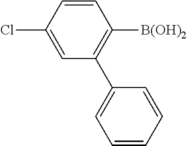 [SMILES: OB(O)c1ccc(Cl)cc1-c1ccccc1] | 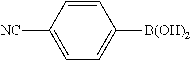 [SMILES: N#Cc1ccc(B(O)O)cc1] | 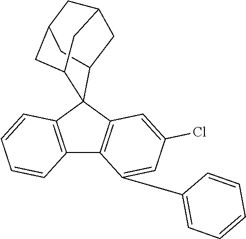 [SMILES: Clc1cc(-c2ccccc2)c2c(c1)C1(c3ccccc3-2)C2CC3CC(C2)CC1C3] |

| | | Yield |
|---|---|---|
| Product synthesized in step (4) | IM F-I | (%) |
| 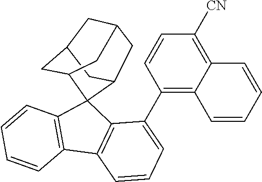 [SMILES: N#Cc1ccc(-c2cccc3c2C2(c4ccccc4-3)C3CC4CC(C3)CC2C4)c2ccccc12] | 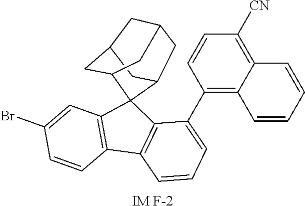 | 56 |
| 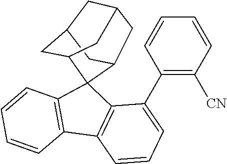 [SMILES: N#Cc1ccccc1-c1cccc2c1C1(c3ccccc3-2)C2CC3CC(C2)CC1C3] | 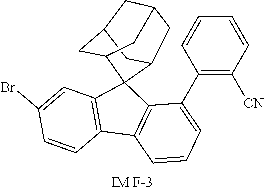 | 53 |
| 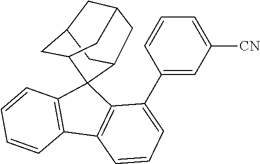 [SMILES: N#Cc1cccc(-c2cccc3c2C2(c4ccccc4-3)C3CC4CC(C3)CC2C4)c1] | 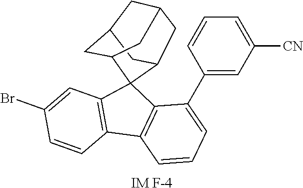 | 56 |
| 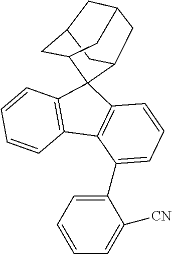 [SMILES: N#Cc1ccccc1-c1cccc2c1-c1ccccc1C21C2CC3CC(C2)CC1C3] | 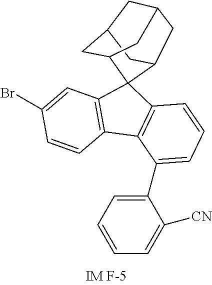 | 48 |
| 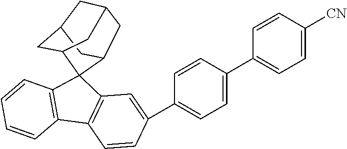 [SMILES: N#Cc1ccc(-c2ccc(-c3ccc4c(c3)C3(c5ccccc5-4)C4CC5CC(C4)CC3C5)cc2)cc1] | 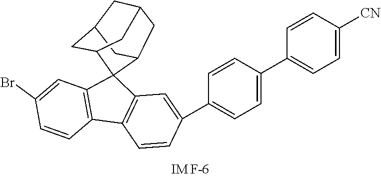 | 51 |
| 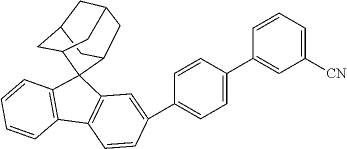 [SMILES: N#Cc1cccc(-c2ccc(-c3ccc4c(c3)C3(c5ccccc5-4)C4CC5CC(C4)CC3C5)cc2)c1] | 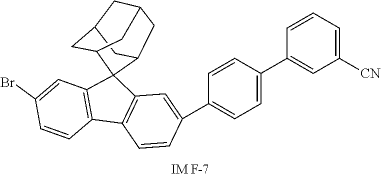 | 50 |
| 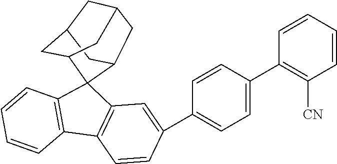 [SMILES: N#Cc1ccccc1-c1ccc(-c2ccc3c(c2)C2(c4ccccc4-3)C3CC4CC(C3)CC2C4)cc1] | 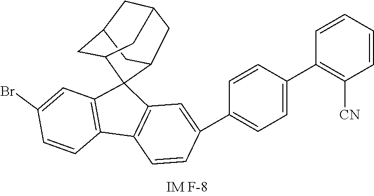 | 49 |
| 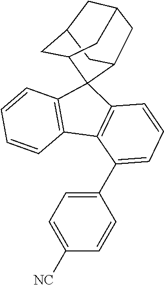 [SMILES: N#Cc1ccc(-c2cccc3c2-c2ccccc2C32C3CC4CC(C3)CC2C4)cc1] | 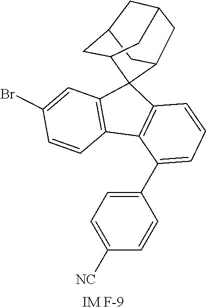 | 62 |
| 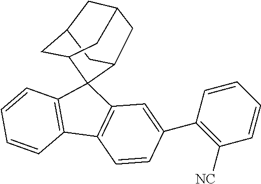 [SMILES: N#Cc1ccccc1-c1ccc2c(c1)C1(c3ccccc3-2)C2CC3CC(C2)CC1C3] | 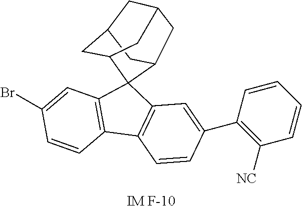 | 59 |
| 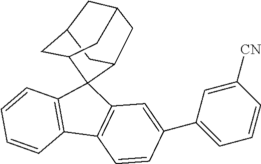 [SMILES: N#Cc1cccc(-c2ccc3c(c2)C2(c4ccccc4-3)C3CC4CC(C3)CC2C4)c1] | 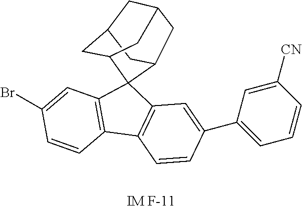 | 62 |
| 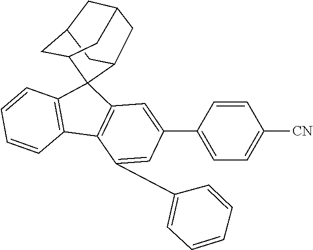 [SMILES: N#Cc1ccc(-c2cc(-c3ccccc3)c3c(c2)C2(c4ccccc4-3)C3CC4CC(C3)CC2C4)cc1] | 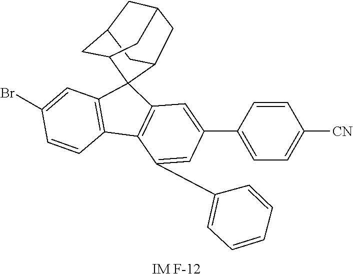 | 46 |

*: The main product synthesized in step (3) refers to a product employed as the raw material in step (4).

### Synthesis of Compound

### Synthesis Example 1: Synthesis of Compound 1

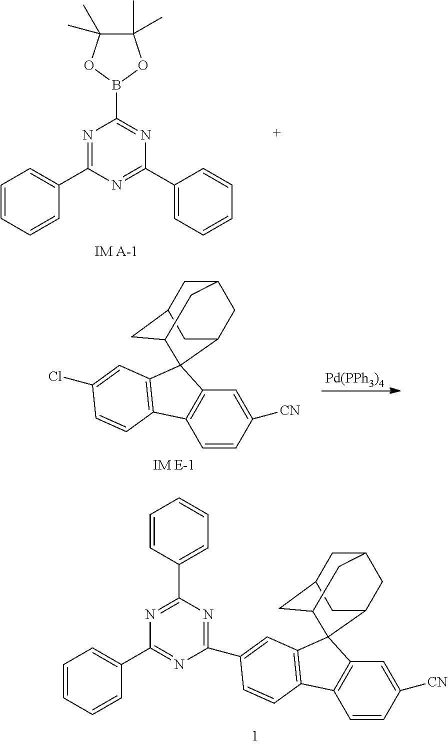

Under nitrogen protection, IM A-1 (3.59 g, 10.0 mmol), IM E-1 (3.45 g, 10.0 mmol), 2.76 g of potassium carbonate, 0.32 g of tetrabutylammonium bromide, 0.12 g of tetrakis(triphenylphosphine)palladium, 40 mL of toluene, 20 mL of ethanol, and 15 mL of water were mixed and heated to 72° C., a reaction was completed in 10 h. The reaction solution was washed with water, then dried by adding magnesium sulfate, and filtered to obtain a filtrate, solvent of the filtrate was removed under reduced pressure. Crude product was purified by recrystallization using a dichloromethane/ethyl acetate system (1 g of a crude product: 3 mL of dichloromethane: 6 mL of ethyl acetate) to obtain an off-white solid compound 1 (3.90 g, yield 72.5%), m/z=543.7[M+H]⁺. Nuclear magnetism data of the compound 1: ¹H NMR (400 MHz, CD₂Cl₂): 8.79 (d, 4H), 8.28 (s, 1H), 8.0 (d, 1H), 7.83 (d, 1H), 7.68-7.56 (m, 8H), 7.48 (s, 1H), 2.6 (s, 2H), 2.07-1.78 (m, 8H), and 1.69 (m, 4H).

### Synthesis Example 2: Synthesis of Compound 8

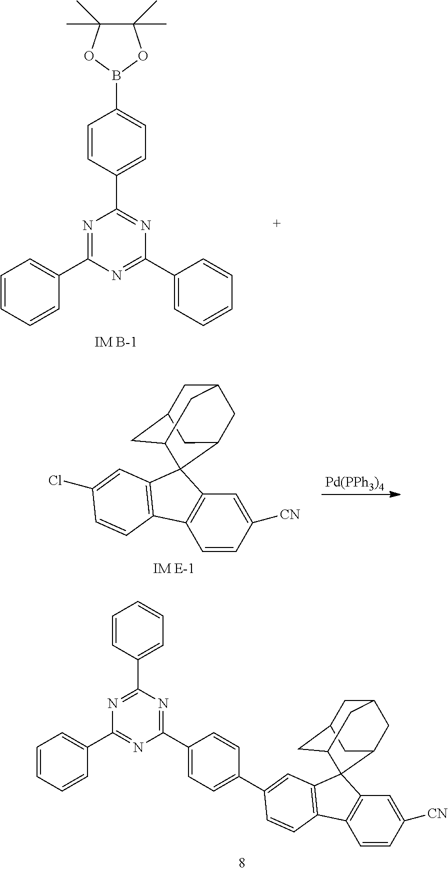

IM B-1 (4.35 g, 10.0 mmol), IM E-1 (3.45 g, 10.0 mmol), 2.76 g of potassium carbonate, 0.32 g of tetrabutylammonium bromide, 0.12 g of tetrakis(triphenylphosphine)palladium, 40 mL of toluene, 20 mL of ethanol, and 15 mL of water were added into a reaction bulb under nitrogen protection, then heated to 72° C. and reacted for 8 h. The reaction solution was washed with water, dried by adding magnesium sulfate, and filtered. Solvent in the filtrate was removed under reduced pressure and then crude product was purified by recrystallization using a dichloromethane/ethyl acetate system (1 g of the crude product: 3 mL of dichloromethane: 6 mL of ethyl acetate) to obtain an off-white solid compound 8 (4.20 g, yield 68%), m/z=619.3[M+H]⁺.

### Synthesis Example 3: Synthesis of Compound 40

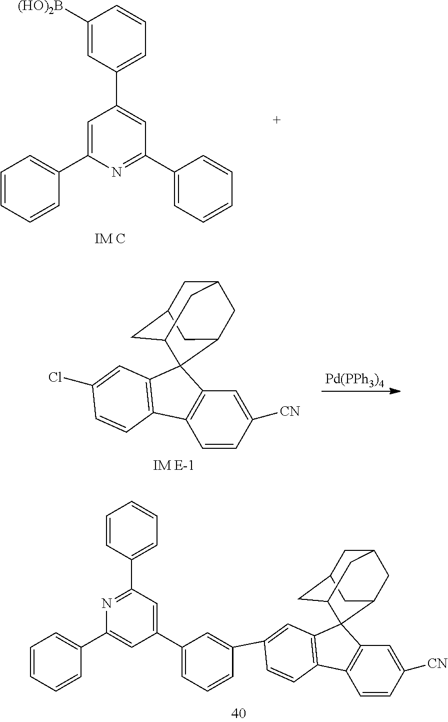

IM C (3.51 g, 10.0 mmol), IM E-1 (3.45 g, 21.0 mmol), 2.76 g of potassium carbonate, 0.32 g of tetrabutylammonium bromide, 0.12 g of tetrakis(triphenylphosphine)palladium, 40 mL of toluene, 20 mL of ethanol, and 15 mL of water were added into a reaction bulb under nitrogen protection, heated to 72° C. and a reaction was completed in 8 h and then cooled down to the room temperature. The reaction solution was washed with water, dried by adding magnesium sulfate, and filtered, and solvent of the filtrate was removed under reduced pressure. A crude product was purified by recrystallization using a dichloromethane/ethyl acetate system (1 g of the crude product: 3 mL of dichloromethane: 6 mL of ethyl acetate) to obtain an off-white solid compound 40 (3.44 g, yield 56%), m/z=617.2[M+H]⁺.

### Synthesis Examples 4-27

The compound is synthesized with reference to the method of synthesis example 1. The differences including: replacing IM A-1 by a raw material I, replacing IM E-1 by a raw material II. The employed main raw materials. the correspondingly synthesized compounds, the yield of the compound, and a mass spectrometry characterization result are shown in Table 5.

TABLE 5

| Synthe- | | |
|---|---|---|
| sis | | |
| exam- | | |
| ple | | |
| No. | Raw material I | Raw material II |
| 4 | 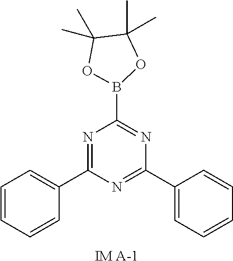 | 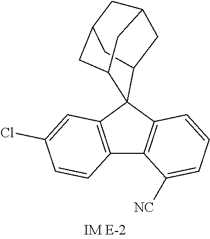 [SMILES: N#Cc1cccc2c1-c1ccc(Cl)cc1C21C2CC3CC(C2)CC1C3] |
| 5 | 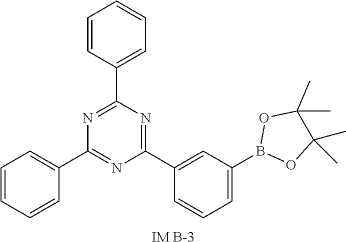 | 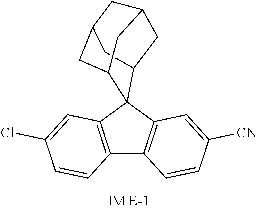 [SMILES: N#Cc1ccc2c(c1)C1(c3cc(Cl)ccc3-2)C2CC3CC(C2)CC1C3] |
| 6 | 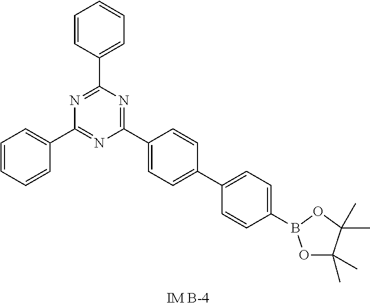 | 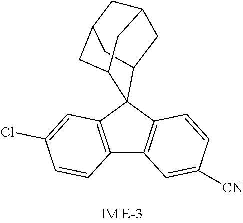 [SMILES: N#Cc1ccc2c(c1)-c1ccc(Cl)cc1C21C2CC3CC(C2)CC1C3] |
| 7 | 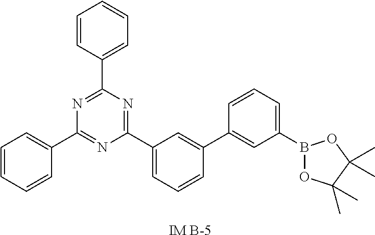 | 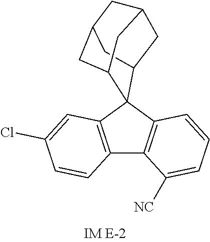 [SMILES: N#Cc1cccc2c1-c1ccc(Cl)cc1C21C2CC3CC(C2)CC1C3] |
| 8 | 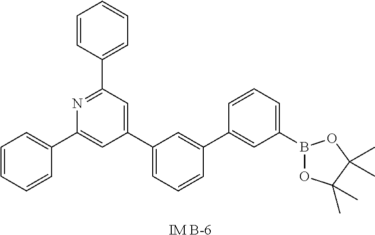 | 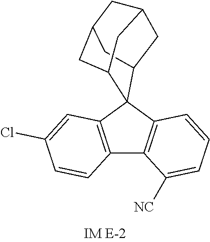 [SMILES: N#Cc1cccc2c1-c1ccc(Cl)cc1C21C2CC3CC(C2)CC1C3] |
| 9 | 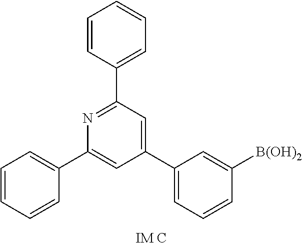 | 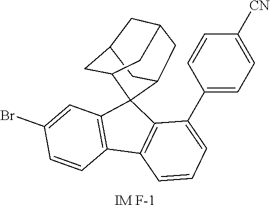 |
| 10 | 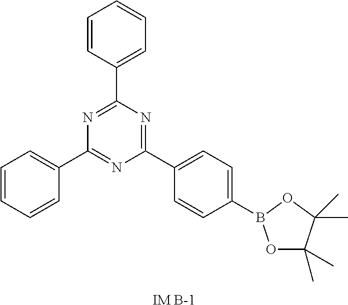 | 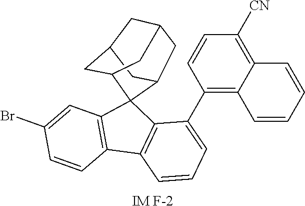 |
| 11 | 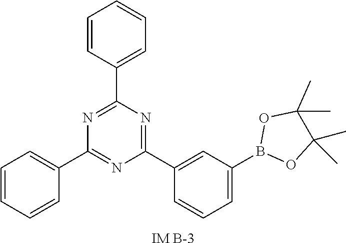 | 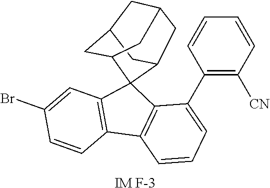 |
| 12 | 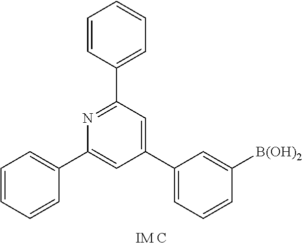 | 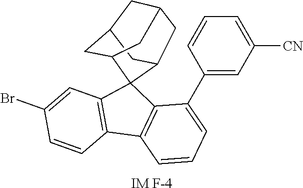 |
| 13 | 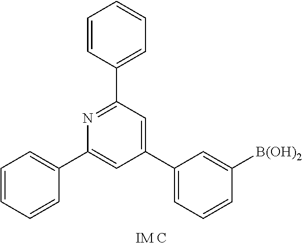 | 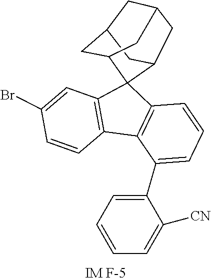 |
| 14 | 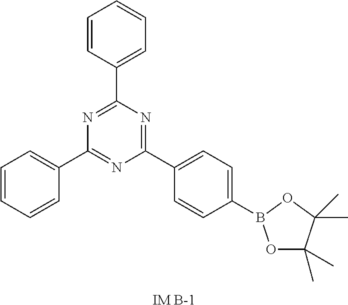 | 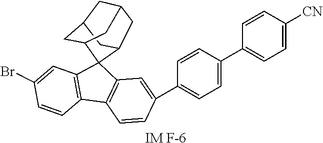 |
| 15 | 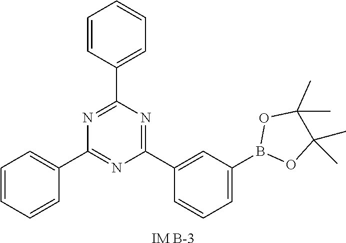 | 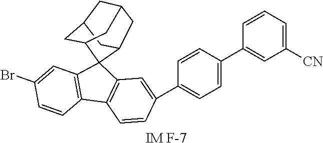 |
| 16 | 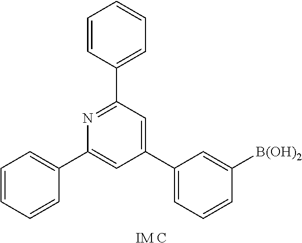 | 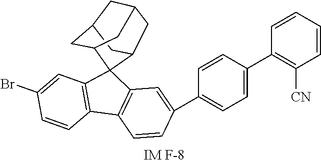 |
| 17 | 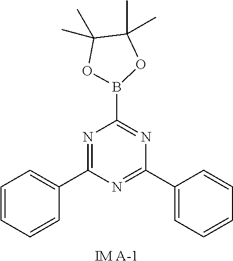 | 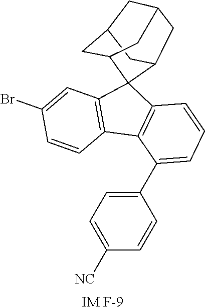 |
| 18 | 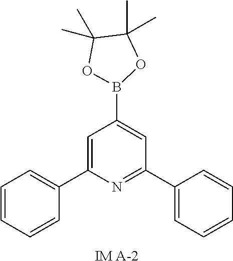 | 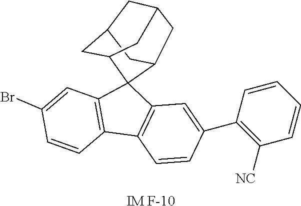 |
| 19 | 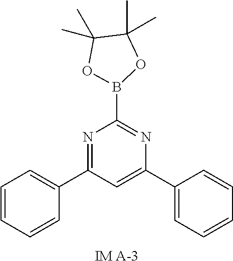 | 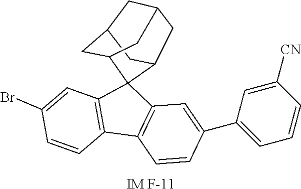 |
| 20 | 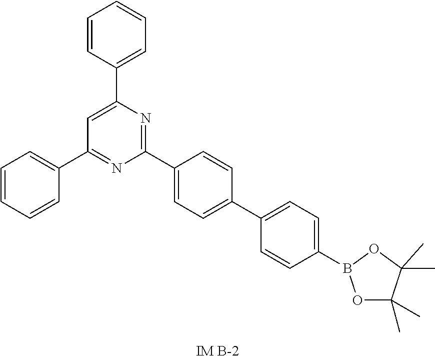 | 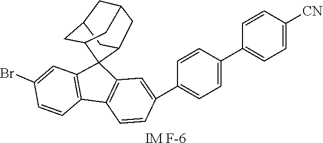 |
| 21 | 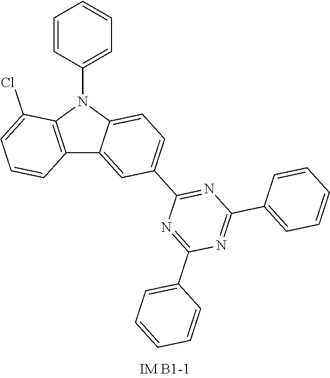 [SMILES: Clc1cccc2c3cc(-c4nc(-c5ccccc5)nc(-c5ccccc5)n4)ccc3n(-c3ccccc3)c12] | 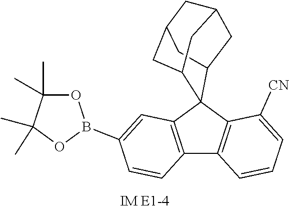 [SMILES: CC1(C)OB(c2ccc3c(c2)C2(c4c(C#N)cccc4-3)C3CC4CC(C3)CC2C4)OC1(C)C] |
| 22 | 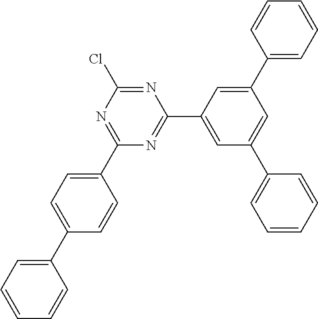 [SMILES: Clc1nc(-c2ccc(-c3ccccc3)cc2)nc(-c2cc(-c3ccccc3)cc(-c3ccccc3)c2)n1] | 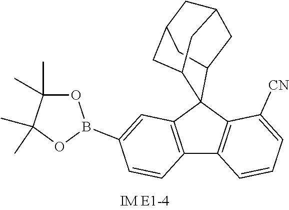 [SMILES: CC1(C)OB(c2ccc3c(c2)C2(c4c(C#N)cccc4-3)C3CC4CC(C3)CC2C4)OC1(C)C] |
| 23 | 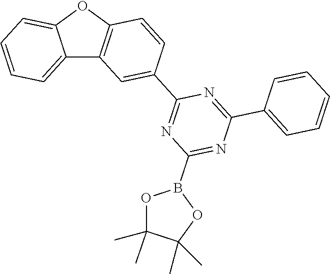 [SMILES: CC1(C)OB(c2nc(-c3ccccc3)nc(-c3ccc4oc5ccccc5c4c3)n2)OC1(C)C] | 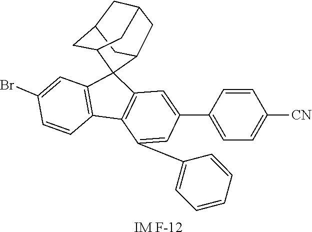 |
| 24 | 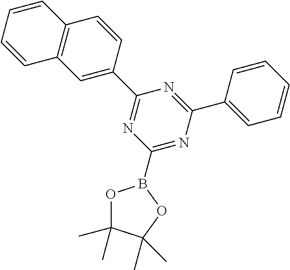 [SMILES: CC1(C)OB(c2nc(-c3ccccc3)nc(-c3ccc4ccccc4c3)n2)OC1(C)C] | 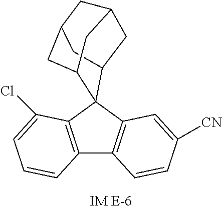 [SMILES: N#Cc1ccc2c(c1)C1(c3c(Cl)cccc3-2)C2CC3CC(C2)CC1C3] |
| 25 | 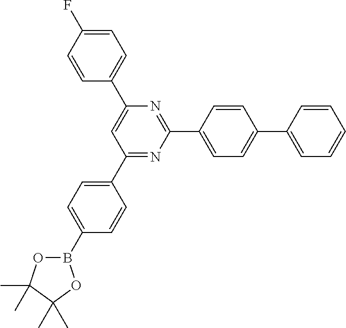 [SMILES: CC1(C)OB(c2ccc(-c3cc(-c4ccc(F)cc4)nc(-c4ccc(-c5ccccc5)cc4)n3)cc2)OC1(C)C] | 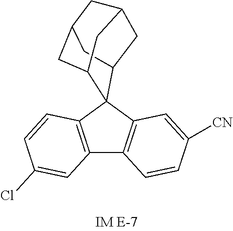 [SMILES: N#Cc1ccc2c(c1)C1(c3ccc(Cl)cc3-2)C2CC3CC(C2)CC1C3] |
| 26 | 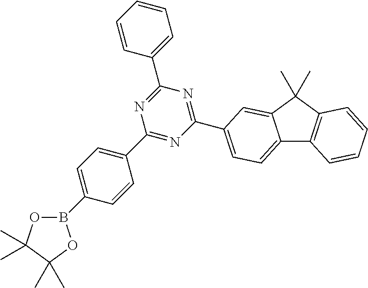 [SMILES: CC1(C)c2ccccc2-c2ccc(-c3nc(-c4ccccc4)nc(-c4ccc(B5OC(C)(C)C(C)(C)O5)cc4)n3)cc21] | 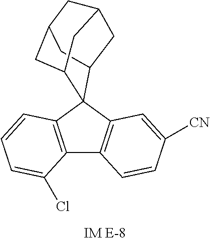 [SMILES: N#Cc1ccc2c(c1)C1(c3cccc(Cl)c3-2)C2CC3CC(C2)CC1C3] |
| 27 | 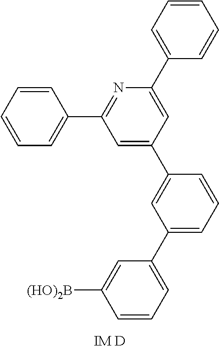 [SMILES: OB(O)c1cccc(-c2cccc(-c3cc(-c4ccccc4)nc(-c4ccccc4)c3)c2)c1] | 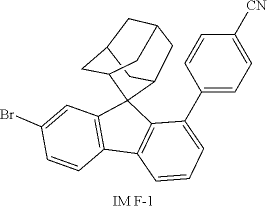 |

| | | | Mass |
|---|---|---|---|
| Synthesis | | | spectrum |
| example | | Yield | (m/z) |
| No. | Compound | (%) | [M + H]⁺ |
| 4 | 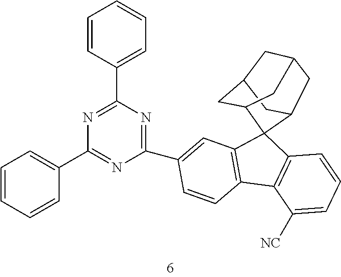 [SMILES: N#Cc1cccc2c1-c1ccc(-c3nc(-c4ccccc4)nc(-c4ccccc4)n3)cc1C21C2CC3CC(C2)CC1C3] | 45 | 543.2 |
| 5 | 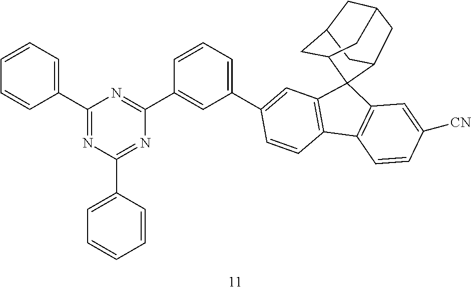 [SMILES: N#Cc1ccc2c(c1)C1(c3cc(-c4cccc(-c5nc(-c6ccccc6)nc(-c6ccccc6)n5)c4)ccc3-2)C2CC3CC(C2)CC1C3] | 42 | 619.2 |
| 6 | 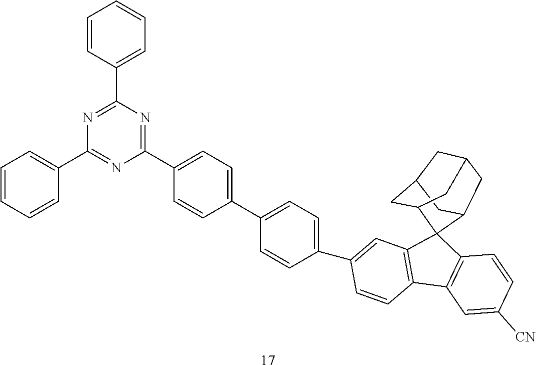 [SMILES: N#Cc1ccc2c(c1)-c1ccc(-c3ccc(-c4ccc(-c5nc(-c6ccccc6)nc(-c6ccccc6)n5)cc4)cc3)cc1C21C2CC3CC(C2)CC1C3 ; "17"] | 40 | 695.4 |
| 7 | 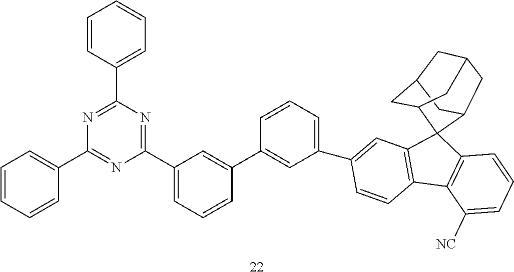 [SMILES: N#Cc1cccc2c1-c1ccc(-c3cccc(-c4cccc(-c5nc(-c6ccccc6)nc(-c6ccccc6)n5)c4)c3)cc1C21C2CC3CC(C2)CC1C3 ; "22"] | 43 | 695.4 |
| 8 | 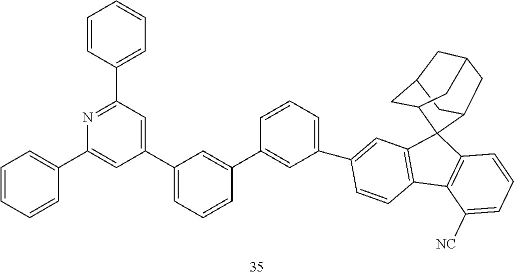 [SMILES: N#Cc1cccc2c1-c1ccc(-c3cccc(-c4cccc(-c5cc(-c6ccccc6)nc(-c6ccccc6)c5)c4)c3)cc1C21C2CC3CC(C2)CC1C3 ; "35"] | 38 | 693.3 |
| 9 | 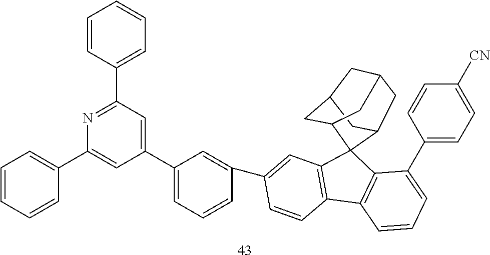 [SMILES: N#Cc1ccc(-c2cccc3c2C2(c4cc(-c5cccc(-c6cc(-c7ccccc7)nc(-c7ccccc7)c6)c5)ccc4-3)C3CC4CC(C3)CC2C4)cc1 ; "43"] | 56 | 693.3 |
| 10 | 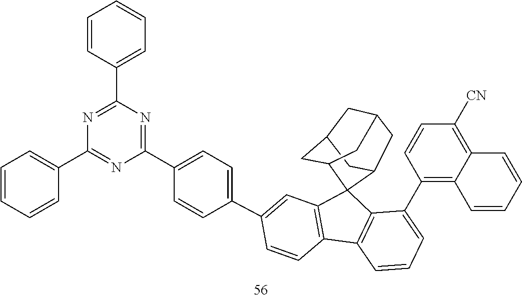 | 45 | 745.6 |
| 11 | 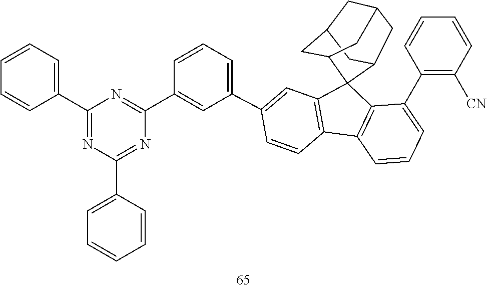 [SMILES: N#Cc1ccccc1-c1cccc2c1C1(c3cc(-c4cccc(-c5nc(-c6ccccc6)nc(-c6ccccc6)n5)c4)ccc3-2)C2CC3CC(C2)CC1C3 ; "65"] | 44 | 695.3 |
| 12 | 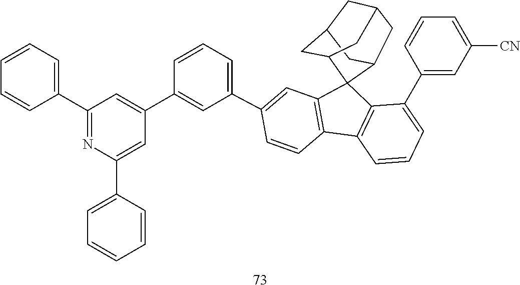 [SMILES: N#Cc1cccc(-c2cccc3c2C2(c4cc(-c5cccc(-c6cc(-c7ccccc7)nc(-c7ccccc7)c6)c5)ccc4-3)C3CC4CC(C3)CC2C4)c1 ; "73"] | 52 | 693.3 |
| 13 | 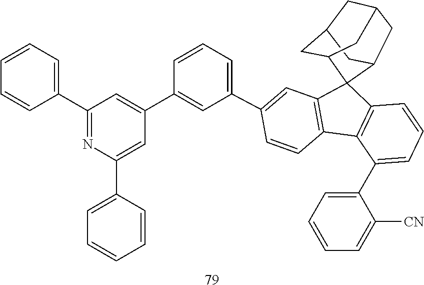 [SMILES: N#Cc1ccccc1-c1cccc2c1-c1ccc(-c3cccc(-c4cc(-c5ccccc5)nc(-c5ccccc5)c4)c3)cc1C21C2CC3CC(C2)CC1C3 ; "79"] | 50 | 693.3 |
| 14 | 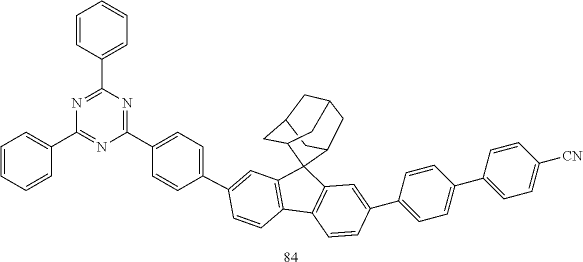 | 43 | 771.4 |
| 15 | 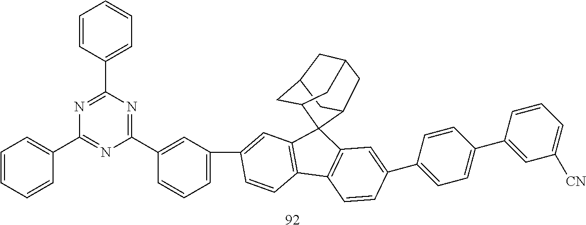 | 37 | 771.4 |
| 16 | 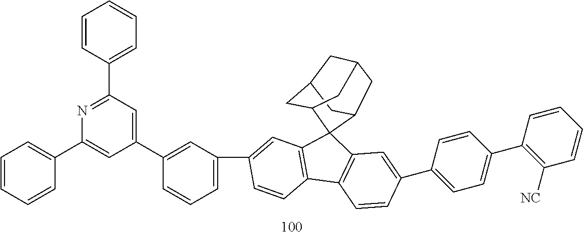 | 36 | 769.5 |
| 17 | 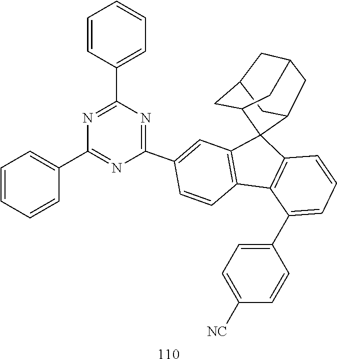 [SMILES: N#Cc1ccc(-c2cccc3c2-c2ccc(-c4nc(-c5ccccc5)nc(-c5ccccc5)n4)cc2C32C3CC4CC(C3)CC2C4)cc1 ; "110"] | 38 | 619.2 |
| 18 | 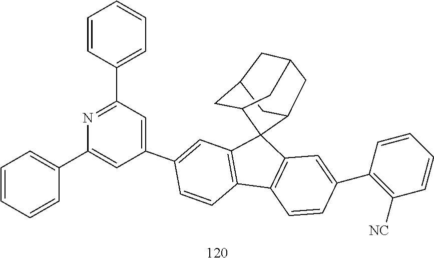 [SMILES: N#Cc1ccccc1-c1ccc2c(c1)C1(c3cc(-c4cc(-c5ccccc5)nc(-c5ccccc5)c4)ccc3-2)C2CC3CC(C2)CC1C3 ; "120"] | 43 | 617.1 |
| 19 | 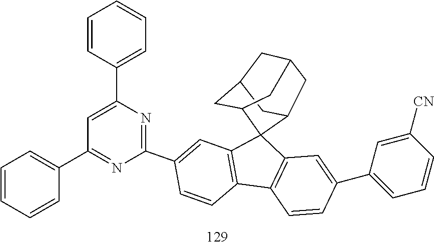 | 47 | 618.8 |
| 20 | 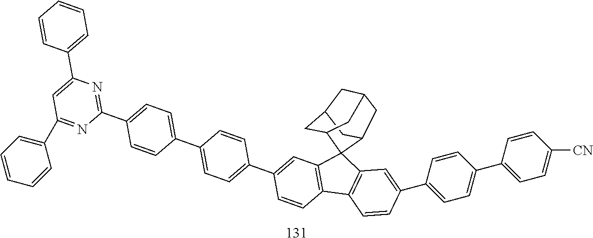 | 44 | 846.4 |
| 21 | 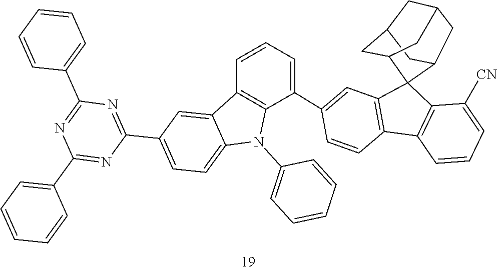 [SMILES: N#Cc1cccc2c1C1(c3cc(-c4cccc5c6cc(-c7nc(-c8ccccc8)nc(-c8ccccc8)n7)ccc6n(-c6ccccc6)c45)ccc3-2)C2CC3CC(C2)CC1C3 ; "19"] | 45 | 784.3 |
| 22 | 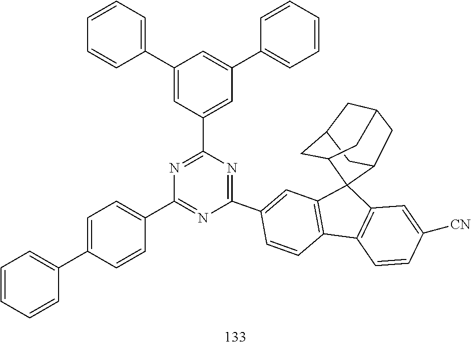 [SMILES: N#Cc1ccc2c(c1)C1(c3cc(-c4nc(-c5ccc(-c6ccccc6)cc5)nc(-c5cc(-c6ccccc6)cc(-c6ccccc6)c5)n4)ccc3-2)C2CC3CC(C2)CC1C3 ; "133"] | 38 | 771.3 |
| 23 | 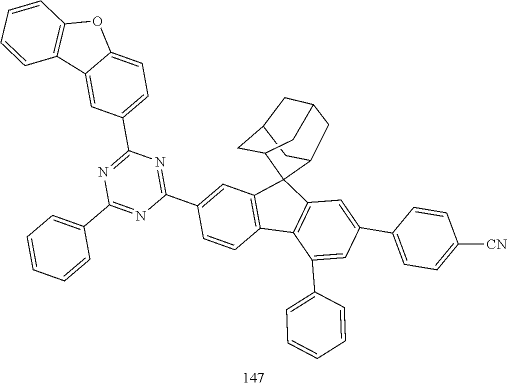 [SMILES: N#Cc1ccc(-c2cc(-c3ccccc3)c3c(c2)C2(c4cc(-c5nc(-c6ccccc6)nc(-c6ccc7oc8ccccc8c7c6)n5)ccc4-3)C3CC4CC(C3)CC2C4)cc1 ; "147"] | 37 | 785.3 |
| 24 | 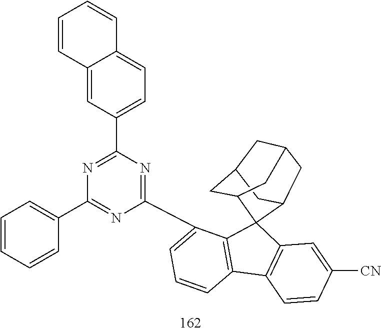 | 43 | 593.3 |
| 25 | 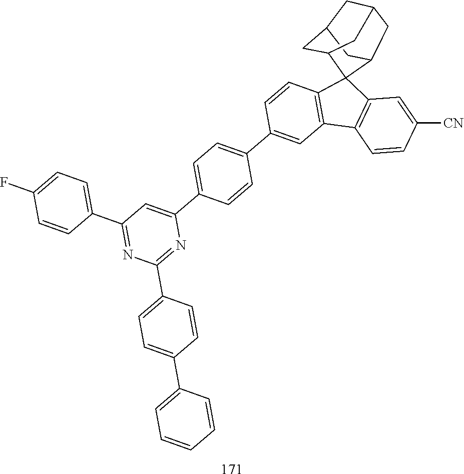 | 48 | 712.3 |
| 26 | 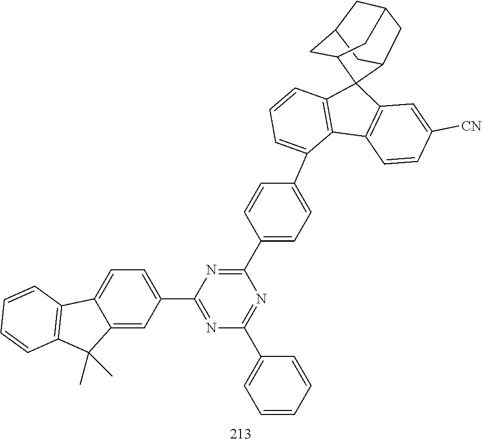 | 46 | 735.3 |
| 27 | 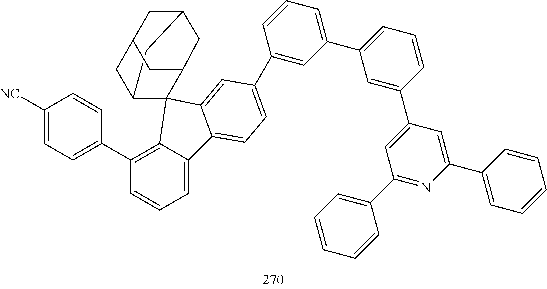 [SMILES: N#Cc1ccc(-c2cccc3c2C2(c4cc(-c5cccc(-c6cccc(-c7cc(-c8ccccc8)nc(-c8ccccc8)c7)c6)c5)ccc4-3)C3CC4CC(C3)CC2C4)cc1] | 52 | 769.4 |

Nuclear magnetism data of a compound 73: ¹H NMR (400 MHz, CD₂Cl₂): 8.29 (s, 1H), 8.18-8.14 (m, 6H), 7.98 (s, 1H), 7.84 (d, 1H), 7.79-7.69 (m, 5H), 7.60-7.37 (m, 11H), 7.26 (d, 1H), 2.11 (s, 2H), 1.78-1.56 (m, 8H), and 1.44-1.35 (m, 4H).

Nuclear magnetism data of a compound 92: ¹H NMR (400 MHz, CD₂Cl₂): 8.64 (d, 4H), 8.55 (s, 1H), 8.32 (d, 1H), 8.0 (s, 1H), 7.83-7.73 (m, 5H), 7.67-7.53 (m, 15H), 7.39 (d, 1H), 2.07 (s, 2H), 1.87-1.66 (m, 7H), and 1.54-1.39 (m, 5H).

Preparation and Evaluation of Organic Electroluminescent Device

### Example 1 Blue Organic Electroluminescent Device

An anode is prepared through the following processes: an ITO substrate with a thickness of 1500 Å (manufactured by Corning) is cut into a size of 40 mm×40 mm×0.7 mm, so as to be prepared into an experimental substrate with a cathode, an anode and an insulating layer pattern by adopting a photoetching process. The surface treatment is performed by utilizing ultraviolet ozone and O₂:N₂ plasma, for increasing work function of the anode (the experiment substrate) and removing scum.

F4-TCNQ is vacuum-evaporated on the experiment substrate (the anode) to form a hole injection layer (HIL) with a thickness of 100 Å, and HT-01 is evaporated on the hole injection layer to form a hole transporting layer with a thickness of 900 Å.

HT-02 is vacuum-evaporated on the hole transporting layer to form an electron blocking layer with a thickness of 150 Å.

With a thickness ratio of 98%:2% in a film, BH-01 and BD-01 are co-evaporated on the electron blocking layer to form a blue light-emitting layer (EML) with a thickness of 200 Å.

The compound 1 and LiQ are mixed at a weight ratio of 1:1, the mixture is evaporated to form an electron transporting layer (ETL) with a thickness of 300 Å, LiQ is evaporated on the electron transporting layer to form an electron injection layer (EIL) with a thickness of 10 Å, and then magnesium (Mg) and silver (Ag) are mixed at an evaporation rate of 1:9 and vacuum-evaporated on the electron injection layer to form a cathode with a thickness of 105 Å.

In addition, a compound CP-01 is evaporated on the above cathode to form an organic capping layer (CPL) with a thickness of 650 Å, thus the organic electroluminescent device is completely manufactured.

### Examples 2 to 27

In addition to replacing the compound 1 with compounds that shown in the following Table 6 to form the electron transporting layer, the organic electroluminescent device is manufactured by utilizing the same method as in Example 1.

### Comparative Examples 1 to 6

In addition to replacing the compound 1 with compound A to compound F that shown in the following Table 6 to form the electron transporting layer, the organic electroluminescent device is manufactured by utilizing the same method as in Example 1.

The main material structures used in the above examples and comparative examples are as follows:

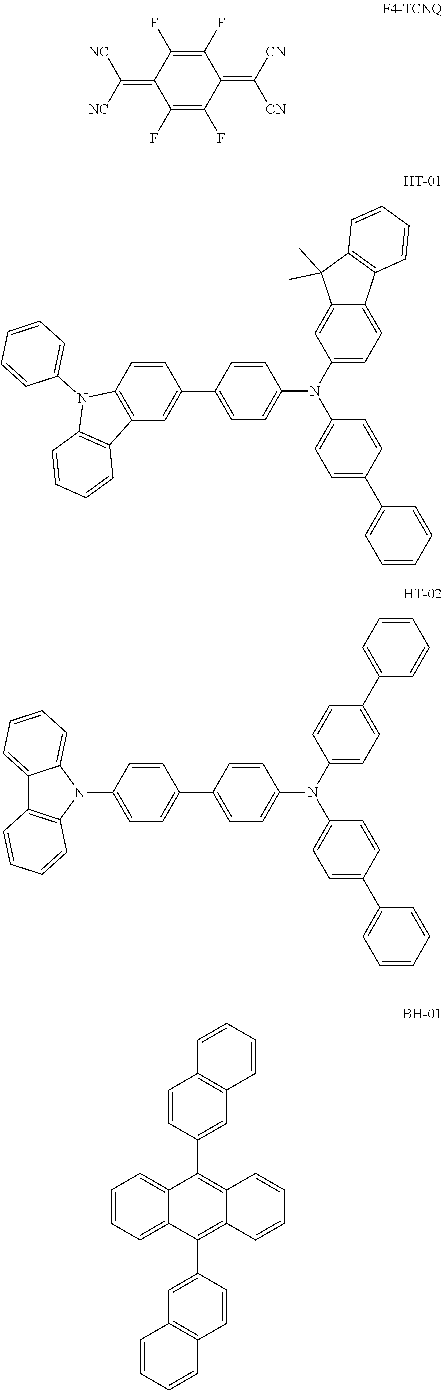

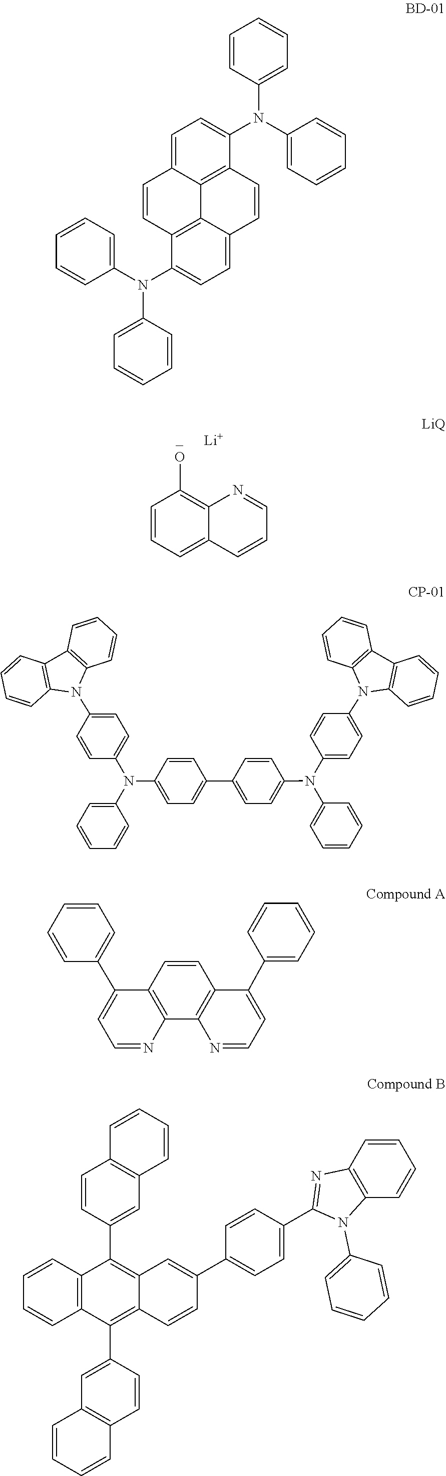 [SMILES: c1ccc(N(c2ccccc2)c2ccc3ccc4c(N(c5ccccc5)c5ccccc5)ccc5ccc2c3c54)cc1 ; [Li+] ; Oc1cccc2cccnc12 ; c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; c1ccc(-c2ccnc3c2ccc2c(-c4ccccc4)ccnc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

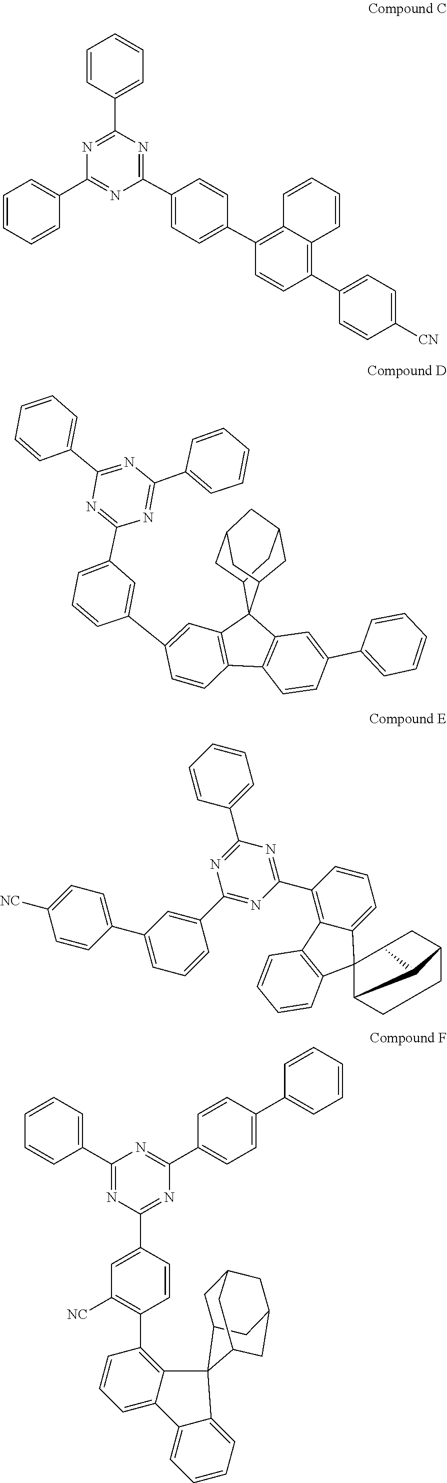

For the organic electroluminescent device prepared as above, the optoelectronic performance (driving voltage, power and chromaticity coordinates) under a condition of 10 mA/cm² and the service life of the device under a condition of 20 mA/cm² are analyzed, and its results are shown in Table 6:

TABLE 6

| | | | Light | | | | External | |
|---|---|---|---|---|---|---|---|---|
| | ETL | Driving | emitting | Power | Chromaticity | Chromaticity | quantum | |
| | layer | voltage | efficiency | efficiency | coordinate | coordinate | efficiency | T95 |
| Example | compound | (V) | (Cd/A) | (lm/W) | CIE-x | CIE-y | EQE % | (hr) |
| Example 1 | Compound 1 | 3.73 | 6.1 | 5.1 | 0.14 | 0.05 | 12.5 | 129 |
| Example 2 | Compound 6 | 3.65 | 6.0 | 5.2 | 0.140 | 0.050 | 12.4 | 130 |
| Example 3 | Compound 8 | 3.72 | 6.0 | 5.1 | 0.140 | 0.050 | 12.4 | 122 |
| Example 4 | Compound 11 | 3.76 | 6.1 | 5.1 | 0.140 | 0.050 | 12.6 | 130 |
| Example 5 | Compound 17 | 3.70 | 6.2 | 5.3 | 0.140 | 0.050 | 12.8 | 131 |
| Example 6 | Compound 19 | 3.79 | 6.1 | 5.1 | 0.140 | 0.050 | 12.6 | 145 |
| Example 7 | Compound 22 | 3.73 | 6.3 | 5.3 | 0.140 | 0.050 | 13.0 | 131 |
| Example 8 | Compound 35 | 3.71 | 5.9 | 5.0 | 0.140 | 0.050 | 12.1 | 144 |
| Example 9 | Compound 40 | 3.60 | 5.9 | 5.1 | 0.140 | 0.050 | 12.0 | 126 |
| Example 10 | Compound 43 | 3.73 | 6.7 | 5.6 | 0.140 | 0.050 | 13.8 | 167 |
| Example 11 | Compound 56 | 3.63 | 6.9 | 6.0 | 0.140 | 0.050 | 14.3 | 150 |
| Example 12 | Compound 65 | 3.67 | 6.8 | 5.8 | 0.140 | 0.050 | 14.0 | 170 |
| Example 13 | Compound 73 | 3.79 | 6.7 | 5.6 | 0.140 | 0.050 | 13.8 | 158 |
| Example 14 | Compound 79 | 3.62 | 6.7 | 5.8 | 0.140 | 0.050 | 13.7 | 156 |
| Example 15 | Compound 84 | 3.75 | 6.7 | 5.6 | 0.140 | 0.050 | 13.7 | 170 |
| Example 16 | Compound 92 | 3.61 | 6.8 | 6.0 | 0.140 | 0.050 | 14.1 | 167 |
| Example 17 | Compound 100 | 3.77 | 6.7 | 5.6 | 0.140 | 0.050 | 13.8 | 159 |
| Example 18 | Compound 110 | 3.68 | 6.8 | 5.8 | 0.140 | 0.050 | 14.0 | 157 |
| Example 19 | Compound 120 | 3.75 | 6.7 | 5.6 | 0.140 | 0.050 | 13.7 | 152 |
| Example 20 | Compound 129 | 3.64 | 6.7 | 5.8 | 0.140 | 0.050 | 13.8 | 154 |
| Example 21 | Compound 131 | 3.77 | 6.6 | 5.5 | 0.140 | 0.050 | 13.6 | 160 |
| Example 22 | Compound 133 | 3.70 | 6.0 | 5.1 | 0.140 | 0.050 | 12.3 | 136 |
| Example 23 | Compound 147 | 3.76 | 6.9 | 5.8 | 0.140 | 0.050 | 14.2 | 162 |
| Example 24 | Compound 162 | 3.74 | 6.3 | 5.3 | 0.140 | 0.050 | 12.9 | 140 |
| Example 25 | Compound 171 | 3.71 | 6.0 | 5.0 | 0.140 | 0.050 | 12.2 | 135 |
| Example 26 | Compound 213 | 3.78 | 6.0 | 5.0 | 0.140 | 0.050 | 12.3 | 144 |
| Example 27 | Compound 270 | 3.71 | 6.9 | 5.8 | 0.140 | 0.050 | 14.1 | 168 |
| Comparative | Compound A | 4.34 | 5.1 | 3.7 | 0.140 | 0.050 | 10.5 | 80 |
| example 1 | | | | | | | | |
| Comparative | Compound B | 4.08 | 5.9 | 4.5 | 0.140 | 0.050 | 12.1 | 97 |
| example 2 | | | | | | | | |
| Comparative | Compound C | 4.08 | 6.0 | 4.6 | 0.140 | 0.050 | 12.4 | 102 |
| example 3 | | | | | | | | |
| Comparative | Compound D | 3.96 | 6.6 | 5.3 | 0.140 | 0.050 | 13.6 | 110 |
| example 4 | | | | | | | | |
| Comparative | Compound E | 3.62 | 6.9 | 6.0 | 0.140 | 0.050 | 14.2 | 109 |
| example 5 | | | | | | | | |
| Comparative | Compound F | 3.77 | 6.9 | 5.8 | 0.140 | 0.050 | 14.3 | 105 |
| example 6 | | | | | | | | |

It can be seen from Table 6 that, as the material of the electron transporting layer, when compared with comparative examples 1 to 6, the organic electroluminescent device prepared with the nitrogen-containing compound of the present disclosure in examples 1 to 27 have improved performances. Comparing examples 1 to 27 with comparative examples 1 to 3, the driving voltage is greatly reduced, and meanwhile the light-emitting efficiency and working life of the device are improved to a certain extent; and compared with comparative examples 4 to 6, the working life of the device in examples 1 to 27 is improved to a great extent. Compared with comparative examples 1 to 6, the service life of the device in examples 1 to 27 is improved by at least about 11%.

1. A nitrogen-containing compound, wherein the structure of the nitrogen-containing compound is selected from Formula 1-1 and Formula 1-2: 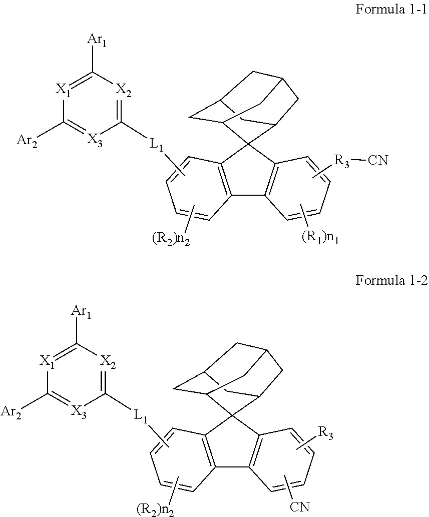
wherein X₁, X₂ and X₃ are the same or different, and each independently represent C(H) or N, and at least one of the three is N;
R₁ and R₂ are selected from an aryl with 6 carbon atoms;
n₁ represents the number of R₁, n₁ is selected from 0 or 1; and n₂ represents the number of R₂, n₂ is selected from 0 or 1;
wherein in the Formula 1-1, 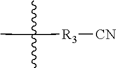
is selected from the group consisting of the following groups: 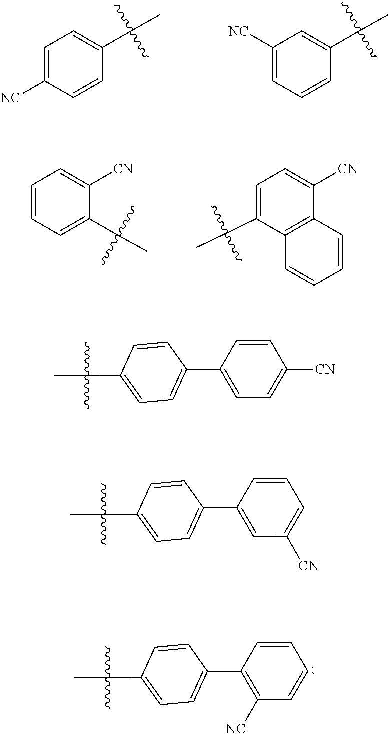
wherein in the Formula 1-2, R₃ is selected from hydrogen;
wherein L₁ is selected from single bond, or an unsubstituted group T₁, wherein the unsubstituted group T₁ is selected from the group consisting of the following groups: 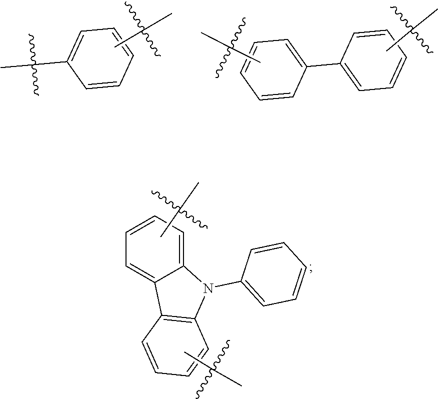
wherein Ar₁ and Ar₂ are the same or different, and are each independently selected from a substituted or unsubstituted group V, wherein the unsubstituted group V is selected from the group consisting of the following groups: 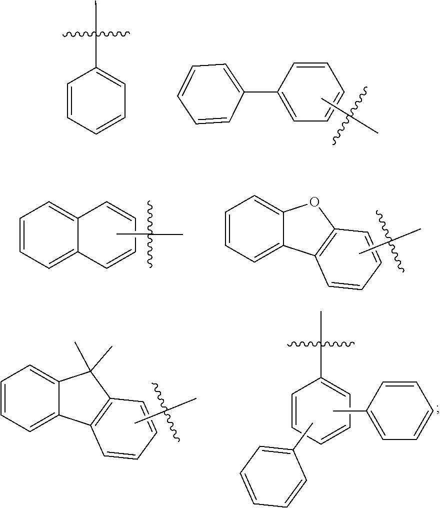
and the substituted group V has one or two or more substituents, and the substituents are each independently selected from deuterium, fluorine, or an alkyl with 1 to 4 carbon atoms.

2-6. (canceled)

7. The nitrogen-containing compound of claim 1, wherein L₁ is selected from single bond, or the group consisting of the following groups:

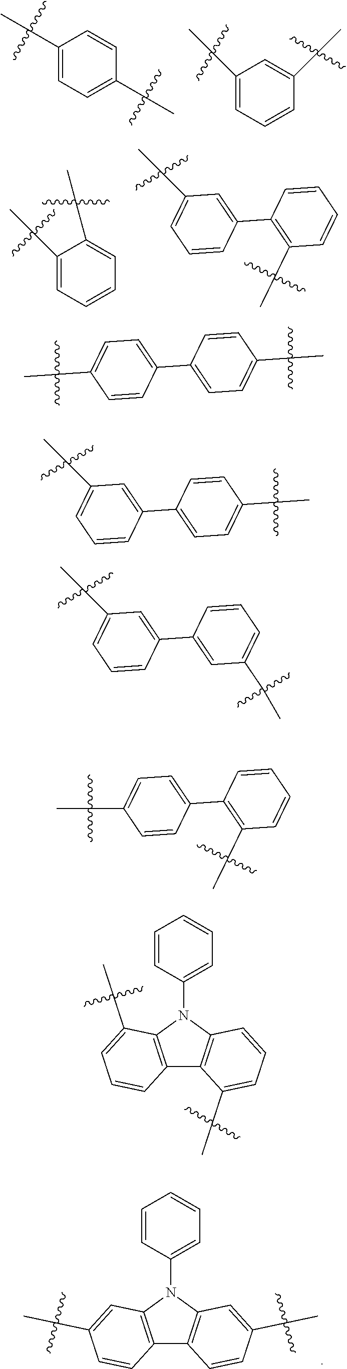

8-10. (canceled)

11. The nitrogen-containing compound of claim 1, wherein Ar₁ and Ar₂ are the same or different, and are each independently selected from the group consisting of the following groups:

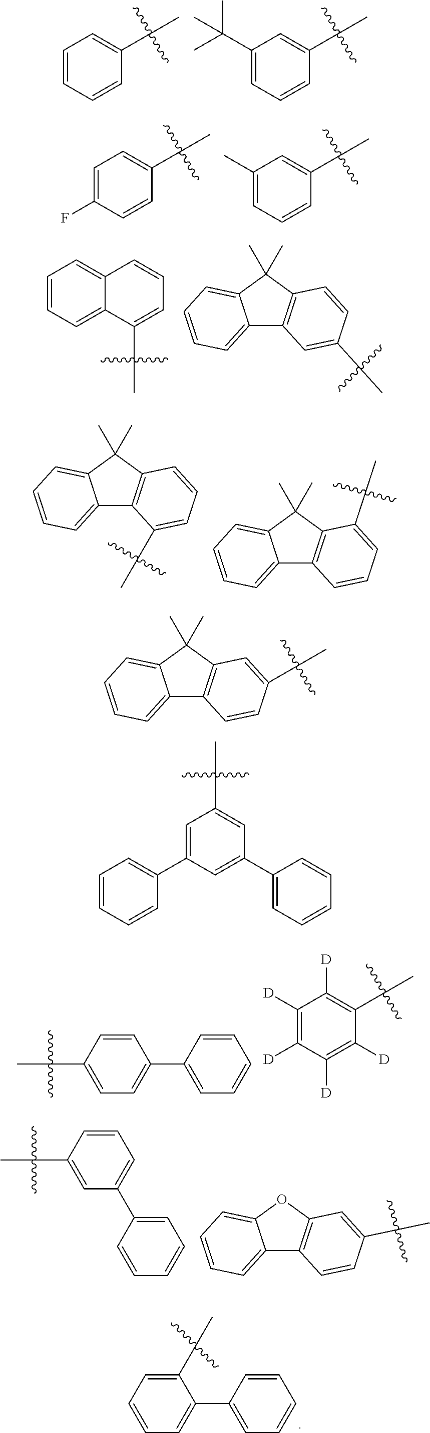

12. (canceled)

13. The nitrogen-containing compound of claim 1, wherein the nitrogen-containing compound is selected from the group consisting of the following compounds:

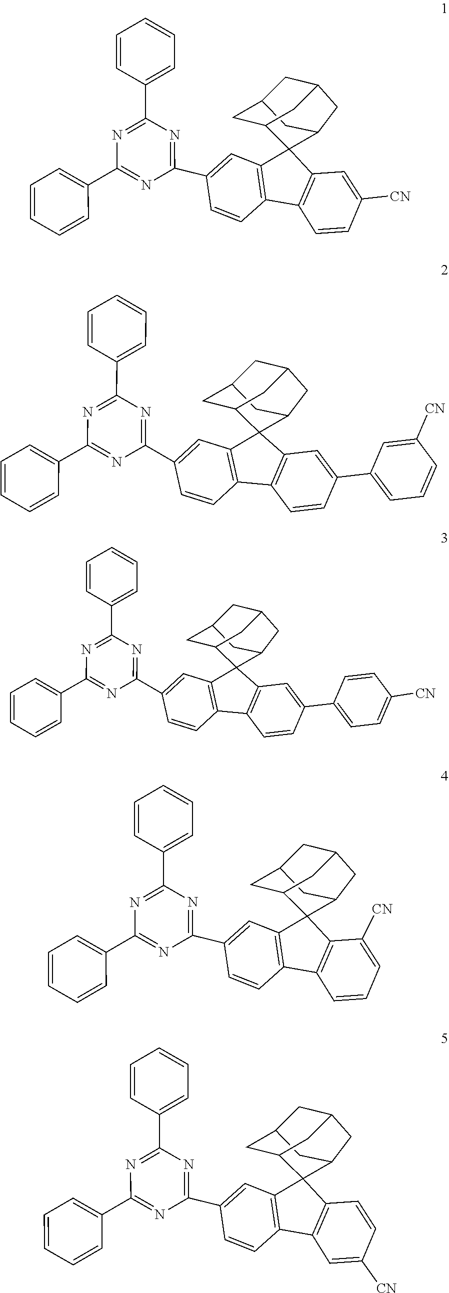 [SMILES: N#Cc1ccc2c(c1)C1(c3cc(-c4nc(-c5ccccc5)nc(-c5ccccc5)n4)ccc3-2)C2CC3CC(C2)CC1C3 ; N#Cc1cccc(-c2ccc3c(c2)C2(c4cc(-c5nc(-c6ccccc6)nc(-c6ccccc6)n5)ccc4-3)C3CC4CC(C3)CC2C4)c1 ; N#Cc1ccc(-c2ccc3c(c2)C2(c4cc(-c5nc(-c6ccccc6)nc(-c6ccccc6)n5)ccc4-3)C3CC4CC(C3)CC2C4)cc1 ; N#Cc1cccc2c1C1(c3cc(-c4nc(-c5ccccc5)nc(-c5ccccc5)n4)ccc3-2)C2CC3CC(C2)CC1C3 ; N#Cc1ccc2c(c1)-c1ccc(-c3nc(-c4ccccc4)nc(-c4ccccc4)n3)cc1C21C2CC3CC(C2)CC1C3]

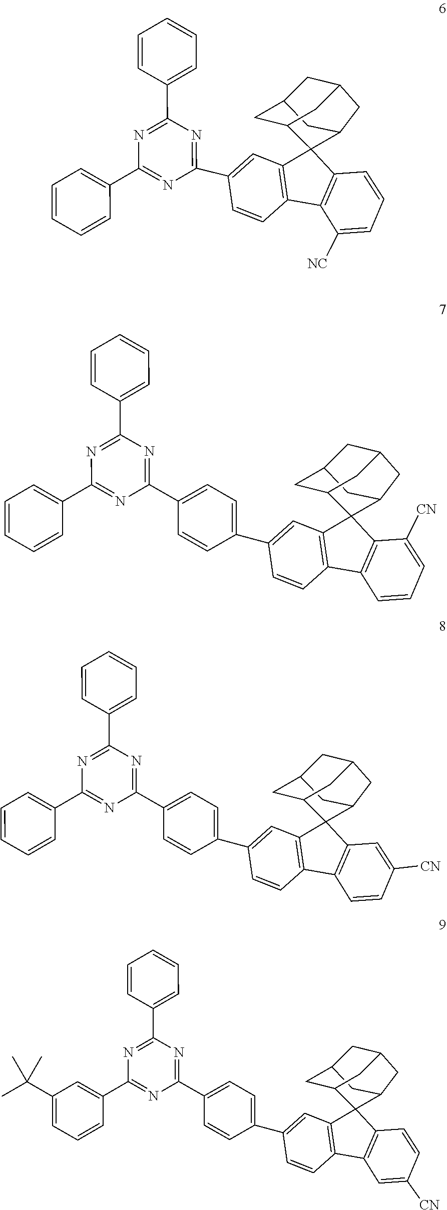 [SMILES: N#Cc1cccc2c1-c1ccc(-c3nc(-c4ccccc4)nc(-c4ccccc4)n3)cc1C21C2CC3CC(C2)CC1C3 ; N#Cc1cccc2c1C1(c3cc(-c4ccc(-c5nc(-c6ccccc6)nc(-c6ccccc6)n5)cc4)ccc3-2)C2CC3CC(C2)CC1C3 ; N#Cc1ccc2c(c1)C1(c3cc(-c4ccc(-c5nc(-c6ccccc6)nc(-c6ccccc6)n5)cc4)ccc3-2)C2CC3CC(C2)CC1C3 ; CC(C)(C)c1cccc(-c2nc(-c3ccccc3)nc(-c3ccc(-c4ccc5c(c4)C4(c6ccc(C#N)cc6-5)C5CC6CC(C5)CC4C6)cc3)n2)c1]

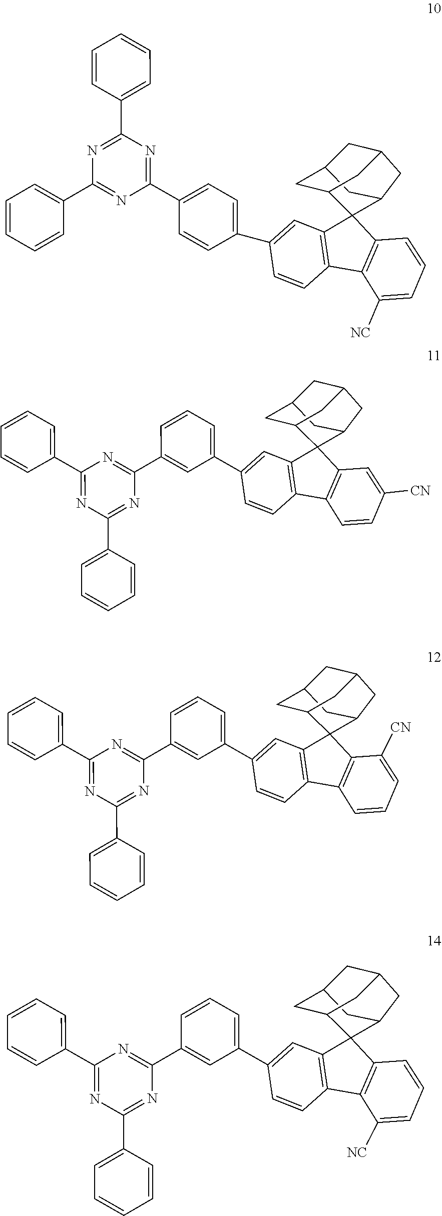 [SMILES: N#Cc1cccc2c1-c1ccc(-c3ccc(-c4nc(-c5ccccc5)nc(-c5ccccc5)n4)cc3)cc1C21C2CC3CC(C2)CC1C3 ; N#Cc1ccc2c(c1)C1(c3cc(-c4cccc(-c5nc(-c6ccccc6)nc(-c6ccccc6)n5)c4)ccc3-2)C2CC3CC(C2)CC1C3 ; N#Cc1cccc2c1C1(c3cc(-c4cccc(-c5nc(-c6ccccc6)nc(-c6ccccc6)n5)c4)ccc3-2)C2CC3CC(C2)CC1C3 ; N#Cc1cccc2c1-c1ccc(-c3cccc(-c4nc(-c5ccccc5)nc(-c5ccccc5)n4)c3)cc1C21C2CC3CC(C2)CC1C3]

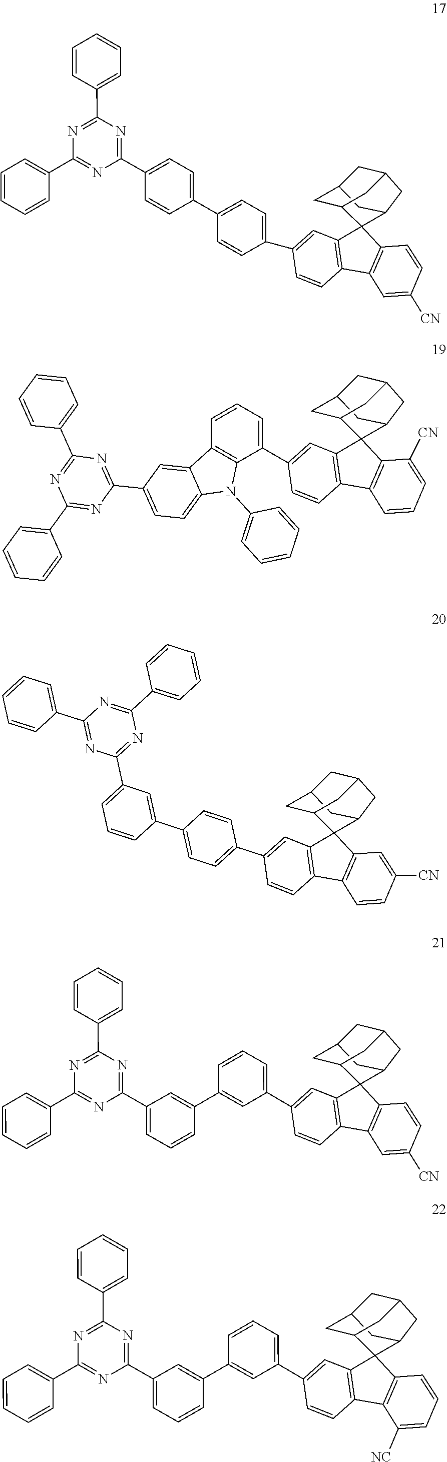

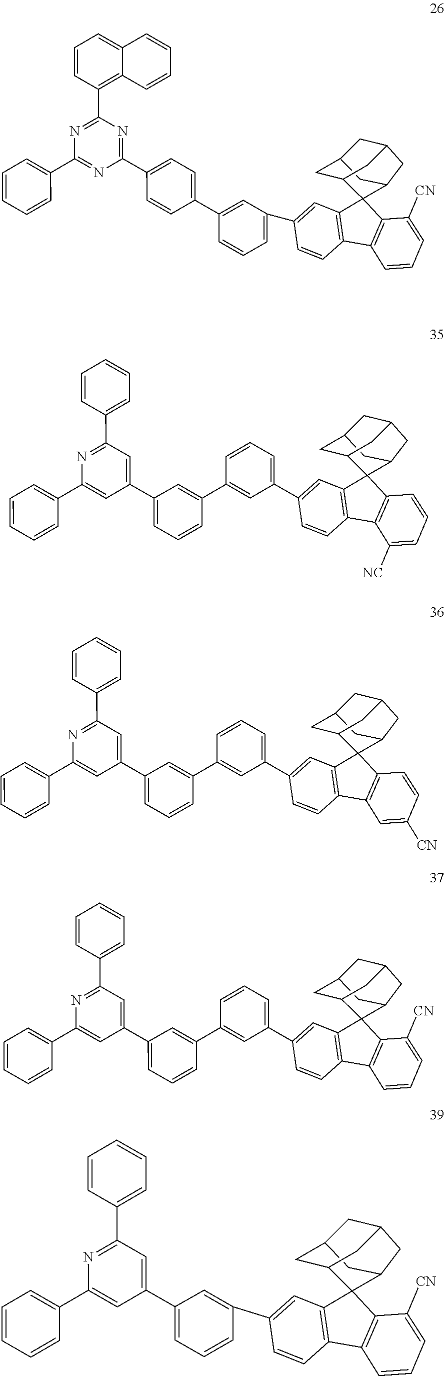 [SMILES: N#Cc1cccc2c1C1(c3cc(-c4cccc(-c5ccc(-c6nc(-c7ccccc7)nc(-c7cccc8ccccc78)n6)cc5)c4)ccc3-2)C2CC3CC(C2)CC1C3 ; N#Cc1cccc2c1-c1ccc(-c3cccc(-c4cccc(-c5cc(-c6ccccc6)nc(-c6ccccc6)c5)c4)c3)cc1C21C2CC3CC(C2)CC1C3 ; N#Cc1ccc2c(c1)-c1ccc(-c3cccc(-c4cccc(-c5cc(-c6ccccc6)nc(-c6ccccc6)c5)c4)c3)cc1C21C2CC3CC(C2)CC1C3 ; N#Cc1cccc2c1C1(c3cc(-c4cccc(-c5cccc(-c6cc(-c7ccccc7)nc(-c7ccccc7)c6)c5)c4)ccc3-2)C2CC3CC(C2)CC1C3 ; N#Cc1cccc2c1C1(c3cc(-c4cccc(-c5cc(-c6ccccc6)nc(-c6ccccc6)c5)c4)ccc3-2)C2CC3CC(C2)CC1C3]

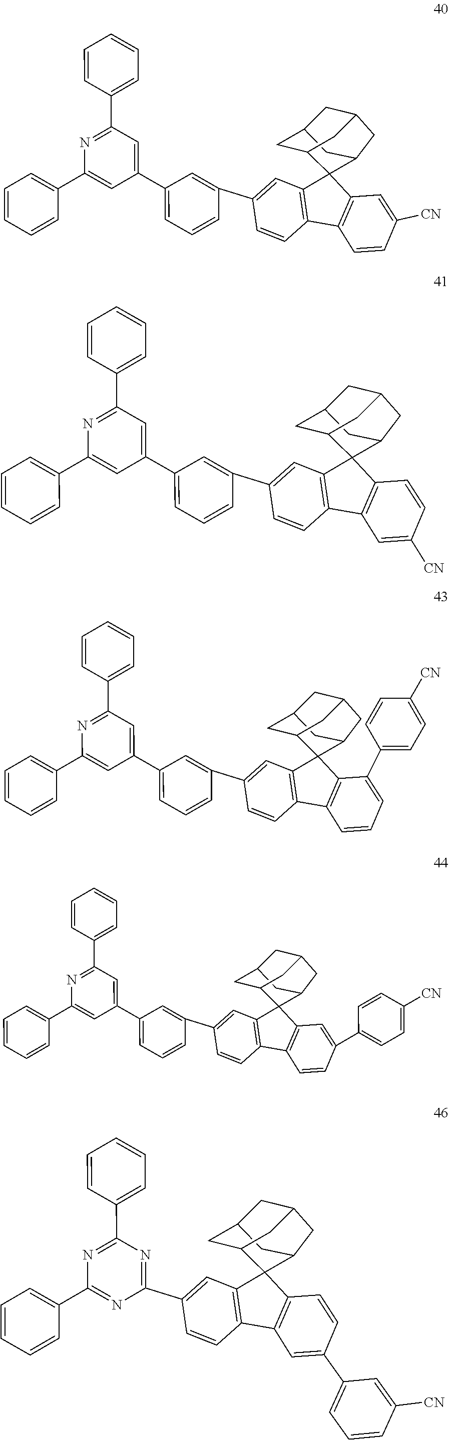 [SMILES: N#Cc1ccc2c(c1)C1(c3cc(-c4cccc(-c5cc(-c6ccccc6)nc(-c6ccccc6)c5)c4)ccc3-2)C2CC3CC(C2)CC1C3 ; N#Cc1ccc2c(c1)-c1ccc(-c3cccc(-c4cc(-c5ccccc5)nc(-c5ccccc5)c4)c3)cc1C21C2CC3CC(C2)CC1C3 ; N#Cc1ccc(-c2cccc3c2C2(c4cc(-c5cccc(-c6cc(-c7ccccc7)nc(-c7ccccc7)c6)c5)ccc4-3)C3CC4CC(C3)CC2C4)cc1 ; N#Cc1ccc(-c2ccc3c(c2)C2(c4cc(-c5cccc(-c6cc(-c7ccccc7)nc(-c7ccccc7)c6)c5)ccc4-3)C3CC4CC(C3)CC2C4)cc1 ; N#Cc1cccc(-c2ccc3c(c2)-c2ccc(-c4nc(-c5ccccc5)nc(-c5ccccc5)n4)cc2C32C3CC4CC(C3)CC2C4)c1]

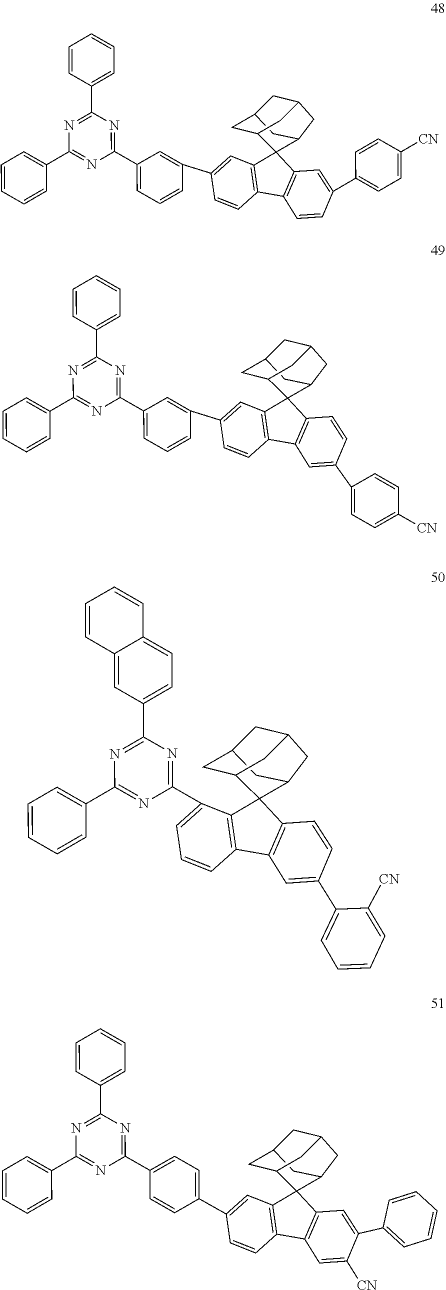 [SMILES: N#Cc1ccc(-c2ccc3c(c2)C2(c4cc(-c5cccc(-c6nc(-c7ccccc7)nc(-c7ccccc7)n6)c5)ccc4-3)C3CC4CC(C3)CC2C4)cc1 ; N#Cc1ccc(-c2ccc3c(c2)-c2ccc(-c4cccc(-c5nc(-c6ccccc6)nc(-c6ccccc6)n5)c4)cc2C32C3CC4CC(C3)CC2C4)cc1 ; N#Cc1ccccc1-c1ccc2c(c1)-c1cccc(-c3nc(-c4ccccc4)nc(-c4ccc5ccccc5c4)n3)c1C21C2CC3CC(C2)CC1C3 ; N#Cc1cc2c(cc1-c1ccccc1)C1(c3cc(-c4ccc(-c5nc(-c6ccccc6)nc(-c6ccccc6)n5)cc4)ccc3-2)C2CC3CC(C2)CC1C3]

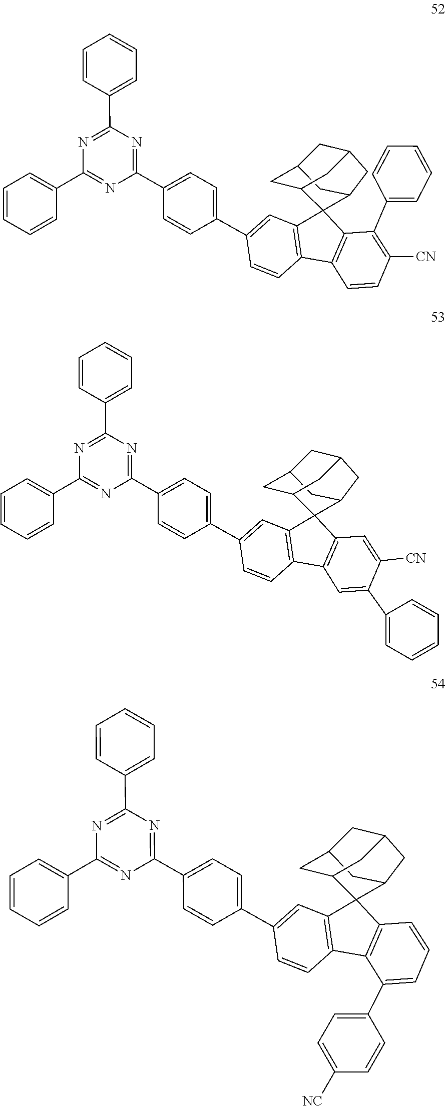

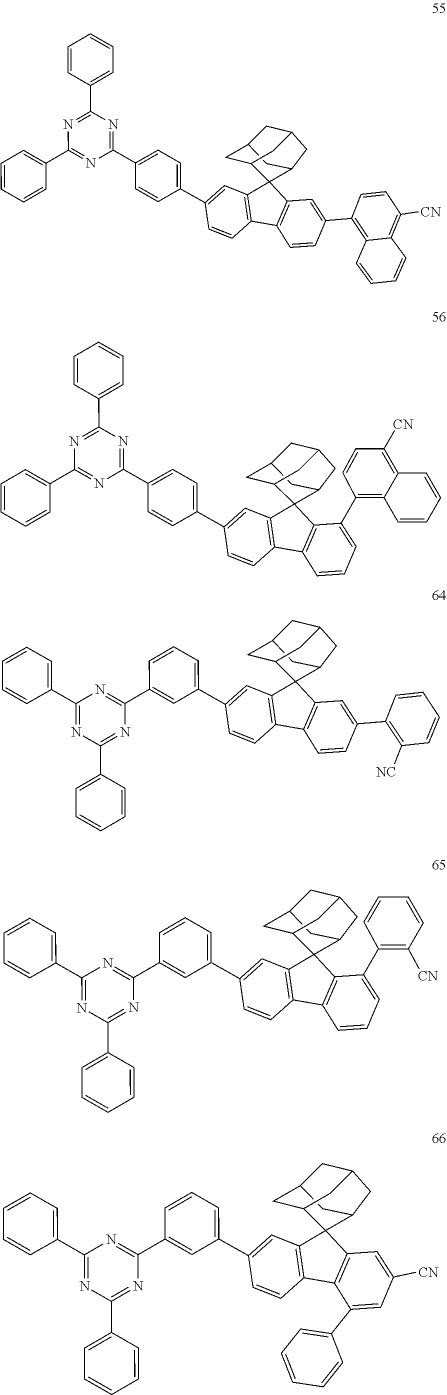

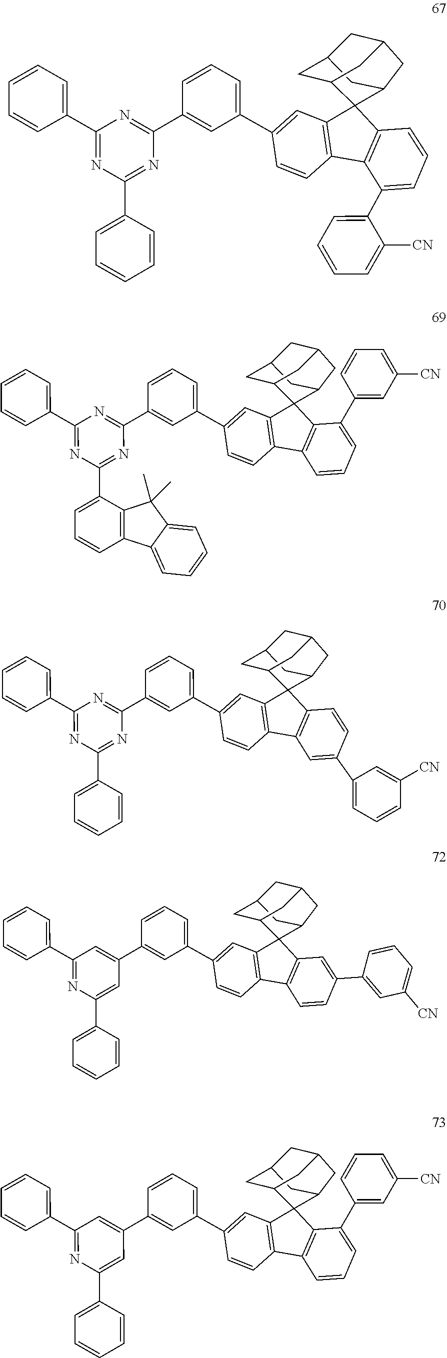

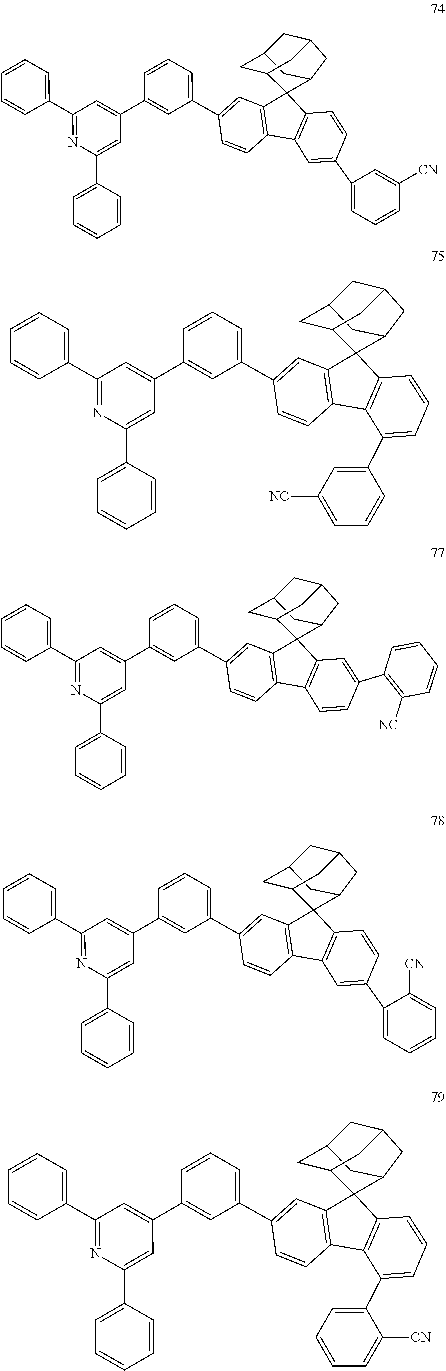 [SMILES: N#Cc1cccc(-c2ccc3c(c2)-c2ccc(-c4cccc(-c5cc(-c6ccccc6)nc(-c6ccccc6)c5)c4)cc2C32C3CC4CC(C3)CC2C4)c1 ; N#Cc1cccc(-c2cccc3c2-c2ccc(-c4cccc(-c5cc(-c6ccccc6)nc(-c6ccccc6)c5)c4)cc2C32C3CC4CC(C3)CC2C4)c1 ; N#Cc1ccccc1-c1ccc2c(c1)C1(c3cc(-c4cccc(-c5cc(-c6ccccc6)nc(-c6ccccc6)c5)c4)ccc3-2)C2CC3CC(C2)CC1C3 ; N#Cc1ccccc1-c1ccc2c(c1)-c1ccc(-c3cccc(-c4cc(-c5ccccc5)nc(-c5ccccc5)c4)c3)cc1C21C2CC3CC(C2)CC1C3 ; N#Cc1ccccc1-c1cccc2c1-c1ccc(-c3cccc(-c4cc(-c5ccccc5)nc(-c5ccccc5)c4)c3)cc1C21C2CC3CC(C2)CC1C3]

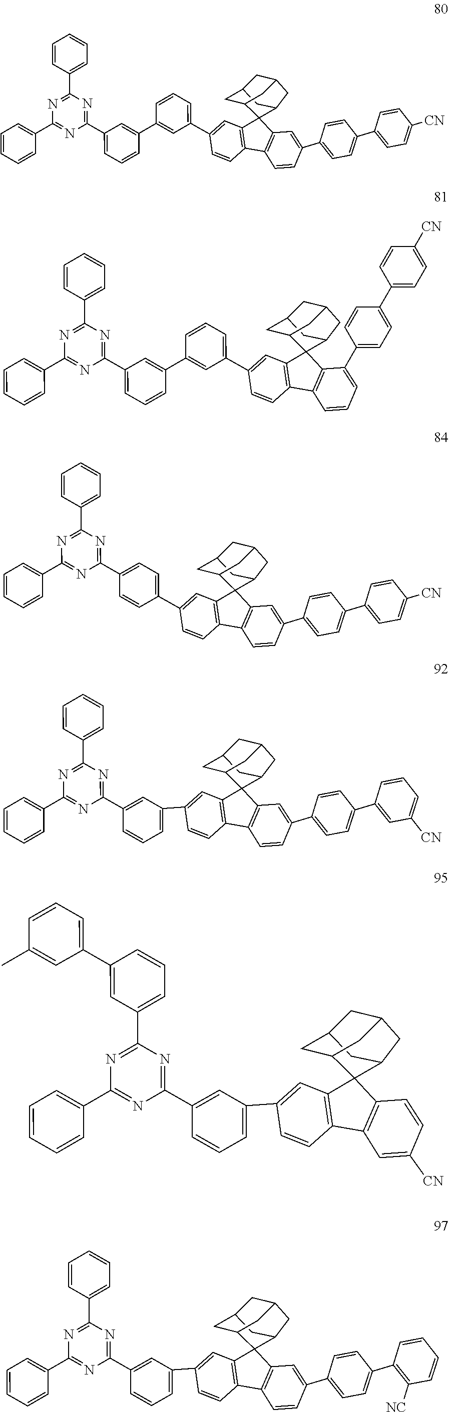

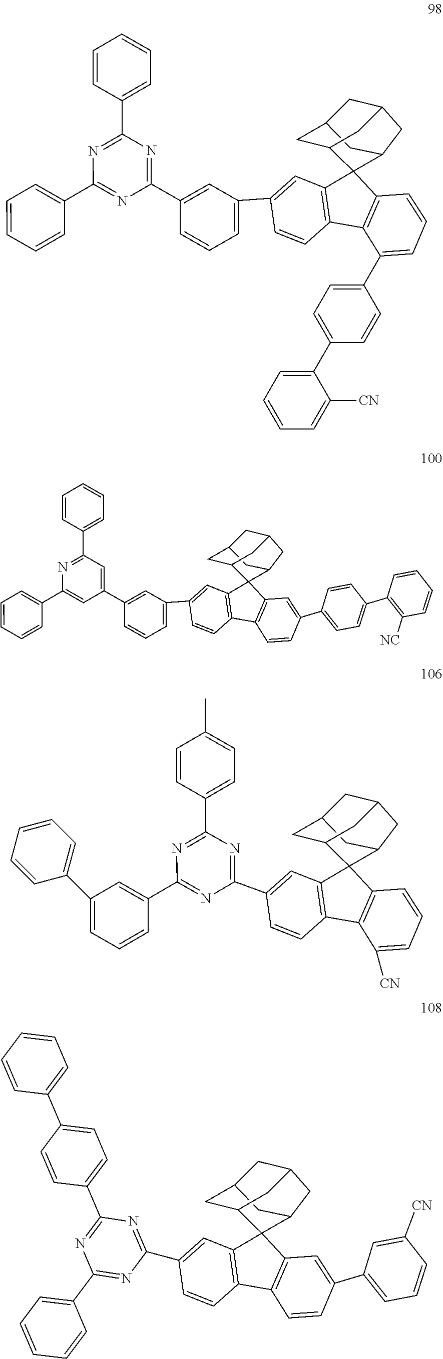 [SMILES: N#Cc1ccccc1-c1ccc(-c2cccc3c2-c2ccc(-c4cccc(-c5nc(-c6ccccc6)nc(-c6ccccc6)n5)c4)cc2C32C3CC4CC(C3)CC2C4)cc1 ; N#Cc1ccccc1-c1ccc(-c2ccc3c(c2)C2(c4cc(-c5cccc(-c6cc(-c7ccccc7)nc(-c7ccccc7)c6)c5)ccc4-3)C3CC4CC(C3)CC2C4)cc1 ; Cc1ccc(-c2nc(-c3cccc(-c4ccccc4)c3)nc(-c3ccc4c(c3)C3(c5cccc(C#N)c5-4)C4CC5CC(C4)CC3C5)n2)cc1 ; N#Cc1cccc(-c2ccc3c(c2)C2(c4cc(-c5nc(-c6ccccc6)nc(-c6ccc(-c7ccccc7)cc6)n5)ccc4-3)C3CC4CC(C3)CC2C4)c1]

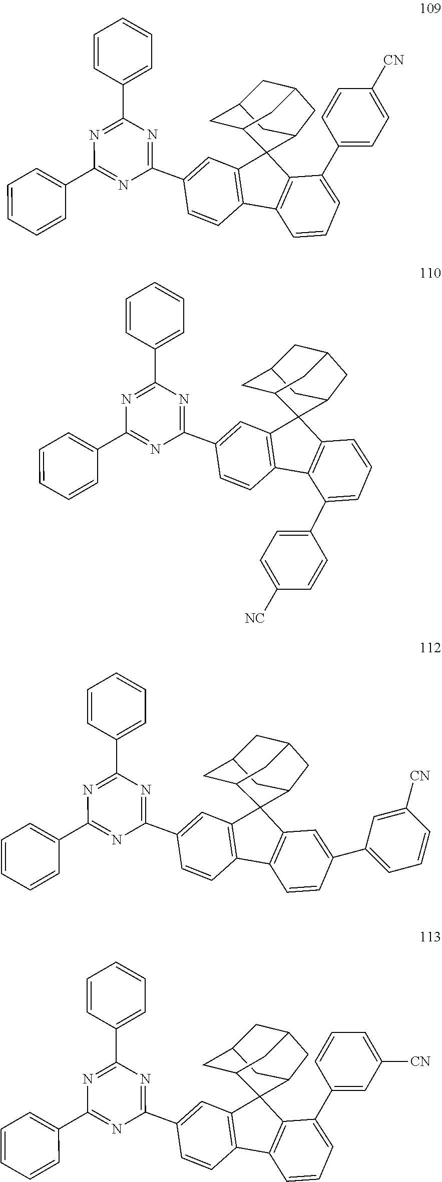 [SMILES: N#Cc1ccc(-c2cccc3c2C2(c4cc(-c5nc(-c6ccccc6)nc(-c6ccccc6)n5)ccc4-3)C3CC4CC(C3)CC2C4)cc1 ; N#Cc1ccc(-c2cccc3c2-c2ccc(-c4nc(-c5ccccc5)nc(-c5ccccc5)n4)cc2C32C3CC4CC(C3)CC2C4)cc1 ; N#Cc1cccc(-c2ccc3c(c2)C2(c4cc(-c5nc(-c6ccccc6)nc(-c6ccccc6)n5)ccc4-3)C3CC4CC(C3)CC2C4)c1 ; N#Cc1cccc(-c2cccc3c2C2(c4cc(-c5nc(-c6ccccc6)nc(-c6ccccc6)n5)ccc4-3)C3CC4CC(C3)CC2C4)c1]

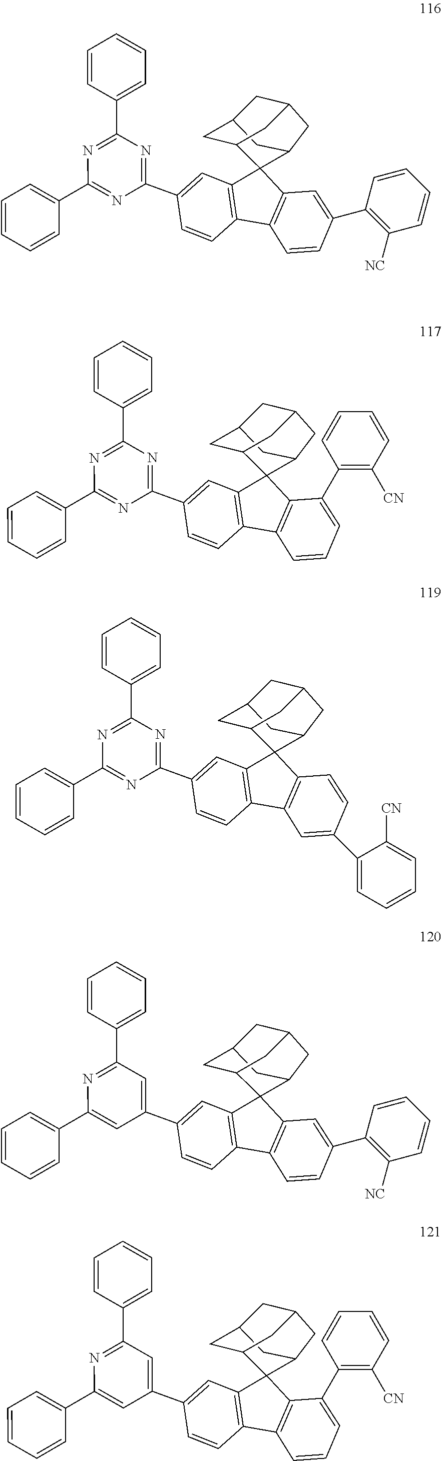

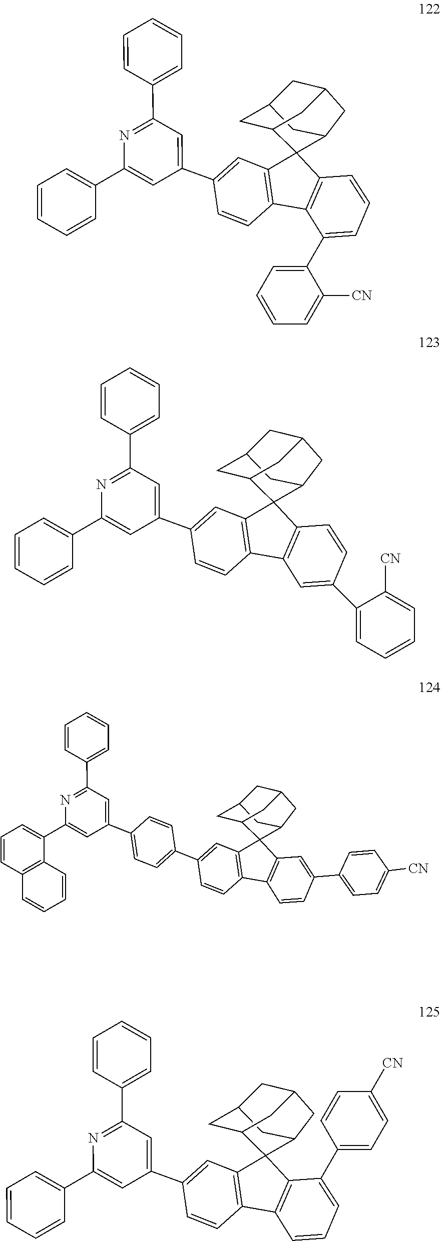 [SMILES: N#Cc1ccccc1-c1cccc2c1-c1ccc(-c3cc(-c4ccccc4)nc(-c4ccccc4)c3)cc1C21C2CC3CC(C2)CC1C3 ; N#Cc1ccccc1-c1ccc2c(c1)-c1ccc(-c3cc(-c4ccccc4)nc(-c4ccccc4)c3)cc1C21C2CC3CC(C2)CC1C3 ; N#Cc1ccc(-c2ccc3c(c2)C2(c4cc(-c5ccc(-c6cc(-c7ccccc7)nc(-c7cccc8ccccc78)c6)cc5)ccc4-3)C3CC4CC(C3)CC2C4)cc1 ; N#Cc1ccc(-c2cccc3c2C2(c4cc(-c5cc(-c6ccccc6)nc(-c6ccccc6)c5)ccc4-3)C3CC4CC(C3)CC2C4)cc1]

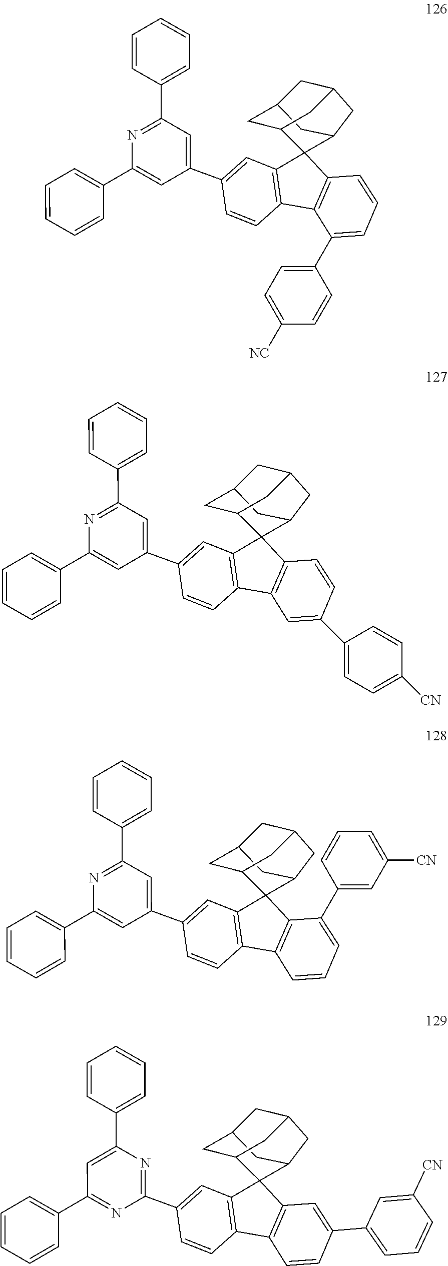 [SMILES: N#Cc1ccc(-c2cccc3c2-c2ccc(-c4cc(-c5ccccc5)nc(-c5ccccc5)c4)cc2C32C3CC4CC(C3)CC2C4)cc1 ; N#Cc1ccc(-c2ccc3c(c2)-c2ccc(-c4cc(-c5ccccc5)nc(-c5ccccc5)c4)cc2C32C3CC4CC(C3)CC2C4)cc1 ; N#Cc1cccc(-c2cccc3c2C2(c4cc(-c5cc(-c6ccccc6)nc(-c6ccccc6)c5)ccc4-3)C3CC4CC(C3)CC2C4)c1 ; N#Cc1cccc(-c2ccc3c(c2)C2(c4cc(-c5nc(-c6ccccc6)cc(-c6ccccc6)n5)ccc4-3)C3CC4CC(C3)CC2C4)c1]

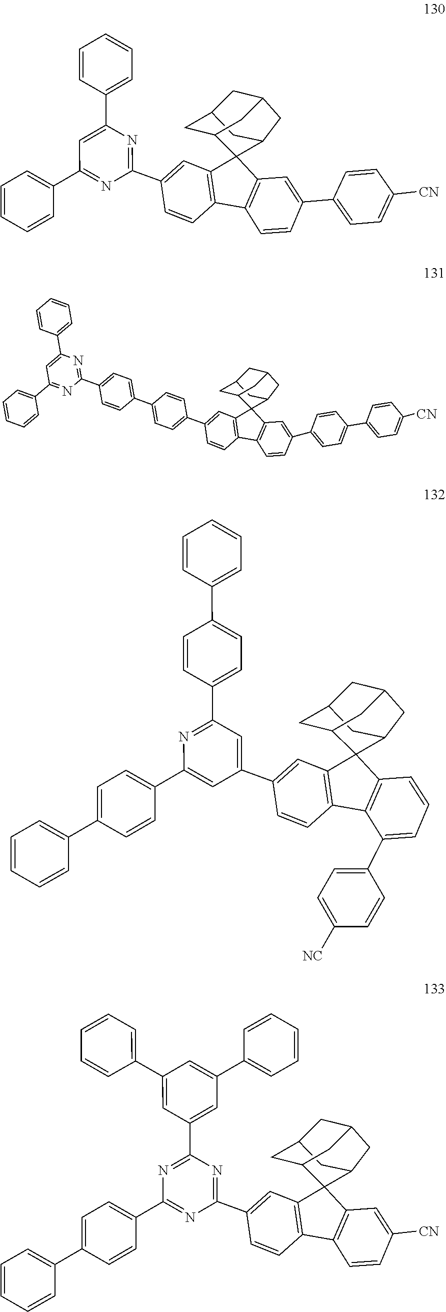 [SMILES: N#Cc1ccc(-c2ccc3c(c2)C2(c4cc(-c5nc(-c6ccccc6)cc(-c6ccccc6)n5)ccc4-3)C3CC4CC(C3)CC2C4)cc1 ; N#Cc1ccc(-c2ccc(-c3ccc4c(c3)C3(c5cc(-c6ccc(-c7ccc(-c8nc(-c9ccccc9)cc(-c9ccccc9)n8)cc7)cc6)ccc5-4)C4CC5CC(C4)CC3C5)cc2)cc1 ; N#Cc1ccc(-c2cccc3c2-c2ccc(-c4cc(-c5ccc(-c6ccccc6)cc5)nc(-c5ccc(-c6ccccc6)cc5)c4)cc2C32C3CC4CC(C3)CC2C4)cc1 ; N#Cc1ccc2c(c1)C1(c3cc(-c4nc(-c5ccc(-c6ccccc6)cc5)nc(-c5cc(-c6ccccc6)cc(-c6ccccc6)c5)n4)ccc3-2)C2CC3CC(C2)CC1C3]

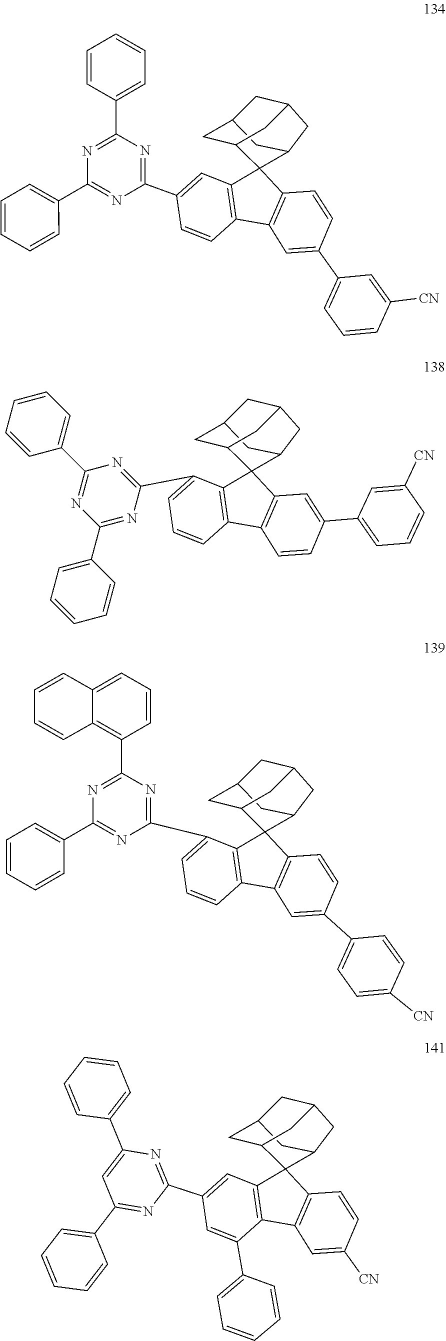 [SMILES: N#Cc1cccc(-c2ccc3c(c2)-c2ccc(-c4nc(-c5ccccc5)nc(-c5ccccc5)n4)cc2C32C3CC4CC(C3)CC2C4)c1 ; N#Cc1cccc(-c2ccc3c(c2)C2(c4c(-c5nc(-c6ccccc6)nc(-c6ccccc6)n5)cccc4-3)C3CC4CC(C3)CC2C4)c1 ; N#Cc1ccc(-c2ccc3c(c2)-c2cccc(-c4nc(-c5ccccc5)nc(-c5cccc6ccccc56)n4)c2C32C3CC4CC(C3)CC2C4)cc1 ; N#Cc1ccc2c(c1)-c1c(-c3ccccc3)cc(-c3nc(-c4ccccc4)cc(-c4ccccc4)n3)cc1C21C2CC3CC(C2)CC1C3]

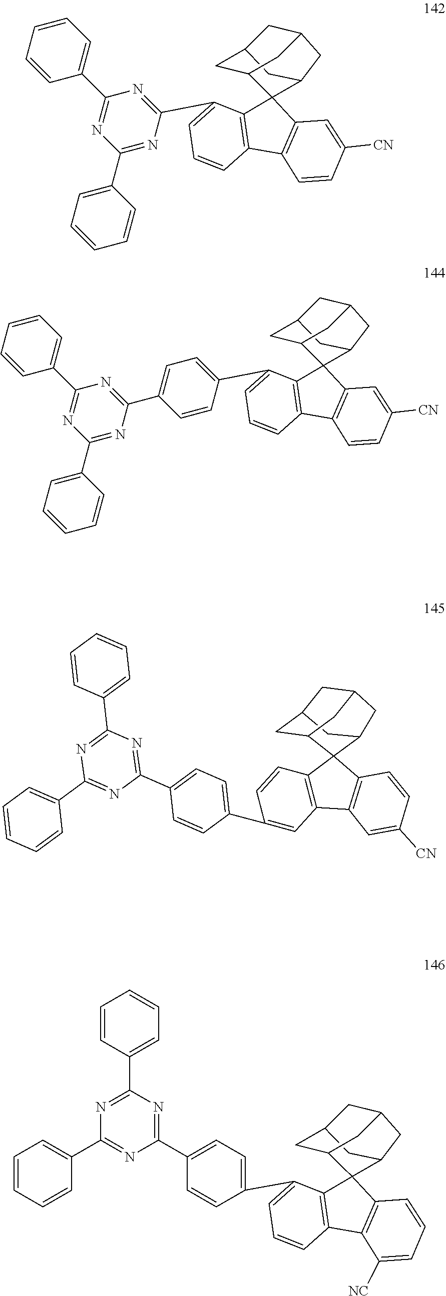 [SMILES: N#Cc1ccc2c(c1)C1(c3c(-c4nc(-c5ccccc5)nc(-c5ccccc5)n4)cccc3-2)C2CC3CC(C2)CC1C3 ; N#Cc1ccc2c(c1)C1(c3c(-c4ccc(-c5nc(-c6ccccc6)nc(-c6ccccc6)n5)cc4)cccc3-2)C2CC3CC(C2)CC1C3 ; N#Cc1ccc2c(c1)-c1cc(-c3ccc(-c4nc(-c5ccccc5)nc(-c5ccccc5)n4)cc3)ccc1C21C2CC3CC(C2)CC1C3 ; N#Cc1cccc2c1-c1cccc(-c3ccc(-c4nc(-c5ccccc5)nc(-c5ccccc5)n4)cc3)c1C21C2CC3CC(C2)CC1C3]

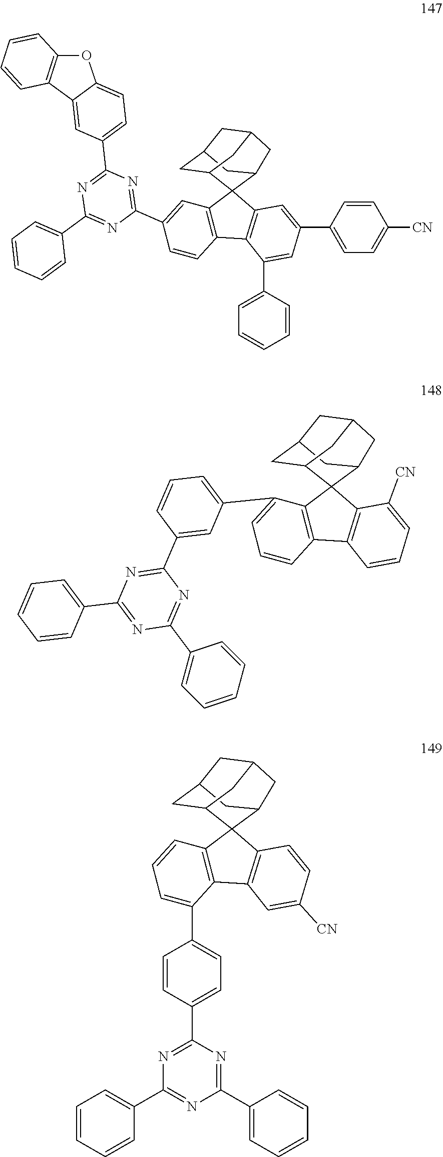 [SMILES: N#Cc1ccc(-c2cc(-c3ccccc3)c3c(c2)C2(c4cc(-c5nc(-c6ccccc6)nc(-c6ccc7oc8ccccc8c7c6)n5)ccc4-3)C3CC4CC(C3)CC2C4)cc1 ; N#Cc1cccc2c1C1(c3c(-c4cccc(-c5nc(-c6ccccc6)nc(-c6ccccc6)n5)c4)cccc3-2)C2CC3CC(C2)CC1C3 ; N#Cc1ccc2c(c1)-c1c(-c3ccc(-c4nc(-c5ccccc5)nc(-c5ccccc5)n4)cc3)cccc1C21C2CC3CC(C2)CC1C3]

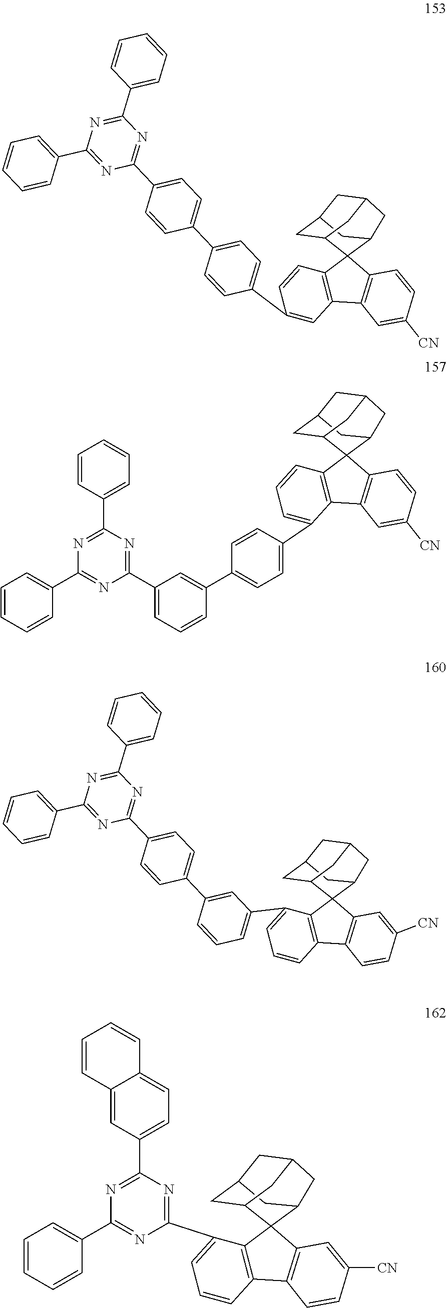 [SMILES: N#Cc1ccc2c(c1)-c1cc(-c3ccc(-c4ccc(-c5nc(-c6ccccc6)nc(-c6ccccc6)n5)cc4)cc3)ccc1C21C2CC3CC(C2)CC1C3 ; N#Cc1ccc2c(c1)-c1c(-c3ccc(-c4cccc(-c5nc(-c6ccccc6)nc(-c6ccccc6)n5)c4)cc3)cccc1C21C2CC3CC(C2)CC1C3 ; N#Cc1ccc2c(c1)C1(c3c(-c4cccc(-c5ccc(-c6nc(-c7ccccc7)nc(-c7ccccc7)n6)cc5)c4)cccc3-2)C2CC3CC(C2)CC1C3 ; N#Cc1ccc2c(c1)C1(c3c(-c4nc(-c5ccccc5)nc(-c5ccc6ccccc6c5)n4)cccc3-2)C2CC3CC(C2)CC1C3]

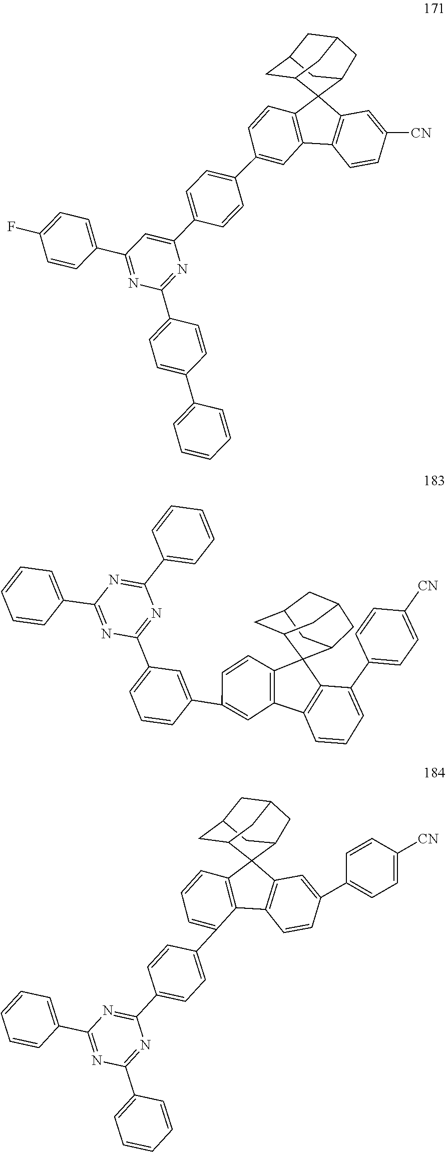 [SMILES: N#Cc1ccc2c(c1)C1(c3ccc(-c4ccc(-c5cc(-c6ccc(F)cc6)nc(-c6ccc(-c7ccccc7)cc6)n5)cc4)cc3-2)C2CC3CC(C2)CC1C3 ; N#Cc1ccc(-c2cccc3c2C2(c4ccc(-c5cccc(-c6nc(-c7ccccc7)nc(-c7ccccc7)n6)c5)cc4-3)C3CC4CC(C3)CC2C4)cc1 ; N#Cc1ccc(-c2ccc3c(c2)C2(c4cccc(-c5ccc(-c6nc(-c7ccccc7)nc(-c7ccccc7)n6)cc5)c4-3)C3CC4CC(C3)CC2C4)cc1]

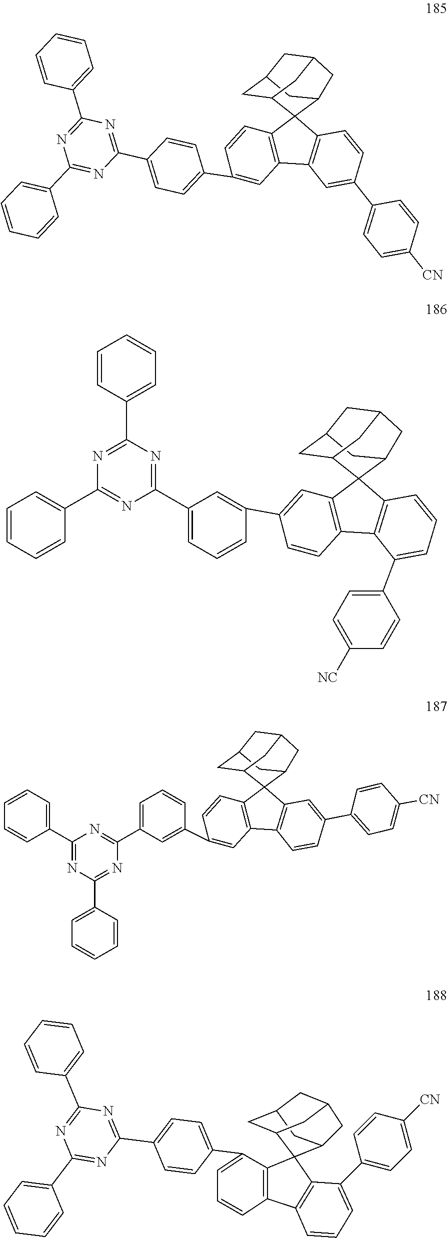 [SMILES: N#Cc1ccc(-c2ccc3c(c2)-c2cc(-c4ccc(-c5nc(-c6ccccc6)nc(-c6ccccc6)n5)cc4)ccc2C32C3CC4CC(C3)CC2C4)cc1 ; N#Cc1ccc(-c2cccc3c2-c2ccc(-c4cccc(-c5nc(-c6ccccc6)nc(-c6ccccc6)n5)c4)cc2C32C3CC4CC(C3)CC2C4)cc1 ; N#Cc1ccc(-c2ccc3c(c2)C2(c4ccc(-c5cccc(-c6nc(-c7ccccc7)nc(-c7ccccc7)n6)c5)cc4-3)C3CC4CC(C3)CC2C4)cc1 ; N#Cc1ccc(-c2cccc3c2C2(c4c(-c5ccc(-c6nc(-c7ccccc7)nc(-c7ccccc7)n6)cc5)cccc4-3)C3CC4CC(C3)CC2C4)cc1]

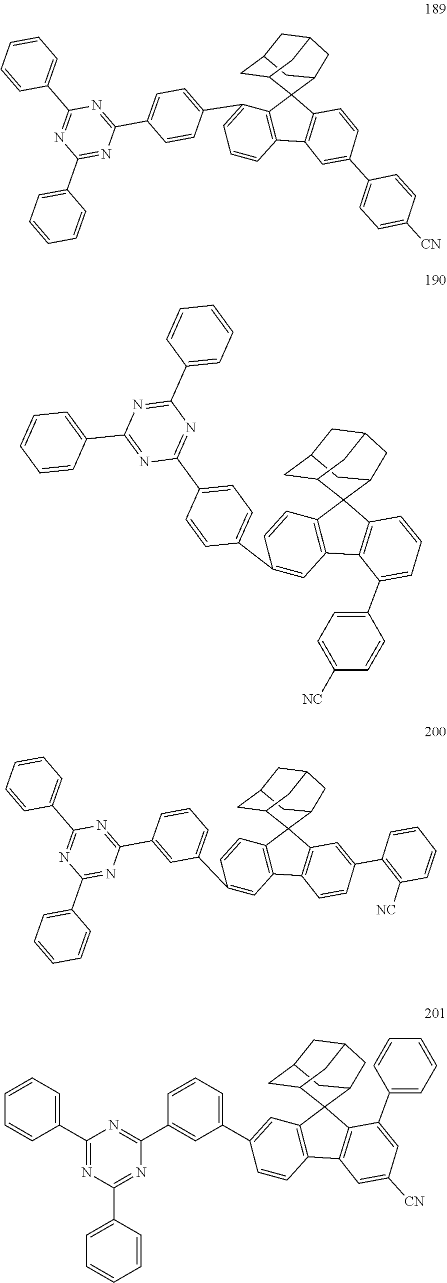 [SMILES: N#Cc1ccc(-c2ccc3c(c2)-c2cccc(-c4ccc(-c5nc(-c6ccccc6)nc(-c6ccccc6)n5)cc4)c2C32C3CC4CC(C3)CC2C4)cc1 ; N#Cc1ccc(-c2cccc3c2-c2cc(-c4ccc(-c5nc(-c6ccccc6)nc(-c6ccccc6)n5)cc4)ccc2C32C3CC4CC(C3)CC2C4)cc1 ; N#Cc1ccccc1-c1ccc2c(c1)C1(c3ccc(-c4cccc(-c5nc(-c6ccccc6)nc(-c6ccccc6)n5)c4)cc3-2)C2CC3CC(C2)CC1C3 ; N#Cc1cc(-c2ccccc2)c2c(c1)-c1ccc(-c3cccc(-c4nc(-c5ccccc5)nc(-c5ccccc5)n4)c3)cc1C21C2CC3CC(C2)CC1C3]

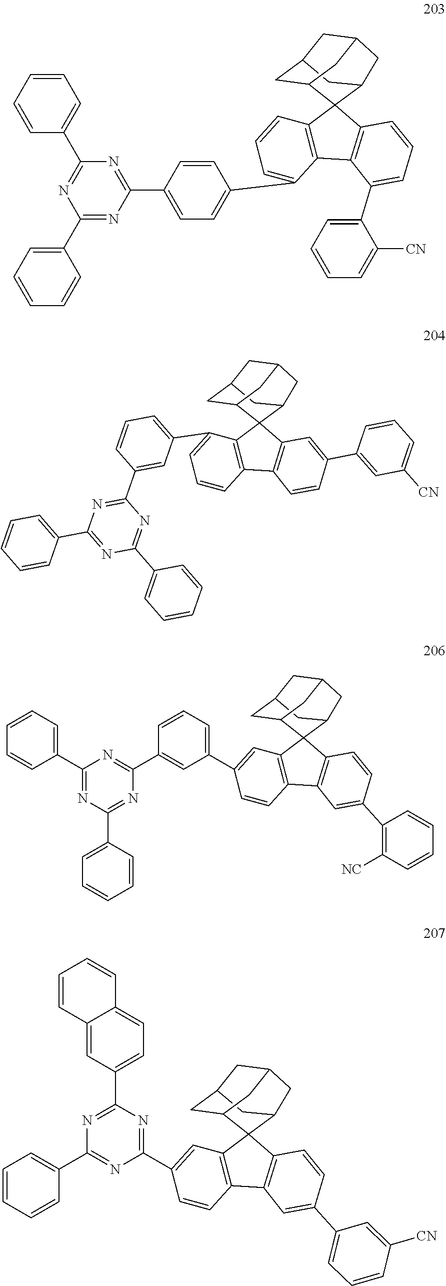 [SMILES: N#Cc1ccccc1-c1cccc2c1-c1c(-c3ccc(-c4nc(-c5ccccc5)nc(-c5ccccc5)n4)cc3)cccc1C21C2CC3CC(C2)CC1C3 ; N#Cc1cccc(-c2ccc3c(c2)C2(c4c(-c5cccc(-c6nc(-c7ccccc7)nc(-c7ccccc7)n6)c5)cccc4-3)C3CC4CC(C3)CC2C4)c1 ; N#Cc1ccccc1-c1ccc2c(c1)-c1ccc(-c3cccc(-c4nc(-c5ccccc5)nc(-c5ccccc5)n4)c3)cc1C21C2CC3CC(C2)CC1C3 ; N#Cc1cccc(-c2ccc3c(c2)-c2ccc(-c4nc(-c5ccccc5)nc(-c5ccc6ccccc6c5)n4)cc2C32C3CC4CC(C3)CC2C4)c1]

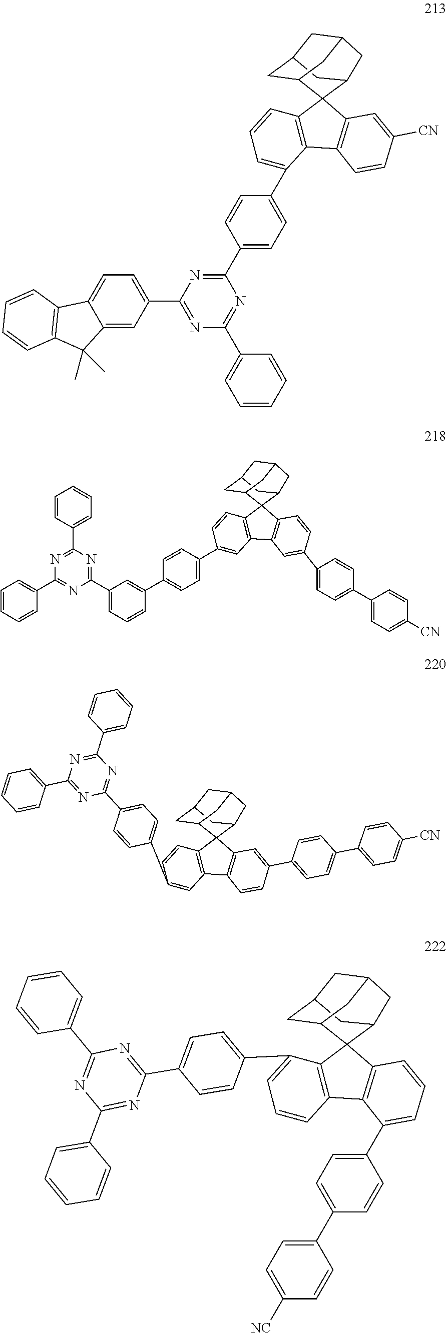 [SMILES: CC1(C)c2ccccc2-c2ccc(-c3nc(-c4ccccc4)nc(-c4ccc(-c5cccc6c5-c5ccc(C#N)cc5C65C6CC7CC(C6)CC5C7)cc4)n3)cc21 ; N#Cc1ccc(-c2ccc(-c3ccc4c(c3)-c3cc(-c5ccc(-c6cccc(-c7nc(-c8ccccc8)nc(-c8ccccc8)n7)c6)cc5)ccc3C43C4CC5CC(C4)CC3C5)cc2)cc1 ; N#Cc1ccc(-c2ccc(-c3ccc4c(c3)C3(c5ccc(-c6ccc(-c7nc(-c8ccccc8)nc(-c8ccccc8)n7)cc6)cc5-4)C4CC5CC(C4)CC3C5)cc2)cc1 ; N#Cc1ccc(-c2ccc(-c3cccc4c3-c3cccc(-c5ccc(-c6nc(-c7ccccc7)nc(-c7ccccc7)n6)cc5)c3C43C4CC5CC(C4)CC3C5)cc2)cc1]

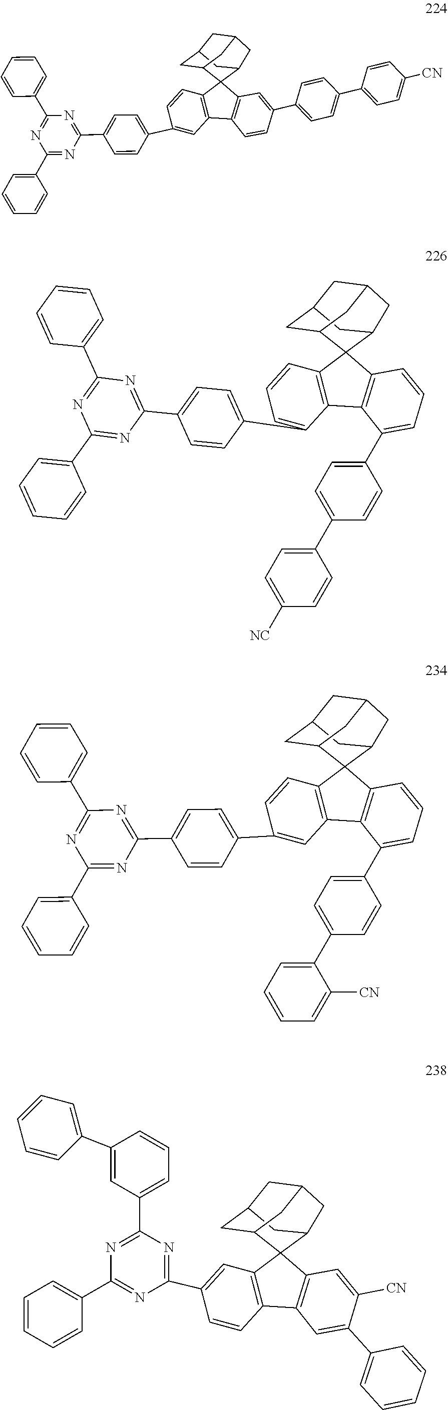 [SMILES: N#Cc1ccc(-c2ccc(-c3ccc4c(c3)C3(c5ccc(-c6ccc(-c7nc(-c8ccccc8)nc(-c8ccccc8)n7)cc6)cc5-4)C4CC5CC(C4)CC3C5)cc2)cc1 ; N#Cc1ccc(-c2ccc(-c3cccc4c3-c3c(-c5ccc(-c6nc(-c7ccccc7)nc(-c7ccccc7)n6)cc5)cccc3C43C4CC5CC(C4)CC3C5)cc2)cc1 ; N#Cc1ccccc1-c1ccc(-c2cccc3c2-c2cc(-c4ccc(-c5nc(-c6ccccc6)nc(-c6ccccc6)n5)cc4)ccc2C32C3CC4CC(C3)CC2C4)cc1 ; N#Cc1cc2c(cc1-c1ccccc1)-c1ccc(-c3nc(-c4ccccc4)nc(-c4cccc(-c5ccccc5)c4)n3)cc1C21C2CC3CC(C2)CC1C3]

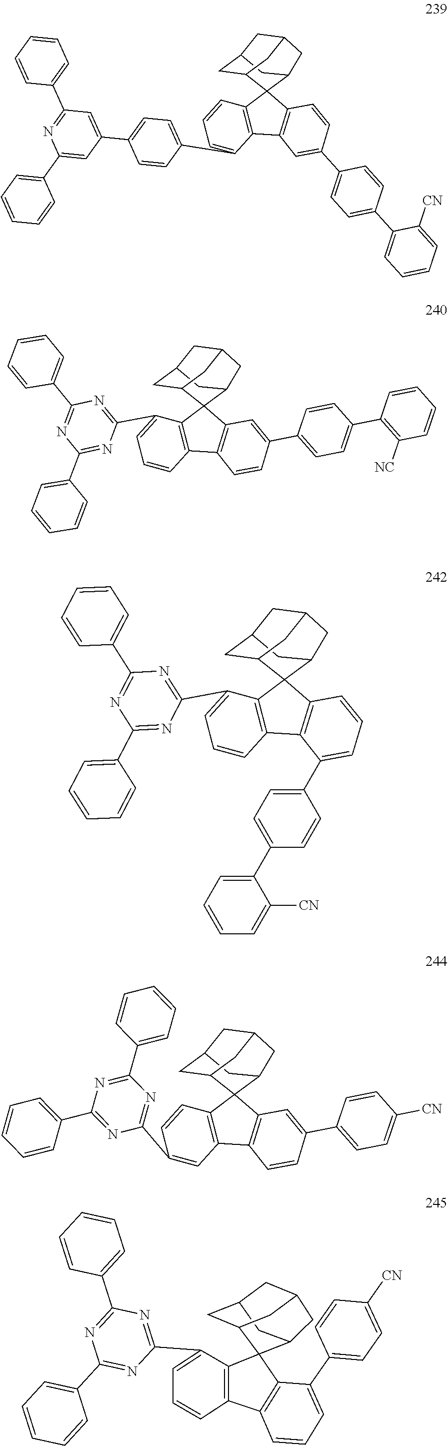 [SMILES: N#Cc1ccccc1-c1ccc(-c2ccc3c(c2)-c2c(-c4ccc(-c5cc(-c6ccccc6)nc(-c6ccccc6)c5)cc4)cccc2C32C3CC4CC(C3)CC2C4)cc1 ; N#Cc1ccccc1-c1ccc(-c2ccc3c(c2)C2(c4c(-c5nc(-c6ccccc6)nc(-c6ccccc6)n5)cccc4-3)C3CC4CC(C3)CC2C4)cc1 ; N#Cc1ccccc1-c1ccc(-c2cccc3c2-c2cccc(-c4nc(-c5ccccc5)nc(-c5ccccc5)n4)c2C32C3CC4CC(C3)CC2C4)cc1 ; N#Cc1ccc(-c2ccc3c(c2)C2(c4ccc(-c5nc(-c6ccccc6)nc(-c6ccccc6)n5)cc4-3)C3CC4CC(C3)CC2C4)cc1 ; N#Cc1ccc(-c2cccc3c2C2(c4c(-c5nc(-c6ccccc6)nc(-c6ccccc6)n5)cccc4-3)C3CC4CC(C3)CC2C4)cc1]

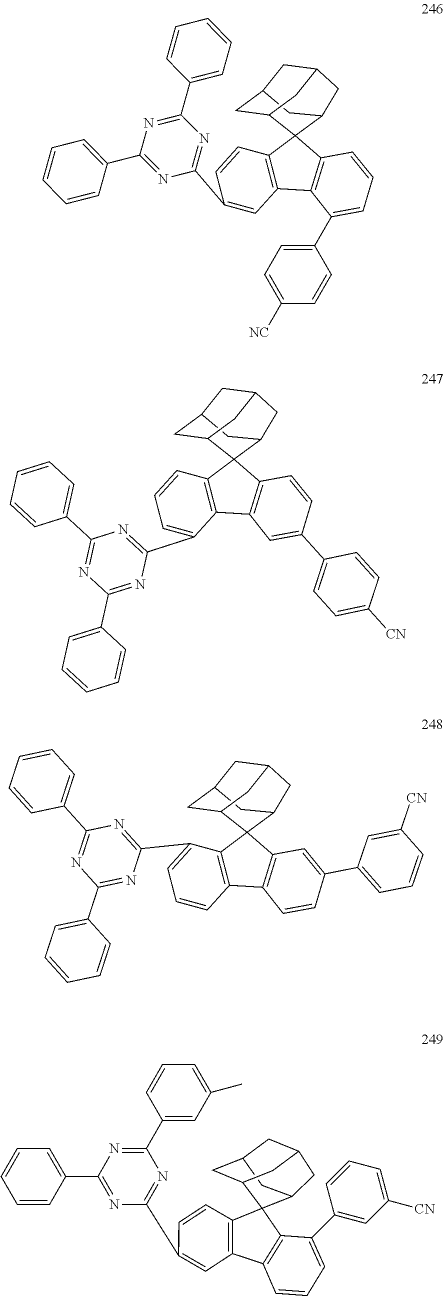 [SMILES: N#Cc1ccc(-c2cccc3c2-c2cc(-c4nc(-c5ccccc5)nc(-c5ccccc5)n4)ccc2C32C3CC4CC(C3)CC2C4)cc1 ; N#Cc1ccc(-c2ccc3c(c2)-c2c(-c4nc(-c5ccccc5)nc(-c5ccccc5)n4)cccc2C32C3CC4CC(C3)CC2C4)cc1 ; N#Cc1cccc(-c2ccc3c(c2)C2(c4c(-c5nc(-c6ccccc6)nc(-c6ccccc6)n5)cccc4-3)C3CC4CC(C3)CC2C4)c1 ; Cc1cccc(-c2nc(-c3ccccc3)nc(-c3ccc4c(c3)-c3cccc(-c5cccc(C#N)c5)c3C43C4CC5CC(C4)CC3C5)n2)c1]

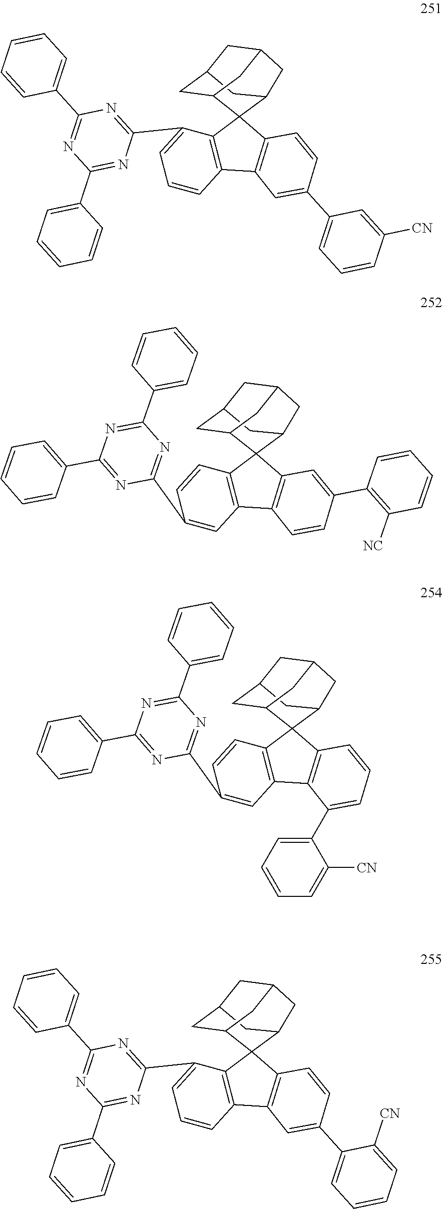 [SMILES: N#Cc1cccc(-c2ccc3c(c2)-c2cccc(-c4nc(-c5ccccc5)nc(-c5ccccc5)n4)c2C32C3CC4CC(C3)CC2C4)c1 ; N#Cc1ccccc1-c1ccc2c(c1)C1(c3ccc(-c4nc(-c5ccccc5)nc(-c5ccccc5)n4)cc3-2)C2CC3CC(C2)CC1C3 ; N#Cc1ccccc1-c1cccc2c1-c1cc(-c3nc(-c4ccccc4)nc(-c4ccccc4)n3)ccc1C21C2CC3CC(C2)CC1C3 ; N#Cc1ccccc1-c1ccc2c(c1)-c1cccc(-c3nc(-c4ccccc4)nc(-c4ccccc4)n3)c1C21C2CC3CC(C2)CC1C3]

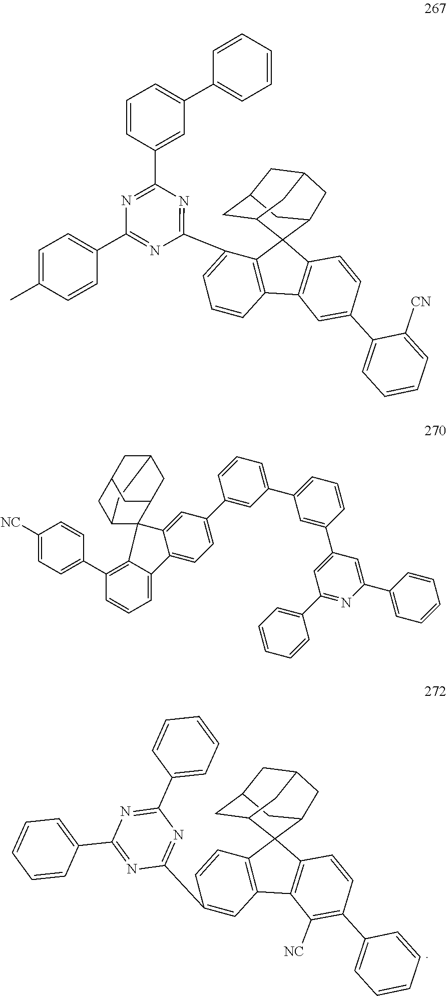 [SMILES: Cc1ccc(-c2nc(-c3cccc(-c4ccccc4)c3)nc(-c3cccc4c3C3(c5ccc(-c6ccccc6C#N)cc5-4)C4CC5CC(C4)CC3C5)n2)cc1 ; N#Cc1ccc(-c2cccc3c2C2(c4cc(-c5cccc(-c6cccc(-c7cc(-c8ccccc8)nc(-c8ccccc8)c7)c6)c5)ccc4-3)C3CC4CC(C3)CC2C4)cc1 ; N#Cc1c(-c2ccccc2)ccc2c1-c1cc(-c3nc(-c4ccccc4)nc(-c4ccccc4)n3)ccc1C21C2CC3CC(C2)CC1C3]

14. An electronic element, comprising an anode and a cathode which are disposed oppositely, and a functional layer disposed between the anode and the cathode; wherein the functional layer contains the nitrogen-containing compound of claim 1.

15. The electronic element of claim 14, wherein the functional layer comprises an electron transporting layer, and the electron transporting layer comprises the nitrogen-containing compound.

16. The electronic element of claim 14, wherein the electronic element is an organic electroluminescent device or a photoelectric conversion device.

17. An electronic device, comprising the electronic element of claim 14.
